(19) United States
(12) Patent Application Publication
(10) Pub. No.: US 2010/0004222 A1
(43) Pub. Date: Jan. 7, 2010

(54) Amide Derivatives as Ion-Channel Ligands and Pharmaceutical Compositions and Methods of Using the Same

(75) Inventors: Michael G. Kelly, Thousand Oaks, CA (US); Carl J. Kaub, San Jose, CA (US); John Kincaid, San Francisco, CA (US); Satyanarayana Janagani, Santa Clara, CA (US); Guoxian Wu, Palo Alto, CA (US); Zhi-Liang Wei, San Mateo, CA (US); Kiran Sahasrabudhe, South San Francisco, CA (US); Matthew Duncton, San Francisco, CA (US); Ravindra B. Upasani, San Jose, CA (US); Yunfeng Fang, San Diego, CA (US); Matthew Cox, San Francisco, CA (US); Jianhua He, Foster City, CA (US)

Correspondence Address: JONES DAY, 222 EAST 41ST ST, NEW YORK, NY, 10017, US

(73) Assignee: Renovis, Inc., South San Fransisco, CA (US)

(21) Appl. No.: 12/224,357

(22) PCT Filed: Feb. 22, 2007
(86) PCT No.: PCT/US2007/004912

§ 371 Date: Jun. 12, 2009

Related U.S. Application Data
(60) Provisional application No. 60/776,106, filed on Feb. 23, 2006.
(60) Provisional application No. 60/775,949, filed on Feb. 23, 2006.
(60) Provisional application No. 60/776,058, filed on Feb. 23, 2006.
(60) Provisional application No. 60/776,057, filed on Feb. 23, 2006.
(60) Provisional application No. 60/775,930, filed on Feb. 23, 2006.
(60) Provisional application No. 60/776,033, filed on Feb. 23, 2006.
(60) Provisional application No. 60/775,945, filed on Feb. 23, 2006.
(60) Provisional application No. 60/776,056, filed on Feb. 23, 2006.
(60) Provisional application No. 60/776,105, filed on Feb. 23, 2006.
(60) Provisional application No. 60/839,903, filed on Aug. 24, 2006.
(60) Provisional application No. 60/839,994, filed on Aug. 24, 2006.
(60) Provisional application No. 60/776,064, filed on Feb. 23, 2006.

(51) Int. Cl. A61K 31/47; C07D 215/38; C07D 513/04; C07D 471/04; C07D 217/02; C07D 265/36; C07D 413/12; C07D 319/16; C07D 277/62; C07D 267/14; C07D 498/04; A61K 31/436; A61K 31/4375; A61K 31/498; A61K 31/404; A61K 31/428; A61K 31/437; A61K 31/553; A61P 25/00; A61P 11/00

(52) U.S. Cl. 514/211.09; 546/159; 546/114; 546/122; 546/143; 544/105; 549/362; 548/178; 540/552; 514/311; 514/302; 514/300; 514/249; 514/415; 514/367

(57) ABSTRACT

Compounds are disclosed that have a formula represented by the following: Formula (I). The compounds may be prepared as pharmaceutical compositions, and may be used for the prevention and treatment of a variety of conditions in mammals including humans, including by way of non-limiting example, pain, inflammation, traumatic injury, and others.

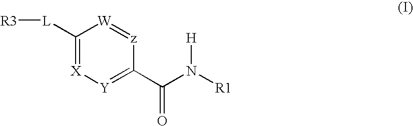

This application claims the benefit of U.S. provisional application Nos. 60/776,106, filed Feb. 23, 2006, 60/775,949, filed Feb. 23, 2006, 60/776,058, filed Feb. 23, 2006, 60/776,057, filed Feb. 23, 2006, 60/775,930, filed Feb. 23, 2006, 60/776,033, filed Feb. 23, 2006,60/775,945, filed Feb. 23, 2006, 60/776,056, filed Feb. 23, 2006, 60/776,105, filed Feb. 23, 2006, 60/776,064, filed Feb. 23, 2006, 60/839,903, filed Aug. 24, 2006, and 60/839,994, filed Aug. 24, 2006, the contents of which are hereby incorporated by reference in their entireties.

### FIELD OF THE INVENTION

This invention relates to novel compounds and to pharmaceutical compositions containing such compounds. This invention also relates to methods for preventing and/or treating pain and inflammation-related conditions in mammals, such as (but not limited to) arthritis, Parkinson's disease, Alzheimer's disease, stroke, uveitis, asthma, myocardial infarction, the treatment and prophylaxis of pain syndromes (acute and chronic or neuropathic), traumatic brain injury, acute spinal cord injury, neurodegenerative disorders, alopecia (hair loss), inflammatory bowel disease, urinary incontinence, chronic obstructive pulmonary disease, irritable bowel disease, osteoarthritis, and autoimmune disorders, using the compounds and pharmaceutical compositions of the invention.

### BACKGROUND OF THE INVENTION

Studies of signaling pathways in the body have revealed the existence of ion channels and sought to explain their role. Ion channels are integral membrane proteins with two distinctive characteristics: they are gated (open and closed) by specific signals such as membrane voltage or the direct binding of chemical ligands and, once open, they conduct ions across the cell membrane at very high rates.

There are many types of ion channels. Based on their selectivity to ions, they can be divided into calcium channel, potassium channel, sodium channel, etc. The calcium channel is more permeable to calcium ions than other types of ions, the potassium channel selects potassium ions over other ions, and so forth. Ion channels may also be classified according to their gating mechanisms. In a voltage-gated ion channel, the opening probability depends on the membrane voltage, whereas in a ligand-gated ion channel, the opening probability is regulated by the binding of small molecules (the ligands). Since ligand-gated ion channels receive signals from the ligand, they may also be considered as “receptors” for ligands.

Examples of ligand-gated ion channels include nAChR (nicotinic acetylcholine receptor) channel, GluR (glutamate receptor) channel, ATP-sensitive potassium channel, G-protein activated channel, cyclic-nucleotide-gated channel, etc.

Transient receptor potential (TRP) channel proteins constitute a large and diverse family of proteins that are expressed in many tissues and cell types. This family of channels mediates responses to nerve growth factors, pheromones, olfaction, tone of blood vessels and metabolic stress et al., and the channels are found in a variety of organisms, tissues and cell types including nonexcitable, smooth muscle and neuronal cells. Furthermore, TRP-related channel proteins are implicated in several diseases, such as several tumors and neurodegenerative disorders and the like. See, for example, Minke, et al., APStracts 9:0006 P (2002).

Nociceptors are specialized primary afferent neurons and the first cells in a series of neurons that lead to the sensation of pain. The receptors in these cells can be activated by different noxious chemical or physical stimuli. The essential functions of nociceptors include the transduction of noxious stimuli into depolarizations that trigger action potentials, conduction of action potentials from primary sensory sites to synapses in the central nervous system, and conversion of action potentials into neurotransmitter release at presynaptic terminals, all of which depend on ion channels.

One TRP channel protein of particular interest is the vanilloid receptor. Also known as VR1, the vanilloid receptor is a non-selective cation channel which is activated or sensitized by a series of different stimuli including capsaicin, heat and acid stimulation and products of lipid bilayer metabolism (anandamide), and lipoxygenase metabolites. See, for example Smith, et al., Nature, 418:186-190 (2002). VR1 does not discriminate among monovalent cations, however, it exhibits a notable preference for divalent cations with a permeability sequence of Ca²⁺>Mg²⁺>Na⁺=K+=Cs⁺. Ca²⁺ is especially important to VR1 function, as extracellular Ca²⁺ mediates desensitization, a process which enables a neuron to adapt to specific stimuli by diminishing its overall response to a particular chemical or physical signal. VR1 is highly expressed in primary sensory neurons in rats, mice and humans, and innervates many visceral organs including the dermis, bones, bladder, gastrointestinal tract and lungs. It is also expressed in other neuronal and non-neuronal tissues including the CNS, nuclei, kidney, stomach and T-cells. The VR1 channel is a member of the superfamily of ion channels with six membrane-spanning domains, with highest homology to the TRP family of ion channels.

VR1 gene knockout mice have been shown to have reduced sensory sensitivity to thermal and acid stimuli. See, for example, Caterina, et al. Science, 14:306-313 (2000). This supports the concept that VR1 contributes not only to generation of pain responses but also to the maintenance of basal activity of sensory nerves. VR1 agonists and antagonists have use as analgesics for the treatment of pain of various genesis or etiology, for example acute, inflammatory and neuropathic pain, dental pain and headache (such as migraine, cluster headache and tension headache). They are also useful as anti-inflammatory agents for the treatment of arthritis, Parkinson's Disease, Alzheimer's Disease, stroke, uveitis, asthma, myocardial infarction, the treatment and prophylaxis of pain syndromes (acute and chronic [neuropathic]), traumatic brain injury, spinal cord injury, neurodegenerative disorders, alopecia (air loss), inflammatory bowel disease, irritable bowel disease and autoimmune disorders, renal disorders, obesity, eating disorders, cancer, schizophrenia, epilepsy, sleeping disorders, cognition, depression, anxiety, blood pressure, lipid disorders, osteoarthritis, and atherosclerosis.

Compounds, such as those of the present invention, which interact with the vanilloid receptor can thus play a role in treating or preventing or ameliorating these conditions.

A wide variety of Vanilloid compounds of different structures are known in the art, for example those disclosed in European Patent Application Numbers EP 0 347 000 and EP 0 401 903, UK Patent Application Number GB 2226313 and International Patent Application, Publication Number WO 92/09285. Particularly notable examples of vanilloid compounds or vanilloid receptor modulators are capsaicin or trans 8-methyl-N-vanillyl-6-nonenamide which is isolated from the pepper plant, capsazepine (Tetrahedron, 53, 1997, 4791) and olvanil or —N-(4-hydroxy-3-methoxybenzyl)oleamide (J. Med. Chem., 36, 1993, 2595).

International Patent Application, Publication Number WO 02/08221 discloses diaryl piperazine and related compounds which bind with high selectivity and high affinity to vanilloid receptors, especially Type I Vanilloid receptors, also known as capsaicin or VR1 receptors. The compounds are said to be useful in the treatment of chronic and acute pain conditions, itch and urinary incontinence.

International Patent Application, Publication Numbers WO 02/16317; WO 02/16318 and WO 02/16319 suggest that compounds having a high affinity for the vanilloid receptor are useful for treating stomach-duodenal ulcers.

International Patent Application, Publication No. WO 2005/046683, published May 26, 2005, commonly owned, discloses a series of compounds that have demonstrated activity as VR-1 antagonists, and that are suggested as being useful for the treatment of conditions associated with VR-1 activity.

U.S. Pat. No. 3,424,760 and U.S. Pat. No. 3,424,761 both describe a series of 3-Ureidopyrrolidines that are said to exhibit analgesic, central nervous system, and pyschopharmacologic activities. These patents specifically disclose the compounds 1-(1-phenyl-3-pyrrolidinyl)-3-phenyl urea and 1-(1-phenyl-3-pyrrolidinyl)-3-(4-methoxyphenyl) urea respectively. International Patent Applications, Publication Numbers WO 01/62737 and WO 00/69849 disclose a series of pyrazole derivatives which are stated to be useful in the treatment of disorders and diseases associated with the NPY receptor subtype YS, such as obesity. WO 01/62737 specifically discloses the compound 5-amino-N-isoquinolin-5-yl-1-[3-(trifluoromethyl)phenyl]-1H-pyrazole-3-carboxamide. WO 00/69849 specifically discloses the compounds 5-methyl-N-quinolin-8-yl-1-[3-(trifluoromethyl)phenyl]-1H-pyrazole-3-carboxamide, 5-methyl-N-quinolin-7-yl-1-[3-trifluoromethyl)phenyl]-1H-pyrazole-3-carboxamide, 5-methyl-N-quinolin-3-yl-1-[3-(trifluoromethyl)phenyl]-1H-pyrazole-3-carboxamide, N-isoquinolin-5-yl-5-methyl-1-[3-(trifluoromethyl)phenyl]-1H-pyrazole-3-carboxamide, 5-methyl-N-quinolin-5-yl-1-[3-(trifluoromethyl)phenyl]-1H-pyrazole-3-carboxamide, 1-(3-chlorophenyl)-N-isoquinolin-5-yl-5-methyl-1H-pyrazole-3-carboxamide, N-isoquinolin-5-yl-1-(3-metoxyphenyl)-5-methyl-1H-pyrazole-3-carboxamide, 1-(3-fluorophenyl)-N-isoquinolin-5-yl-5-methyl-1H-pyrazole-3-carboxamide, 1-(2-chloro-5-trifluoromethylphenyl)-N-isoquinolin-5-yl-5-methyl-1N-pyrazole-3-carboxamide, 5-methyl-N-(3-methylisoquinolin-5-yl)-1-[3-(trifluoromethyl)phenyl]-1N-pyrazole-3-carboxamide, 5-methyl-N-(1,2,3,4-tetrahydroisoquinolin-5-yl)-1-[3-(trifluoromethyl)phenyl]-1H-pyrazole-3-carboxamide.

German Patent Application Number 2502588 describes a series of piperazine derivatives. This application specifically discloses the compound N-[3-[2-(diethylamino) ethyl]-1,2-dihydro-4-methyl-2-oxo-7-quinolinyl]-4-phenyl-1-piperazinecarboxamide.

We have now discovered that certain compounds have surprising potency and selectivity as VR-1 antagonists. The compounds of the present invention are considered to be particularly beneficial as VR-1 antagonists as certain compounds exhibit improved aqueous solubility and metabolic stability.

### SUMMARY OF THE INVENTION

It has now been found that compounds set forth herein, are capable of modifying mammalian ion channels such as the VR1 cation channel. Accordingly, compounds provided herein are potent VR1 antagonists with analgesic activity by systemic administration. The compounds of the present invention may show low toxicity, good absorption, good half-life, good solubility, low protein binding affinity, low drug-drug interaction, low inhibitory activity at HERG channel, low QT prolongation and good metabolic stability. This finding leads to novel compounds having therapeutic value. It also leads to pharmaceutical compositions having the compounds of the present invention as active ingredients and to their use to treat, prevent or ameliorate a range of conditions in mammals such as but not limited to pain of various genesis or etiology, for example acute, chronic, inflammatory and neuropathic pain, dental pain and headache (such as migraine, cluster headache and tension headache).

Accordingly, in a first aspect of the invention, compounds are provided having a formula I:

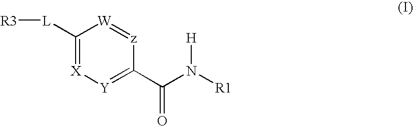

wherein:

each of W, Z, and X is independently N or CR⁴; and Y is CR^{4″};

L is —(CR⁵═CR⁶)— or —(C≡C)—;

R¹ is substituted or unsubstituted bicycloaryl or bicycloheteroaryl;

R³ is CR^{6′} R⁷R⁸;

each R⁴ is independently hydrogen, C₁-C₆ alkyl, hydroxyl C₁-C₆ alkyl, C₁-C₆ alkylamino, C₁-C₆ alkoxy, amino C₁-C₆ alkoxy, substituted amino C₁-C₆ alkoxy, di C₁-C₆ alkylamino C₁-C₆ alkoxy, cycloalkyl C₁-C₆ alkoxy, C₁-C₆ alkoxycarbonyl, C₁-C₆ alkylarylamino, aryl C₁-C₆ alkyloxy, amino, aryl, aryl C₁-C₆ alkyl, sulfoxide, sulfone, sulfanyl, aminosulfonyl, arylsulfonyl, sulfuric acid, sulfuric acid ester, azido, carboxy, carbamoyl, cyano, cycloheteroalkyl, di C₁-C₆ alkylamino, halo, heteroaryloxy, heteroaryl, heteroalkyl, hydroxyl, nitro or thio;

R^{4″} is alkyl, trihaloalkyl, alkoxy, sulfone or halo;

each of R⁵ and R⁶ is independently H, or C₁-C₆ alkyl; and

R^{6′} is hydrogen, halo or C₁-C₆ alkyl; each of R⁷ and R⁸ is independently halo or C₁-C₆ alkyl; or R⁷ and R⁸ together form a C₃-C₈ cycloalkyl ring;

or a pharmaceutically acceptable salt, solvate or prodrug thereof;
and stereoisomers and tautomers thereof. Compounds according to formula I are capable of modifying ion channels in vivo.

In a further embodiment of the invention, provided are compounds of formula IA wherein R³-L represents is CR³R⁶═CR⁵. Such compounds are hereinafter referred to as compounds of formula IA′:

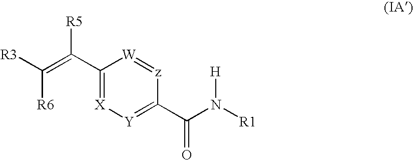

wherein R³ is as defined for compounds of formula I and R⁵ and R⁶ are independently selected from hydrogen and C₁-C₆ alkyl.

In compounds of formula IA′, R⁵ and R⁶ may, for example, independently represent hydrogen, or Me. Preferably R⁵ and R⁶ represent hydrogen.

In another embodiment, provided are compounds of formula IA wherein R³-L is R³C≡C—. Hereinafter, such compounds are referred to as compounds of formula IA″:

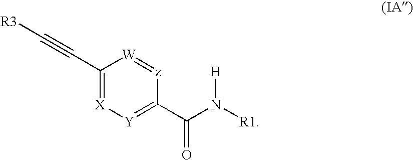

wherein R³ is as defined for compounds of formula I

Generally in compounds of formula I, L is preferably —(C═C)— or —C≡C—. Thus in certain embodiments, L is —(C═C)—. In certain embodiments, L is —C≡C—.

In compounds of formula I, IA′ and IA″, R³ may for example represent CR^{6′}R⁷R⁸ wherein R^{6′} represents hydrogen, halo, C₁-C₆ alkyl or hydroxyl C₁-C₆ alkyl; each of R⁷ and R⁸ is independently halo, C₁-C₆ alkyl or hydroxyl C₁-C₆ alkyl; or R⁷ and R⁸ together form a substituted or unsubstituted C₃-C₈ cycloalkyl ring. For example R⁷ may represent lower alkyl (e.g. methyl). For example R⁸ may represent lower alkyl (e.g. methyl). In particular examples, R^{6′} may represent hydrogen and R⁷ and R⁸ may represent methyl. Alternatively each of R^{6′}, R⁷ and R⁸ may represent methyl. Alternatively each of R^{6′}, R⁷ and R⁸ may represent fluoro. Alternatively R^{6′} may represent hydrogen and R⁷ and R⁸ together form a cyclopropyl ring.

In a first alternative embodiment of the compounds of formula I, R³ is CF₃, i-propyl, t-Bu or cycloalkyl. In another embodiment R³ is CF₃, t-Bu, cyclopropyl, cyclobutyl, cyclopentyl or cyclohexyl. In yet another embodiment R³ is CF₃, t-Bu, or cyclopropyl.

In yet another particular embodiment, with respect to the compounds of formula I, R³ may be substituted or unsubstituted cyclopropyl.

In yet further particular embodiment, with respect to the compounds of formula I, R³ may be CF₃.

In yet further particular embodiment, with respect to the compounds of formula I, R³ may be t-Bu.

With respect to the compounds of formula I, R¹ may be substituted or unsubstituted naphthyl, or alternatively, substituted or unsubstituted tetrahydronaphthyl. Further, R¹ may also be substituted or unsubstituted bicycloheteroaryl, and in a particular embodiment, the bicycloheteroaryl may be selected from the group consisting of tetrahydroquinoline, tetrahydroisoquinoline, benzodioxane, benzopyran, indole and benzimidazole. More particularly, the bicycloheteroaryl may be quinoline, isoquinoline, benzodioxane, and benzoxazine. In a particular embodiment, the substitution on the bicycloheteroaryl is selected from the group consisting of hydrogen, alkyl, trifluoromethyl, halo, methoxy, trifluoromethoxy, amino and carboxy. In a yet further particular embodiment, the substitution on bicycloheteroaryl is selected from the group consisting of tert-butyl, cyano, trifluoroalkyl, halo, nitro, methoxy, amino and carboxy.

In yet another particular embodiment, with respect to the compounds of formula I, R¹ may be substituted or unsubstituted isoquinolin-5-yl, quinolin-3-yl, benzodioxan-6-yl or benzoxazin-6-yl.

In yet another particular embodiment, with respect to the compounds of formula I, R¹ may be substituted or unsubstituted

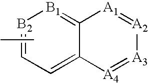

wherein each of A¹, A², A³, A⁴, B¹ and B² is independently CR^{4′} and N; and each of R^{4′} is independently H, C₁-C₆ alkyl, halo, or hydroxy C₁-C₆ alkyl.

In yet another particular embodiment, with respect to the compounds of formula I, R¹ may be substituted or unsubstituted

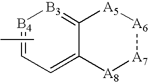

wherein each of A⁵ and A⁸ is independently CR^{4′} R^{4′}, NR^{4′}, O, S, SO or SO₂;
each of A⁶ and A⁷ is independently CR^{4′}, NR⁴, CR⁴R^{4′} or CO; each of B³ and B⁴ is independently CR^{4′} and N; when R^{4′} is attached to C, each of R^{4′} is independently H, C₁-C₆ alkyl, halo, or hydroxy C₁-C₆ alkyl, and when R^{4′} is attached to N, each of R^{4′} is independently H or C₁-C₆ alkyl; and the dotted bond represents a single or a double bond.

In yet another particular embodiment, with respect to the compounds of formula I, R¹ may be substituted or unsubstituted

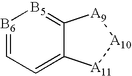

wherein each of A⁹, A¹⁰ and A¹¹ is independently CR^{4′}, CR^{4′}R^{4′}, CO, CS, N, NR^{4′}, O, S, SO or SO₂; each of B⁵ and B⁶ is independently CR⁴ and N;
when R^{4′} is attached to C, each of R^{4′} is independently H, C₁-C₆ alkyl, halo, or hydroxy C₁-C₆ alkyl, and when R^{4′} is attached to N, each of R^{4′} is independently H, or C₁-C₆ alkyl; and each of the dotted bonds independently represents a single or a double bond.

In yet another particular embodiment, with respect to the compounds of formula I, R¹ may be substituted or unsubstituted:

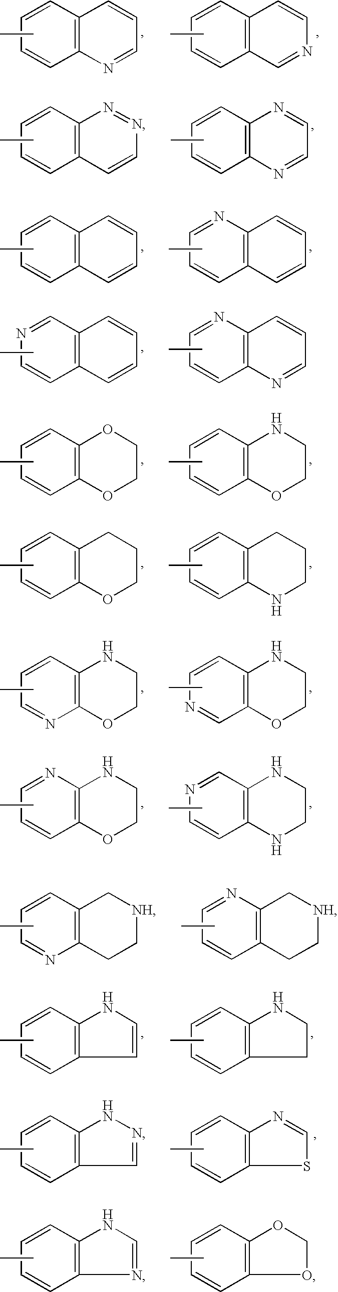

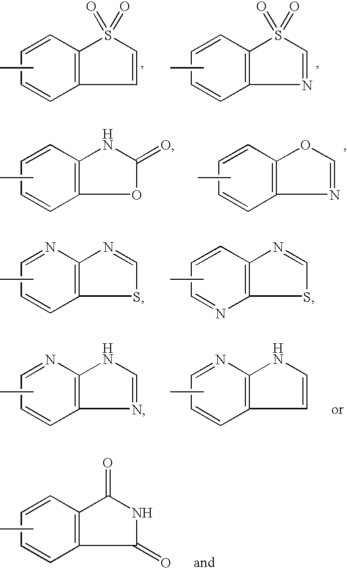

wherein, the ring may be further substituted with R^{4′}, and R^{4′} is as described above; and when feasible, the ring N can further be substituted with H or C₁-C₆ alkyl.

In yet another particular embodiment, with respect to the compounds of formula I, R¹ may be substituted or unsubstituted:

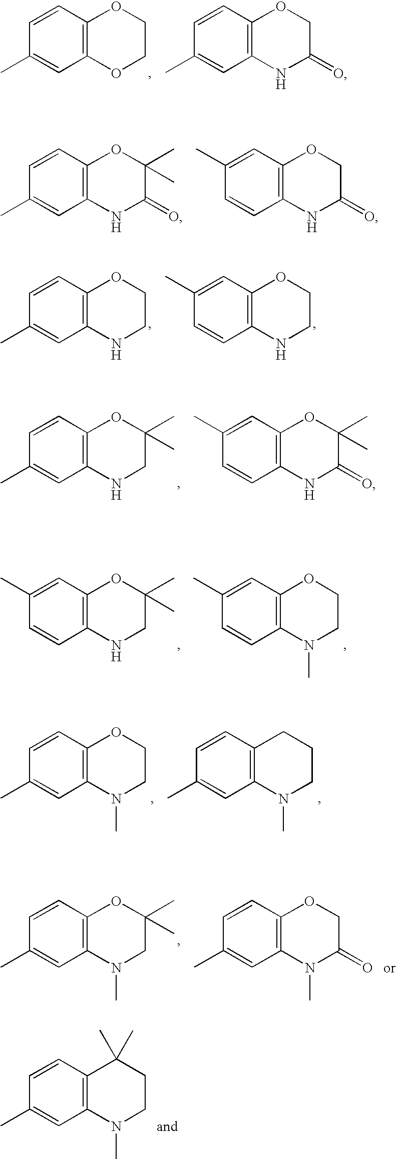

wherein, the ring may be further substituted with R^{4′}, and R^{4′} is as described above; and when feasible, the ring N can further be substituted with H or C₁-C₆ alkyl.

In yet another particular embodiment, with respect to the compounds of formula I, R¹ may be substituted or unsubstituted:

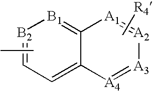

wherein each of A¹, A², A³, A⁴, B¹ and B² is independently CH and N;

and R^{4′} is C₁-C₆ alkyl or hydroxy C₁-C₆ alkyl.

In yet another particular embodiment, with respect to the compounds of formula I, R¹ may be substituted or unsubstituted:

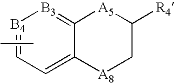

wherein each of A⁵ and A⁸ is independently CH₂, CHMe, NH, NMe, O, S, SO or SO₂; and R^{4′} is C₁-C₆ alkyl or hydroxy C₁-C₆ alkyl.

In yet another particular embodiment, with respect to the compounds of formula I, R¹ may be substituted or unsubstituted:

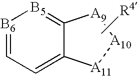

wherein each of A⁹, A¹⁰ and A¹¹ is independently CH, CH₂, N, NH, O, or S; each of B⁵ and B⁶ is independently CH and N; each of R^{4′} is independently H, C₁-C₆ alkyl or hydroxy C₁-C₆ alkyl; and each of the dotted bonds independently represents a single or a double bond.

In yet another particular embodiment, with respect to the compounds of formula I, R¹ may be

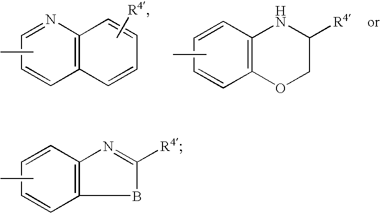

and wherein R^{4′} is as described in the preceding paragraphs.

In one particular embodiment, with respect to the compounds of formula I, R¹ is as described in the preceding paragraphs and R^{4′} is alkyl or substituted alkyl. In yet another embodiment R^{4′} is substituted alkyl. In yet another particular embodiment R^{4′} is hydroxy alkyl. In yet another particular embodiment R⁴ is hydroxymethyl, hydroxylethyl or hydroxypropyl. In yet another particular embodiment R^{4′} is hydroxymethyl.

In one particular embodiment, with respect to the compounds of formula I, R¹ is

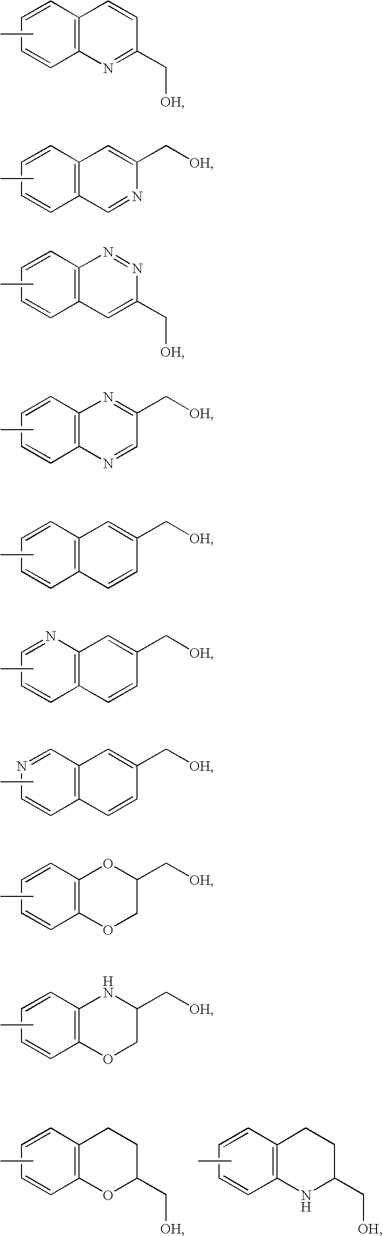

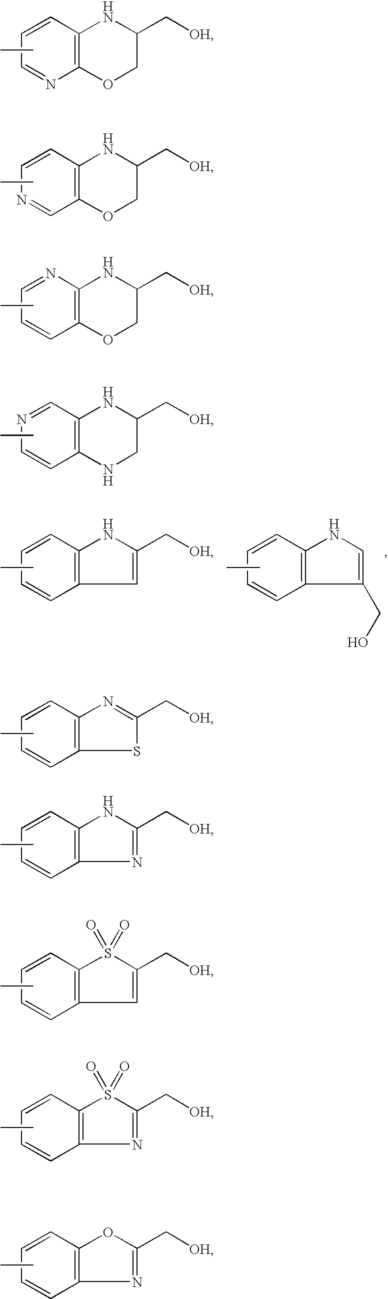

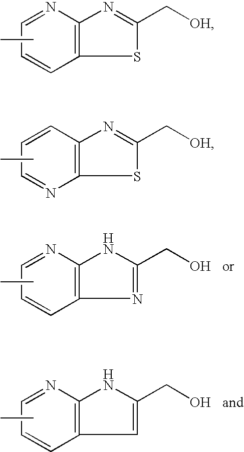

wherein, when feasible, the ring N can further be substituted with H or C₁-C₆ alkyl.

In one embodiment, with respect to the compounds of formula I, R¹ is

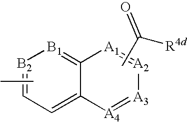

wherein each of A¹, A², A³, A⁴, B¹ and B² is independently CR^{4′} or N;
each of R^{4′} is independently H, substituted or unsubstituted lower alkyl, or halo;
R^{4d} is alkyl, hydroxyl, alkoxy, or a group —NR^{4e}R^{4f}; R^{4e} and R^{4f} are independently H, alkyl, substituted alkyl; or R^{4e} and R^{4f} together form a substituted or unsubstituted cycloheteroalkyl ring of 4-8 atoms. In a particular embodiment the ring

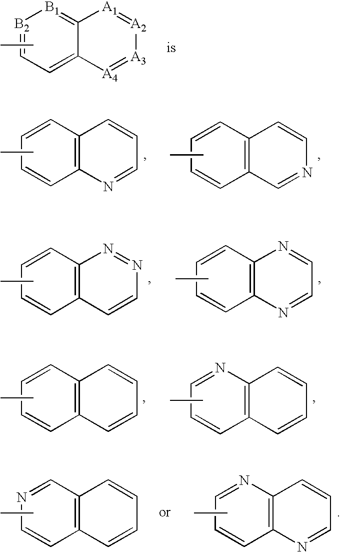

In yet another particular embodiment, R^{4d} may for example represent —NMe₂, methoxy, hydroxyl, methyl, or ethyl. In yet another particular embodiment, R^{4d} may for example represent —NR^{4e}R^{4e} and wherein R^{4e} is H or Me, —CH₂—CH₂—OH; and R^{4f} is H, Me, —CH₂—CH₂—OH, —CH₂—CH₂—OMe, —CH₂—CH₂—NMe₂, —CH₂—C(OH)H—CH₂OH, —CH₂—CH₂—Cy¹, or CH₂—C(OH)H—CH₂—Cy¹; and Cy¹ is

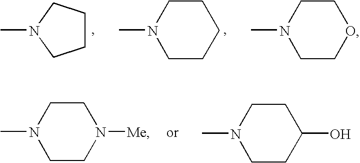

In yet another particular embodiment, R^{4d} may for example represent Cy1 and Cy¹ is

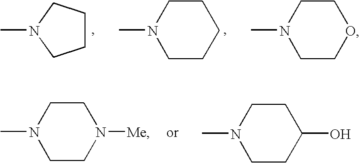

In one embodiment, with respect to the compounds of formula I, R¹ is

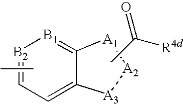

wherein each of A¹, A², and A³, is independently CR^{4′}, S, O, N, NR4′; B¹ and B² is independently CR^{4′} or N;
each of R^{4′} is independently H, substituted or unsubstituted lower alkyl, or halo;
R^{4d} is alkyl, hydroxyl, alkoxy, or a group —NR^{4e}R^{4f}; R^{4e} and R^{4f} are independently H, alkyl, substituted alkyl; or R^{4e} and R^{4f} together form a substituted or unsubstituted cycloheteroalkyl ring of 4-8 atoms. In a particular embodiment the ring

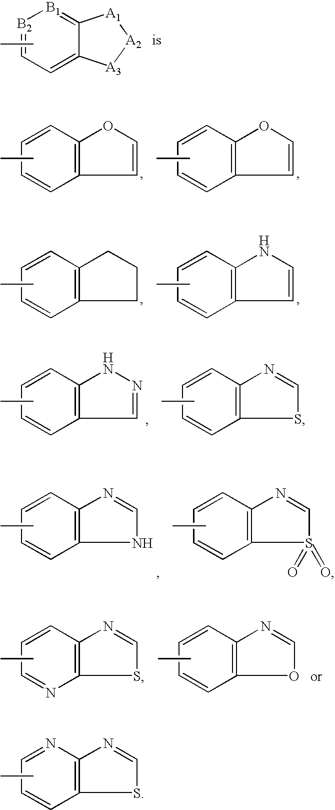

In yet another particular embodiment, R^{4d} may for example represent —NMe₂, methoxy, hydroxyl, methyl, or ethyl. In yet another particular embodiment, R^{4d} may for example represent —NR^{4e}R^{4e} and wherein R^{4e} is H or Me, —CH₂—CH₂—OH; and R^{4f} is H, Me, —CH₂—CH₂—OH, —CH₂—CH₂—OMe, —CH₂—CH₂—NMe₂, —CH₂—C(OH)H—CH₂OH, —CH₂—CH₂—Cy¹, or —CH₂—C(OH)H—CH₂—Cy¹; and Cy¹ is

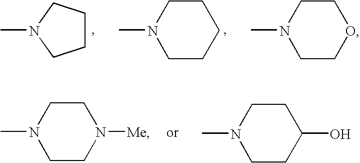

In yet another particular embodiment, R^{4d} may for example represent Cy1 and Cy¹ is

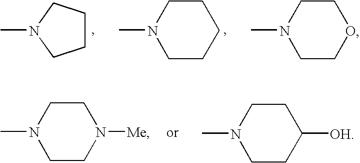

In one embodiment, with respect to the compounds of formula I, R¹ is

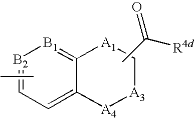

wherein each of A¹, A³ and A⁴ is independently CR^{4′}R^{4′}, O, NR^{4′}, S, SO or SO₂; B¹ and B² is independently CR^{4′} or N;
each of R^{4′} is independently H, substituted or unsubstituted lower alkyl, or halo;
R^{4d} is alkyl, hydroxyl, alkoxy, or a group —NR^{4e}R^{4f}; R^{4e} and R^{4f} are independently H, alkyl, substituted alkyl; or R^{4e} and R^{4f} together form a substituted or unsubstituted cycloheteroalkyl ring of 4-8 atoms. In a particular embodiment the ring

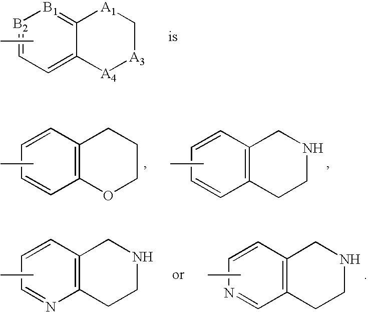

In yet another particular embodiment, R^{4d} may for example represent —NMe₂, methoxy, hydroxyl, methyl, or ethyl. In yet another particular embodiment, R^{4d} may for example represent —NR^{4e}R^{4e} and wherein R^{4e} is H or Me, —CH₂—CH₂—OH; and R^{4f} is H, Me, —CH₂—CH₂—OH, —CH₂—CH₂—OMe, —CH₂—CH₂—NMe₂, —CH₂—C(OH)H—CH₂OH, —CH₂—CH₂—Cy¹, or —CH₂—C(OH)H—CH₂—Cy¹; and Cy¹ is

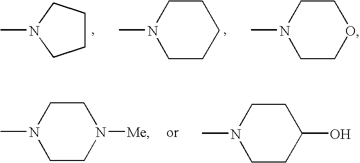

In yet another particular embodiment, R^{4d} may for example represent Cy1 and Cy¹ is

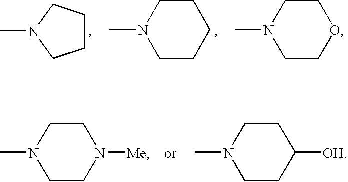

In one embodiment, with respect to the compounds of formula I, R¹ is

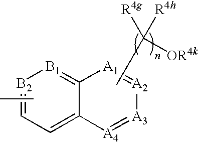

wherein each of A¹, A², A³, A⁴, B¹ and B² is independently CR^{4′} or N;
each of R^{4′} is independently H, substituted or unsubstituted lower alkyl, or halo;
R^{4k} is hydrogen, alkyl, substituted alkyl, acyl, substituted acyl, aminocarbonyl, or substituted aminocarbonyl; R^{4g} and R^{4h} are independently H, alkyl, substituted alkyl; or R^{4g} and R^{4h} together form a substituted or unsubstituted cycloalkyl or cycloheteroalkyl ring of 3-6 atoms; and n is 0-4. In a particular embodiment the ring

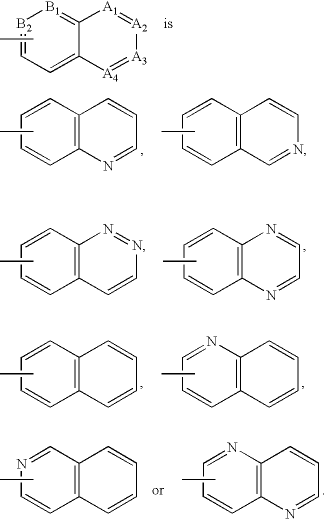

In one embodiment, n is 0-4. In another embodiment, n is 0-3. In yet another embodiment, n is 0-2. In a particular embodiment n is 0 or 2.

In one embodiment, each of R^{4g} and R^{4h} is H. In another embodiment one of R^{4g} and R^{4h} is Me. In yet another embodiment, each of R^{4g} and R^{4h} is Me.

In one embodiment, R^{4k} may for example represent H, Me or Et. In another embodiment, R^{4k} is i—Pr, —CH₂—CH₂—OH, —CH₂—CH₂—OMe, —CH₂—CH₂—NMe₂, COMe, COCH₂NMe₂, COCH₂OH, COC(Me₂)OH, COCH₂OMe, CONHMe, CONMe₂, CONHCH₂CH₂OH, CON(CH₂CH₂OH)₂, COCy¹, or COCH₂Cy¹; and Cy¹ is

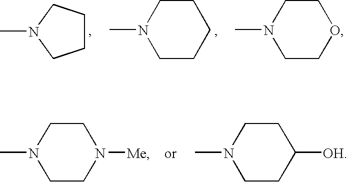

In one embodiment, with respect to the compounds of formula I, R¹ is

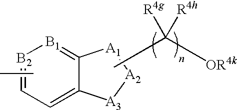

wherein each of A¹, A², and A³, is independently CR^{4′}, CR^{4′}R^{4′}, S, SO, SO₂, O, N, NR^{4′}; B¹ and B² is independently CR⁴ or N;
each of R^{4′} is independently H, substituted or unsubstituted lower alkyl, or halo;
R^{4k} is hydrogen, alkyl, substituted alkyl, acyl, substituted acyl, aminocarbonyl, or substituted aminocarbonyl; R^{4g} and R^{4h} are independently H, alkyl, substituted alkyl; or R^{4g} and R^{4h} together form a substituted or unsubstituted cycloalkyl or cycloheteroalkyl ring of 3-6 atoms; and n is 0-4. In a particular embodiment the ring

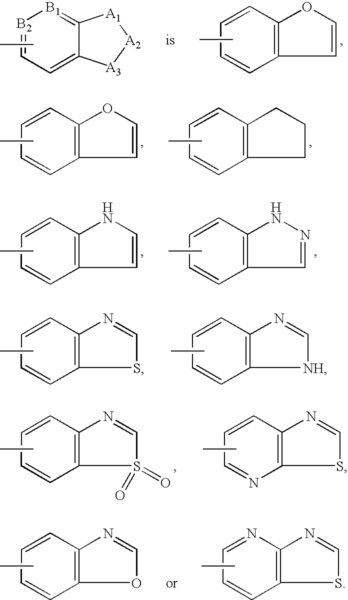

In one embodiment, n is 0-4. In another embodiment, n is 0-3. In yet another embodiment, n is 0-2. In a particular embodiment n is 0 or 2.

In one embodiment, each of R^{4g} and R^{4h} is H. In another embodiment one of R^{4g} and R^{4h} is Me. In yet another embodiment, each of R^{4g} and R^{4h} is Me.

In one embodiment, R^{4k} may for example represent H, Me or Et. In another embodiment, R^{4k} is i—Pr, —CH₂—CH₂—OH, —CH₂—CH₂—OMe, —CH₂—CH₂—NMe₂, COMe, COCH₂NMe₂, COCH₂OH, COC(Me₂)OH, COCH₂OMe, CONHMe, CONMe₂, CONHCH₂CH₂OH, CON(CH₂CH₂OH)₂, COCy¹, or COCH₂Cy¹; and Cy¹ is

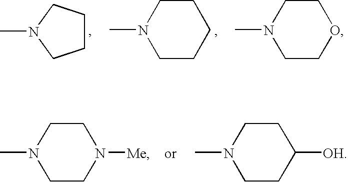

In one embodiment, with respect to the compounds of formula I, R¹ is

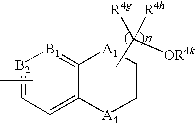

wherein each of A¹ and A⁴ is independently CR^{4′}R^{4′}, O, NR^{4′} or S; B¹ and B² is independently CR^{4′} or N;
each of R^{4′} is independently H, substituted or unsubstituted lower alkyl, or halo;
R^{4k} is hydrogen, alkyl, substituted alkyl, acyl, substituted acyl, aminocarbonyl, or substituted aminocarbonyl; R^{4g} and R^{4h} are independently H, alkyl, substituted alkyl; or R^{4g} and R^{4h} together form a substituted or unsubstituted cycloalkyl or cycloheteroalkyl ring of 3-6 atoms; and n is 0-4. In a particular embodiment the ring

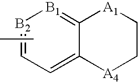

is selected from

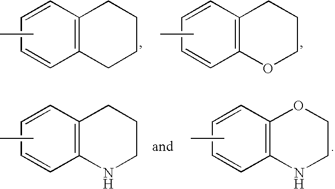

In one embodiment, n is 0-4. In another embodiment, n is 0-3. In yet another embodiment, n is 0-2. In a particular embodiment n is 0 or 2.

In one embodiment, each of R^{4g} and R^{4h} is H. In another embodiment one of R^{4g} and R^{4h} is Me. In yet another embodiment, each of R^{4g} and R^{h} is Me.

In one embodiment, R^{4k} may for example represent H, Me or Et. In another embodiment, R^{4k} is i—Pr, —CH₂—CH₂—OH, —CH₂—CH₂—OMe, —CH₂—CH₂—NMe₂, COMe, COCH₂NMe₂, COCH₂OH, COC(Me₂)OH, COCH₂OMe, CONHMe, CONMe₂, CONHCH₂CH₂OH, CON(CH₂CH₂OH)₂, COCy¹, or COCH₂Cy¹; and Cy¹ is

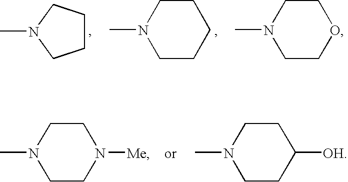

In one embodiment, with respect to the compounds of formula I, R¹ is

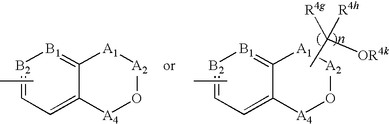

wherein A¹ is CR^{4′}R^{4′}; each of A² and A⁴ is independently CR^{4′}R^{4′} or CO;
B¹ and B² is independently CR^{4′} or N;
each of R^{4′} is independently H, substituted or unsubstituted lower alkyl, or halo;
R^{4k} is hydrogen, alkyl, substituted alkyl, acyl, substituted acyl, aminocarbonyl, or substituted aminocarbonyl; R^{4g} and R^{4h} are independently H, alkyl, substituted alkyl; or R^{4g} and R^{4h} together form a substituted or unsubstituted cycloalkyl or cycloheteroalkyl ring of 3-6 atoms; and n is 0-4. In a particular embodiment the ring

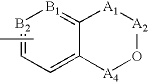

is selected from

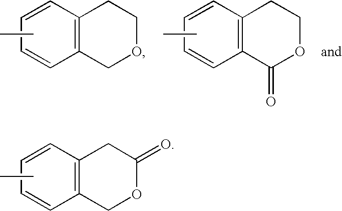

In one embodiment, n is 0-4. In another embodiment, n is 0-3. In yet another embodiment, n is 0-2. In a particular embodiment n is 0 or 2.

In one embodiment, each of R^{4g} and R^{4h} is H. In another embodiment one of R^{4g} and R^{4h} is Me. In yet another embodiment, each of R^{4g} and R^{4h} is Me.

In one embodiment, R^{4k} may for example represent H, Me or Et. In another embodiment, R^{4k} is i—Pr, —CH₂—CH₂—OH, —CH₂—CH₂—OMe, —CH₂—CH₂—NMe₂, COMe, COCH₂NMe₂, COCH₂OH, COC(Me₂)OH, COCH₂OMe, CONHMe, CONMe₂, CONHCH₂CH₂OH, CON(CH₂CH₂OH)₂, COCy¹, or COCH₂Cy¹; and Cy¹, is

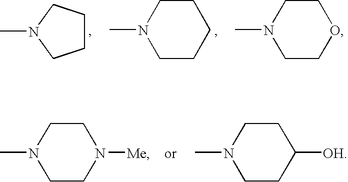

In one embodiment, with respect to the compounds of formula I, R¹ is

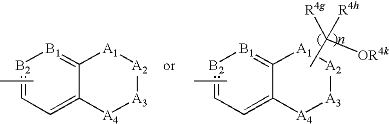

wherein A¹ is CR^{4′}R^{4′}; each of A² and A⁴ is independently CR^{4′}R⁴ or CO; A³ is S, SO or SO₂; and B¹ and B² is independently CR^{4′} or N;
each of R^{4′} is independently H, substituted or unsubstituted lower alkyl, or halo;
R^{4k} is hydrogen, alkyl, substituted alkyl, acyl, substituted acyl, aminocarbonyl, or substituted aminocarbonyl; R^{4g} and R^{4h} are independently H, alkyl, substituted alkyl; or R^{4g} and R^{4h} together form a substituted or unsubstituted cycloalkyl or cycloheteroalkyl ring of 3-6 atoms; and n is 0-4. In a particular embodiment the ring

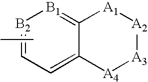

is selected from

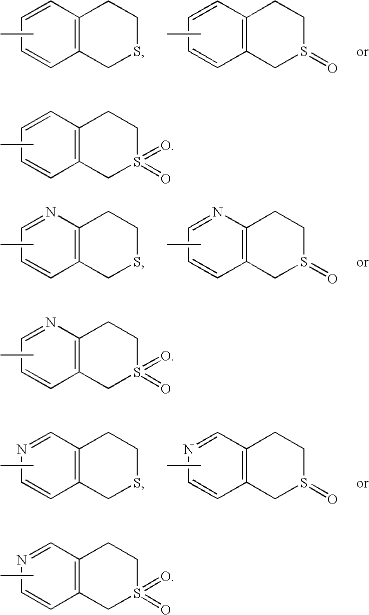

In one embodiment, n is 0-4. In another embodiment, n is 0-3. In yet another embodiment, n is 0-2. In a particular embodiment n is 0 or 2.

In one embodiment, each of R^{4g} and R^{4h} is H. In another embodiment one of R^{4g} and R^{4h} is Me. In yet another embodiment, each of R^{4g} and R^{4h} is Me.

In one embodiment, R^{4k} may for example represent H, Me or Et. In another embodiment, R^{4k} is i—Pr, —CH₂—CH₂—OH, —CH₂—CH₂—OMe, —CH₂—CH₂—NMe₂, COMe, COCH₂NMe₂, COCH₂OH, COC(Me₂)OH, COCH₂OMe, CONHMe, CONMe₂, CONHCH₂CH₂OH, CON(CH₂CH₂OH)₂, COCy¹, or COCH₂Cy1; and Cy¹ is

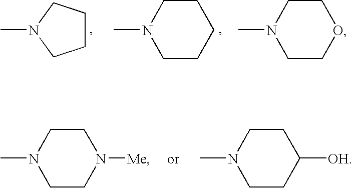

In one embodiment, with respect to the compounds of formula I, R¹ is

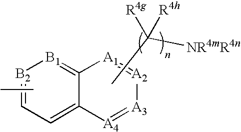

wherein each of A¹, A², A³, A⁴, B¹ and B² is independently CR^{4′} or N;
each R^{4′} is independently H, substituted or unsubstituted lower alkyl, or halo;
R^{4m} is hydrogen, alkyl, substituted alkyl, acyl, substituted acyl, aminocarbonyl, or substituted aminocarbonyl; R⁴ⁿ is independently H, or substituted or unsubstituted lower alkyl;
R^{4g} and R^{4h} are independently H, alkyl, substituted alkyl; or R^{1g} and R^{4h} together form a substituted or unsubstituted cycloalkyl or cycloheteroalkyl ring of 3-6 atoms; and n is 0 or 1. In a particular embodiment the ring

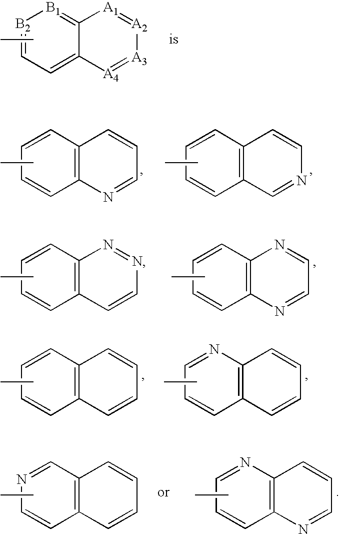

In one embodiment, n is 0-4. In another embodiment, n is 0-3. In yet another embodiment, n is 0-2. In a particular embodiment n is 0 or 2.

In one embodiment, each of R^{4g} and R^{4h} is H. In another embodiment one of leg and R^{4h} is Me. In yet another embodiment, each of R^{4g} and R^{4h} is Me.

In one embodiment, R^{4m} is H, Me, or —CH₂—CH₂—OH. In another embodiment R⁴ⁿ is H, Me, —CH₂—CH₂—OH, —CH₂—CH₂—OMe, or —CH₂—CH₂—NMe₂. In yet another embodiment the group —NR^{4m}R⁴ⁿ is

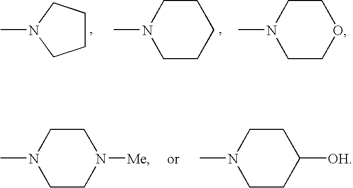

In one particular embodiment with respect to the formula (I), the compound is

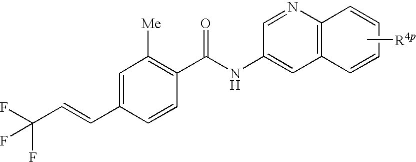

or a pharmaceutically acceptable salt, solvate or prodrug thereof, or stereoisomers, isotopic variants and tautomers thereof and wherein R^{4p} is independently H, C₁-C₆ alkyl, halo, hydroxyl, carbalkoxy [C(O)(C₁-C₆ alkoxy)], acyl [C(O)(C₁-C₆ alkyl)] or hydroxy C₁-C₆ alkyl. In one embodiment R^{4p} is H or Me. In a particular embodiment R^{4p} is H.

In one particular embodiment with respect to the formula (I), the compound is

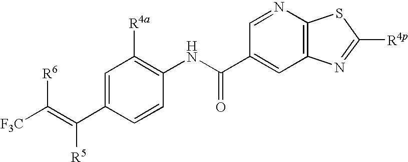

or a pharmaceutically acceptable salt, solvate or prodrug thereof, and stereoisomers, isotopic variants and tautomers thereof, wherein:
R^{4a} is C₁-C₆ alkyl, halo C₁-C₆ alkyl, C₁-C₆ alkoxy, sulfone [S(O)₂(C₁-C₆ alkyl)] or halo;
R^{4p} is independently H, C₁-C₆ alkyl, halo, hydroxyl, carbalkoxy [C(O)(C₁-C₆ alkoxy)], acyl
[C(O)(C₁-C₆ alkyl)] or hydroxy C₁-C₆ alkyl; and each of R⁵ and R⁶ is independently H, or C₁-C₆ alkyl. In one embodiment each of R⁵ and R⁶ is H. In another embodiment one of R⁵ and R⁵ is Me. In one embodiment R^{4a} is Me. In another embodiment R^{4p} is H, Me or CH₂OH. In a particular embodiment R^{4p} is H. In yet another particular embodiment, R^{4a} is Me, R^{4p} is CH₂OH and each of R⁵ and R⁶ is H.

In one particular embodiment with respect to the formula (I), the compound is

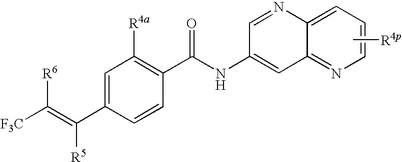

or a pharmaceutically acceptable salt, solvate or prodrug thereof, and stereoisomers, isotopic variants and tautomers thereof, wherein:
R^{4a} is C₁-C₆ alkyl, halo C₁-C₆ alkyl, C₁-C₆ alkoxy, sulfone [S(O)₂(C₁-C₆ alkyl)] or halo;
R^{4p} is independently H, C₁-C₆ alkyl, halo, hydroxyl, carbalkoxy [C(O)(C₁-C₆ alkoxy)], acyl [C(O)(C₁-C₆ alkyl)] or hydroxy C₁-C₆ alkyl; and each of R⁵ and R⁶ is independently H, or C₁-C₆ alkyl. In one embodiment each of R⁵ and R⁶ is H. In another embodiment one of R⁵ and R⁶ is Me. In one embodiment R^{4a} is Me. In another embodiment R^{4p} is H or Me. In a particular embodiment R^{4p} is H. In yet another particular embodiment, R^{4a} is Me, R^{4p} is H and each of R⁵ and R⁶ is H.

In compounds of formula I, IA′ and IA″, W, Z, and X may for example each represent CR⁴, especially CH. Alternatively X may represent N and W, and Z may each represent CR⁴. In another exemplary set of compounds, each of X and Z represents CR⁴, especially CH. In another example set of compounds W is N. In yet another exemplary set of compounds, Y is N.

In compounds of formula I, IA′ and IA″, each of W, X, and Z is CR⁴ and R⁴ is selected from H, halo, alkoxy, sulfo, alkyl, haloalkyl or hydroxyalkyl.

In compounds of formula I, IA′ and IA″, each of W, X, and Z is CR⁴ and R⁴ is selected from H, halo, or alkyl.

In compounds of formula I, IA′ and IA″, each of W, X, and Z is CR⁴ and R⁴ is selected from H, F, Cl or Me.

In compounds of formula I, IA′ and IA″, Y is CR^{4″} and wherein R^{4″} is independently selected from C₁-C₆ alkyl, trihalo C₁-C₆ alkyl and halo.

In compounds of formula I, IA′ and IA″, Y is CR^{4″} and wherein R^{4″} is independently selected from CH₃, CF₃, Cl, or F.

In compounds of formula I, IA′ and IA″, each of W and X is CH; and each of Y and Z is independently is C—CH₃, C—Cl, or C—F.

In compounds of formula I, IA′ and IA″, each of W and X is CH; and each of Y and Z is independently is C—CH₃ or C—F.

In yet further particular embodiments, the compounds of the invention are set forth and may be selected from a comprehensive listing of such compounds, set forth later on herein in Table 1. The Table contains in excess of 100 compounds that have been or can be synthesized and have as a group, demonstrated activity in their capacity of modifying ion channels, in vivo, and thereby functioning in the therapeutic applications set forth herein in relation to capsaicin and the vanilloid receptor.

The compounds of the present invention are useful for the treatment of inflammatory pain and associated hyperalgesia and allodynia. They are also useful for the treatment of neuropathic pain and associated hyperalgesis and allodynia (e.g. trigeminal or berpetic neuralgia, diabetic neuropathy, causalgia, sympathetically maintained pain and deafferentation syndromes such as brachial plexus avulsion). The compounds of the present invention are also useful as anti-inflammatory agents for the treatment of arthritis, and as agents to treat Parkinson's Disease, Alzheimer's Disease, stroke, uveitis, asthma, myocardial infarction, traumatic brain injury, spinal cord injury, neurodegenerative disorders, alopecia (hair loss), inflammatory bowel disease and autoimmune disorders, renal disorders, obesity, eating disorders, cancer, schizophrenia, epilepsy, sleeping disorders, cognition, depression, anxiety, blood pressure, lipid disorders, and atherosclerosis.

In one aspect, this invention provides compounds which are capable of modifying ion channels, in vivo. Representative ion channels so modified include voltage-gated channels and ligand-gated channels, including cation channels such as vanilloid channels.

In a further aspect, the present invention provides pharmaceutical compositions comprising a compound of the invention, and a pharmaceutical carrier, excipient or diluent. In this aspect of the invention, the pharmaceutical composition can comprise one or more of the compounds described herein.

In a further aspect of the invention, a method is disclosed for treating mammals, including humans, as well as lower mammalian species, susceptible to or afflicted with a condition from among those listed herein, and particularly, such condition as may be associated with e.g. arthritis, uveitis, asthma, myocardial infarction, traumatic brain injury, acute spinal cord injury, alopecia (hair loss), inflammatory bowel disease and autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. Compounds have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

In additional method of treatment aspects, this invention provides methods of treating a mammal susceptible to or afflicted with neurodegenerative diseases and disorders such as, for example Parkinson's disease, Alzheimer's disease and multiple sclerosis; diseases and disorders which are mediated by or result in neuroinflammation such as, for example traumatic brain injury, stroke, and encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; irritable bowel syndrome, over active bladder, respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; alopecia (hair loss); obesity; lipid disorders; cancer; blood pressure; spinal cord injury; and renal disorders method comprises administering an effective condition-treating or condition-preventing amount of one or more of the pharmaceutical compositions just described.

In additional aspects, this invention provides methods for synthesizing the compounds of the invention, with representative synthetic protocols and pathways disclosed later on herein.

Other objects and advantages will become apparent to those skilled in the art from a consideration of the ensuing detailed description, in conjunction with the following illustrative drawings.

### BRIEF DESCRIPTION OF THE DRAWINGS

FIG. 1: Graph depicts significant inhibition of the Capsaicin induced intracellular calcium response, under described experimental conditions, by 3 nM of Compound having Id No. 225.

FIG. 2: Graph depicts significant inhibition of the Capsaicin induced intracellular calcium response, under described experimental conditions, by 3 nM of Compound having Id No. 187.

FIG. 3: Graph depicts significant inhibition of the Capsaicin induced intracellular calcium response, under described experimental conditions, by 3 nM of Compound having Id No. 96.

FIG. 4: Graph depicts significant inhibition of the Capsaicin induced intracellular calcium response, under described experimental conditions, by 3 nM of Compound having Id No. 45.

FIG. 5: Graph depicts significant inhibition of the Capsaicin induced intracellular calcium response, under described experimental conditions, by 3 nM of Compound having Id No. 233.

FIG. 6: Graph depicts significant inhibition of the Capsaicin induced intracellular calcium response, under described experimental conditions, by 3 nM of Compound having Id No. 167.

### DETAILED DESCRIPTION OF THE INVENTION

### Definitions

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope. By way of non-limiting example, such substituents may include e.g. halo (such as fluoro, chloro, bromo), —CN, —CF₃, —OH, —OCF₃, C₂-C₆ alkenyl, C₃-C₆ alkynyl, C₁-C₆ alkoxy, aryl and di-C₁-C₆ alkylamino.

“Acyl” refers to a radical —C(O)R, where R is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cylcohexylcarbonyl, cyclohexylmethylcarbonyl, benzoyl, benzylcarbonyl and the like.

“Acylamino” refers to a radical —NR′C(O)R, where R¹ is hydrogen, alkyl, cycloalkyl, cycloheteroalkyl, aryl, arylalkyl, heteroalkyl, heteroaryl, heteroarylalkyl and R is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)R where R is hydrogen, alkyl, aryl or cycloalkyl.

“Substituted alkenyl” includes those groups recited in the definition of “substituted” herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR where R is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, heteroaryl, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonylamino” refers to the group —NRC(O)OR′ where R is hydrogen, alkyl, aryl or cycloalkyl, and R¹ is alkyl or cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkenylene, alkynyl and alkynylene. Aliphatic groups typically have from 1 or 2 to about 12 carbon atoms.

“Alkyl” refers to monovalent saturated aliphatic hydrocarbyl groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyls” as defined below.

“Substituted alkyl” includes those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-(O)₂—, and aryl-S(O)₂—.

“Alkylene” refers to divalent saturated aliphatic hydrocarbyl groups particularly having up to about 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” includes those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkenyl” refers to monovalent olefinically unsaturated hydrocarbyl groups preferably having up to about 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkenylene” refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically unsaturated hydrocarbyl groups particularly having up to about 11 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Substituted alkynyl” includes those groups recited in the definition of “tsubstituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkanoyl” or “acyl” as used herein refers to the group R—C(O)—, where R is hydrogen or alkyl as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

“Substituted Aryl” includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Fused Aryl” refers to an aryl having two of its ring carbon in common with a second aryl ring or with an aliphatic ring.

“Alkaryl” refers to an aryl group, as defined above, substituted with one or more alkyl groups, as defined above.

“Aralkyl” or “arylalkyl” refers to an alkyl group, as defined above, substituted with one or more aryl groups, as defined above.

“Aryloxy” refers to —O-aryl groups wherein “aryl” is as defined above.

“Alkylamino” refers to the group alkyl-NR′R″, wherein each of R′ and R″ are independently selected from hydrogen and alkyl.

“Arylamino” refers to the group aryl-NR′R″, wherein each of R′ and R″ are independently selected from hydrogen, aryl and heteroaryl.

“Alkoxyamino” refers to a radical —N(H)OR where R represents an alkyl or cycloalkyl group as defined herein.

“Alkoxycarbonyl” refers to a radical —C(O)-alkoxy where alkoxy is as defined herein.

“Alkylarylamino” refers to a radical —NRR′ where R represents an alkyl or cycloalkyl group and R¹ is an aryl as defined herein.

“Alkylsulfonyl” refers to a radical —S(O)₂R where R is an alkyl or cycloalkyl group as defined herein. Representative examples include, but are not limited to, methylsulfonyl, ethylsulfonyl, propylsulfonyl, butylsulfonyl and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR where R is an alkyl or cycloalkyl group as defined herein that may be optionally substituted as defined herein Representative examples include, but are not limited to, methylthio, ethylthio, propylthio, butylthio, and the like.

“Amino” refers to the radical —NH₂.

“Substituted amino” includes those groups recited in the definition of “substituted” herein, and particularly refers to the group —N(R) where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R)₂ is an amino group.

“Aminocarbonyl” refers to the group —C(O)NRR where each R is independently hydrogen, alkyl, aryl and cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Arylalkyloxy” refers to an —O-arylalkyl radical where arylalkyl is as defined herein.

“Arylamino” means a radical —NHR where R represents an aryl group as defined herein.

“Aryloxycarbonyl” refers to a radical —C(O)—O-aryl where aryl is as defined herein.

“Arylsulfonyl” refers to a radical —S(O)₂R where R is an aryl or heteroaryl group as defined herein.

“Azido” refers to the radical —N₃.

“Carbamoyl” refers to the radical —C(O)N(R)₂ where each R group is independently hydrogen, alkyl, cycloalkyl or aryl, as defined herein, which may be optionally substituted as defined herein.

“Carboxy” refers to the radical —C(O)OH.

“Carboxyamino” refers to the radical —N(H)C(O)OH.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Substituted cycloalkyl” includes those groups recited in the definition of “substituted” herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-(O)—, alkyl-S(O)₂— and aryl-S(O)₂—.

“Cycloalkoxy” refers to the group —OR where R is cycloalkyl. Such cycloalkoxy groups include, by way of example, cyclopentoxy, cyclohexoxy and the like.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Substituted cycloalkenyl” includes those groups recited in the definition of “substituted” herein, and particularly refers to a cycloalkenyl group having I or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂ and aryl-S(O)₂—.

“Fused Cycloalkenyl” refers to a cycloalkenyl having two of its ring carbon atoms in common with a second aliphatic or aromatic ring and having its olefinic unsaturation located to impart aromaticity to the cycloalkenyl ring.

“Cyanato” refers to the radical —OCN.

“Cyano” refers to the radical —CN.

“Dialkylamino” means a radical —NRR′ where R and R′ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

“Ethenyl” refers to substituted or unsubstituted —(C═C)—.

“Ethylene” refers to substituted or unsubstituted —(C—C)—.

“Ethynyl” refers to —(C≡C)—.

“Halo” or “halogen” refers to fluoro, chloro, bromo and iodo. Preferred halo groups are either fluoro or chloro.

“Hydroxy” refers to the radical —OH.

“Nitro” refers to the radical —NO₂.

“Substituted” refers to a group in which one or more hydrogen atoms are each independently replaced with the same or different substituent(s). Typical substituents include, but are not limited to, —X, —R¹⁴, —O⁻, ═O, —OR¹⁴, —SR¹⁴, —S⁻, ═S, —NR¹⁴R¹⁵, ═NR¹⁴, —CX₃, —CF₃, —CN, —OCN, —SCN, —NO, —NO₂, ═N₂, —N₃, —S(O)₂O⁻, —S(O)₂OH, —S(O)₂R¹⁴, —OS(O₂)O⁻, —OS(O)₂R¹⁴, —P(O)(O⁻)₂, —P(O)(OR¹⁴)(O), —OP(O)(OR¹⁴)(OR¹⁵), —C(O)R¹⁴, —C(S)R¹⁴, —C(O)OR¹⁴, —C(O)NR¹⁴R¹⁵, —C(O)O⁻; —C(S)OR¹⁴, —NR¹⁶C(O)NR¹⁴R¹⁵, —NR¹⁶C(S)NR¹⁴R¹⁵, —NR¹⁷C(NR¹⁶)NR¹⁴R¹⁵ and —C(NR¹⁶)NR¹⁴R¹⁵, where each X is independently a halogen; each R¹⁴, R¹⁵, R¹⁶ and R¹⁷ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR¹⁸R¹⁹, —C(O)R¹⁸ or —S(O)₂R¹⁸ or optionally R¹⁸ and R¹⁹ together with the atom to which they are both attached form a cycloheteroalkyl or substituted cycloheteroalkyl ring; and R¹⁸ and R¹⁹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

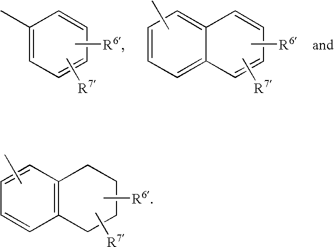

In these formulae one of R^{6′} and R^{7′} may be hydrogen and at least one of R^{6′} and R^{7′} is each independently selected from alkyl, alkenyl, alkynyl, cycloheteroalkyl, alkanoyl, alkoxy, aryloxy, heteroaryloxy, alkylamino, arylamino, heteroarylamino, NR¹⁰COR¹¹, NR¹⁰SOR¹¹,NR¹⁰SO₂R¹⁴, COOalkyl, COOaryl, CONR¹⁰R¹¹, CONR¹⁰OR¹¹, NR¹⁰R¹¹, SO₂NR¹⁰R¹¹, S-alkyl, S-alkyl, SOalkyl, SO₂alkyl, Saryl, SOaryl, SO₂aryl; or R^{6′} and R^{7′} may be joined to form a cyclic ring (saturated or unsaturated) from 5 to 8 atoms, optionally containing one or more heteroatoms selected from the group N, O or S. R¹⁰, R¹¹, and R¹² are independently hydrogen, alkyl, alkenyl, alkynyl, perfluoroalkyl, cycloalkyl, cycloheteroalkyl, aryl, substituted aryl, heteroaryl, substituted or hetero alkyl or the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. cycloheteroalkyl, aryl, e.g. heteroaryl, cycloalkenyl, cycloheteroalkenyl, and the like having from 1 to 5, and especially from 1 to 3 heteroatoms.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Particular heteroaryl groups are those derived from thiophene, pyrrole, benzothiophene, benzofuran, indole, pyridine, quinoline, imidazole, oxazole and pyrazine.

Examples of representative heteroaryls include the following:

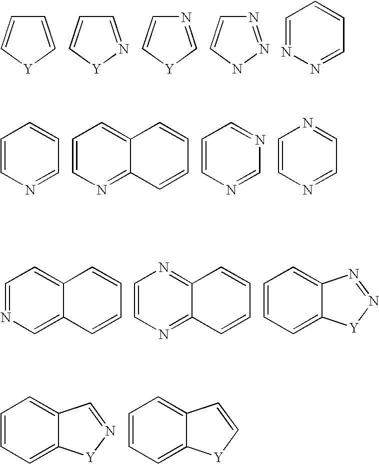

wherein each Y is selected from carbonyl, N, NR⁴, O, and S.

Examples of representative cycloheteroalkyls include the following

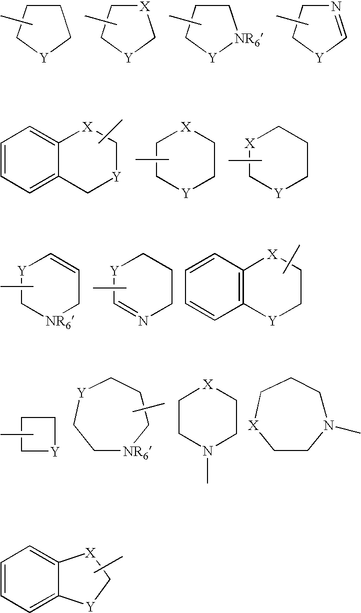

wherein each X is selected from CR⁴₂, NR⁴, 0 and S; and each Y is selected from NR⁴, O and S, and where R^{6′} is R².

Examples of representative cycloheteroalkenyls include the following:

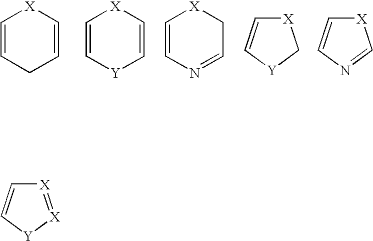

wherein each X is selected from CR⁴, NR⁴, O and S; and each Y is selected from carbonyl, N, NR⁴, O and S.

Examples of representative aryl having hetero atoms containing substitution include the following:

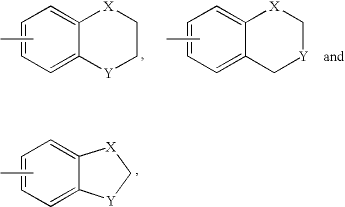

wherein each X is selected from C—R⁴, CR⁴₂, NR⁴, O and S; and each Y is selected from carbonyl, NR⁴, O and S.

“Hetero substituent” refers to a halo, O, S or N atom-containing functionality that may be present as an R⁴ in a R⁴C group present as substituents directly on A, B, W, X, Y or Z of the compounds of this invention or may be present as a substituent in the “substituted” aryl and aliphatic groups present in the compounds.

Examples of hetero substituents include:

-halo,

—NO₂, —NH₂, —NHR, —N(R)₂,

—NRCOR, —NRSOR, —NRSO₂R, OH, CN,

—CO₂H,

—R—OH, —O—R, —COOR,

—CON(R)₂, —CONROR,

—SO₃H, —R—S, —O₂N(R)₂,

—S(O)R, —S(O)₂R, wherein each R is independently an aryl or aliphatic, optionally with substitution. Among hetero substituents containing R groups, preference is given to those materials having aryl and alkyl R groups as defined herein. Preferred hetero substituents are those listed above.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples:

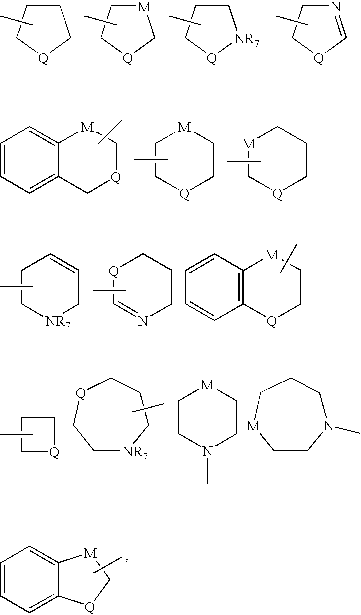

optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives. In the examples, M is CR⁷, NR², O, or S; Q is O, NR² or S. R⁷ and R⁸ are independently selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-(O)₂— and aryl-S(O)₂—.

“Dihydroxyphosphoryl” refers to the radical —PO(OH)₂.

“Substituted dihydroxyphosphoryl” includes those groups recited in the definition of “substituted” herein, and particularly refers to a dihydroxyphosphoryl radical wherein one or both of the hydroxyl groups are substituted. Suitable substituents are described in detail below.

“Aminohydroxyphosphoryl” refers to the radical —PO(OH)NH₂.

“Substituted aminohydroxyphospboryl” includes those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

“Thioalkoxy” refers to the group —SR where R is alkyl.

“Substituted thioalkoxy” includes those groups recited in the definition of “substituted” herein, and particularly refers to a thioalkoxy group having I or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Sulfanyl” refers to the radical HS—. “Substituted sulfanyl” refers to a radical such as RS— wherein R is any substituent described herein.

“Sulfonyl” refers to the divalent radical —S(O₂)—. “Substituted sulfonyl” refers to a radical such as R—(O₂)S— wherein R is any substituent described herein. “Aminosulfonyl” or “Sulfonamide” refers to the radical H₂N(O₂)S—, and “substituted aminosulfonyl” “substituted sulfonamide” refers to a radical such as R₂N(O₂)S— wherein each R is independently any substituent described herein.

“Sulfone” refers to the group —SO₂R. In particular embodiments, R is selected from H, lower alkyl, alkyl, aryl and heteroaryl.

“Thioaryloxy” refers to the group —SR where R is aryl.

“Thioketo” refers to the group ═S.

“Thiol” refers to the group —SH.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, furmaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

“Solvate” refers to forms of the compound that are associated with a solvent, usually by a solvolysis reaction. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates.

“Subject” includes humans. The terms “human,” “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g. stabilization of a discernible symptom), physiologically, (e.g., stabilization of a physical parameter), or both. In yet another embodiment, “treating” or “treatment” refers to delaying the onset of the disease or disorder.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of 71 electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Representative enol—keto structures and equilibrium are illustrated below:

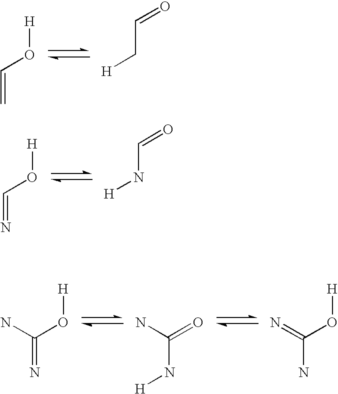

Tautomeric forms may be relevant to the attainment of the optimal chemical reactivity and biological activity of a compound of interest.

The compounds of this invention may possess one or more asymmetric centers; such compounds can therefore be produced as individual (R)— or (S)— stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art

### Compounds

Compounds provided herein are useful for preventing and/or treating a broad range of conditions, among them, arthritis, Parkinson's disease, Alzheimer's disease, stroke, uveitis, asthma, myocardial infarction, the treatment and prophylaxis of pain syndromes (acute and chronic or neuropathic), traumatic brain injury, acute spinal cord injury, neurodegenerative disorders, alopecia (hair loss), inflammatory bowel disease and autoimmune disorders or conditions in mammals.

In order that the invention described herein may be more fully understood, the following structures representing compounds typical of the invention are set forth. It should be understood that these examples are for illustrative purposes only and are not to be construed as limiting this invention in any manner.

Accordingly, additional groups of particular compounds are provided. Thus, and as discussed earlier herein, suitable compounds capable of modifxing ion channels in vivo, may be selected from those listed in Tables 1-1 and 1-2, below, and may be prepared either as shown or in the form of a pharmaceutically acceptable salt, solvate or prodrug thereof; and stereoisomers and tautomers thereof. All such variants are contemplated herein and are within the scope of the present invention.

In certain aspects, the present invention provides prodrugs and derivatives of the compounds according to the formulae above. Prodrugs are derivatives of the compounds of the invention, which have cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

### Assay Methods

### Chronic Constriction Injury Model (CCI Model):

Male Sprague-Dawley rats (270-300 g; B. W., Charles River, Tsukuba, Japan) are used. The chronic constriction injury (CCI) operation is performed according to the method described by Bennett and Xie (Bennett, G. J. and Xie, Y. K. Pain, 33:87-107, 1988). Briefly, animals are anesthetized with sodium pentobarbital (64.8 mg/kg, i.p.) and the left common sciatic nerve is exposed at the level of the middle of the thigh by blunt dissection through the biceps femoris. A portion of the sciatic nerve proximal to its trifurcation is freed of adhering tissue and 4 ligatures (4-0 silk) are tied loosely around it with about 1 mm space. A sham operation is performed as same as CCI surgery except for sciatic nerve ligation. Two weeks after surgery, mechanical allodynia is evaluated by application of von Frey hairs (VFHs) to the plantar surface of the hind paw. The lowest amount of force of VFH required to elicit a response is recorded as the paw withdrawal threshold (PWT). VFH testing is performed at 0.5, 1 and 2 hr post-dosing. Experimental data are analyzed using Kruskal-Wallis test followed by Dunn's test for multiple comparisons or Mann-Whitney U-test for paired comparison.

### Caco-2 Permeability

Caco-2 permeability is measured according to the method described in Shiyin Yee, Pharmaceutical Research, 763 (1997).

Caco-2 cells are grown on filter supports (Falcon HTS multiwell insert system) for 14 days. Culture medium is removed from both the apical and basolateral compartments and the monolayers are preincubated with pre-warmed 0.3 ml apical buffer and 1.0 ml basolateral buffer for 0.75 hour at 37° C. in a shaker water bath at 50 cycles/min. The apical buffer consists of Hanks Balanced Salt Solution, 25 mM D-glucose monohydrate, 20 mM MES Biological Buffer, 1.25 mM CaCl₂ and 0.5 mM MgCl₂ (pH 6.5). The basolateral buffer consists of Hanks Balanced Salt Solution, 25 mM D-glucose monohydrate, 20 mM HEPES Biological Buffer, 1.25 mM CaCl₂ and 0.5 mM MgCl2 (pH 7.4). At the end of the preincubation, the media is removed and test compound solution (10 μM) in buffer is added to the apical compartment. The inserts are moved to wells containing fresh basolateral buffer and incubated for 1 hr. Drug concentration in the buffer is measured by LC/MS analysis.

Flux rate (F, mass/time) is calculated from the slope of the cumulative appearance of substrate on the receiver side and apparent permeability coefficient (Papp) is calculated from the following equation:

Papp(cm/sec)=(F*VD)/(SA*MD)

where SA is surface area for transport (0.3 cm²), VD is the donor volume (0.3 ml), MD is the total amount of drug on the donor side at t=0. All data represent the mean of 2 inserts. Monolayer integrity is determined by Lucifer Yellow transport.

### Human Dofetilide Binding

Cell paste of BEK-293 cells expressing the HERG product can be suspended in 10-fold volume of 50 mM Tris buffer adjusted at pH 7.5 at 25° C. with 2 M HCl containing 1 mM MgCl₂, 10 mM KCl. The cells are homogenized using a Polytron homogenizer (at the maximum power for 20 seconds) and centrifuged at 48,000 g for 20 minutes at 4° C. The pellet is resuspended, homogenized and centrifuged once more in the same manner. The resultant supernatant is discarded and the final pellet is resuspended (10-fold volume of 50 mM Tris buffer) and homogenized at the maximum power for 20 seconds. The membrane homogenate is aliquoted and stored at −80° C. until use. An aliquot is used for protein concentration determination using a Protein Assay Rapid Kit and ARVO SX plate reader (Wallac). All the manipulation, stock solution and equipment are kept on ice at all time. For saturation assays, experiments are conducted in a total volume of 200 μl. Saturation is determined by incubating 20 μl of [3H]-dofetilide and 160 μl of membrane homogenates (20-30 μg protein per well) for 60 min at room temperature in the absence or presence of 10 μM dofetilide at final concentrations (20 μl) for total or nonspecific binding, respectively. All incubations are terminated by rapid vacuum filtration over polyetherimide (PEI) soaked glass fiber filter papers using Skatron cell harvester followed by two washes with 50 mM Tris buffer (pH 7.5 at 25° C.). Receptor-bound radioactivity is quantified by liquid scintillation counting using a Packard LS counter.

For the competition assay, compounds are diluted in 96 well polypropylene plates as 4-point dilutions in semi-log format. All dilutions are performed in DMSO first and then transferred into 50 mM Tris buffer (pH 7.5 at 25° C.) containing 1 mM MgCl₂, 10 mM KCl so that the final DMSO concentration became equal to 1%. Compounds are dispensed in triplicate in assay plates (4 μl). Total binding and nonspecific binding wells are set up in 6 wells as vehicle and 10 μM dofetilide at final concentration, respectively. The radioligand is prepared at 5.6× final concentration and this solution is added to each well (36 μl). The assay is initiated by addition of YSi poly-L-lysine Scintillation Proximity Assay (SPA) beads (50 μl, 1 mg/well) and membranes (110 μl, 20 μg/well). Incubation is continued for 60 min at room temperature. Plates are incubated for a further 3 hours at room temperature for beads to settle. Receptor-bound radioactivity is quantified by counting Wallac MicroBeta plate counter.

### HERG Assay

HEK 293 cells which stably express the HERG potassium channel are used for electrophysiological study. The methodology for stable transfection of this channel in HEK cells can be found elsewhere (Z. Zhou et al., 1998, Biophysical Journal, 74, pp 230-241). Before the day of experimentation, the cells are harvested from culture flasks and plated onto glass coverslips in a standard Minimum Essential Medium (MEM) medium with 10% Fetal Calf Serum (FCS). The plated cells are stored in an incubator at 37° C. maintained in an atmosphere of 95% O₂/5% CO₂. Cells are studied between 15-28 hrs after harvest.

HERG currents are studied using standard patch clamp techniques in the whole-cell mode. During the experiment the cells are superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings are made using a patch clamp amplifier and patch pipettes which have a resistance of 1-3 MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15MΩ and seal resistances >1 GΩ is accepted for further experimentation. Series resistance compensation is applied up to a maximum of 80%. No leak subtraction was done. However, acceptable access resistance depended on the size of the recorded-currents and the level of series resistance compensation that can safely be used. Following the achievement of whole cell configuration and sufficient time for cell dialysis with pipette solution (>5 min), a standard voltage protocol was applied to the cell to evoke membrane currents. The voltage protocol is as follows. The membrane was depolarized from a holding potential of −80 mV to +40 mV for 1000 ms. This is followed by a descending voltage ramp (rate 0.5 mV msec-1) back to the holding potential. The voltage protocol is applied to a cell continuously throughout the experiment every 4 seconds (0.25 Hz). The amplitude of the peak current elicited around −40 mV during the ramp is measured. Once stable evoked current responses are obtained in the external solution, vehicle (0.5% DMSO in the standard external solution) is applied for 10-20 min by a peristaltic pump. Provided there were minimal changes in the amplitude of the evoked current response in the vehicle control condition, the test compound of either 0.3, 1, 3, 10 mM is applied for a 10 min period. The 10 min period included the time which supplying solution is passing through the tube from solution reservoir to the recording chamber via the pump. Exposure time of cells to the compound solution is more than 5 min after the drug concentration in the chamber well reaches the intended concentration. There is a subsequent wash period of a 10-20 min to assess reversibility. Finally, the cells are exposed to high dose of dofetilide (5 mM), a specific IKr blocker, to evaluate the insensitive endogenous current.

All experiments are performed at room temperature (23±1° C.). Evoked membrane currents are recorded on-line on a computer, filtered at 500-1 KHz (Bessel −3 dB) and sampled at 1-2 KHz using the patch clamp amplifier and a specific data analyzing software. Peak current amplitude, which generally occurs at around −40 mV, is measured off line on the computer.

The arithmetic mean of the ten values of amplitude is calculated under vehicle control conditions and in the presence of drug. Percent decrease of IN in each experiment is obtained by the normalized current value using the following formula: IN=(1−ID/IC)×100, where ID is the mean current value in the presence of drug and IC is the mean current value under control conditions. Separate experiments are performed for each drug concentration or time-matched control, and arithmetic mean in each experiment is defined as the result of the study.

Mono-Iodoacetate (MIA)-Induced OA model

Male 6-weeks-old Sprague-Dawley (SD, Japan SLC or Charles River Japan) rats are anesthetized with pentobarbital. Injection site (knee) of MIA is shaved and cleaned with 70% ethanol. Twenty-five ml of MIA solution or saline is injected in the right knee joint using a 29 G needle. The effect of joint damage on the weight distribution through the right (damaged) and left (untreated) knee is assessed using an incapacitance tester (Linton Instrumentation, Norfolk, UK). The force exerted by each hind limb is measured in grams. The weight-bearing (WB) deficit is determined by a difference of weight loaded on each paw. Rats are trained to measure the WB once a week until 20 days post MIA-injection. Analgesic effects of compounds are measured at 21 days after the MIA injection. Before the compound administration, the “pre value” of WB deficit is measured. After the administration of compounds, attenuation of WB deficits is determined as analgesic effects.

### Complete Freund's Adjuvant (CFA) Induced Thermal and Mechanical Hyperalgesia in Rats

### Thermal Hyperalgesia

Male 6-week-old SD rats are used. Complete Freund's adjuvant (CFA, 300 mg of Mycobacterium Tuberculosis H37RA (Difco, Mich.) in 100 μL of liquid paraffin (Wako, Osaka, Japan)) is injected into the plantar surface of a hind paw of the rats. Two days after CFA-injection, thermal hyperalgesia is determined by method described previously (Hargreaves et al., 1988) using the plantar test apparatus (Ugo-Basil, Varese, Italy). Rats are adapted to the testing environment for at least 15 minutes prior to any stimulation. Radiant heat is applied to the plantar surface of a hind paw and paw withdrawal latencies (PWL, seconds) are determined. The intensity of radiant heat is adjusted to produce the stable PWL of 10 to 15 seconds. The test compound is administered in a volume of 0.5 mL per 100 g body weight. PWL are measured after 1, 3 or 5 hours after drug administration.

### Mechanical Hyperalgesia

Male 4-week-old SD rats are used. CFA (300 mg of Mycobacterium Tuberculosis H37RA (Difco, Mich.) in 100 μL of liquid paraffin (Wako, Osaka, Japan)) is injected into the plantar surface of a hind paw of the rats. Two days after CFA-injection, mechanical hyperalgesia is tested by measuring paw withdrawal threshold (PWT, grams) to pressure using the analgesy-Meter (Ugo-Basile, Varese, Italy). The animals are gently restrained, and steadily increasing pressure is applied to the dorsal surface of a hind paw via a plastic tip. The pressure required to elicit paw withdrawal is determined. The test compound is administered in a volume of 0.5 mL per 100 g body weight. PWT are measured after 1, 3 or 5 hours after drug administration.

### Pharmaceutical Compositions

When employed as pharmaceuticals, the amide compounds of this invention are typically administered in the form of a pharmaceutical composition. Such compositions can be prepared in a manner well known in the pharmaceutical art and comprise at least one active compound.

Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of this invention can be administered by a variety of routes including by way of non limiting example, oral, rectal, transdermal, subcutaneous, intravenous, intramuscular and intranasal. Depending upon the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions of this invention. The present invention, however, is not limited to the following pharmaceutical compositions.

### Formulation 1

### Tablets

A compound of formula I is admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2

### Capsules

A compound of formula I is admixed as a dry powder with a starch diluent in an approximate 1:1 weight ratio. The mixture is filled into 250 mg capsules (125 mg of active compound per capsule).

### Formulation 3

### Liquid

A compound of formula I (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4

### Tablets

The compound of formula I is admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5

### Injection

The compound of formula I is dissolved or suspended in a buffered sterile saline injectable aqueous medium to a concentration of approximately 5 mg/ml.

### Formulation 6

### Topical

Stearyl alcohol (250 g) and a white petrolatum (250 g) are melted at about 75° C. and then a mixture of a compound of formula I (50 g) methylparaben (0.25 g), propylparaben (0.15 g), sodium lauryl sulfate (10 g), and propylene, glycol (120 g) dissolved in water (about 370 g) is added and the resulting mixture is stirred until it congeals.

### Methods Of Treatment

The present compounds are used as therapeutic agents for the treatment of conditions in mammals. Accordingly, the compounds and pharmaceutical compositions of this invention find use as therapeutics for preventing and/or treating neurodegenerative, autoimmune and inflammatory conditions in mammals including humans.

In a method of treatment aspect, this invention provides a method of treating a mammal susceptible to or afflicted with a condition associated with arthritis, uveitis, asthma, myocardial infarction, traumatic brain injury, acute spinal cord injury, alopecia (hair loss), inflammatory bowel disease and autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. Compounds have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

In additional method of treatment aspects, this invention provides methods of treating a mammal susceptible to or afflicted with neurodegenerative diseases and disorders such as, for example Parkinson's disease, Alzheimer's disease and multiple sclerosis; diseases and disorders which are mediated by or result in neuroinflammation such as, for example traumatic brain injury, stroke, and encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; alopecia (hair loss); obesity; lipid disorders; cancer; blood pressure; spinal cord injury; and renal disorders method comprises administering an effective condition-treating or condition-preventing amount of one or more of the pharmaceutical compositions just described.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as neurodegenerative and autoimmune conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound or its derivative, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of a neurodegenerative, autoimmune or inflammatory condition, the compounds or their derivatives of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other active derivatives. A VR1 antagonist may be usefully combined with another pharmacologically active compound, or with two or more other pharmacologically active compounds, particularly in the treatment of pain. For example, a VR1 antagonist, particularly a compound of formula (I), or a pharmaceutically acceptable salt or solvate thereof, as defined above, may be administered simultaneously, sequentially or separately in combination with one or more agents selected from:

an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;

a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;

a barbiturate sedative, e.g. amobarbital, aprobarbital, butabarbital, butabital, mephobarbital, metharbital, methohexital, pentobarbital, phenobartital, secobarbital, talbutal, theamylal or thiopental;

a benzodiazepine having a sedative action, e.g. chlordiazepoxide, clorazepate, diazepam, flurazepam, lorazepam, oxazepam, temazepam or triazolam;

an H1 antagonist having a sedative action, e.g. diphenhydramine, pyrilamine, promethazine, chlorpheniramine or chlorcyclizine;

a sedative such as glutethimide, meprobamate, methaqualone or dichloralphenazone;

a skeletal muscle relaxant, e.g. baclofen, carisoprodol, chlorzoxazone, cyclobenzaprine, methocarbamol or orphrenadine;

an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;

an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;

a tricyclic antidepressant, e.g. desipramine, imipramine, damitriptyline or nortriptyline;

an anticonvulsant, e.g. carbamazepine, lamotrigine, topiratmate or valproate;

a tachykinin (NK) antagonist, particularly an NK-3, NK-2 or NK-1 antagonist, e.g. (aR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);

a muscarinic antagonist, e.g. oxybutynin, tolterodine, propiverine, tropism chloride, darifenacin, solifenacin, temiverine and ipratropium;

a COX-2 selective inhibitor, e.g. celecoxib, rofecoxib, parecoxib, valdecoxib, deracoxib, etoricoxib, or lumiracoxib;

a coal-tar analgesic, in particular paracetarnol;

a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisuipride, balaperidone, palindore, eplivanserin, osanetant, rimonabant, meclinertant, Miraxion® or sarizotan;

a beta-adrenergic such as propranolol;

a local anaesthetic such as mexiletine;

a corticosteroid such as dexamethasone;

a 5-HT receptor agonist or antagonist, particularly a 5-HT1B/1D agonist such as eletriptan, sumatriptan, naratriptan, zolmitriptan or rizatriptan;

a 5-HT2A receptor antagonist such as R(+)-alpha-(2,3-dimethoxy-phenyl)-1-[2-(4-fluorophenylethyl)]-4-piperidinemethanol (MDL-100907);

a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;

Tramadol®;

a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′, 1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;

an alpha-2-delta ligand such as gabapentin, pregabalin, 3-methylgabapentin, (1a,3a,5a)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;

a cannabinoid;

a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;

a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;

a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;

an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol,

2-[[(1R,3S)-3-amino-4-hydroxy-1-(5-thiazolyl) butyl]thio]-6-(trifluoromethyl)-3pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;

an acetylcholinesterase inhibitor such as donepezil;

a prostaglandin E2 subtype 4 (EP4) antagonist such as N—[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;

a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,

a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);

a sodium channel blocker, such as lidocaine;

a 5-HT3 antagonist, such as ondansetron;

and the pharmaceutically acceptable salts and solvates thereof.

In as much as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

### Preparation of the Compounds

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The target compounds are synthesized by known reactions outlined in the following schemes. The products are isolated and purified by known standard procedures. Such procedures include (but are not limited to) recrystallization, column chromatography or HPLC.

In this specification, especially in “General Synthesis” and “Examples”, the following abbreviations can be used:

DCM dichloromethane
DME 1,2-dimethoxyethane, dimethoxyethane

### DMF N,N-dimethylformamide

DMSO dimethyl sulfoxide
EDC 1-ethyl-3-(3′-dimethylaminopropyl)carbodiimide hydrogen chloride)
EtOAc ethyl acetate
EtOH ethanol
HOBt 1-hydroxybenzotriazole
MeOH methanol
THF tetrahydrofuran
TFA trifluoroacetic acid

### Preparation of Acid Building Blocks

### Preparation of Substituted Benzoic Acids

### Intermediate 1

### Preparation of 2-chloro-6-(3,3-dimethylbut-1-ynyl)nicotinic acid

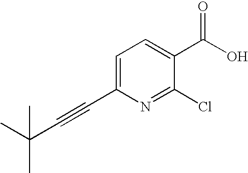

2,6-dichloropyridine-3-carboxylic acid (2.0 g, 10.42 mmol), 3,3-dimethylbut-1-yne (1.4 mL, 11.46 mmol), copper(I) iodide (0.198 g, 1.04 mmol) and bis(triphenylphosphine) palladium(II) chloride (1.46 g, 2.08 mmol) were stirred in 40 mL triethylamine at room temperature for 24 h. The solvent was removed in vacuo and the residue was purified by column chromatography using 10-50% MeOH/EtOAc to furnish 125 mg (5%) of the title compound as an orange solid. m/z=236 (M−1).

### Intermediate 2

### Preparation of (e)-2-methyl-4-(3,3,3-trifluoroprop-1-enyl)benzoic acid

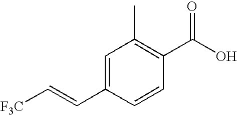

A mixture of 4-bromo-2-methylbenzoic acid (25 g, 0.12 mol), tri-o-tolylphosphine (7.1 g, 0.023 mol), tetra-N-butylammonium chloride (9.7 g, 0.035 mol), potassium acetate (22.8 g, 0.232 mol), 3,3,3-trifluoroprop-1-ene (89 g, 0.93 mol), palladium acetate (1.3 g, 0.0058 mol) and N,N-dimethylacetamide (150 mL, 1.6 mol) was sealed in a Parr instrument and stirred at 180° C. for 120 h. After cooling, the reaction mixture was filtered through celite and the filtrate was partitioned between EtOAc and 1N HCl (pH 2-3). The organic layer was separated and washed with brine, dried (Na₂SO₄) and concentrated under vacuum. The residue was purified by column chromatography on silica gel to give a crude product (which contained a small amount of the corresponding (Z)-isomer). The (2)-isomer and other impurities could be removed by column after transforming the acid into the corresponding methyl ester. Saponification of the methyl ester gave the pure acid as a white solid (16.5 g, 62%).

### Intermediate 3

### Preparation of 4-(cyclopentylethenyl)-2-fluorobenzoic acid

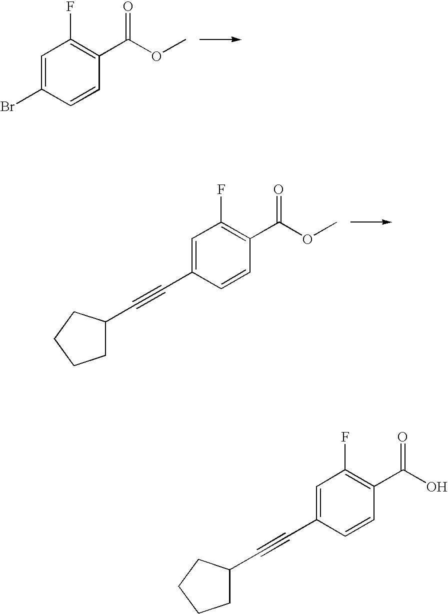

### Methyl 4-(cyclopentylethenyl)-2-fluorobenzoate

4-Bromo-2-fluorobenzoic acid methyl ester (1.0 g, 4.0 mmol) was dissolved in triethylamine (5 mL). To the mixture was added copper iodide (38 mg, 5 mol %), followed by PdCl₂(PPh₃)₂ (140 mg, 5 mol %) and ethynylcyclopentane (0.85 mL, 6.3 mmol). The mixture was heated in a sealed pressure tube at 80° C. for 3 hours. After completion of the reaction, the triethylamine was removed under vacuum and the residue was dissolved in EtOAc and filtered through celite. The organic layer was washed with water, brine, and dried (Na₂SO₄), filtered and the mixture concentrated under vacuum. The residue was purified using column chromatography on silica using EtOAc-hexane (0-100% gradient) as eluent to give the product (0.92 g).

### 4-(cyclopentylethenyl)-2-fluorobenzoic acid

Methyl 4-(cyclopentylethenyl)-2-fluorobenzoate was dissolved in 10 mL of MeOH and 10 mL of 2N LiOH and the mixture was refluxed overnight. The MeOH was removed under vacuum and the basic layer was washed with EtOAC, acidified, and re-extracted with EtOAC. The organic layer was washed with brine, dried (Na₂SO₄), filtered and concentrated under vacuum to give the desired product (645 mg) as a beige solid. m/z=233 (M+1).

### Intermediate 4

### Preparation of 2-chloro-6-(cyclopropylethynyl)nicotinic acid

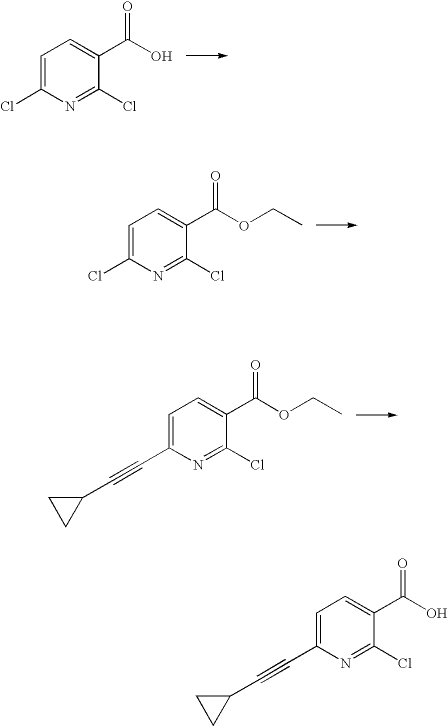

### Ethyl 2,6-dichloropyridine-3-carboxylate

2,6-dichloropyridine-3-carboxylic acid (2.0 g, 10.42 mmol) was placed in 100 mL EtOH, 2 mL conc. H₂SO₄ was added and the mixture was refluxed for 18 h. The reaction mixture was cooled and the pH adjusted to 5 with satd. aqueous NaHCO₃ and then extracted with EtOAc. The organic layer was separated and dried (Na₂SO₄). Removal of solvent in vacuo furnished 2.1 g of the ethyl ester which was used in the next step without further purification. m/z=220.6 (M+1).

### Ethyl 2-chloro-6-(2-cyclopropylethynyl)pyridine-3-carboxylate

Ethyl 2,6-dichloropyridine-3-carboxylate (2.0 g, 9.1 mmol), ethynylcyclopropane (1.6 mL of a 70% w/v solution in toluene, 13.63 mmol), copper(I) iodide (173 mg, 0.9 mmol), bis(triphenylphosphine) palladium(II) chloride (1.28 g, 1.82 mmol) were stirred in 40 mL triethylamine at room temperature for 24 h. The solvent was removed in vacuo and the residue was purified by column chromatography using 10-50% EtOAc/hexane to give the product (0.7 g, 31%) as a brown oil. m/z=250 (M+1).

### 2-chloro-6-(cyclopropylethynyl)nicotinic acid

The ester was hydrolyzed as follows: Ethyl 2-chloro-6-(2-cyclopropylethynyl)pyridine-3-carboxylate (0.7 g, 2.8 mmol) and lithium hydroxide (0.4 g, 16.86 mmol) were refluxed in a mixture of 30 mL MeOH and 10 mL water. The mixture was cooled and the methanol was removed in vacuo. The remaining solution was acidified to pH 2 with 1M HCl at 0° C. The precipitate was filtered and dried to give 0.4 g (57%) of the title compound. m/z=222.4 (M+1).

### Intermediate 5

### Preparation of (Z)-2-methoxy-4-(3,3,3-trifluoroprop-1-enyl)benzoic acid and preparation of (E)-2-methoxy-4-(3,3,3-trifluoromethylprop-1-enyl)benzoic acid

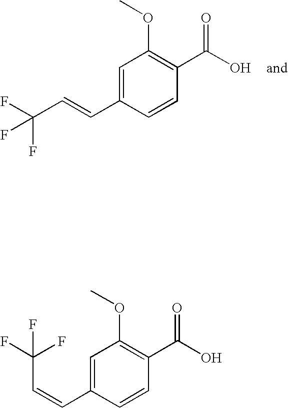

### Methyl 4-formyl-2-methoxybenzoate

A slow stream of CO was passed into a suspension of methyl 4-bromo-2-methoxybenzoate (2.4 g, 0.010 mol), bis(triphenylphosphine)palladium(11) chloride (140 mg, 0.00020 mol), sodium formate (1.02 g, 0.0150 mol), and dry DMF (10 mL). The mixture was vigorously stirred at 110° C. for 2 h. After cooling, the mixture was treated with aqueous Na₂CO₃ solution and extracted with EtOAc. The extract was washed with brine, dried (Na₂SO₄), and concentrated. The residue was purified by column chromatography on silica gel with AcOEt-hexane as eluent (0 to 50%) to give a colorless oil.

### Methyl (E)-4-(3,3,3-trifluoroprop-1-enyl)-2-methoxybenzoate and methyl (Z)-4-(3,3,3-trifluoroprop-1-enyl)-2-methoxybenzoate

MS 4 Å (powder, 16 g) was added to a 1 M solution of TBAF in THF (20 mL, 20 mmol), and the mixture was stirred at room-temperature overnight under an argon atmosphere. To the mixture were added a solution of methyl 4-formyl-2-methoxybenzoate (420 mg, 0.0022 mol) and 2,2,2-trifluoroethyldiphenylphosphine oxide (1.23 g, 0.00432 mol) in THF (20 mL). After the mixture was stirred for 2 h, MS 4 Å was removed by filtration. The filtrate was concentrated and water (120 mL) was added. The mixture was extracted with AcOEt. The extract was washed with brine, dried (Na₂SO₄), and concentrated. The residue was purified by column chromatography on silica gel using AcOEt-hexane (0-15%) as eluent to give (E)-methyl 4-(3,3,3-trifluoroprop-1-enyl)-2-methoxybenzoate as a white solid, followed by (Z)-methyl 4-(3,3,3-trifluoropropl-1-enyl)-2-methoxybenzoate as a colorless oil.

### (E)-4-(3,3,3-Trifluoroprop-1-enyl)-2-methoxybenzoic acid

A mixture of (E)-methyl 4-(3,3,3-trifluoroprop-1-enyl)-2-methoxybenzoate (340 mg, 0.0013 mol), MeOH (20 mL), and 2 N aqueous NaOH solution (1.5 mL) was stirred at 65° C. overnight. The solvents were removed under reduced pressure and the residue was treated with water, acidified with 1N HCl to pH 2-3, and extracted with EtOAc (50 mL×3). The combined organic layers were washed with brine, dried (Na₂SO₄), filtered and concentrated under vacuum to give the product as a white solid. LC-MS: 2.59 min, 244.8 (M−1).

### (Z)-4-(3,3,3-trifluoroprop-1-enyl)-2-methoxybenzoic acid

A mixture of (Z)-methyl 4-(3,3,3-trifluoroprop-1-enyl)-2-methoxybenzoate (60.0 mg, 0.000230 mol), MeOH (10 mL), and 2 N aqueous NaOH solution (0.5 mL) was stirred at 65° C. for 5 h. After cooling the mixture, the solvent was removed under reduced pressure. The residue was treated with water, acidified with 1N HCl to pH 2-3, and extracted with EtOAc (30 mL×3). The combined organic layers were washed with brine, dried (Na₂SO₄), filtered and concentrated under vacuum to give the product as a syrup which became an off-white solid while standing at room temperature for a long time. LC-MS: 2.49 min, 244.8 (M−1).

### Intermediate 6

### Preparation of 4-(cyclopropylethynyl)-2-methylbenzoic acid

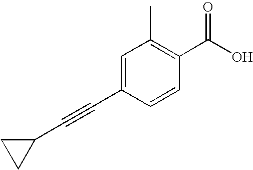

### Methyl-4-bromo-2-methylbenzoate

4-Bromo-2-methylbenzoic acid (5.0 g, 23 mmol) was suspended in methanol (30 mL). To the mixture was added a solution of HCl in diethylether (1.0M, 30 mL). The mixture was refluxed for 24 hours and concentrated to dryness. The residue was dissolved in EtOAc and washed with saturated sodium bicarbonate. The organic layer was washed with brine, dried (Na₂SO₄), filtered and concentrated under vacuum to give the desired compound (5.5 g) as a brown oil.

### 4-(cyclopropylethynyl)-2-methylbenzoic acid

Methyl 4-Bromo-2-methylbenzoate (1.0 g, 4.4 mmol) was dissolved in triethylamine (5 mL). To the mixture was added copper iodide (43 mg, 5 mol %), followed by PdCl₂(PPh₃)₂ (157 mg, 5 mol %) and ethynylcyclopropane (1.43 ml, 12 mmol). The mixture was heated in a sealed pressure tube at 80° C. for 3 hours. After completion of the reaction, the triethylamine was evaporated and the residue was dissolved in EtOAc and filtered through celite. The organic layer was washed with water, brine, and dried (Na₂SO₄), then filtered and concentrated under vacuum. The residue was purified by column chromatography on sililca gel using EtOAc-hexane (0-100% gradient) as eluent to give the desired product (630 mg). The product was dissolved in 10 mL of MeOH and 10 mL of 2N LiOH and the mixture was refluxed overnight. The MeOH was evaporated and the basic layer was washed with EtOAC, acidified, and re-extracted with EtOAC. The organic layer was washed with brine, dried (Na₂SO₄), filtered and concentrated under vacuum to give the desired product as a beige solid (461 mg). m/z 201 (M+1).

### Intermediate 7

### Preparation of 4-(cyclopropylethynyl)-2-fluorobenzoic acid

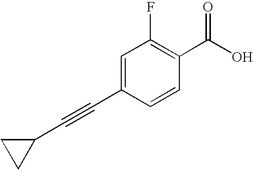

This compound was prepared using the same method as for 4-(3,3-dimethylbut-1-ynyl)-2-methylbenzoic acid, with the exception that cyclopopylacetylene was used as the alkyne coupling partner.

### Intermediate 8

### Preparation of 4-(3,3-dimethylbut-1-ynyl)-2-methoxybenzoic acid

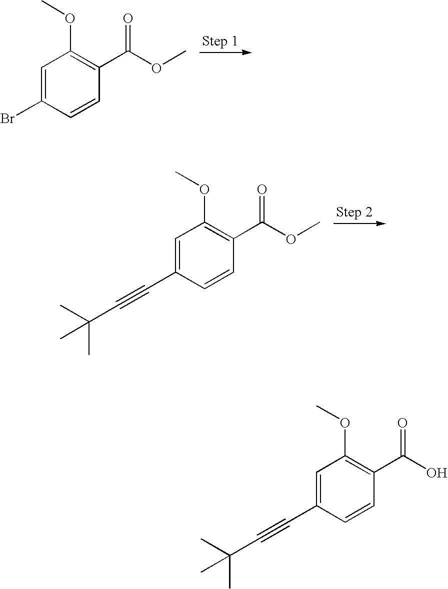

### Methyl 2-methoxy-4-(3,3-dimethylbut-1-ynyl)benzoate

A mixture of methyl 4-bromo-2-methoxybenzoate (1.2 g, 0.0049 mol), copper(I) iodide (0.093 g, 0.00049 mol), 3,3-dimethyl-1-butyne (0.70 mL, 0.0059 mol) and bis(triphenylphosphine)palladium(II) chloride (0.34 g, 0.00049 mol) in Et₃N (10 mL) was heated at 100° C. in a 50 mL sealed reaction vessel for 16 hours. After cooling, the mixture was filtered through celite and the filter cake was washed repeatedly with ethyl acetate. The filtrate was concentrated under vacuum and the residue was purified by column chromatography on silica gel to give a viscous oil (1.10 g, 91%).

### 2-Methoxy-4-(3,3-dimethylbut-1-ynyl)benzoic acid

A mixture of methyl 2-methoxy-4-(3,3-dimethylbut-1-ynyl)benzoate (1.10 g, 0.00447 mol), MeOH (20 mL), and 2N aqueous NaOH solution (5 mL) was stirred at 65° C. overnight. After allowing to cool, the mixture was concentrated under vacuum. The residue was treated with water, and extracted with hexane. The aqueous layer was acidified with 1N HCl to pH 2-3, and extracted with EtOAc (50 mL×3). The combined organic layers were washed with brine, dried (Na₂O₄), filtered and concentrated under vacuum to give the product (870 mg, 840%) as a white solid. LC-MS: 3.22 min, 233.4 (M+1).

### Intermediate 9

### Preparation of 4-(cylopropylethynyl)-2,6-difluorobenzoic acid

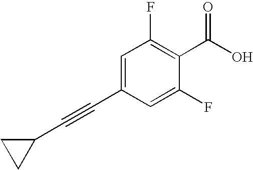

4-Bromo-2,6-difluoro-benzoic acid methyl ester (200 mg, 0.8 mmol) was dissolved in triethylamine (5 mL) and dichloropalladium(bis)triphenylphosphine (29 mg, 5 mol %) was added followed by copper iodide (8 mg, 5 mol %) and cyclpropylacetylene (0.09 mL, 0.96 mmol). The mixture was heated at reflux in a sealed tube for 1 hour. The mixture was cooled to room temperature and filtered through celite and evaporated. The residue was dissolved in dichloromethane and purified using a 0-100% EtOAc/Hexane gradient to give 178 mg (94%) of the ester compound. m/z=237 (M+1). The ester was hydrolyzed using the methodology outlined for 4-(cyclopentylethenyl)-2-fluorobenzoic acid to give the desired acid product.

### Intermediate 10

### Preparation of 4-(cyclopentylethynyl)-2-methylbenzoic acid

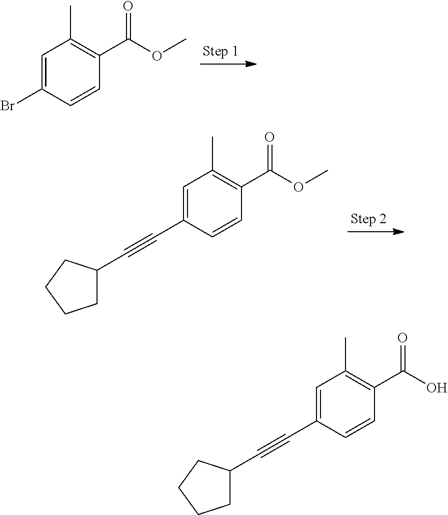

### Methyl 4-(cyclopentylethynyl)-2-methylbenzoate

Methyl 4-bromo-2-methylbenzoate (1.0 g, 4.4 mmol) was dissolved in triethylamine (5 mL). To the mixture was added copper iodide (43 mg, 5 mol %), followed by PdCl₂(PPh₃)₂ (157 mg, 5 mol %) and ethynylcyclopentane (0.75 mL, 5.3 mmol). The mixture was heated in a sealed pressure tube at 80° C. for 3 hours. After completion of the reaction, the triethylamine was evaporated and the residue was dissolved in EtOAc and filtered through celite. The organic layer was washed with water, brine, and dried (Na₂SO₄), then filtered and concentrated under vacuum. The residue was purified by column chromatography on sililca gel using EtOAc-hexane (0-100% gradient) as eluet to give the desired product.

### 4-(cyclopentylethynyl)-2-methylbenzoic acid

The product from step 1 was dissolved in 10 mL of MeOH and 10 mL of 2N LiOH and the mixture was refluxed overnight. The MeOH was evaporated and the basic layer was washed with EtOAC, acidified, and re-extracted with EtOAC. The organic layer was washed with brine, dried (Na₂SO₄), filtered and concentrated under vacuum to give the desired product (461 mg) as a beige solid. m/z=243 (M+1).

### Intermediate 11

### Preparation of 4-(3,3-dimethylbut-1-ynyl)-2-methylbenzoic acid

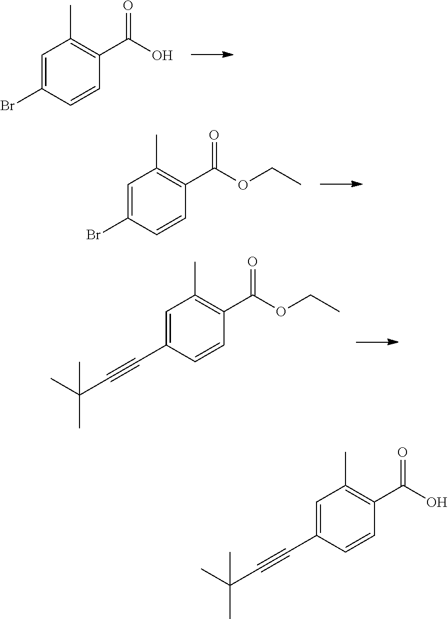

### Ethyl 4-bromo-2-methylbenzoate

4-bromo-2-methylbenzoic acid (10 g, 46.5 mmol) was dissolved in 200 mL EtOH, 5 mL conc H₂SO₄ was added and the mixture was refluxed for 18 h. The reaction volume was reduced in vacuo to 50 mL, and neutralized to pH 7 with satd. aqueous NaHCO₃ and extracted with EtOAc. The organic layer was dried (Na₂SO₄), filtered and the filtrate was concentrated to give the product (6.5 g) as an oil.

### Ethyl 2-methyl-4-(3,3-dimethylbut-1-ynyl)benzoate

Ethyl 4-bromo-2-methylbenzoate (6 g, 0.02 mol), 1-butyne, 3,3-dimethyl-(4.56 mL, 0.0382 mol) copper(I) iodide (0.47 g, 0.0025 mol) and bis(triphenylphosphine)palladium(II) chloride (3.46 g, 0.00493 mol) were placed in 40 mL triethylamine and stirred at room temperature overnight in a sealed tube. The reaction mixture was diluted with MeOH and filtered through celite. The filtrate was concentrated to a brown residue. The residue was purified by column chromatography on silica gel using hexanes as eluent to give the product (4.8 g, 42%) as a brown oil.

### 4-(3,3-dimethylbut-1-ynyl)-2-methylbenzoic acid

Ethyl 2-methyl-4-(3,3-dimethylbut-1-ynyl)benzoate (4.8 g, 0.020 mol) and lithium hydroxide (2.8 g, 0.058 mol) were placed in 3:1 mixture of methanol:water (80 mL) and heated at 60° C. for 3.5 h. TLC and LCMS indicated product formation. The reaction was cooled and concentrated in vacuo to a volume of 20 mL. The mixture was placed in an ice-water bath and acidified to pH 5 with conc. HCl. A white solid crashed out which was filtered and washed thoroughly with water. The solid was dried in a vacuum oven to give the product (4.1 g, 97%) as a solid. m/z=215.1 (M−1).

### Intermediate 12

### Preparation of (E)-2-fluoro-4-(3,3,3-trifluoroprop-1-enyl)benzoic acid

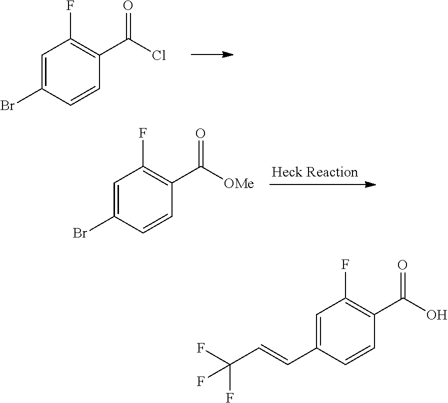

### Methyl-2-fluoro-4-bromobenzoate

4-Bromo-2-fluorobenzoyl chloride (45.0 g, 0.190 mol) was slowly added to a solution of methanol (31 mL, 0.76 mol) and triethylamine (53 mL, 0.38 mol) at 0° C. and the mixture was stirred at room temperature overnight. The mixture was washed with water, dried (Na₂SO₄), and concentrated to give a white solid.

### (E-2-Fluoro-4-(3,3,3-trifluoroprop-1-enyl)benzoic acid

A mixture of methyl-2-fluoro-4-bromobenzoate (5.0 g, 0.021 mol), tri-o-tolylphosphine (1.31 g, 0.00429 mol), tetra-N-butylammonium bromide (2.08 g, 0.00644 mol), potassium acetate (4.2 g, 0.043 mol), 3,3,3-trifluoropropl-ene (20 g, 0.2 mol), palladium acetate (0.24 g, 0.0011 mol) was sealed in a Parr instrument and stirred at 180° C. for 96 h. After cooling, the reaction mixture was filtered through Celite and the filtrate was partitioned between EtOAc and 1 N aq. HCl. The organic layer was separated and washed with brine, dried (Na₂SO₄) and concentrated. The residue was chromatographed with hexane-EtOAc(5% AcOH) (0 to 60%) to give the product as a white solid. LC-MS: t=2.98 min, m/z=233.2 (M−1).

### Intermediate 13

### Preparation of (Z)-2-fluoro-4-(3,3,3-trifluoroprop-1-enyl)benzoic acid

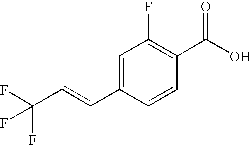

### tert-Butyl 4-bromo-2-fluorobenzoate

To a stirred solution of 4-bromo-2-fluorobenzoic acid (3.0 g, 0.014 mol) in THF (50 mL) at 0° C. was added DMF (0.1 mL) and oxalyl chloride (1.5 mL, 0.018 mol). The mixture was stirred at 0° C. for 1 h and then warmed to rt. The solvent was removed under reduced pressure. The obtained acid chloride was added to a mixture of tert-butyl alcohol (5.0 g, 0.067 mol), pyridine (10 mL), and CH₂Cl₂ (50 mL) at 0° C. The mixture was stirred at rt for 3 h, and then at 50° C. overnight. The mixture was washed with water, 2 N NaOH, and brine, dried (MgSO₄), and concentrated under vacuum. The residue was purified by column to give a colorless oil (1.5 g, 45%).

### tert-Butyl 2-fluoro-4-formylbenzoate

To a stirred solution of tert-butyl 4-bromo-2-fluorobenzoate (1.5 g, 5.45 mmol) in THF (70 mL) at −100° C. under argon was carefully added BuLi (2.5 M in hexane, 2.3 mL, 5.75 mmol). The mixture was kept at −100° C. to −80° C. for 1 h and then DMF (1.0 mL) in THF (5 mL) was added. After 1 h, the mixture was warmed to 0° C. and quenched by adding sat. aq NH₄Cl, and extracted with EtOAc. The organic layer was separated, washed with brine, dried (MgSO₄), and concentrated under vacuum. The residue was purified by column chromatography on silica gel using EtOAc/hexane (0-10%) as eluent to give the product (750 mg, 61%) as a white solid.

### (E)-tert-Butyl 2-fluoro-4-(3,3,3-trifluoroprop-1-enyl)benzoate and (Z)-tert-butyl 2-fluoro-4-(3,3,3-trifluoroprop-1-enyl)benzoate

Molecular sieves 4 Å (powder, 24 g) was added to a 1 M solution of TBAF in THF (30 mL, 30 mmol), and the mixture was stirred at room-temperature overnight under an argon atmosphere. To the mixture were added a solution of tert-butyl 2-fluoro-4-formylbenzoate (750 mg, 0.0033 mol) and 2,2,2-trifluoroethyldiphenylphosphine oxide (1.9 g, 0.0067 mol) in THF (30 mL). After the mixture was stirred for 2 h it was filtered. The filtrate was concentrated under vacuum and water (120 mL) was added. The mixture was extracted with AcOEt and the organic extract was washed with brine, dried (Na₂SO₄), and concentrated under vacuum. The residue was purified by column chromatography on silica gel using AcOEt-hexane (0-15%) as eluent to give (E)-tert-butyl 2-fluoro-4-(3,3,3-trifluoroprop-1-enyl)benzoate as a colorless oil (620 mg, 64%), followed by (Z)-tert-butyl 2-fluoro-4-(3,3,3-trifluoroprop-1-enyl)benzoate as a colorless oil (80 mg, 8%).

### (E)-2-fluoro-4-(3,3,3-trifluoroprop-1-enyl)benzoic acid

A solution of (E)-tert-butyl 2-fluoro-4-(3,3,3-trifluoroprop-1-enyl)benzoate (500 mg, 0.002 mol) in CH₂Cl₂ (10 mL) and TFA (1.0 mL) was stirred at room temperature for 2 h. The solvent was removed under reduced pressure to give a white solid. LC-MS: 2.99 min, 233.2 (M−1).

### (Z)-2-fluoro-4-(3,3,3-trifluoroprop-1-enyl)benzoic acid

A solution of (Z)-tert-butyl 2-fluoro-4-(3,3,3-trifluoroprop-1-enyl)benzoate (35 mg, 0.12 mmol) in CH₂Cl₂ (5 mL) and TFA (0.5 mL) was stirred at room temperature for 2 h. The solvent was removed under reduced pressure to give a white solid. LC-MS: 2.86 min, 233.2 (M−1).

### Intermediate 14

### Preparation of 4-(3,3-dimethylbut-1-ynyl)-2-fluorobenzoic acid

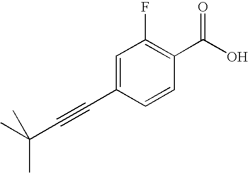

### 4-Bromo-2-fluoro-benzoic acid methyl ester

4-Bromo-2-fluorobenzoic acid (10 g, 0.04 mol) was suspended in 1,2-dichloroethane (60 mL, 0.8 mol) to which was added thionyl chloride (10 mL, 0.1 mol) followed by a drop of DMF. The mixture was heated to reflux for 1 hour. Excess thionyl chloride and 1,2-dichloroethane were stripped off and the crude product was treated with methanol (50 mL, 1 mol) and heated to reflux for an hour. The mixture was concentrated to dryness, dissolved in dichloromethane, treated with cold sat. sodium bicarbonate solution. The organic layer was dried, then concentrated under vacuum to obtain the title compound as a white solid.

### 4-(3,3-Dimethyl-but-1-ynyl)-2-fluoro-benzoic acid methyl ester

In a sealed reaction vessel was added bis(triphenylphosphine)palladium(II) chloride (1.03 g, 0.00145 mol) N,N-diisopropylethylamine (9.0 mL, 0.050 mol), copper(I) iodide (0.353 g, 0.00186 mol), and 1,4-dioxane (70 mL, 0.8 mol) in that order. 1-butyne, 3,3-dimethyl-(6.1 mL, 0.050 mol) was added and the vessel was allowed to stir at room temperature for 24 hrs. The mixture was filtered through celite and concentrated in vacuo. The mixture was chromatographed using a 0-20% ethyl acetate:hexanes gradient. The combined pure fractions were reduced in vacuo and dried on high vacuum to yield a light brown solid.

### 4-(3,3-Dimethyl-but-1-ynyl)-2-fluoro-benzoic acid

Methyl 2-fluoro-4-(3,3-dimethylbut-1-ynyl)benzoate (8.2 g, 0.035 mol) was suspended in a 3:1 mixture of H₂O and methanol to which was added lithium hydroxide (2.5 g, 0.10 mol) all at once and the mixture was agitated over-night at ambient temperature. The mixture was then concentrated to ¾ the volume and acidified with 1N HCl until the pH read just acidic. The white precipitate was filtered, washed with water and vacuum dried at 80° C. for several hours. m/z=218.9 (M−1).

### Intermediate 15

### Preparation of 2-chloro-4-(3,3-dimethylbut-1-ynyl)benzoic acid

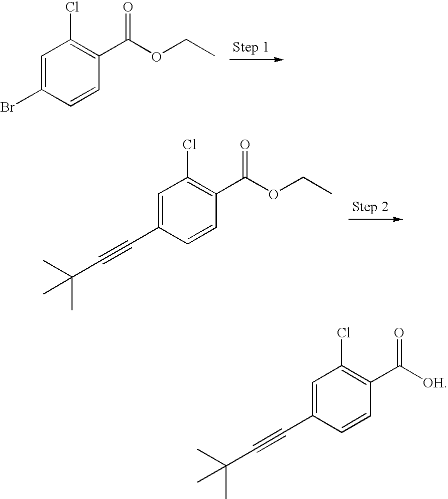

### Methyl 2-chloro-4-(3,3-dimethylbut-1-ynyl)benzoate

A mixture of methyl 4-bromo-2-chlorobenzoate (400 mg, 0.0016 mol), copper(I) iodide (30 mg, 0.00016 mol), 3,3-dimethyl-1-butyne (0.29 mL, 0.0024 mol) and bis(triphenylphosphine)palladium(II) chloride (110 mg, 0.00016 mol) in Et₃N (5 mL) and DMF (2 mL) was heated at 100° C. in a 50 ml sealed reaction vessel for 32 hours. After cooling, the mixture was filtered through celite and the filter cake was washed repeatedly with ethyl acetate. The organic phase was washed with brine, dried (Na₂SO₄), and concentrated under vacuum. The residue was purified by column chromatography on silica gel to give the product (330 mg, 82%) as a light yellow oil.

### 2-Chloro-4-(3,3-dimethylbut-1-ynyl)benzoic acid

A mixture of methyl 2-chloro-4-(3,3-dimethylbut-1-ynyl)benzoate (330 mg, 0.0013 mol), 2N aq. NaOH (3.0 mL), THF (5 mL), and MeOH (5 mL) was stirred at rt for 5 h. The mixture was concentrated under vacuum and the residue was treated with water and acidified with 1N HCl to pH 2-3, and extracted with EtOAc. The organic layer was washed with brine, dried (Na₂SO₄), and concentrated under vacuum to give the product (305 mg, 98%) as a white solid. LC-MS: 3.56 min, 234.9 & 236.9 (M−1).

### Intermediate 16

### Preparation of 2-chloro-4-(cyclopropylethynyl)benzoic acid

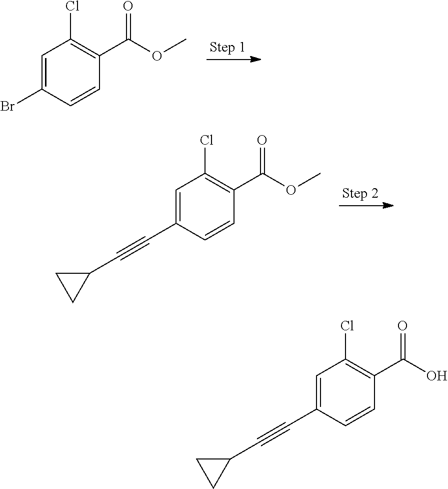

### Methyl 2-chloro-4-(2-cyclopropylethynyl)benzoate

A mixture of methyl 4-bromo-2-chlorobenzoate (450 mg, 0.0018 mol), copper(I) iodide (34 mg, 0.00018 mol), 70% solution of cyclopropylacetylene (0.26 g, 0.0027 mol) in toluene and bis(triphenylphosphine)palladium(II) chloride (130 mg, 0.00018 mol) in Et₃N (5 mL) and DMF (3 mL) was heated at 100° C. in a 50 mL sealed reaction vessel for 36 hours. After cooling, the mixture was filtered through celite and the filter cake was washed repeatedly with ethyl acetate. The organic phase was washed with brine, dried (Na₂SO₄), filtered and concentrated under vacuum. The residue was purified by column chromatography on silica gel to give the product (320 mg, 76%) as a brown oil.

### 2-Chloro-4-(2-cyclopropylethynyl)benzoic acid

A mixture of methyl 2-chloro-4-(2-cyclopropylethynyl)benzoate (310 mg, 0.0013 mol), 2N aq. NaOH (3.0 mL), THF (5 mL), and MeOH (5 mL) was stirred at rt for 5 h. The mixture was concentrated under vacuum and the residue was treated with water and acidified with 1N HCl to pH 2-3, and extracted with EtOAc. The organic layer was washed with brine, dried (Na₂SO₄), filtered and concentrated under vacuum to give the product (270 mg, 93%) as a yellow solid. LC-MS: 3.18 min, 218.9 & 220.9 (M−1).

### Intermediate 17

### Preparation of (E)-2-chloro-4-(3,3,3-trifluoroprop-1-enyl)benzoic acid

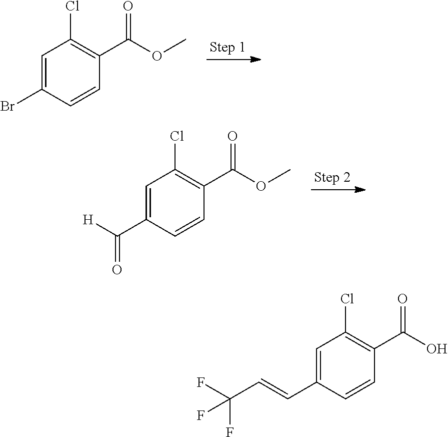

### Methyl 2-chloro-4-formylbenzoate

A slow stream of CO was passed into a suspension of methyl 4-bromo-2-chlorobenzoate (1.50 g, 0.00601 mol), bis(triphenylphosphine)palladium(II) chloride (80 mg, 0.0001 mol), sodium formate (613 mg, 0.00902 mol), and dry DMF (10 mL). The mixture was vigorously stirred at 110° C. for 2 h. After cooling, the mixture was treated with aqueous Na₂CO₃ solution and extracted with EtOAc. The extract was washed with brine, dried (Na₂SO₄), and concentrated. The residue was chromatographed on silica gel with AcOEt-hexane to give the product as a colorless oil (becomes a white solid when stored in a refrigerator).

### 2-Chloro-4-((E)-3,3,3-trifluoroprop-1-enyl)benzoic acid

4 Å molecular sieves (powder, 16 g) was added to a 1 M solution of TBAF in THF (20 mL, 20 mmol), and the mixture was stirred at room-temperature overnight under an argon atmosphere. To the mixture were added a solution of methyl 2-chloro-4-formylbenzoate (210 mg, 0.0010 mol) and 2,2,2-trifluoroethyldiphenylphosphine oxide (600 mg, 0.0021 mol) in THF (15 mL). After the mixture was stirred for 2 h, the molecular sieves were removed by filtration. The filtrate was concentrated and water (120 mL) was added. The mixture was extracted with AcOEt. The organic extract was washed with brine, dried (Na₂SO₄), and concentrated. The residue was chromatographed on silica gel with AcOEt [1% HOAc]-hexane to give the product as a white solid. LC-MS: t=3.12 min, m/z=248.9 & 250.9 (M−1).

### Intermediate 18

### Preparation of 4-(cyclopropylethynyl)-2-(methylsulfonyl)benzoic acid

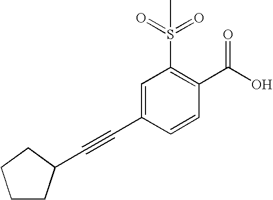

4-Bromo-2-methanesulfonyl acid methyl ester (250 mg, 0.85 mmol) was dissolved in triethylamine (5 mL). To the mixture was added copper iodide (9.0 mg, 5 mol %), followed by PdCl₂(PPh₃)₂ (32 mg, 5 mol %) and ethynylcyclopentane (0.135 ml, 1.0 mmol). The mixture was heated in a sealed pressure tube at 80° C. for 3 hours. After reaction completion, the triethylamine was removed under vacuum and the residue was dissolved in EtOAc and filtered through celite. The organic layer was washed with water, brine, and dried (Na₂SO₄). After filtration and concentration under vacuum, the residue was purified by column chromatography on sililca gel using EtOAc-hexane (0-100% gradient) as eluent to give methyl 4-(cyclopropylethynyl)-2-(methylsulfonyl)benzoate (240 mg). The product was dissolved in 10 mL of MeOH and 10 mL of 2N LiOH and the mixture was refluxed overnight. The MeOH was evaporated and the basic layer was washed with EtOAC, acidified, and re-extracted with EtOAc. The organic layer was washed with brine, dried (Na₂SO₄), filtered and concentrated under vacuum to give the product (165 mg) as a beige solid. m/z 293 (M+1).

### Intermediate 19

### Preparation of 4-(3,3-dimethylbut-1-ynyl)-2,6-difluorobenzoic acid

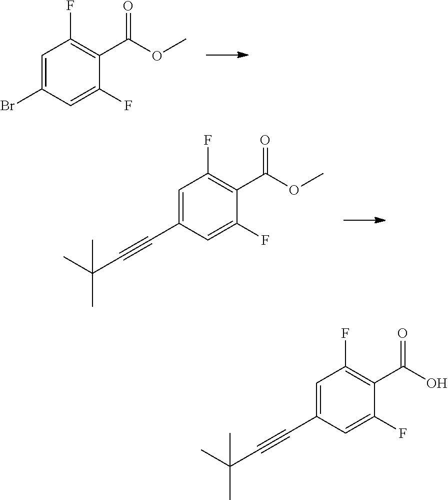

### Methyl 4-bromo-2,6-difluorobenzoate

4-bromo-2,6-difluorobenzoic acid (7 g, 0.03 mol) methyl iodide (2.8 mL, 0.045 mol) and potassium carbonate (12.22 g, 0.08842 mol) were placed in 100 mL acetone in a sealed tube and heated at 50° C. overnight. The reaction was cooled, partitioned between EtOAc and water. The organic layer was dried (Na₂SO₄), filtered and the filtrate was concentrated to an oil. Purification by column chromatography on silica gel gave the product (1.3 g, 177%) along with 5 g of starting material.

### Methyl 2,6-difluoro-4-(3,3-dimethylbut-1-ynyl)benzoate

Methyl 2,6-difluoro-4-(3,3-dimethylbut-1-ynyl)benzoate. Methyl 4-bromo-2,6-difluorobenzoate (1.3 g, 0.0052 mol), 1-butyne, 3,3-dimethyl-(0.96 mL, 0.0080 mol), copper(I) iodide (200 mg, 0.001 mol) and bis(triphenylphosphine)palladium(II) chloride (0.73 g, 0.0010 mol) were placed in 50 mL triethylamine and stirred in a sealed tube at room temperature for 20 h. The reaction was diluted with MeOH and filtered through celite. The filtrate was concentrated to an oil and purified by column chromatography om silica gel using hexane as eluent to give the product (1.0 g, 80%) as a yellow oil.

### 4-(3,3-dimethylbut-1-ynyl)-2,6-difluorobenzoic acid

Methyl 2,6-difluoro-4-(3,3-dimethylbut-1-ynyl)benzoate (1.0 g, 0.004 mol) and lithium hydroxide (0.57 g, 0.012 mol) were placed in a 3:1 mixture of methanol:water (60 mL) and heated at 60° C. for 3.5 h. TLC and LCMS indicated product formation. The reaction was cooled and concentrated in vacuo to a volume of 20 mL. The mixture was placed in an ice-water bath and acidified to pH 5 with coc. HCl. A white solid crashed out which was filtered and washed thoroughly with water. The solid was dried in the vacuum oven to give the product (0.79 g, 84%) as a solid. m/z=237.1 (M−1).

### Intermediate 20

### Preparation of 4-(3,3-dimethyl-1-ynyl)-2-fluoro-3-methoxybenzoic acid

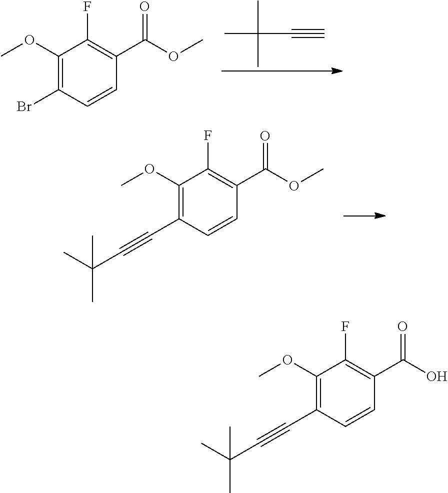

### Methyl 2-fluoro-3-methoxy-4-(3,3-dimethylbut-1-ynyl)benzoate

Methyl 4-bromo-2-fluoro-3-methoxybenzoate (960 mg, 3.5 mmol), copper(I) iodide (70 mg, 0.4 mmol), and bis(triphenylphosphine)palladium(11) chloride (300 mg, 0.4 mmol) were suspended in Et₃N (10 mL) and DMF (4 mL). 1-Butyne, 3,3-dimethyl-(440 mg, 5.2 mmol) was added and the mixture was heated from room temperature to 100° C. in a sealed tube for 60 h. Solvent was removed, and the residue was dissolved in EtOAc, washed with water, brine and dried over Na₂SO₄. Purified by column chromatography on silica gel to give the product as a light yellow oil (760 mg, 79%).

### 2-Fluoro-3-methoxy-4-(3,3-dimethylbut-1-ynyl)benzoic acid

Methyl 2-fluoro-3-methoxy-4-(3,3-dimethylbut-1-ynyl)benzoate (760 mg, 2.7 mmol) was dissolved in MeOH (10 mL), NaOH (in 10 mL water) was added and stirred at 50° C. for 1 h. Solvent was removed, more water was added, neutralized by HCl till pH ˜2, white solid thus formed was filtered out, dried in vacuum oven (at 65° C.). Product was obtained as a white solid (760 mg, 93%).

### Intermediate 21

### Preparation of 2-chloro-4-(3,3-dimethylbut-1-ynyl)-5-fluorobenzoic acid

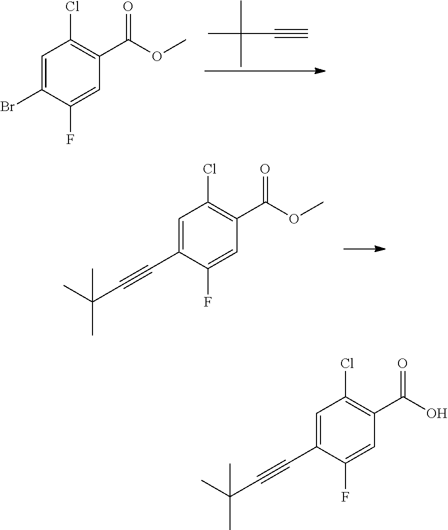

### Methyl 2-chloro-5-fluoro-4-(3,3-dimethylbut-1-ynyl)benzoate

Methyl 4-bromo-2-chloro-5-fluorobenzoate (9.1 g, 32 mmol), copper(I) iodide (0.62 g, 3.2 mmol) and bis(triphenylphosphine)palladium(II) chloride (2.3 g, 3.2 mmol) were suspended in Et₃N (100 mL) and DMF (40 mL), 1-butyne, 3,3-dimethyl-(4.1 g, 48 mmol) was added and then the mixture was stirred at 100° C. in a sealed tube for 40 h. Solvent was removed, residue was dissolved in EtOAc, washed by water and brine, purified by column, product was obtained as a light yellow oil (6.1 g, 69%).

### 2-Chloro-5-fluoro-4-(3,3-dimethylbut-1-ynyl)benzoic acid

Methyl 2-chloro-5-fluoro-4-(3,3-dimethylbut-1-ynyl)benzoate (6.1 g, 22 mmol) was dissolved in MeOH (30 mL), sodium hydroxide (1.3 g, 33 mmol) (in 20 mL, water) was added and stirred at 60° C. overnight. Solvent was removed, residue was dissolved in water, neutralized by HCl till pH<2, extracted by EtOAc, washed by water, brine and dried over Na₂SO₄. Product was obtained as a beige solid (3.1 g, 52%).

### Intermediate 22

### Preparation of (E)-4-(3,3-dimethylbut-1-enyl)-2-methylbenzoic acid

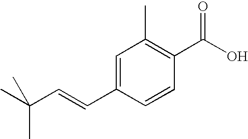

### 4-Bromo-2-methyl-benzoic acid methyl ester

To a suspension of 4-bromo-2-methylbenzoic acid (10.0 g, 0.0465 mol) in 1,2-dichloroethane (60 mL, 0.8 mol) was added thionyl chloride (28 g, 0.23 mol) and the mixture heated to reflux for 1 hour. The mixture was concentrated to dryness and vacuum dried. The crude acid chloride was dissolved in methanol (100 mL, 2 mol) and the solution was treated with triethylamine (4.7 g, 0.046 mol). The mixture was heated to reflux for an hour and then concentrated to dryness. The crude ester was dissolved in EtOAc, washed consecutively with sat. sodium bicarbonate solution and water. The organic phase was dried and concentrated to obtain the title ester.

### (E)-4-(3,3-dimethylbut-1-enyl)-2-methylbenzoic acid methyl ester

A mixture of methyl 4-bromo-2-methylbenzoate (10.0 g, 0.0436 mol), tri-o-tolylphosphine (1.31 g, 0.00429 mol), cesium carbonate (6.99 g, 0.0214 mol), tetra-N-butylammonium chloride (1.79 g, 0.00644 mol), 1-butene, 3,3-dimethyl-(20 g, 0.2 mol), palladium acetate (0.24 g, 0.0011 mol) was sealed in a glas vessel and stirred at 150° C. for 96 h. After cooling, the reaction mixture was filtered through Celite and the filtrate was partitioned between EtOAc and water. The organic layer was separated and washed with brine, dried (Na₂SO₄) and concentrated. The residue was chromatographed with hexane-EtOAc to give the title compound as a white solid.

### (E-4-(3,3-dimethyl-but-1-enyl)-2-methylbenzoic acid

A solution of (E)-4-(3,3-dimethylbut-1-enyl)-2-methylbenzoic acid methyl ester (6.5 g, 0.028 mol) and lithium hydroxide (3.4 g, 0.14 mol) in a mixture of methanol (50 mL, 1 mol) and water (150 mL, 8.3 mol) was heated to reflux for 3 hours. Most of the methanol was stripped of and the aqueous solution was carefully acidified with conc. HCl. The white precipitate was filtered, washed with water and vacuum dried. m/z=217.1 (M−1).

### Intermediate 23

### Preparation of 3-methyl-4-(3,3,3-trifluoroprop-1-ynyl)benzoic acid

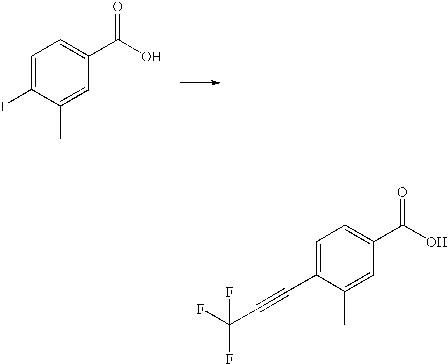

The method is based upon a procedure detailed by Yoneda et al in Bulletin Chemical Society Japan 1990, 63, 2124-2126. A solution of n-butyl lithium (2.5M in hexanes; 1 eq) was added carefully to a solution of 3,3,3-trifluoroprop-1-yne (1 eq) in THF at 78° C. under nitrogen. The mixture was stirred at −78° C. for 30 min then a solution of ZnCl₂ (3 eq) in THF was added slowly. The mixture was allowed to warm to room temperature, stirred for 30 min then Pd(Ph₃P)₄ (5 mol %) was added, followed by 4-iodo-3-methylbenzoic acid (0.5 eq). The mixture was heated to 50° C. and stirred for 15 h, then heated further to 80° C. for 5 h, and finally at 100° C. overnight. After allowing to cool to room temperature the mixture was concentrated under vacuum to a crude residue. The residue was purified by column chromatography on silica gel to give the product as a solid. m/z=227 (M−1).

### Preparation of Amine Building Blocks

### Intermediate 24

### Preparation of 2-((cyclopropylmethoxy)methyl)-2,3-dihydrobenzo[b][1,4]dioxin-6-amine

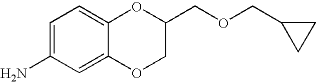

### 2-((cyclopropylmethoxy)methyl)-2,3-dihydro-6-nitrobenzo[b][1,4]dioxine

(2,3-dihydro-6-nitrobenzo[b][1,4]dioxin-2-yl)methanol (500 mg, 0.002 mol) and sodium hydride (0.28 g, 0.0070 mol) were placed in a flask under nitrogen. The flask was placed in an ice bath and 25 mL DMF was added. The reaction was stirred at 0° C. for 10 minutes and then (chloromethyl)cyclopropane (440 μL, 0.0048 mol) was added. The mixture was warmed to room temp over 20 min then tetra-N-butylammonium bromide (1.53 g, 0.00475 mol) was added to the mixture and the reaction was stirred at room temperature overnight. The reaction was partitioned between EtOAc and water. The organic layer was separated, washed with brine, dried (Na₂SO₄), filtered and the filtrate was concentrated under vacuum to an oil. The oil was purified by column chromatography on silica gel using EtOAc/hexanes (10%) as eluent to give a yellow solid (0.33 g, 50%) as a solid. m/z=266 (M+1).

### 2-((cyclopropylmethoxy)methyl)-2,3-dihydrobenzo[b][1,4]dioxin-6-amine

2-((cyclopropylmethoxy)methyl)-2,3-dihydro-6-nitrobenzo[N][1,4]dioxine (0.33 g, 0.0012 mol) was dissolved in 20 mL dioxane. Sodium dithionite (2.2 g, 0.013 mol was suspended in water (4 mL) and NH₄OH (2 mL) and then added to the dioxane solution. The reaction was stirred at room temp for 6 hrs. The mixture was filtered through a filter paper and the filtrate concentrated under vacuum to a white solid. The solid was suspended in 10% EtOAc/hexanes and filtered. The filtrate was concentrated to a white solid and used for the next reaction without further purification. Yield of the title compound is 0.29 g (98%). m/z=235.8 (M+1).

### Intermediate 25

### Preparation of 1-methyl-1,2,3,4-tetrahydroquinolin-7-ylamine

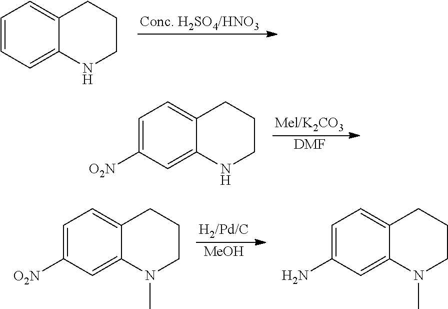

### 7-Nitro-1,2,3,4-tetrahydroquinoline

To a solution of 1,2,3,4-tetrahydroquinoline (6.5 g, 0.049 mol) in conc. sulfuric acid (118 mL) at 0° C. was added a solution of con. nitric acid (4.9 mL) in cone. Sulfuric acid (12 mL) drop-wise over 3 hours so as to maintain the temperature <5° C. The reaction mixture was then poured onto crushed ice and neutralized with solid potassium carbonate. The mixture was extracted with EtOAc (2×500 mL), the combined organic extracts were washed with water, dried and concentrated to give the crude product which was purified by column chromatography on silica-gel using EtOAc/hexane as eluent to obtain the title compound as an orange solid.

1-Methyl-7-nitro-1,2,3,4-tetrahydroquinoline. To a solution of the 7-nitro-1,2,3,4-tetrahydroquinoline (4.5 g, 25.25 mmol) in DMF (50 mL) was added potassium carbonate (15 g) followed by iodomethane (5.54 g, 39.0 mMol) and the mixture was agitated overnight at ambient temperature. The mixture was poured onto water and extracted with ether (3×200 mL). The combined ethereal extracts were washed with brine, dried and concentrated to give the crude product which was purified by column chromatography on silica-gel to obtain the title compound as an orange liquid.

### 1-Methyl-1,2,3,4-tetrahydroquinolin-7-ylamine

A mixture of the 1-methyl-7-nitro-1,2,3,4-tetrahydroquinoline (4.0 g, 20.81 mMol), Pd/C (2 g) in methanol (100 mL) was hydrogenated at 10 PSI for 2 hours. The catalyst was filtered off, and the filtrate was concentrated under vacuum to give the crude product which was used as such without further purification.

### Intermediate 26

### Preparation of 3,4-dihydro-2H-benzo[b]I[1,4]oxazin-6-amine

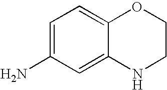

### 6-Nitro-2H-benzo[b][1,4]oxazin-3(4H)-one

Bromoacetyl bromide (4.84 g, 24 mmol, in 10 mL CHCl₃) was added dropwise to the suspension of 2-amino-4-nitrophenyl (3.08 g, 20 mmol), benzyltriethylammonium chloride (TEBA, 4.56 g, 20 mmol) and NaHCO₃ (6.72 g, 80 mmol) in 30 mL CHCl₃ with ice bath cooling. The mixture was stirred with ice bath cooling for 1.5 h then at 60° C. overnight. The solvent was removed under vacuum and water was added to the residue. A solid precipitated which was filtered and dried under vacuum to give the product (3.45 g, 89%) as a beige solid.

### 6-Amino-2H-benzo[b][1,4]oxazin-3(4H)-one

Pd/C (10%) was added to a suspension of 6-nitro-2H-benzo[b][1,4]oxazin-3(4H)-one (1.5 g) in MeOH (20 mL) and the reaction mixture was stirred under an atmosphere of hydrogen overnight. The mixture was filtered through celite and the filtrate was concentrated under vacuum to give the product (0.705 g, 56%) as a beige solid.

### 3,4-Dihydro-2H-benzo[b][1,4]oxazin-6-amine

6-Amino-2H-benzo[b][1,4]oxazin-3(4H)-one (590 mg, 3.6 mmol) was added to a THF solution of borane tetrahydrofuran complex (9 mL, 1M solution) and the reaction mixture was refluxed for 2.5 h. EtOH (2 mL) was added and stirred at 70° C. for 1 h before 1 mL HCl (conc.) was added. The mixture was stirred at 80° C. overnight then the volatiles were removed under vacuum to leave a crude reside. The residue was dissolved in water, NaOH was added until pH ˜10, and the mixture was extracted with CH₂Cl₂. The organic phase was washed with water and the solvent was removed under vacuum. The residue was purified by column chromatography on silica gel to give the product (274 mg, 51%) as a colorless oil.

### Intermediate 27

### Preparation of 3,4-dihydro-2H-benzo[b][1,4]oxazin-7-amine

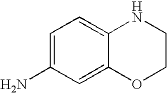

The above was prepared using the same procedure as for 3,4-dihydro-2H-benzo[b][1,4]oxazin-6-amine, except 2-amino-5-nitrophenol was used as starting material.

### Intermediate 28

### Preparation of 4-methyl-3,4-dihydro-2H-benzo[b][1,4]oxazin-7-amine

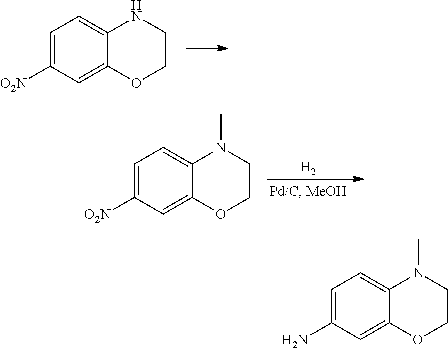

Potassium carbonate (800 mg, 6 mmol) and methyl iodide (1.3 g, 9 mmol) were added to a solution of 3,4-dihydro-7-nitro-2H-benzo[b][1,4]oxazine (540 mg, 3 mmol) in DMF (10 mL). The reaction mixture was stirred at room temperature overnight. Sodium hydride (100 mg, 95%) and methyl iodide (1.0 g) were added and the reaction mixture was stirred at room temperature overnight. The solvent was removed under vacuum and the residue was suspended in water. A solid precipitated which was filtered and washed with water. The bright yellow solid was then suspended in MeOH (20 mL) and Pd/C (10%) was added. The suspension was stirred under an atmosphere of hydrogen overnight, then filtered through celite and the filtrate concentrated under vacuum to give the product (470 mg) as a purple oil.

### Intermediate 29

### Preparation of 6-amino-2,2-dimethyl-2H-benzo[b][1,4]oxazin-3(4H)-one and 2,2-dimethyl-3,4-dihydro-2H-benzo[b][1,4]oxazin-6-amine

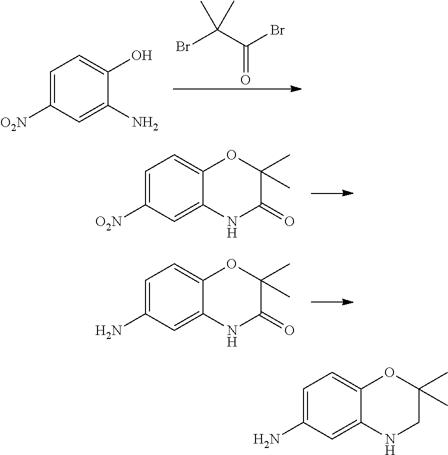

### 2,2-Dimethyl-6-nitro-2H-benzo[b][1,4]oxazin-3(4H)-one

2-Bromoisobutyryl bromide (10.3 g, 45 mmol, in 20 mL chloroform) was added dropwise to a suspension of 2-amino-4-nitrophenol (4.62 g, 30 mmol) and sodium bicarbonate (10.1 g, 120 mmol) in chloroform (250 mL) under nitrogen with ice bath cooling. The reaction mixture was stirred from 0° C. to room temperature overnight then the solvent was removed under vacuum. The residue was suspended in DMF (150 mL) and potassium carbonate (5.98 g, 45 mmol) was added, then the reaction mixture was stirred at 80° C. overnight. The solvent was removed under vacuum and water was added to the residue. The precipitate that emerged was filtered and dried under vacuum to give the product (4.5 g, 68%) as a light brown solid.

The remainder of the synthesis (hydrogenation of the nitro group and then borane reduction of the lactam) was performed using the general procedure described for 3,4-dihydro-2H-benzo[b][1,4]oxazin-6-amine.

### Intermediate 30

### Preparation of 7-amino-2,2-dimethyl-2H-benzo[b][1,4]oxazin-3(4H)-one and 2,2-dimethyl-3,4-dihydro-2H-benzo[b][1,4]oxazin-7-amine

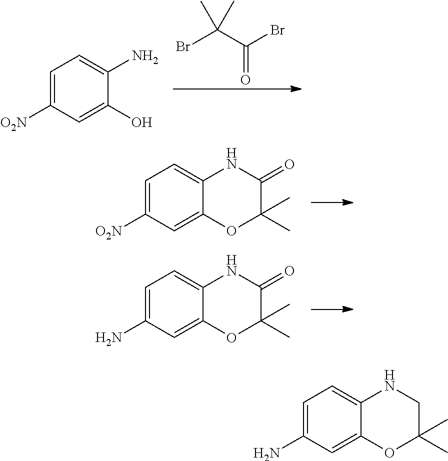

The above was prepared using the same procedures for 6-amino-2,2-dimethyl-2H-benzo[b][1,4]oxazin-3(4H)-one and 2,2-dimethyl-3,4-dihydro-2H-benzo[b][1,4]oxazin-6-amine except 2-amino-5-nitrophenol was used as the starting material.

### Intermediate 31

### Preparation of 6-chloro-3,4-dihydro-2H-benzo[b][1,4]oxazin-7-amine

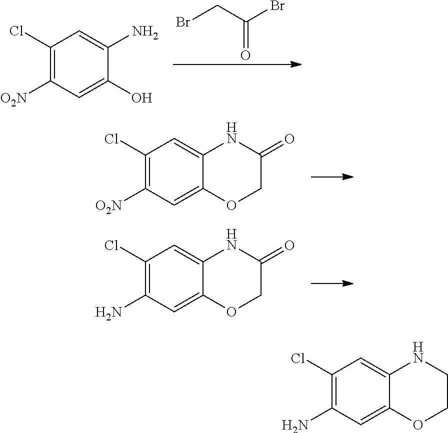

### 6-chloro-7-nitro-2H-benzo[b][1,4]oxazin-3(4H)-one

This compound was prepared using the general procedure described for 2,2-Dimethyl-6-nitro-2H-benzo[b][1,4]oxazin-3(4H)-one above except 2-amino-4-chloro-5-nitrophenol was used as starting material.

### 7-Amino-6-chloro-2H-benzo[b][1,4]oxazin-3(4H)-one

Stannous chloride dihydrate (30 g, 0.13 mol) was added in portion to a solution of 6-chloro-7-nitro-2H-benzo[b][1,4]oxazin-3(4H)-one (6.7 g, 0.026 mol) in DMF (100 mL) with ice bath cooling. The mixture was allowed to warm to room temperature and was then stirred overnight. EtOAc (300 mL) and MeOH (300 mL) were added to the reaction mixture, Et₃N was added until pH>8 and the resulting suspension was filtered through celite. The solvent was removed under vacuum and the residue was suspended in water, extracted with EtOAc, dried (Na₂SO₄), filtered and concentrated under vacuum. The residue was triturated with ether to give the product (2.5 g, 45%) as a yellow solid.

### 6-chloro-3,4-dihydro-2H-benzo[b][1,4]oxazin-7-amine

Borane reduction performed using general procedure described above for 3,4-dihydro-2H-benzo[b][1,4]oxazin-6-amine except 7-Amino-6-chloro-2H-benzo[b][1,4]oxazin-3(4H)-one was used as starting material.

### Intermediate 32

### Preparation of (6-Amino-3H-imidazo[4,5-b]pyridin-2-yl)-methanol

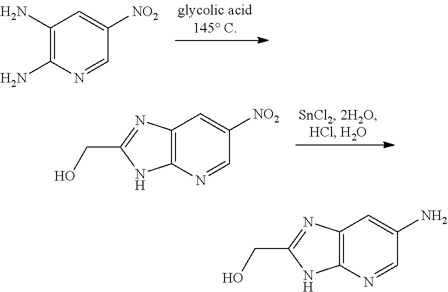

### (6-Nitro-3H-imidazo[4,5-b]pyridin-2-yl)-methanol

Solid 2,3-Diamino-5-nitropyridine (prepared according to J. Med. Chem. 1997, 40, 3679-3686; 610 mg, 0.0040 mol) and solid glycolic acid (750 mg, 0.0099 mol) were combined in a sealed tube (left open) and heated to 145° C. and stirred for approx. 30-45 min (solid fuses together, liquefies then re-solidifies). After allowing to cool to rt the solid was extracted with 1N HCl. The aqueous mixture was concentrated under vacuum to leave a crude solid that was basified using conc. NH₃ solution. The ammonia solution was concentrated under vacuum to leave a crude solid that was dry-loaded on to silica and purified by column chromatography (using the ISCO system) to give a solid (450 mg) that was used directly in the next step.

### (6-Amino-3H-imidazo[4,5-b]pyridin-2-yl)-methanol

Stannous chloride dihydrate (1.6 g, 0.0070 mol) was added in one portion to a stirred solution of (6-Nitro-3H-imidazo[4,5-b]pyridin-2-yl)-methanol (450 mg, 0.0023 mol) in 10% aqueous hydrochloric acid (20 mL) at 50° C. The mixture was stirred at 50° C. for approx. 2 hours then allowed to cool to room temperature. The mixture was cooled further to 0° C. and then basified to ca. pH 8 using conc. NH₃ solution. The aqueous layer was then filtered through Celite® to remove tin salts and the filtrate was concentrated under vacuum to leave a crude solid (380 mg; yield assumed quantitative) which was used directly in the next step (amide formation).

### Intermediate 33

Preparation of (3-iminoquinolin-7-yl)methanol (prepared using the general procedure from J. Am. Chem. Soc. 1997, 119, 5591)

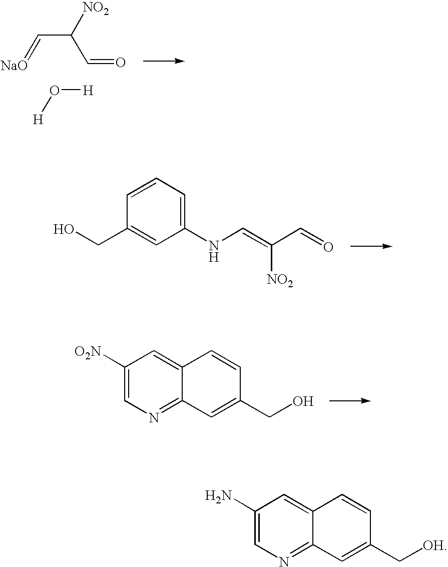

### 3-(3-(hydroxymethyl)phenylamino)-2-nitroacrylaldehyde

3-Aminobenzyl alcohol (4.97 g, 0.0404 mol) was dissolved in 4 mL conc HCl. Sodium nitromalonaldehyde monohydrate (prepared from mucobromic acid according to the procedure in Organic Syntheses Vol IV, pp 844, 1963) (4.25 g, 0.0269 mol) was dissolved in 35 mL water and added to the amine solution (a yellow precipitate formed immediately)—a further 80 mL of water being added to aid stirring. After 10 min, the precipitate was filtered, washed with water and air dried overnight to give the product (4.3 g) as a yellow solid.

### (3-nitroquinolin-7-yl)methanol

3-(3-(hydroxymethyl)phenylamino)-2-nitroacrylaldehyde (4.3 g, 19.4 mmol) was placed in 20 mL HOAc. 4.8 g of 3-aminobenzyl alcohol (4.8 g, 38.7 mmol) was dissolved in 5 mL conc HCl, then 20 mL HOAC was added to the HCl solution. This mixture was added to the reaction flask containing the 3-(3-(hydroxymethyl)phenylamino)-2-nitroacrylaldehyde in HOAc. The mixture was heated to reflux under nitrogen and after 20 min, benzene thiol (0.19 mL, 0.19 mmol) was added. The mixture was refluxed for 28 h (m/z=208.1). After allowing to cool, acid was removed under vacuum. The residue was dissolved in EtOAc/MeOH and loaded on a silica gel cartridge. Purification by column chromatography on silica gel using hexane/EtOAc (0-50%) then 10% MeOH/EtOAc as eluent gave the product (500 mg, 9%) as a brown solid.

### (3-aminoquinolin-7-yl)methanol

(3-nitroquinolin-7-yl)methanol (1.2 g, 0.0059 mol) and 400 mg of Pd/C (10% wt) were placed in 60 mL dry THF. The mixture was stirred under a hydrogen atmosphere (balloon) overnight. The reaction was filtered through celite and the filtrate concentrated to an oil. Purification by column chromatography on silica gel using MeOH/CH₂Cl₂ (0-10%) as eluent provided 0.9 g of an oily product. m/z=216.9 (+acetic acid). The product was suspended in MeOH and K₂CO₃ (200 mg) was added. This mixture was stirred at room temperature for 4 h. m/z=175.1. The mixture was filtered and the filtrate was concentrated under vacuum to give the product (172 mg, 19%) as a moist solid. ¹H NMR (d₄-MeOD) δ 8.32 (1H, d), 7.69 (1H, s), 7.55 (1H, d), 7.34 (1H, dd), 7.23 (1H, d), 5.40 (2H, s).

### Intermediate 34

### Preparation of (6-amino-1H-indazol-3-yl)methanol

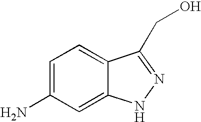

6-nitro-1H-indazole-3-carbaldehyde (500 mg, 0.003 mol) was dissolved in 50 mL THF. Lithium tetrahydroaluminate (400 mg, 0.01 mol) was added in 3 portions and the reaction mixture was stirred at room temperature overnight. Water (400 μL), 15% NaOH solution (400 μL), then water (1.2 mL) was added, and then the crystalline brown-yellow precipitate was filtered off. The filtrate was concentrate to an oil which was used directly in the next step without further purification. m/z=164.0. ¹H NMR (d₄-MeOH) δ 7.2 (1H, d), 7.05 (1H, d), 6.85 (1H, dd), 4.74 (2H, s).

### Intermediate 35

### Preparation of (7-aminoquinolin-3-yl)methanol

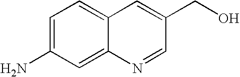

### 2-Dimethylaminomethylene-1,3-bis(dimethylammonio)propane bis(tetrafluoroborate)

To a 3-neck flask equipped with a reflux condenser was added bromoacetic acid (25 g, 0.18 mol) and phosphoryl chloride (50 mL, 0.54 mol). The solution was cooled to 0° C. and N,N-dimethylformamide (84 mL, 1.1 mol) was added dropwise over 30 min. The resulting solution was heated at 110° C. for 3 h. As the mixture was heated, it began to exotherm and evolve CO₂. The mixture was then cooled to 0° C. and a solution of aqueous 50% tetrafluoroboric acid (63 g, 0.36 mol) in MeOH (100 mL) was added slowly over 1 h via an addition funnel. Isopropanol (100 mL) was added to the dark viscous solution. Solids precipitated and the slurry was stirred at 0° C. for 2 h. The solids were collected by filtration to provide the product (64 g, 72%) as a pale yellow solid.

### Benzyl 3-aminophenylcarbamate

To a stirred solution of m-phenylenediamine (5.0 g, 0.046 mol) and N,N-diisopropylethylamine (8.0 mL, 0.046 mol) in CH₂Cl₂ (150 mL) at 0° C. was added slowly benzyl chloroformate (6.6 mL, 0.046 mol). The mixture was stirred at 0° C. for 2 h and then warmed to rt for 2 h. Aq. NaHCO₃ solution was added and the organic phase was separated, washed with brine, dried (Na₂SO₄) and concentrated. The residue was purified by column chromatography on silica gel to give the desired product (8.0 g, 711%) as a syrup. LC-MS: 2.11 min, 243.0 (M+1).

### Benzyl 3-formylquinolin-7-ylcarbamate

A slurry of benzyl 3-aminophenylcarbamate (8.0 g, 0.033 mol) and 2-dimethylaminomethylene-1,3-bis(dimethylammonio)propane bis(tetrafluoroborate) (31 g, 0.087 mol) in ethanol (400 mL) was heated at reflux for 24 h. The solution was concentrated under vacuum and the residue was dissolved in THF (200 mL) and 1N HCl (200 mL). The reaction mixture was stirred at rt overnight, then poured into a saturated solution of sodium bicarbonate (200 mL), and extracted with EtOAc (2×). The combined organic layers were washed with brine, dried (Na₂SO₄), and concentrated under vacuum to afford the desired product (10.0 g, 99%) as a yellow solid. LC-MS: 2.84 min, 307.1 (M+1).

### Benzyl 3-(hydroxymethyl)quinolin-7-ylcarbamate

To a stirred mixture of benzyl 3-formylquinolin-7-ylcarbamate (2.0 g, 0.0065 mol), THF (50 mL), MeOH (50 mL), and water (50 mL) was added sodium tetrahydroborate. (0.25 g, 0.0065 mol). The mixture was stirred at rt until LC-MS indicated no SM. The mixture was acidified with 1N HCl and concentrated under vacuum, and then treated with aq. NaHCO₃ solution and EtOAc. The organic layer was separated and washed with brine, dried (Na₂SO₄), and evaporated. The residue was purified by column chromatography on silica gel using MeOH-EtOAc (0-10%) as eluent to give the product (1.3 g, 64%) as a light yellow solid. LC-MS: 1.83 min, 309.2 (M+1).

### (7-Aminoquinolin-3-yl)methanol

A mixture of benzyl 3-(hydroxymethyl)quinolin-7-ylcarbamate (480 mg, 0.0016 mol), 10% Pd—C (50 mg), and MeOH (50 mL) was stirred under H₂ (1 atm) for 1 h. The catalyst was filtered-off and the filtrate was concentrated to give the product as a yellow solid. LC-MS: 0.34 min, 175.1 (M+1).

### Intermediate 36

### Preparation of quinolin-7-amine

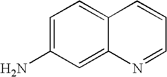

A mixture of 7-nitroquinoline (0.30 g, 0.0017 mol; Specs, Inc.), 10% Pd—C (50 mg), and MeOH (20 mL) was stirred under H2 (1 atm) for 2 h. The mixture was filtered and the filtrate was concentrated to give a yellow solid (235 mg, 95%). LC-MS: 0.33 min, 145.1 (M+1). ¹H NMR (DMSO-d₆): 8.58 (1H, dd, J=4.4, 1.6 Hz), 8.00 (1H, dd, J=8.0, 1.2 Hz), 7.60 (1H, d, J=8.8 Hz), 7.07 (1H, dd, J=8.0, 4.4 Hz), 6.98 (1H, dd, J=8.8, 2.0 Hz), 6.93 (1H, d, J=2.0 Hz), 5.75 (s, 2H).

### Intermediate 37

### Preparation of 5-amino-3-methylisoquinoline

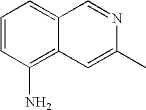

### 5-amino-3-methylisoquinoline

A mixture of 3-methyl-5-nitroisoquinoline (1.3 g, 0.0069 mol—prepared according to the procedure in WO 2004/024710), 10% Pd—C (100 mg) and MeOH (100 mL) was stirred under an atmosphere of hydrogen (1 atm) at rt for 2 h. The mixture was filtered and the filtrate was concentrated under vacuum to give a light yellow solid (1.1 g, 100%). LC-MS: 0.64 min, 159.1 (M+1).

### Intermediate 38

### Preparation of 1-chloroisoquinolin-5-amine

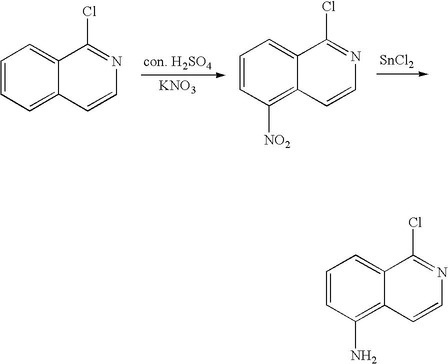

### 1-chloro-5-nitroisoquinoline

A mixture of 1-chloroisoquinoline (6.0 g, 0.037 mol) in conc. H₂SO₄ (35 mL) was treated with a solution of fuming HNO₃ (10 mL) and potassium nitrate (4.0 g, 0.040 mol) in conc. H₂SO₄ (35 mL) at 0-5° C. The mixture was stirred at 0° C. for a further 90 min, and then poured into ice. The precipitate was collected, washed and dried to give the product as a yellow solid. LC-MS: 3.68 min, 209.2 & 211.1 (M+1).

### 1-chloroisoquinolin-5-amine

A mixture of 1-chloro-5-nitroisoquinoline (450 mg, 0.0022 mol), stannous chloride dihydrate (2.4 g, 0.011 mol), and EtOAc (50 mL) was stirred under reflux under an atmosphere of nitrogen for 3 h. After cooling, the mixture was poured into ice-water and basified to pH 10.0 with aq. Na₂CO₃. The organic phase was separated and the aqueous phase was extracted with EtOAc. The combined organic layers were washed with brine, dried (Na₂SO₄) and concentrated under vacuum. The residue was purified by column chromatography on silica gel to give the product as a light yellow solid. LC-MS: 3.17 min, 179.2 & 181.2 (M+1).

### Intermediate 39

### Preparation of 7-amino-3,4-dihydro-2H-benzo[b][1,4]oxazin-3-yl)methanol and 8-amino-2,3,4,5-tetrahydrobenzo[b][1,4]oxazepin-3-ol

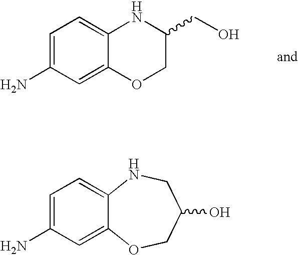

### 3,4-dihydro-7-nitro-2H-benzo[b][1,4]oxazin-3-yl)methanol and 8-amino-2,3,4,5-tetrahydrobenzo[b][1,4]oxazepin-3-ol

A mixture of 2-amino-5-nitrophenol (10.0 g, 0.0649 mol), potassium carbonate (13.4 g, 0.0973 mol), cesium fluoride (2.0 g, 0.013 mol) and 1-bromo-2,3-epoxypropane (5.37 mL, 0.0649 mol) in DMF (120 mL) was stirred under N₂ at rt overnight and then heated at 100° C. for 10 h. After cooling, the solvent was removed under vacuum and the residue was partitioned between water and EtOAc. The organic layer was washed with brine, dried (Na₂SO₄) and concentrated. The residue was purified by column with CH₂Cl₂-EtOAc (containing 5% Et₃N) (0 to 40%) to give an orange solid. LC-MS: 2.30 min, 211.1 (M+1).

### 7-amino-3,4-dihydro-2H-benzo[b][1,4]oxazin-3-yl)methanol and 8-amino-2,3,4,5-tetrahydrobenzo[b][1,4]oxazepin-3-ol

(3,4-dihydro-7-nitro-2H-benzo[b][1,4]oxazin-3-yl)methanol (3.8 g, 0.018 mol) was hydrogenated at 40 PSi for 2 hours over 100% Pd/C. The mixture was filtered through celite and the filtrate was concentrated under vacuum to afford the crude product. Purification by column chromatography on silica-gel (EtOAc) gave the product as a dark brown oil. LC-MS: 0.36 min, 181.1 (M+1). ¹H NMR (DMSO-d₆): 6.32 (1H, d, J=9.2 Hz), 6.01-5.97 (2H, m), 4.82-4.76 (2H, m), 4.29 (2H, s), 4.08 (1H, dd, J=10.4, 1.6 Hz), 3.79 (1H, dd, J=10.4, 6.8 Hz), 3.35 (2H, m), 3.17 (1H, m). 8-Amino-2,3,4,5-tetrahydrobenzo[b][1,4]oxazepin-3-ol was also isolated from above procedure as a minor byproduct.

### Intermediate 40

### Preparation of (S)-(3,4-dihydro-7-nitro-2H-benzo[b][1,4]oxazin-3-yl)methanol

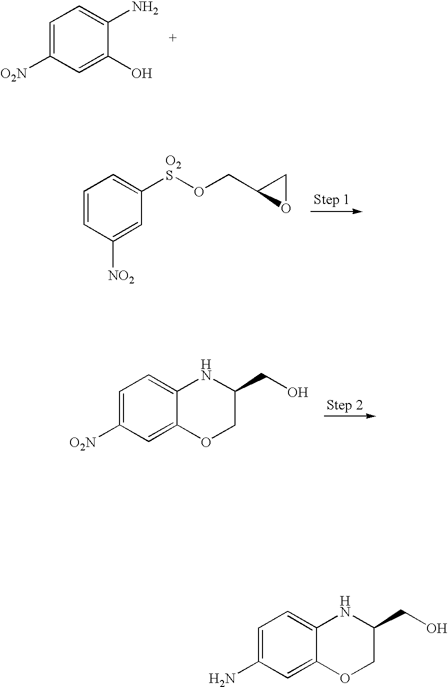

### (S)-(3,4-dihydro-7-nitro-2H-benzo[b][1,4]oxazin-3-yl)methanol

Sodium hydride (0.810 g, 0.0202 mol) was added slowly to a mixture of 2-amino-5-nitrophenol (3.0 g, 0.019 mol) in dmf (50 ml) at 0° C. The mixture was stirred at rt for 1 h and then (r)-(oxiran-2-yl)methyl 3-nitrobenzenesulfonate (5.0 g, 0.019 mol) was added. The mixture was stirred at room temperature overnight and then DMF was removed under vacuum. The residue was partitioned between water and EtOAc. The organic layer was washed with aqueous Na₂CO₃ solution, brine, dried (Na₂SO₄) and concentrated under vacuum to give a brown solid (5.2 g). A mixture of the above brown solid, K₂CO₃ (2.0 g) and DMF (200 ml) was stirred at 120° C. under N₂ overnight. After cooling, the solvent was removed in vacuo and the residue was partitioned between water and EtOAc. The organic layer was washed with brine, dried (Na₂SO₄) and concentrated under vacuum. The residue was purified by column chromatography on silica gel with CH₂Cl₂-EtOAc (containing 5% et3n−0 to 60%) to give the product as a soft brown solid. LC-MS: 2.30 min, 211.1 (m+1).

### (S)-(7-Amino-3,4-dihydro-2H-benzo[b][1,4]oxazin-3-yl)methanol

A mixture of (s)-(3,4-dihydro-7-nitro-2h-benzo[b][1,4]oxazin-3-yl)methanol (340 mg, 0.0016 mol), 10% Pd/C (50 mg) and MeOH (50 ml) were stirred under an atmosphere of hydrogen (1 atm) for 3 h. LC-MS indicated completion of reaction. The mixture was filtered and the filtrate was concentrated under vacuum to give the product as a brown syrup. LC-MS: 0.36 min, 181.1 (m+1).

(R)-(7-amino-3,4-dihydro-2h-benzo[b][1,4]oxazin-3-yl)methanol was prepared using the same procedure as for (s)-(3,4-dihydro-7-nitro-2h-benzo[b][1,4]oxazin-3-yl)methanol, except (s)-(oxiran-2-yl)methyl 3-nitrobenzenesulfonate was used as starting material.

### Intermediate 41

### Preparation of (7-amino-2,3-dihydro-benzo[1,4]dioxin-2-yl)-methanol 9 see 43P-Intermediate 19

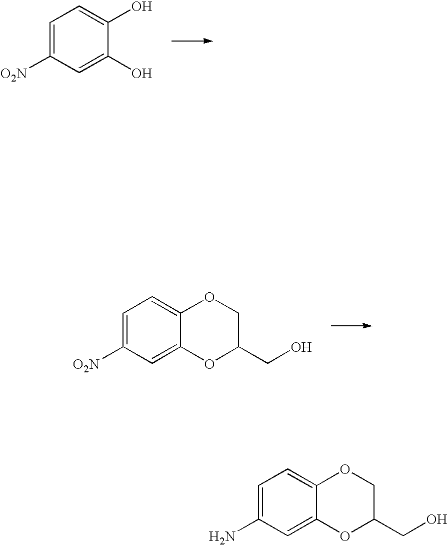

### (7-Nitro-2,3-dihydro-benzo[1,4]dioxin-2-yl)-methanol

3.0 g of sodium hydrogen carbonate was suspended in 90 mL DMF. At 0° C. a solution of 5.15 g of 4-nitrocatechol was added dropwise over 15 min. Subsequently, 3.9 g of epichlorohydrin in 10 mL DMF were added over 15 min. Stirring was continued at room temperature, then at 80° C. overnight. The mixture was diluted with water and extracted three times with ethyl acetate, dried (anhyd. Na₂SO₄), filtered and concentrated under vacuum to give a yellow oil. The oil was purified by column chromatography on silica gel using EtOAc-hexanes (0-100% gradient) to give the product (2.8 g) as a yellow solid.

### (7-amino-2,3-dihydro-benzo[1,4]dioxin-2-yl)-methanol

(7-nitro-2,3-dihydro-benzo[1,4]dioxin-2-yl)-methanol (1.0 g, 4.7 mmol) was dissolved in methanol (30 ml) and palladium on activated carbon was added (0.10 g, 5% wt). The mixture was shaken on a parr shaker under H₂(g) atmosphere (60 psi) for 24 hours. The mixture was filtered through celite and evaporated to give 722 mg of material as a white solid (86%), which was used as such for the next step. M/z=182 (m+1). Lc: 0.82 minutes.

### Intermediate 42

### Preparation of (6-Amino-2,3-dihydro-benzo[1,4]dioxin-2-yl)-methanol

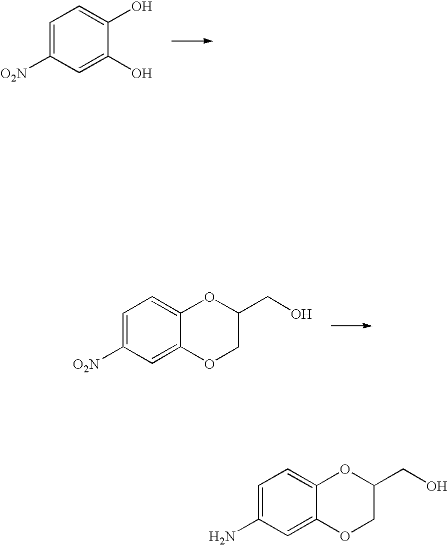

### (6-Nitro-2,3-dihydro-benzo[1,4]dioxin-2-yl)-methanol

1.93 g of 60% sodium hydride was suspended in 90 mL DMF. At 0° C. a solution of 5.15 g of 4-nitrocatechol was added dropwise over 15 min. Subsequently, 3.9 g of epichlorohydrin in 10 mL DMF were added over 15 min. Stirring was continued at room temperature, then at 80° C. overnight. The mixture was diluted with water and extracted three times with ethyl acetate, dried (Na₂SO₄), filtered and concentrated under vacuum to give a yellow oil. The oil was purified by column chromatography on silica gel using a EtOAc-hexanes (0-100% gradient) to give the product (2.3 g) as a yellow solid.

### (6-Amino-2,3-dihydro-benzo[1,4]dioxin-2-yl)-methanol

(6-Nitro-2,3-dihydro-benzo[1,4]dioxin-2-yl)-methanol (1.0 g, 4.7 mmol) was dissolved in methanol (30 mL) and palladium on activated carbon was added (0.10 g, 5% wt). The mixture was shaken on a Parr Shaker under H₂(g) atmosphere (60 PSI) for 24 hours. The mixture was filtered through Celite® and evaporated to give 646 mg of material as a white solid (77%), which was used as such for the next step. m/z=182 (M+1). LC: 0.82 minutes.

### Intermediate 43

### Preparation of (7-amino-3,4-dihydro-2H-pyrido[3,2-b][1,4]oxazin-3-yl)methanol

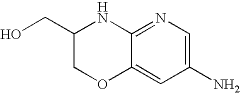

### 2-amino-3-methoxy-5-nitropyridine

Into a 250 mL sealed tube were combined 2-chloro-3-methoxy-5-nitropyridine (0.50 g, 0.00265 mol), concentrated ammonium hydroxide (5 mL, 0.1 mol) and ethanol (20 mL). The mixture was heated to 80° C. and stirred overnight. After allowing to cool to room temperature, the mixture was reduced in vacuo and the residue was taken up in ethyl acetate (50 mL), then washed with equal amounts of brine and water (1×50 mL each). The organic layer was dried (Na₂SO₄), filtered and concentrated under vacuum to leave a solid (0.312 g, 69%) which was used directly in the next step without further purification. LC-MS1.94 min. M/Z=171.0 (M+1).

### 2-amino-3-hydroxy-5-nitropyridine

Into a 500 mL round bottom flask were combined 2-amino-3-methoxy-5-nitropyridine (0.300 g, 0.00177 mol) and solid pyridine hydrochloride (8.8 g, 0.076 mol). The solid mixture was heated at 150° C. upon which the solids fused (the evolution of a gas was also apparent). The mixture was held at 150° C. for three hours upon which reaction was deemed complete by LC-MS. After allowing to cool to 80° C., the mixture was poured on to ice and the aqueous layer was extracted with ethyl acetate (3×100 ml). The combined organic extracts were washed with water (2×100 mL), dried (Na₂SO₄), filtered and concentrated under vacuum to leave a crude residue. The residue was purified by column chromatography on silica gel using a methanol:methylene chloride (0-10%) gradient as eluent to give the product as a solid (0.138 g, 49%) which was used directly in the next step. LC-MS1.28 min. m/z=155.9 (M+1).

### (7-nitro-3,4-dihydro-2H-pyrido[3,2-b][1,4]oxazin-3-yl)methanol

Into a 75 mL sealed tube were combined 2-amino-3-hydroxy-5-nitropyridine (0.138 g, 0.000890 mol), N,N-dimethylformamide (4.1 mL) and potassium carbonate (0.39 g, 0.0028 mol). The mixture was allowed to stir at room temperature for 10 minutes then 1-bromo-2,3-epoxypropane (0.12 g, 0.00089 mol) was added in one portion. The flask was sealed, then heated to 110° C. and stirred overnight. After allowing to cool, the mixture was concentrated under vacuum to give a crude solid which was dissolved in EtOAc (75 mL), washed with water and brine, then dried (Na₂SO₄), filtered and concentrated under vacuum to leave a crude residue. The residue was purified by column chromatography on silica gel using MeOH/CH₂Cl₂ (0-10% gradient) as eluent to give a solid (0.092 g, 46%). LC-MS1.92 min. M/Z=212.0 (M+1). ¹H NMR (d₆-DMSO) δ 8.8 (d, 1H), 7.8 (d, 1H), 5.1 (t, 1H), 4.2 (m, 1H), 4.0 (m, 1H), 3.62 (m, 1H), 3.45 (m, 1H), 3.21 (m, 1H).

### (7-amino-3,4-dihydro-2H-pyrido[3,2-b][1,4]oxazin-3-yl)methanol

Into a 500 mL round bottom flask were combined (7-nitro-3,4-dihydro-2H-pyrido[3,2-b][1,4]oxazin-3-yl)methanol (0.320 g, 0.00152 mol), 10%-palladium on carbon (0.06 g, 0.0005 mol) and methanol (50 mL). The apparatus was evacuated, then hydrogen was introduced and the mixture was allowed to stir overnight (at 1 atm pressure). The mixture was then filtered through celite and the filtrate was concentrated under vacuum to yield an oil (0.252 g, 89%) which was used directly in the next step without further purification. (0.252 g, 89%) LC-MS 0.29 min. M/Z=181.9 (M+1).

### Intermediate 44

### Preparation of (5-amino-1H-indol-2-yl)methanol

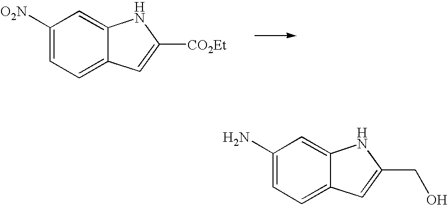

2-Ethoxycarbonyl-5-nitroindole (500 mg, 0.002 mol) was dissolved in 50 mL THF, added lithium tetrahydroaluminate (341 mg, 0.00898 mol) in 3 portions and stirred at room temperature overnight. Water (341 μL), 15% NaOH solution (341 mL), and water (1.1 mL) were added cautiously and the mixture was filtered. The filtrate was concentrated under vacuum to give the product (300 mg, 98%) as an oil. m/z=162.9.

### Intermediate 45

### Preparation of (5-amino-1H-indazol-3-yl)methanol

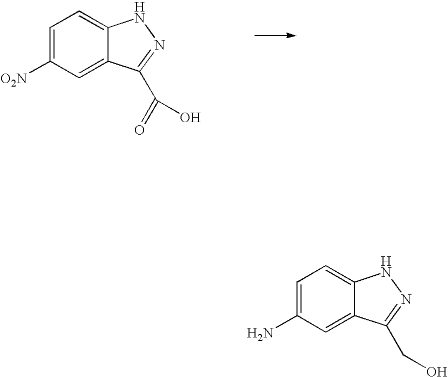

5-nitro-1H-indazole-3-carboxylic acid (500 mg, 0.002 mol) was dissolved in 50 mL THF, added lithium tetrahydroaluminate (366 mg, 0.00964 mol) in 3 portions and stirred at room-temperature overnight. 65 mg (15%). Water (366 μL), 15% NaOH solution (366 μL), and water (1.1 mL) were added cautiously and the mixture was filtered. The filtrate was concentrated under vacuum to give the product (65 mg, 15%) as an oil. m/z=160.0.

### Intermediate 46

### Preparation of 2-methylthiazolo[5,4-b]pyridin-6-amine

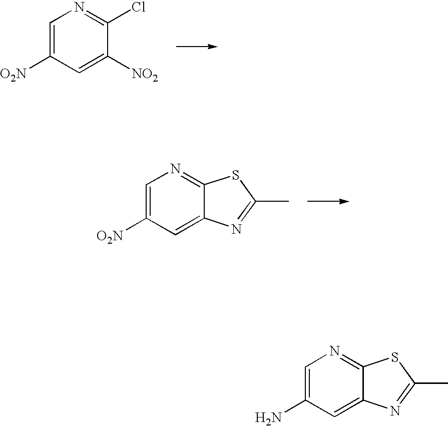

### a. 2-Methyl-6-nitrothiazolo[5,4-b]pyridine

2-Chloro-3,5-dinitropyridine (2.5 g, 1.2 mmol) and thioacetamide (3.75 g, 5.0 mmol) were combined in sulfolane (13 mL) and heated at 100° C. for 2 h. After cooling, water (25 mL) was added and the mixture was filtered. The filter cake was triturated with boiling EtOH (60 mL) and filtered. The filtrate was allowed to cool overnight, then filtered to give the nitro-thiazolopyridine derivative (1.05 g, 43%). ¹H NMR (400 MHz; d₆-DMSO) δ 9.38 (1H, d), 9.25 (1H, d), 2.91 (3H, s).

### b. 2-Methylthiazolo[5,4-b]pyridin-6-amine

2-Methyl-6-nitrothiazolo[5,4-b]pyridine (400 mg, 2.0 mmol) was suspended in conc. HCl (10 mL) and the mixture was heated to 50° C. Stannous chloride, dihydrate (1.62 g, 7.2 mmol) was added to the reaction mixture in two portions. The sides of the flask were washed down with EtOAc (25 mL) and the biphasic mixture was stirred at 50° C. for 2 h (monitoring by LCMS). After allowing to cool to room temperature, SN NaOH (1 mL) was added followed by water (10 mL). The mixture was cooled to 4° C. and the pH was adjusted to 9 by addition of more 5N NaOH. The mixture was partitioned between EtOAc and water and the organic layer was washed with water, brine, dried, filtered and concentrated under vacuum. Purification by column chromatography on silica gel using EtOAc/hexane as eluent (0-75%) gave the product (134 mg, 40%) as a solid. m/z=165.9 (M+1). ¹H NMR (400 MHz; d₆-DMSO) δ 7.97 (1H, d), 7.35 (1H, d), 5.52 (2H, bs), 2.75 (3H, s).

### Intermediate 47

### Preparation of (6-aminothiazolo[5,4-b]pyridin-2-yl)methyl pivalate

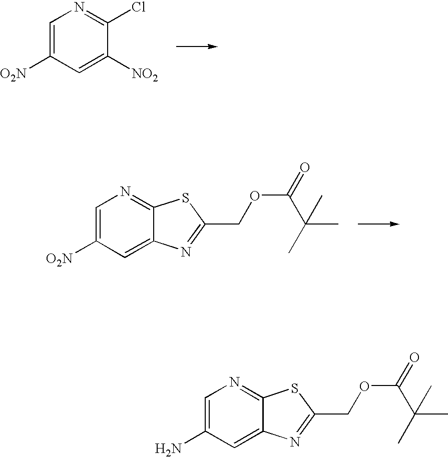

### a. (6-Nitrothiazolo[5,4-b]pyridin-2-yl)methyl pivalate

A mixture of 2-chloro-3,5-dinitropyridine (5.1 g, 25 mmol) and 2-amino-2-thioxoethylpivalate (8.8 g, 50 mmol) in sulfolane (50 mL) was heated to 100-110° C. under a nitrogen atmosphere and stirred for approximately 2 hours. After allowing to cool to room temperature, the mixture was poured in to EtOAc (150 mL) and the organic layer was washed with H₂O (3×200 mL) and brine (1×100 mL). The organic layer was dried (MgSO₄), filtered and the solvent removed under vacuum to leave a crude oil. The oil was purified by filtration through a plug of silica eluting with EtOAc/hexane (10% EtOAc to 20% EtOAc) to give a solid (ca. 6-7 g). The solid was then triturated with MeOH (ca. 20 mL) and filtered to give the desired product (2.17 g). Further product was obtained by concentrating the filtrate under vacuum and purifying by column chromatography on silica gel using 0 to 20% EtOAc/hexane as eluent to give a solid (1.1 g). The solid was triturated with MeOH to give further product (0.5 g). Total yield of (6-nitrothiazolo[5,4-b]pyridin-2-yl)methyl pivalate=2.67 g (36%). m/z=296.5 (M+1). ¹H NMR (400 MHz; CDCl₃) δ 9.46 (1H, d), 9.00 (1H, d), 5.54 (2H, s), 1.32 (9H, s).

### b. (6-Aminothiazolo[5,4-b]pyridin-2-yl)methyl pivalate

(6-Nitrothiazolo[5,4-b]pyridin-2-yl)methyl pivalate (650 mg, 2.2 mmol) was suspended in conc. HCl (20 mL) and heated to 50° C. Stannous chloride, dihydrate (1.8 g, 7.7 mmol) was added, followed by ethyl acetate (45 mL). The reaction was heated at 50° C. for about 10-15 minutes, then cooled in an ice bath (TLC indicated complete reaction at this stage). H₂O (30 mL) and EtOAc (30 mL) were added then SN NaOH was added cautiously until the pH was adjusted to ca. 7 (all the time keeping the flask in the ice bath and stirring vigorously. The internal temperature was kept at <10° C. for the neutralization). Water (50 mL) was added, then the product was extracted in to EtOAc (2×30 mL). The combined organics were washed with brine (1×25 mL), dried (MgSO₄), filtered and concentrated under vacuum to leave a crude residue. The residue was purified by column chromatography on silica gel using 50-75% EtOAc/hexane as eluent to give the desired product (330 mg, 55%) as a solid. ¹H NMR (400 MHz; CDCl₃) δ 8.14 (1H, d), 7.51 (1H, d), 5.44 (2H, s), 1.29 (9H, s).

### Intermediate 48

### Preparation of 6-aminothiazolo[5,4-b]pyridine

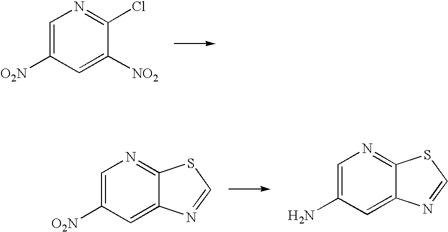

### a. 6-Nitrothiazolo[5,4-b]pyridine

The procedure reported for 3-nitro-1,3-benzotriazole in WO2005028445 was used. A mixture of 2-chloro-3,5-dinitropyridine (8 g, 39 mmol) and N,N-dimethylthioformamide (14.5 mL, 178 mmol) was heated at 60° C. for 3 h. A yellow precipitate was formed. Xylene (20 mL) was added to the reaction mixture and the mixture was heated to reflux for 4 h, and then stirred at room temperature for 18 h. The mixture was diluted with EtOH (12 mL), filtered and the brown solid was recrystallized from EtOH to give the product (800 mg) as a solid. ¹H NMR (400 MHz; acetone-d₆) δ 9.6 (1H, s), 9.44 (1H, s), 9.05 (1H, s).

### b. 6-Aminothiazolo[5,4-b]pyridine

6-Nitrothiazolo[5,4-b]pyridine (800 mg, 4.4 mmol) was dissolved in conc. HCl (10 mL) and heated to 50° C. Stannous chloride, dihydrate (3.49 g, 15.5 mmol) was added in two portions, at 50° C., and the sides of the flask were then ‘washed-down’ with EtOAc (50 mL). The mixture was stirred at 50° C. for 60 min. The mixture was cooled in an ice bath, then 5 N NaOH (1 mL) was added, followed by water (5 mL), then more 5 N NaOH until the pH was adjusted to ca. 9. The mixture was filtered and the filtrate was partitioned between EtOAc and water. The organic layer was separated and dried, filtered and concentrated under vacuum to an oil (300 mg, 45%). The oil was used directly in the next step without further purification—it appeared to be ca. 90% pure by nmr. ¹H NMR (400 MHz; d₆-DMSO) δ 9.32 (1H, s), 8.15 (1H, d), 7.5 (1H, d), 4.55 (2H, bs).

### Intermediate 49

### Preparation of ethyl 6-aminothiazolo[5,4-b]pyridin-2-carboxylate

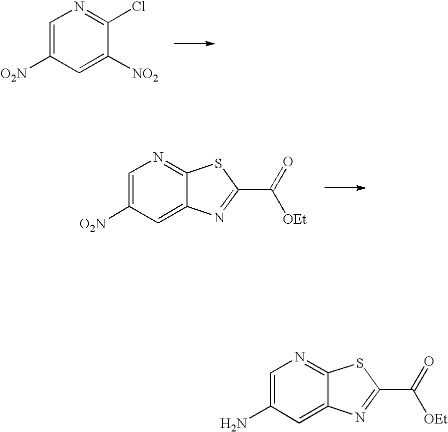

### a. Ethyl 6-nitrothiazolo[5,4-b]pyridin-2-carboxylate

2-Chloro-3,5-dinitropyridine (200 mg, 1.0 mmol) and ethyl thioamidooxalate (133 mg, 1.0 mmol) were combined under nitrogen and sulfolane (3 mL) was added. The mixture was heated to 100° C. and stirred for 3 hours. TLC indicated some product form—added another 1 eq of ethyl thioamidooxalate (133 mg). The mixture was stirred overnight at 100° C. TLC indicated complete reaction so after allowing to cool to rt, the mixture was poured in to H₂O (50 mL) and EtOAc (30 mL). The organic and aqueous layers were partitioned and the aqueous layer was extracted with EtOAc (2×20 mL). The combined organic extracts were washed with brine (1×30 mL), dried (MgSO₄), filtered and the solvent removed under vacuum to leave a crude oil. The oil was purified by column chromatography on silica gel using 5-20% EtOAc/hexane as eluent to give the desired product (60 mg, 20%) as a solid. ¹H NMR (400 MHz; CDCl₃) δ 9.59 (1H, d), 9.23 (1H, d), 4.62 (2H, q), 1.53 (3H, t).

### b. Ethyl 6-aminothiazolo[5,4-b]pyridin-2-carboxylate

Ethyl 6-nitrothiazolo[5,4-b]pyridin-2-carboxylate (400 mg, 1.6 mmol) was placed in 10 mL conc HCl (10 mL) and heated to 50° C. Stannous chloride, dihydrate (1.25 g, 5.53 mmol) was added in two portions, at 50° C., and the sides of the flask were ‘washed down’ with EtOAc (50 mL). The mixture was stirred at 50° C. for 60 min. The mixture was cooled in an ice bath, then 5 N NaOH (1 mL) was added, followed by water (15 mL), then more 5 N NaOH until the pH was adjusted to ca. 9. The mixture was partitioned and the organic layer was washed with H₂O, brine, then dried (MgSO₄) and concentrated under vacuum to a crude oil. The oil was purified by column chromatography on silica gel to give the desired compound (100 mg, %) as a solid. ¹H NMR (400 MHz; d₆-DMSO) δ 8.25 (1H, d), 7.59 (1H, d), 5.85 (2H, s), 4.45 (2H, q), 1.35 (3H, t).

### Intermediate 50

### Preparation of 5-amino-2-(2-hydroxyethyl)isoindoline-1,3-dione

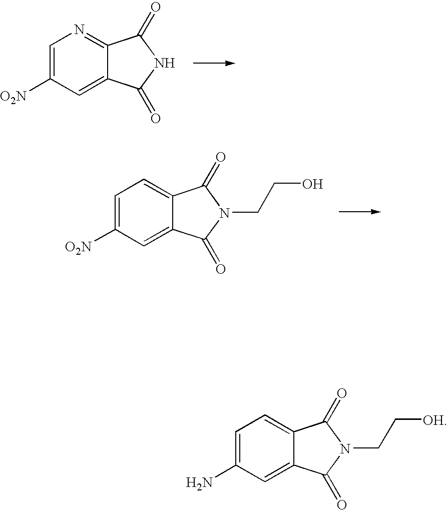

### a. 2-(2-Hydroxyethyl)-5-nitroisoindoline-1,3-dione

K₂CO₃ (5 g, 40 mmol), 4-nitrophthalimide (1.5 g, 7.8 mmol) and 2-bromoethanol (1 mL, 20 mmol) in acetone (20 mL) was heated to 120° C. under microwave irradiation and stirred for 90 min. After allowing to cool, the mixture was partitioned between H₂O and EtOAc and the aqueous layer was extracted with EtOAc (×2). The combined organic extracts were dried (MgSO₄), filtered and the solvent removed under vacuum to leave a crude residue. The residue was purified by column chromatography on silica gel using 1-10% MeOH/CH₂Cl₂ as eluent to give the product (1.02 g, 58%) as a solid. m/z=237.1 (M+1). ¹H NMR (400 MHz; CDCl₃) δ 8.72-8.63 (2H, m), 8.11-8.05 (1H, m), 3.00-3.87 (4H, m).

### b. 5-Amino-2-(2-hydroxyethyl)isoindoline-1,3-dione

2-(2-Hydroxyethyl)-5-nitroisoindoline-1,3-dione (0.62 g, 2.6 mmol) and palladium (10% wt. on calcium carbonate; 0.4 g, 1.9 mmol) in MeOH was hydrogenated (1 atm) overnight. The mixture was then filtered through celite and the filtrate concentrated under vacuum to leave the product (0.5 g, 94%) as a solid. m/z=207.2 (M+1). ¹H NMR (400 MHz; d₆-DMSO) δ 7.46 (1H, d), 6.90 (1H, d), 6.77 (1H, dd), 6.43 (2H, s), 4.81 (1H, t), 3.55-3.49 (4H, m).

### Intermediate 51

### Preparation of (5-aminobenzo[d]oxazol-2-yl)methanol

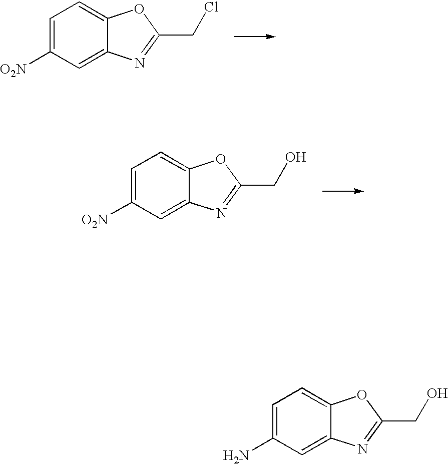

### a. (5-Nitrobenzo[d]oxazol-2-yl)methanol

NaOH (0.2 g, 6 mmol) in H₂O (5 mL) was added to a solution of 2-(Chloromethyl)-5-nitrobenzo[d]oxazole (0.6 g, 2.8 mmol) in THF (20 mL) and the mixture stirred overnight. The organics were removed under vacuum and the residue was diluted with H₂O then acidified using 1N HCl. The mixture was extracted with EtOAc and the organic layer was dried (Na₂SO₄), filtered and concentrated under vacuum to leave a crude product. The crude product was dissolved in CH₂Cl₂, filtered and then concentrated under vacuum to leave the product (0.43 g, 80%) as a solid. ¹H NMR (400 MHz; CDCl₃) δ 8.15 (1H, s), 7.93 (1H, dd), 7.33 (1H, d), 7.26 (1H, s), 7.08 (1H, d), 4.70 (2H, s).

### b. (5-Aminobenzo[d]oxazol-2-yl)methanol

(5-Nitrobenzo[d]oxazol-2-yl)methanol (0.43 g, 2.2 mmol) and palladium (10% wt. on calcium carbonate; 0.43 g, 2.1 mmol) in MeOH was hydrogenated (1 atm) overnight. The mixture was then filtered through celite and the filtrate concentrated under vacuum to leave the product (0.27 g, 75%) as a solid. m/z=165.0 (M+1). ¹H NMR (400 MHz; CDCl₃) 7.55 (1H, s), 6.78 (1H, d), 6.33 (1H, dd), 6.14 (1H, d), 4.58 (2H, s), 3.41-3.58 (2H, m).

### Intermediate 52

### Preparation of 6-aminooxazolo[4,5-b]pyridin-2(3H)-one

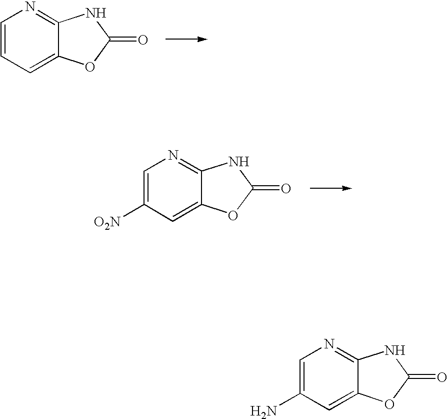

### a. 6-Nitrooxazolo[4,5-b]pyridin-2(3H)-one

2,3-Dihydropyrido[2,3-d][1,3]oxazol-2-one (1.20 g, 8.8 mmol) was introduced to sulfuric acid (3.55 mL) at ca. −3° C. The mixture was stirred for about 1 hr (temperature kep below 5° C.). The mixture was re-cooled to 0° C. and fuming nitric acid was added dropwise. The mixture was stirred overnight then heated to 40-45° C. and stirred for a further 18 hr. The mixture was quenched by pouring on to ice and the emerging precipitate was filtered and washed with H2O to provide the product (0.69 g, 43%) as a solid. m/z=−181.9 (M+1). ¹H NMR (400 MHz; d₆-MeOH) δ 8.24 (d, 1H), 7.54 (d, 1H).

### b. 6-Aminooxazolo[4,5-b]pyridin-2(3H)-one

6-Nitro-oxazolo[4,5-b]pyridin-2(3H)-one (0.69 g, 3.8 mmol) and palladium (10% wt. on calcium carbonate; 0.40 g, 1.9 mmol) were combined in MeOH and hydrogenated (1 atm) overnight. The mixture was filtered through celite and the filtrate was concentrated under vacuum to leave the product (0.15 g, 30%). m/z=152.0 (M+1). ¹H NMR (400 MHz; d₆-DMSO) δ 11.79 (br s, 1H), 7.40 (d, 1H), 6.89 (d, 1H), 5.11 (s, 2H).

### Intermediate 53

### Preparation of 7-Amino-quinoline-3-carboxylic acid methyl ester

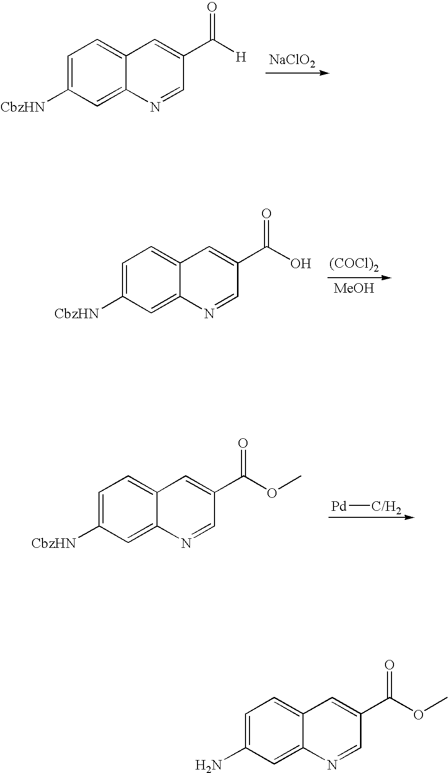

### a. 7-(Benzyloxycarbonylamino)quinoline-3-carboxylic acid

To a stirred solution of benzyl 3-formylquinolin-7-ylcarbamate (see US 2006194801; 2.7 g, 8.8 mmol) in acetonitrile (50 mL) was added an aqueous solution of potassium dihydrogen phosphate (1.25 M; 35.2 mL, 44 mmol), followed by sodium chlorite (2.4 g, 26 mmol). The slurry was stirred at rt overnight. An aqueous solution of sodium hydrogen sulfite (1 M; 50 mL) was added and the mixture was stirred at rt for 1 h. 1N HCl was added to adjust the pH to 3-4. The emerging precipitate was collect by filtration and washed with water and dried to give the crude product as a solid. The filtrate was extracted with EtOAc (100 mL) and the organic layer was washed with brine, dried (Na₂SO₄), and evaporated to give additional crude product. The combined crude product (2.6 g, 92%) was used for the next step reaction without further purification. m/z=321.2 (M−1); rt=2.37 min.

### b. Methyl 7-(benzyloxycarbonylamino)quinoline-3-carboxylate

To a stirred mixture of 7-(benzyloxycarbonylamino)quinoline-3-carboxylic acid (1.20 g, 3.7 mmol), THF (100 mL), and DMF (0.1 mL) at 0° C. was added oxalyl chloride (0.63 mL, 7.4 mmol). The mixture was stirred at rt for 3 h, and then MeOH (1.51 mL, 37.2 mmol) was added followed by Et₃N (2.6 mL, 19 mmol). The mixture was stirred at rt overnight. The mixture was concentrated under vacuum and then treated with aq. NaHCO₃ (20 mL) and EtOAc (100 mL). The organic and aqueous layers were partitioned, and the organic layer washed with brine, dried Na₂SO₄), and evaporated under vacuum. The residue was purified by column chromatography on silica gel to give the product (1.02 g, 81%) as a solid. m/z=337.4 (M+1); rt=3.02 min. ¹H NMR (400 MHz; d₆-DMSO) δ 10.41 (s, 1H), 9.24 (d, 1H, J=2.4 Hz), 8.88 (d, 1H, J=2.4 Hz), 8.31 (d, 1H, 1.6 Hz), 8.12 (d, 1H, J=9.2 Hz), 7.75 (dd, 1H, J=9.2, 2.0 Hz), 7.50-7.34 (m, 5H), 5.23 (s, 2H), 3.93 (s, 3H).

### c. 7-Amino-quinoline-3-carboxylic acid methyl ester

A mixture of methyl 7-(benzyloxycarbonylamino)quinoline-3-carboxylate (420 mg, 1.2 mmol), 10% Pd—C (100 mg), and MeOH (100 mL) was stirred under H₂ (1 atm) for 2 h. The mixture was filtered through celite and the filtrate was concentrated to give the product (240 mg, 95%) as a solid. m/z=203.3 (M+1); rt=1.43 min.

### Intermediate 54

### Preparation of 2-(7-Aminoquinolin-3-yl)propan-2-ol

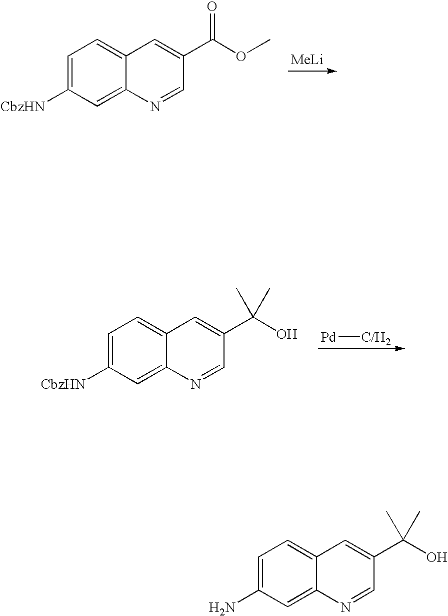

### a. Benzyl 3-(2-hydroxypropan-2-yl)quinolin-7-ylcarbamate

To a stirred solution of methyl 7-(benzyloxycarbonylamino)quinoline-3-carboxylate (170 mg, 0.50 mmol) in THF (15 mL) at −78° C. under nitrogen was added a solution of MeLi in Et₂O (1.6 M; 1.0 mL, 1.6 mmol). The reaction mixture was slowly warmed to rt and then quenched by addition of sat. aq. NH₄Cl solution (10 mL), and extracted with EtOAc (3×50 mL). The combined organic layers were washed with brine, dried (Na₂SO₄), and concentrated under vacuum. The residue was purified by column chromatography on silica gel using EtOAc/hexane (0-100% EtOAc) as eluent to give the product (95 mg, 56%) as a solid m/z=337.1 (M+1); rt=1.89 min.

### b. 2-(7-Aminoquinolin-3-yl)propan-2-ol

A mixture of benzyl 3-(2-hydroxypropan-2-yl)quinolin-7-ylcarbamate (95 mg, 0.28 mmol), 10% Pd—C (10 mg) and MeOH (10 mL) was stirred under H₂ (1 atm) for 1 h. The mixture was filtered through celite and the filtrate was concentrated under vacuum to give the product (56 mg, 98%) as a solid. m/z=203.3 (M+1); rt=1.32 min.

### Intermediate 55

### Preparation of 1-(7-aminoquinolin-3-yl)ethanol

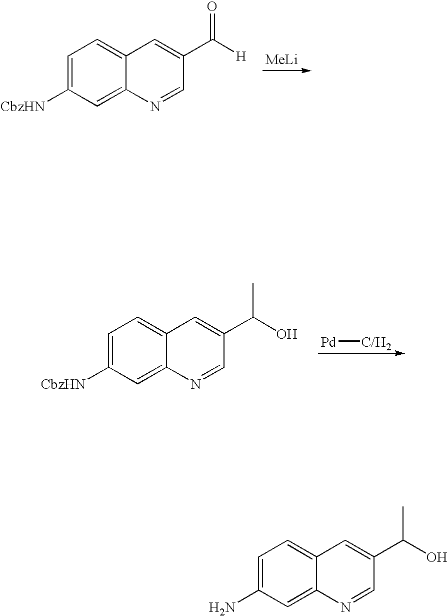

### a. Benzyl 3-(1-hydroxyethyl)quinolin-7-ylcarbamate

To a stirred solution of benzyl 3-formylquinolin-7-ylcarbamate (0.50 g, 1.6 mmol) in THF (40 mL) at −78° C. under nitrogen was added a solution of MeLi in Et₂O (1.6 M; 2.1 mL, 3.36 mmol). The reaction mixture was slowly warmed to rt, and then quenched by adding sat. aq. NH₄Cl solution (10 mL) and extracted with EtOAc (3×50 mL). The combined organic layers were washed with brine, dried (Na₂SO₄), and concentrated under vacuum. The residue was purified by column chromatography on silica gel using EtOAc as eluent to give the product (350 mg, 66%) as a foam. m/z=323.0 (M+1); rt=1.86 min. ¹H NMR (400 MHz; d₆-DMSO) δ10.15 (s, 1H), 8.82 (d, 1H, J=2.0 Hz), 8.20 (s, 1H), 8.13 (d, 1H, J=2.0 Hz), 7.88 (d, 1H, J=9.2 Hz), 7.64 (dd, 1H, J=9.2, 2.0 Hz), 7.49-7.33 (m, 5H), 5.41 (d, 1H, J=4.4 Hz), 5.21 (s, 2H), 4.93 (m, 11H), 1.44 (d, 3H, J=6.4 Hz).

### b. 1-(7-Aminoquinolin-3-yl)ethanol

A mixture of benzyl 3-(1-hydroxyethyl)quinolin-7-ylcarbamate (180 mg, 0.56 mmol), 10% Pd—C (20 mg), and MeOH (20 mL) was stirred under H₂ (1 atm) for 1 h. The mixture was filtered through celite and the filtrate was concentrated to give the product (97 mg, 92%) as a solid. m/z=189.0 (M+1); rt=1.08 min.

### Intermediate 56

### Preparation of 3-((2-(tert-Butyldimethylsilyloxy)ethoxy)methyl)quinolin-7-amine

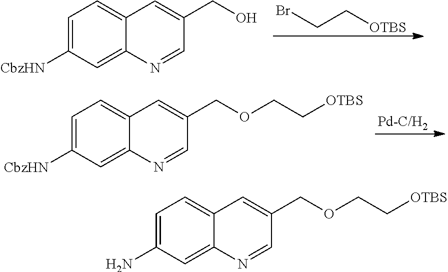

### a. Benzyl 3-((2-(tert-butyldimethylsilyloxy)ethoxy)methyl)quinolin-7-ylcarbamate

To a stirred mixture of benzyl 3-(hydroxymethyl)quinolin-7-ylcarbamate (500 mg, 1.6 mmol) in DMF (10 mL) was added sodium hydride (60% dispersion in oil; 260 mg, 6.5 mmol). The mixture was stirred at rt for 2 h and then (2-bromoethoxy)-tert-butyldimethylsilane (580 mg, 2.4 mmol) was added. After stirring at rt overnight, the reaction mixture was quenched by adding aq. NH₄Cl solution and extracted with EtOAc (3×30 mL). The combined organic layers were washed with brine, dried (Na₂SO₄) and concentrated under vacuum. The residue was purified by column chromatography on silica gel using 0-50% EtOAc/hexane as eluent to give the product (95 mg, 120%) as a solid. m/z=467.4 (M+1); rt=3.46 min.

### b. 3-((2-(tert-Butyldimethylsilyloxy)ethoxy)methyl)quinolin-7-amine

A mixture of benzyl 3-((2-(tert-butyldimethylsilyloxy)ethoxy)methyl)quinolin-7-ylcarbamate (95 mg, 0.20 mmol), 10% Pd—C (10 mg), and MeOH (15 mL) was stirred under H₂ (1 atm) for 1 h. The mixture was filtered through celite and the filtrate was concentrated under vacuum to give the product. m/z=333.1 (M+1); rt=2.14 min.

### Intermediate 57

### Preparation of 1-(7-Aminoquinolin-3-yl)ethane-1,2-diol

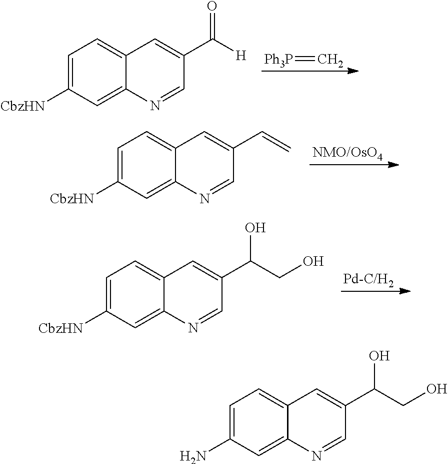

### a. Benzyl 3-vinylquinolin-7-ylcarbamate

A suspension of methyltriphenylphosphonium bromide (4.33 g, 12.1 Mmol) in anhydrous THF (50 mL) at −50° C. was treated with a solution of n-butyllithium in hexane (1.6M; 7.6 mL, 12.1 mmol) over 20 min, and the resulting solution was warmed to −10° C. After 1 h, the mixture was cooled to −70° C. and a solution of benzyl 3-formylquinolin-7-ylcarbamate (1.06 g, 3.5 mmol) in THF (20 mL) was added over 15 min. The reaction mixture was warmed to rt, stirred overnight, then quenched by the addition of water (100 mL), and extracted with EtOAc (3×50 mL). The combined organic layers were washed with brine, dried (Na₂SO₄) and concentrated under vacuum. The residue was purified by column chromatography on silica gel using 30-100% EtOAc/hexane as eluent to give the product (1.0 g, 94%) as a solid. m/z=305.8 (M+1); rt=2.46 min. ¹H NMR (400 MHz; d₆-DMSO) a 10.21 (s, 1H), 9.01 (d, 1H, J=2.4 Hz), 8.27 (d, 1H, J=1.6 Hz), 8.21 (s, 1H), 7.87 (d, 1H, J=8.8 Hz), 7.65 (dd, 1H, J=8.8, 2.4 Hz), 7.49-7.33 (m, 5H), 6.90 (dd, 1H, J=17.6, 11.6 Hz), 6.09 (d, 1H, J=17.6 Hz), 5.43 (d, 1H, J=11.6 Hz), 5.22 (s, 2H).

### b. Benzyl 3-(1,2-dihydroxyethyl)quinolin-7-ylcarbamate

To a suspension of benzyl 3-vinylquinolin-7-ylcarbamate (850 mg, 2.8 mmol) in tert-butyl alcohol (15 mL) was added N-methylmorpholine N-oxide (360 mg, 3.1 mmol) and water (15 mL). To this slurry at rt was then added 4% w/w osmium tetraoxide (440 mg, 0.07 mmol) solution in water. After 5 h, the reaction was complete and homogeneous. The mixture was extracted with EtOAc (3×50 mL), and the combined organic layers were washed with water, brine, dried (Na₂SO₄), and concentrated under vacuum. The residue was purified by column chromatography using 0-15% MeOH/EtOAc as eluent to give the product (580 mg, 610%) as a foam. m/z=339.0 (M+1); rt=1.78 min.

### c. 1-(7-Aminoquinolin-3-yl)ethane-1,2-diol

A mixture of benzyl 3-(1,2-dihydroxyethyl)quinolin-7-ylcarbamate (570 mg, 1.7 mmol), 10% Pd—C (100 mg) and MeOH (50 mL) was stirred under H₂ (1 atm) for 2 h. The mixture was filtered through celite and the filtrate was concentrated under vacuum to give the product (320 mg, 93%) as a solid. m/z=205.1 (M+1); rt=0.53 min.

### Intermediate 58

### Preparation of 8-amino-1,2,3,4-tetrahydronaphthylen-2-ol

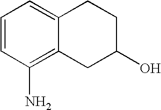

This compound was prepared using the procedure described in WO 2005/040119: ‘Tetrahydronaphthalene and urea derivatives’.

### Preparation of (3-Amino-7,8-dihydro-5H-pyrano[4,3-b]pyridin-7-yl)methanol

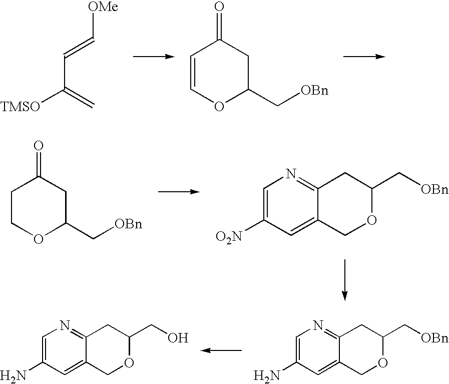

### a. 2-Benzyloxymethyl-2,3-dihydropyran-4-one

A solution of benzyloxyacetaldehyde (8.9 g, 58 mmol) and 1-methoxy-3-(trimethylsiloxy)-1,3-butadiene (10 g, 58 mmol) in toluene (80 mL) was stirred for 30 minutes, then cooled to 0° C. Zinc chloride in tetrahydrofuran (0.5 M; 58.0 mL, 30 mmol) was added over 30 minutes. The reaction was allowed to warm slowly to room temperature and then heated at 50° C. for 2 hours. After cooling, the mixture was evaporated to dryness, then dissolved in EtOAc (100 mL). The solution was washed with 2N HCl (50 mL) NaHCO₃ (3×50 mL) and brine (3×50 mL), dried (MgSO₄), filtered and concentrated under vacuum. Purification by column chromatography on silica gel using 0 to 20% EtOAc/hexane as eluent gave the title compound (8.24 g, 65%) as an oil. m/z=219 m/z (M+1); r.t.=2.65 min. ¹H NMR (400 MHz; CDCl₃) δ 7.39-7.26 (m, 6H), 5.42 (dd, 1H), 4.63 (m, 3H), 3.71 (m, 2H), 2.75 (dd, 1H), 2.41 (dd, 1H).

### b. 2-Benzyloxymethyltetrahydropyran-4-one

To a solution of 2-benzyloxymethyl-2,3-dihydropyran-4-one (8.24 g, 37 mmol) in ethanol (100 mL) was added 10% palladium on carbon (40 mg, 0.38 mmol). The flask was evacuated and purged with hydrogen six times and then stirred for 72 hours under a hydrogen atmosphere. The reaction mixture was filtered through celite, washing with EtOH (100 mL), and evaporated to dryness. Purification by column chromatography on silica gel using 0 to 30% EtOAc/hexane as eluent gave the title compound (6.2 g, 75%) as an oil. m/z=no mass ion observed; r.t.=2.59 min. ¹H NMR (400 MHz; CDCl₃) δ 7.37-7.28 (m, 5H), 4.61 (s, 2H), 4.35 (ddd, 1H), 3.87-3.82 (m, 1H), 3.62 (dt, 1H), 3.58-3.52 (m, 2H), 2.67-2.58 (m, 1H), 2.53-2.47 (m, 1H), 2.37-2.31 (m, 2H).

### c. 7-benzyloxymethyl-3-nitro-7,8-dihydro-5H-pyrano[4,3-b]pyridine

A stirred suspension of 1-methyl-3,5-dinitro-1H-pyridin-2-one (1.48 g, 7.4 mmol) and 2-benzyloxymethyltetrahydropyran-4-one (1.64 g, 7.4 mmol) in 1M ammonia in methanol (70 mL) was stirred at 55° C. for 5 hours. After cooling, the reaction mixture was poured in to water (100 mL) and the product extracted in to EtOAc (4×50 mL). The combined organic extracts were dried (MgSO₄), filtered and concentrated under vacuum to leave a crude residue. Purification by column chromatography on silica gel using 0 to 20% EtOAc/hexane as eluent, followed by trituration with Et₂O/hexane gave the title compound (840 mg, 37%) as a solid. m/z=301 (M+1); r.t.=3.12 min. ¹H NMR (400 MHz; CDCl₃) δ 9.27 (d, 1H), 8.15 (d, 1H), 7.37-7.29 (m, 5H), 5.04 (d, 1H), 4.88 (d, 1H), 4.65 (d, 2H), 4.09-4.03 (m, 1H), 3.70 (d, 2H), 3.13-3.00 (m, 2H).

### d. 7-benzyloxymethyl-7,8-dihydro-5H-pyrano[4,3-b]pyridin-3-ylamine

A flask containing 7-benzyloxymethyl-3-nitro-7,8-dihydro-5H-pyrano[4,3-b]pyridine (840 mg, 2.8 mmol) and 10% palladium on carbon (30 mg, 0.3 mmol) was evacuated and purged with hydrogen four times, then stirred for 16 hours under a hydrogen atmosphere. The reaction mixture was filtered through celite, washing with EtOH (100 mL), then evaporated to dryness to give the title compound (650 mg, 84%) as a solid. m/z=270 (M+1); r.t.=1.63 min. ¹H NMR (400 MHz; d₆-DMSO) δ 7.76 (d, 11H), 7.41-7.26 (m, 5H), 6.57 (d, 1H), 5.12 (s, 2H), 4.61 (q, 2H), 4.54 (s, 2H), 3.93-3.86 (m, 1H), 3.60-3.52 (m, 2H), 2.58-2.54 (m, 2H).

### e. (3-Amino-7,8-dihydro-5H-pyrano[4,3-b]pyridin-7-yl)methanol

To a stirred solution of 7-benzyloxymethyl-7,8-dihydro-5H-pyrano[4,3-b]pyridin-3-ylamine (100 mg, 0.4 mmol) in CH₂Cl₂ (25 mL) at −78° C. was added boron tribromide (175 μL, 18.5 mmol). The reaction was allowed to warm slowly to room temperature and stirred for 2 hours. Water (10 mL) was added, then the reaction mixture was evaporated on to silica gel. Purification by column chromatography on silica gel using 0 to 15% MeOH/CH₂Cl₂ as eluent gave the title compound (35 mg, 50%) as a solid. m/z=181 (M+1); r.t.=0.26 min.

### Intermediate 59

### Preparation of 3-amino-5,6,7,8-tetrahydroquinolin-6-ol

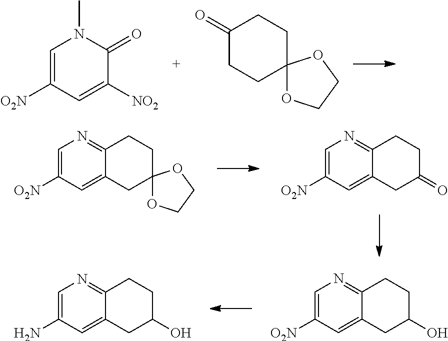

### a. 3-Nitro-7,8-dihydro-5H-quinolin-6-one ethylene ketal

A stirred suspension of 1-methyl-3,5-dinitro-1H-pyridin-2-one (1.0 g, 5 mmol) and 1,4-dioxaspiro[4,5]decan-8-one (941 mg, 6 mmol) in 1M ammonia in methanol (50 mL) was stirred at 55° C. for 16 hours. After cooling, the reaction mixture was poured in to water (100 mL) and extracted with EtOAc (4×50 mL). The combined organic extracts were dried (MgSO₄), filtered and concentrated under vacuum. Purification by column chromatography on silica gel using 30-40% EtOAc/hexane as eluent gave the title compound (650 mg, 50%) as a solid. m/z=237 (M+1); r.t.=2.36 min. ¹H NMR (400 MHz; CDCl₃) δ 9.21 (d, 1H), 8.15 (d, 1H), 4.06 (s, 4H), 3.23 (t, 2H), 3.10 (s, 2H), 2.12 (t, 2H).

### b. 3-Nitro-7,8-dihydro-5H-quinolin-6-one

To a solution of 3-nitro-7,8-dihydro-5H-quinolin-6-one ethylene ketal (500 mg, 2 mmol) in CH₂Cl₂ (50 mL) was added trifluoroacetic acid (10 mL). The reaction was heated to reflux and stirred for 5 days. After cooling, the mixture was poured in to saturated NaHCO₃ solution (100 mL) and extracted with CH₂Cl₂ (3×50 mL). The combined organic extracts were washed with brine (100 mL), dried (Na₂SO₄), filtered and concentrated under vacuum. Purification by column chromatography on silica gel using 0 to 5% MeOH in CH₂Cl₂ as eluent gave the title compound (260 mg, 60%) as a solid. ¹H NMR (400 MHz; CDCl₃) δ 9.29 (d, 1H), 8.25 (d, 1H), 3.74 (s, 2H), 3.41 (t, 2H), 2.75 (t, 2H). m/z=no mass ion observed; r.t.=2.36 min.

### c. 3-Nitro-5,6,7,8-tetrahydroquinolin-6-ol

To a stirred solution of 3-nitro-7,8-dihydro-5H-quinolin-6-one (270 mg, 1.4 mmol) in MeOH (40 mL) was added sodium borohydride (79 mg, 2.1 mmol). The reaction was stirred at room temperature for 1 hour, then the reaction mixture was poured in to saturated NaHCO₃ solution (100 mL) and extracted in to EtOAc (3×50 mL). The combined organic extracts were washed with brine (50 mL), dried (MgSO₄), filtered and concentrated under vacuum to give the title compound (200 mg, 70%) as a solid. m/z=195 (M+1); r.t.=1.85 min. ¹H NMR (400 MHz, d₆-DMSO) a 9.13 (d, 1H), 8.33 (d, 1H), 4.99 (d, 1H), 4.10-4.04 (m, 1H), 3.09-3.02 (m, 2H), 2.97-2.89 (m, 1H), 2.80 (dd, 1H), 2.00-1.93 (m, 1H), 1.91-1.84 (m, 1H).

### d. 3-Amino-5,6,7,8-tetrahydroquinolin-6-ol

To a solution of 3-nitro-5,6,7,8-tetrahydroquinolin-6-ol (200 mg, 1 mmol) in EtOH (25 mL) was added 10% palladium on carbon (20 mg, 0.1 mmol). The reaction mixture was evacuated and purged with hydrogen six times, then stirred for 16 hours under a hydrogen atmosphere. The reaction mixture was filtered through celite, washing with EtOH (100 mL) and the filtrate was evaporated to dryness, giving the title compound (160 mg, 90%) as a solid. m/z=165 (M+1); r.t.=0.29 min. ¹H NMR (400 MHz, d₆-DMSO) δ 7.70 (d, 1H), 6.58 (d, 1H), 4.95 (s, 2H), 4.76 (d, 1H), 3.88-3.84 (m, 1H), 2.80-2.70 (m, 2H), 2.65-2.57 (m, 1H), 2.52-2.46 (m, 1H), 1.90-1.86 (m, 1H), 1.71-1.62 (m, 1H).

### Intermediate 60

### Preparation of (7-Amino-1,5-naphthyridin-3-yl)methanol

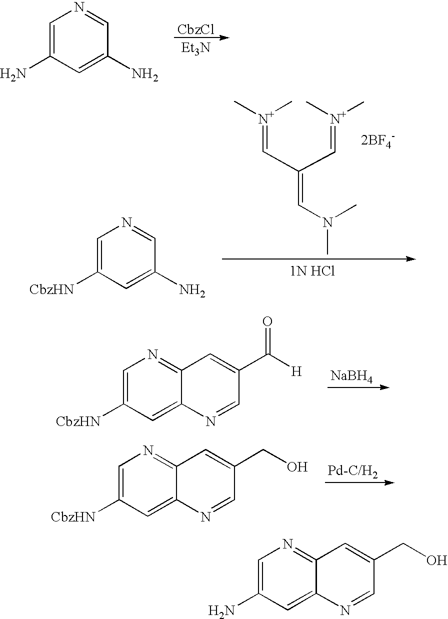

### a. Benzyl 5-aminopyridin-3-ylcarbamate

To a stirred solution of pyridine-3,5-diamine hydrochloride (240 mg, 1.6 mmol) in DMF (10 mL) and CH₂Cl₂ (10 mL) at −40° C. was added pyridine (1 drop), triethylamine (0.46 mL, 3.3 mmol) and benzyl chloroformate (0.24 mL, 1.6 mmol). The mixture was slowly warmed to rt and stirred at rt over weekend. Aq. NaHCO₃ solution (20 mL) and EtOAc (150 mL) were added. The organic phase was separated and washed with brine, dried (Na₂SO₄) and concentrated under vacuum. The residue was purified by column chromatography on silica gel using 50-100% EtOAc/hexane as eluent to give the product (170 mg, 42%) as a solid. m/z=243.8 (M+1); rt=1.62 min. ¹H NMR (400 MHz; d₆-DMSO) δ 9.68 (s, 1H), 7.78 (d, 1H, J=2.4 Hz), 7.57 (d, 1H, J=2.4 Hz), 7.45-7.32 (m, 5H), 7.17 (s, 1H), 5.33 (s, 2H), 5.14 (s, 2H).

### b. Benzyl 7-formyl-1,5-naphthyridin-3-ylcarbamate

A slurry of benzyl 5-aminopyridin-3-ylcarbamate (150 mg, 0.62 mmol) and 2-dimethylaminomethylene-1,3-bis(dimethylammonio)propane bis(tetrafluoroborate) (660 mg, 1.8 mmol) in n-butanol (10 mL) was heated at reflux for 24 h. The solution was concentrated under vacuum and the residue was dissolved in THF (20 mL) and 1N HCl (20 mL). The reaction mixture was stirred at rt overnight, then poured into a saturated solution of sodium bicarbonate (20 mL), and extracted with EtOAc (2×50 mL). The combined organic layers were washed with brine, dried (Na₂SO₄), and concentrated under vacuum. The residue was purified by column chromatography on silica gel using 0-100% EtOAc/hexane as eluent to give the product (45 mg, 24%) as a solid. m/z=308.3 (M+1); rt=2.72 min. ¹H NMR (400 MHz; d₆-DMSO) δ 10.76 (s, 1H), 10.27 (s, 1H), 9.31 (d, 1H, J=2.0 Hz), 9.10 (d, 1H, J=2.0 Hz), 8.85 (d, 1H, J=2.0 Hz), 8.63 (d, 1H, J=2.0 Hz), 7.51-7.35 (m, 5H), 5.27 (s, 2H).

### c. Benzyl 7-(hydroxymethyl)-1,5-naphthyridin-3-ylcarbamate

To a stirred mixture of benzyl 7-formyl-1,5-naphthyridin-3-ylcarbamate (110 mg, 0.36 mmol), MeOH (55 mL), and H₂O (1 mL) was added sodium tetrahydroborate (27 mg, 0.72 mmol). The mixture was stirred at rt until LC-MS indicated no starting material remained. The mixture was acidified with 1N HCl and concentrated under vacuum, and then treated with aq. Na₂CO₃ solution and EtOAc (100 mL). The organic layer was separated and washed with brine, dried (Na₂SO₄), and concentrated under vacuum. The residue was purified by column chromatography on silica gel using 0-5% MeOH/EtOAc as eluent to give the product (85 mg, 77%) as a solid. m/z=310.1 (M+1). rt=2.22 min.

### d. (7-Amino-1,5-naphthyridin-3-yl)methanol

A mixture of benzyl 7-(hydroxymethyl)-1,5-naphthyridin-3-ylcarbamate (55 mg, 0.18 mmol), 10% Pd—C (5 mg), MeOH (5 mL) was stirred under H₂ (1 atm) for 1 h. The mixture was filtered through celite and the filtrate was concentrated under vacuum to give the product (30 mg, 54%) as a solid.

### Intermediate 61

### Preparation of 1,5-naphthyridin-3-amine

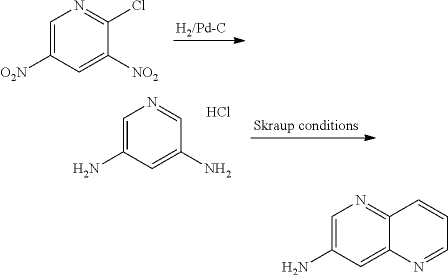

### a. Pyridine-3,5-diamine hydrochloride

A mixture of 2-chloro-3,5-dinitropyridine (4.6 g, 22 mmol), EtOAc (100 mL), chloroform (30 mL), and 10% Pd—C (500 mg) was hydrogenated at 60 psi for 24 h. The mixture was filtered through celite and the filtrate was concentrated under vacuum to give the product (3.4 g, 98%) as a solid. 7/z=110.0 (M+1); rt=0.26 min. ¹H NMR (400 MHz; d₆-DMSO) δ 15.02 (bs, 1H), 7.23 (d, 2H, J=2.4 Hz), 6.68 (t, 1H, 3=2.4 Hz), 6.17 (bs, 4H).

### b. 1,5-Naphthyridin-3-amine

1,2,3-Propanetriol (15 mL) was thoroughly mixed with pyridine-3,5-diamine hydrochloride (3.3 g, 23 mmol), sodium 3-nitrobenzenesulfonate (18 g, 81 mmol) and H₂O (20.5 mL, 1.14 mol). Concentrated H₂SO₄ (22 mL) was then added cautiously with stirring. The reaction mixture was heated by a heat-gun and the temperature was raised; the reaction was initiated at ca. 136° C. and the heat-gun was removed. After the initial violent ebullition had ceased, the temperature was kept there at for 1 h. After cooling, the mixture was poured into H₂O (300 mL), neutralized with K₂CO₃ and extracted with EtOAc (×3). The organic extracts were combined, washed with brine, dried (Na₂SO₄), and concentrated under vacuum. The residue was purified by basic aluminum oxide column using EtOAc as eluent to give the product (0.95 g, 29%) as a solid. m/z=146.0 (M+1); rt=0.46 min. ¹H NMR (400 MHz; d₆-DMSO) δ 8.70 (dd, 1H, J=4.4, 1.6 Hz), 8.51 (d, 1H, J=2.4 Hz), 8.12 (ddd, 1H, J=8.4, 1.6, 0.8 Hz), 7.33 (dd, 1H, J=8.4, 4.4 Hz), 7.20 (dd, 1H, J=2.4, 0.8 Hz), 6.07 (s, 2H).

### Intermediate 62

### Preparation of 1-(7-Amino-1,5-naphthyridin-3-yl)ethanol

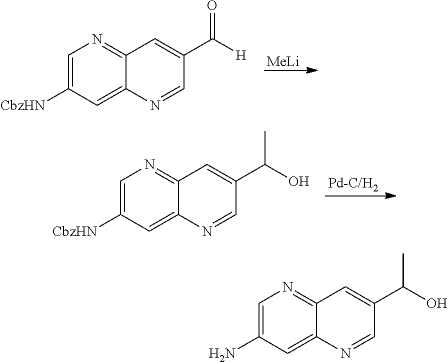

### a. Benzyl 7-(1-hydroxyethyl)-1,5-naphthyridin-3-ylcarbamate

To a stirred solution of benzyl 7-formyl-1,5-naphthyridin-3-ylcarbamate (310 mg, 1.0 mmol) in THF (20 mL) at −78° C. under N₂ was added a solution of MeLi in Et₂O (1.6 M; 1.5 mL, 2.4 mmol). The reaction mixture was slowly warmed to rt, and then quenched by the addition of sat. aq. NH₄Cl solution (10 mL) and extracted with EtOAc (3×50 mL). The combined organic layers were washed with brine, dried (Na₂SO₄), and concentrated under vacuum. The residue was purified by column chromatography using EtOAc as eluent to give the product (185 mg, 57%) as a solid. m/z=323.8 (M+1). rt=2.32 min.

### b. 1-(7-Amino-1,5-naphthyridin-3-yl)ethanol

A mixture of benzyl 7-(1-hydroxyethyl)-1,5-naphthyridin-3-ylcarbamate (185 mg, 0.57 mmol), 10% Pd—C (20 mg) and MeOH (20 mL) was stirred under H₂ (1 atm) for 1 h. The mixture was filtered through celite and the filtrate was concentrated under vacuum to give the product (155 mg) as a solid. m/z=189.9 (M+1). rt=0.60 min.

### Intermediate 79

### Preparation of (3-aminoquinolin-7-yl)methanol

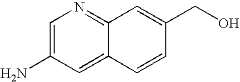

This compound was prepared using the procedure described in US 2006194801.

### Intermediate 63

### Preparation of 1-(7-Amino-3,4-dihydroquinolin-1(2H)-yl)ethanone

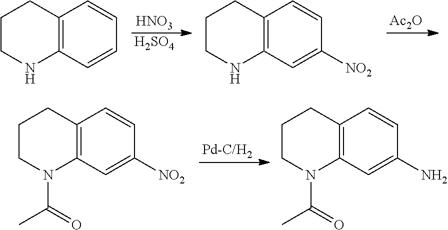

### a. 7-Nitro-1,2,3,4-tetrahydroquinoline

1,2,3,4-tetrahydroquinoline (8.0 g, 60 mmol) was slowly added to concentrated H₂SO₄ (160 mL) while cooled with an ice-bath. To the stirred solution was slowly added a solution of concentrated HNO₃ (6.0 mL) in sulfuric acid (20 mL) at 0-5° C. over 30 min. On completion of addition, the reaction mixture was poured onto crushed ice and then neutralized with solid K₂CO₃. EtOAc (600 mL) was added and the mixture was filtered to remove undissolved solids. The aqueous phase was extracted with EtOAc (300 mL×3). The combined organic layers were washed with water, dried (Na₂SO₄), and concentrated under vacuum. The residue was purified by column chromatography on silica gel and recrystallized from hexane-EtOAc to give the product (7.2 g, 67%) as a solid. m/z=179.2 (M+1); rt=3.09 min. ¹H NMR (400 MHz; CDCl₃) δ 7.40 (dd, 1H, J=8.0, 2.0 Hz), 7.28 (d, 1H, J=2.0 Hz), 7.02 (d, 1H, J=8.0 Hz), 4.30 (br s, 1H), 3.36 (t, 2H, J=5.6 Hz), 2.81 (t, 2H, J=6.0 Hz), 1.99-1.92 (m, 2H).

### b. 1-(7-Nitro-3,4-dihydroquinolin-1(2H)-yl)ethanone

A solution of 7-nitro-1,2,3,4-tetrahydroquinoline (350 mg, 2.0 mmol) and acetic anhydride (600 mg, 6.0 mmol) in pyridine (5 mL) was stirred at 70° C. overnight. The solvent was removed under vacuum and the residue was purified by column chromatography on silica gel to give the product (350 mg, 81%) as a solid. m/z=221.1 (M+1); rt=2.55 nm in.

### c. 1-(7-Amino-3,4-dihydroquinolin-1(2H)-yl)ethanone

A mixture of 1-(7-nitro-3,4-dihydroquinolin-1(2H)-yl)ethanone (350 mg, 1.6 mmol), 10% Pd—C (30 mg) and MeOH (10 mL) was stirred under an atmosphere of hydrogen (1 atm) for 3 h. The mixture was filtered through celite and the filtrate was concentrated under vacuum to give the product (300 mg, 100%) as a ‘syrup’. m/z=191.0 (M+1); rt=0.98 min.

### Intermediate 64

### Preparation of (E)-2-Methyl-4-(3,3,3-trifluoro-2-methylprop-1-enyl)benzoic acid

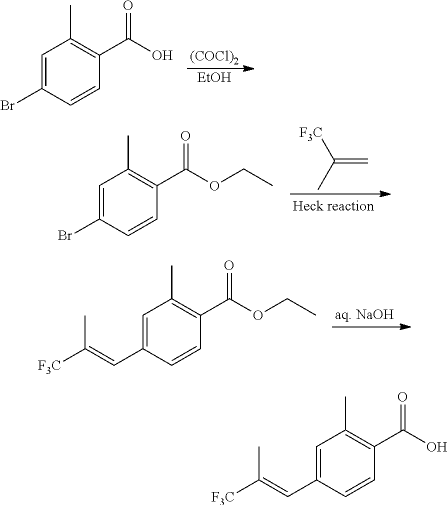

### a. Ethyl 4-bromo-2-methylbenzoate

Oxalyl chloride (10.6 g, 83.7 mmol) was added slowly to a mixture of 4-bromo-2-methylbenzoic acid (12.0 g, 55.8 mmol) in CH₂Cl₂ (200 mL) and DMF (0.2 mL) at 0° C. The mixture was stirred at 0° C. for 1 h, and then warmed to rt and stirred overnight. The mixture was concentrated under vacuum to give the acid chloride as a solid. The obtained acid chloride was redissolved in CH₂Cl₂ (200 mL) and dry ethanol (20 g, 0.4 mol) was added. The mixture was stirred at rt for 5 h, and then concentrated under vacuum to give the product (13.5 g, 100%) as an oil.

### b. (E)-Ethyl 2-methyl-4-(3,3,3-trifluoro-2-methylprop-1-enyl)benzoate

3,3,3-trifluoro-2-methylprop-1-ene (7.2 g, 66 mmol) was introduced to a dry ice cooled mixture of ethyl 4-bromo-2-methylbenzoate (4.0 g, 16 mmol), tri-o-tolylphosphine (1.00 g, 3.3 mmol), cesium carbonate (5.36 g, 16.4 mol), tetra-N-butylammonium chloride (1.37 g, 4.9 mmol), palladium acetate (180 mg, 0.82 mol), and N,N-dimethylacetamide (30 mL). The reaction mixture was flushed with N7 and sealed in a steel Parr instrument and stirred at 160° C. for 48 h. After cooling, the reaction mixture was filtered through celite and the filtrate was partitioned between EtOAc (200 mL) and water (100 mL). The organic layer was separated and washed with brine, dried (Na₂SO₄) and concentrated under vacuum. The residue was purified by chromatography on silica gel using EtOAc/hexane as eluent to give the product as an oil.

### c. (E)-2-Methyl-4-(3,3,3-trifluoro-2-methylprop-1-enyl)benzoic acid

A mixture of (E)-ethyl 2-methyl-4-(3,3,3-trifluoro-2-methylprop-1-enyl)benzoate (3.0 g, 7.7 mmol), 2 N aq. NaOH (25 mL), and MeOH (50 mL) was stirred at 40° C. overnight. The mixture was concentrated under vacuum and the residue was treated with H₂O and acidified with 1N HCl to pH 2-3. The mixture was extracted with EtOAc (100 mL×2) and the combined organic layers were washed with brine, dried (Na₂SO₄), and concentrated under vacuum to give the product as a solid. m/z=242.7 (M−1); rt=3.29 min. ¹H NMR (400 MHz; d₆-DMSO) δ 12.96 (s, 1H), 7.87 (d, 1H, J=8.0 Hz), 7.39 (s, 1H), 7.37 (d, 1H, J=8.0 Hz), 7.17 (s, 1H), 2.55 (s, 3H), 2.02 (s, 3H).

### Preparation of Amido Compounds

### Amide Formation

### Method A: A Representative Synthesis of Benzamides Using an Automated Parallel Synthesis Method

The appropriate benzoic acid (2 mmol) is dissolved or suspended in 15 ml of chloroform and treated with 20 mmol of thionyl chloride. The reaction mixture is refluxed for fifteen minutes and the solvents are removed under vacuum. The residue is dissolved in 4 ml of anhydrous chloroform and 60 μl (30 μmole) of this solution is added to each well of the 96 well glass plates. Appropriate amine is then added to the corresponding well (60 μmole), followed by n,n-diisopropylethylamine (120 μmole). The plate is then heated at 65° C. for 15 minutes. The solvents are removed using an ht-12 genevac centrifugal evacuator and 100 μl of dmso is added to each well and the compounds are transferred to a 96-well polypropylene reaction plate. The plates are then sealed using an abgene plate sealer and submitted to lc-ms purification.

### Method B: A Representative Synthesis of Benzamides Using an Automated Parallel Synthesis Method

In one well of a 96-well polypropylene reaction plate was added the appropriate benzoic acid (6.03 mg, 30 μmol) in 15 μl of anhydrous pyridine. To the reaction was added TFFH (TFFH is fluoro-N,N,N′,N′-tetramethylformamidinium hexafluorophosphate; 12 mg, 45 μmol), followed by diisopropylethylamine (6.0 mg, 45 μmol), followed by the appropriate amine (60 μmol). The reaction plate was heated at 50° C. for 15 minutes and the solvent was evaporated. The residue was dissolved in DMSO and purified using LC-MS based purification (50 mm×10 mm Phenomenex Gemini Column using a 10-100% acetonitrile-water gradient).

### Method C:

To a mixture of the acid (0.4 mmol), N-(3-dimethylaminopropyl)-N′-ethylcarbodiimide hydrochloride (0.8 mmol), 1-Hydroxybenzotriazole hydrate (0.24 mmol) and CH₂Cl₂ (5 mL) was added the appropriate amine (0.5 mmol) and DIPEA (0.2 mL). The mixture was stirred at room temperature overnight, diluted with EtOAc, washed with brine, dried (Na₂SO₄), and concentrated. The residue was purified by column chromatography on silica gel to give the product.

### Method D:

To a mixture of acid (1.0 mmol), N-(3-Dimethylaminopropyl)-N′-ethylcarbodiimide hydrochloride (385 mg, 2.0 mmol), 1-hydroxybenzotriazole hydrate (0.5-1.0 mmol), DMF (2 mL) and CH₂Cl₂ (5 mL) was added amine (1.2 mmol) and diisopropylethylamine (0.5 mL). The mixture was stirred at room temperature overnight, diluted with EtOAc, washed with brine, dried Na₂SO₄), and concentrated. The residue was purified by column to give the amide.

### Method E:

To a stirred solution of acid (1.0 mmol) in dry CH₂Cl₂ (10 mL) and DMF (2 drops) at 0° C. was added oxalyl chloride (1.5 mmol). The mixture was stirred at 0° C. for 1 h and then warmed to rt for 3 h. The solvent was removed in vacuo. A solution of the obtained acid chloride in CH₂Cl₂ (2 mL) was added to a solution of amine (1.0 mmol) in CH₂Cl₂ (3 mL) and pyridine (2 mL) at 0° C. The reaction mixture was stirred at rt overnight, and then diluted with EtOAc. The organic phase was washed with aq. NaHCO₃ solution and brine, dried (Na₂SO₄), and concentrated. The residue was purified by chromatography to give the amide.

### Method F:

To a stirred solution of acid (0.25 mmol) in dry THF or CH₂Cl₂ (5 mL) and DMF (1 drop) at 0° C. was added oxalyl chloride (0.40 mmol). The mixture was stirred at 0° C. for 1 h and then warmed to rt. The solvent was removed in vacuo. A solution of the obtained acid chloride in CH₂Cl₂ (2 mL) was added to a solution of amine (0.25 mmol) in CH₂Cl₂ (10 mL), Et₃N (0.2 mL), DMAP (5 mg) at 0° C. The reaction mixture was stirred at rt overnight, and then diluted with EtOAc (100 mL). The organic phase was washed with aq. NaHCO₃ solution and brine, dried, and concentrated. The residue was purified by chromatography to give the amide.

### Method G:

To a cooled (0° C.) and well stirred suspension of the appropriate acid (1 eq) in CH₂Cl₂ (ca. 3 mL per mmol) and DMF (catalytic quantity) is added oxalyl chloride (1.5 eq) slowly drop-wise and the mixture is agitated for one hour. The mixture is concentrated under vacuum and the residue re-suspended in CH₂Cl₂. The appropriate amine (0.5-1.0 eq) is then added and the mixture is stirred for 1-48 hours before being worked-up and purified.

### Method H:

N,N-Diisopropylethylamine (1 eq) was added in one portion to a stirred mixture of 2-methyl-4-(3,3-dimethylbut-1-ynyl)benzoic acid (1 eq) and N,N,N′,N′-tetramethyl-O-(7-azabenzotriazol-1-yl)uronium hexafluorophosphate (1.05 eq) in N,N-dimethylformamide (ca. 3 mL per 0.5 mmol of starting acid) at room temperature. The mixture was stirred at room temperature for approx. 2 hours then a solution of the appropriate amine (1 eq) in DMF (1 mL) was added in one portion. The mixture was stirred overnight then worked-up by pouring in to H₂O (30 mL) and EtOAc (30 mL). The aqueous and organic layers were partitioned and the aqueous was extracted with EtOAc (2×30 mL). The combined organic extracts were washed with brine (1×30 mL), dried (Na₂SO₄), filtered and the solvent removed under vacuum to leave a crude residue. Appropriate purification was employed to furnish the desired final compound.

### Method I:

A mixture of the acid (1 mmol), N-(3-dimethylaminopropyl)-N′ethylcarbodiimide hydrochloride (3 mmol), 1-hydroxybenzotriazole hydrate (1.5 mmol) and the amine (2 mmol) was stirred in DMF at room temperature overnight. The mixture was partitioned between EtOAc and water. The organic layer was separated and washed with saturated aqueous NaHCO₃, water, brine, dried Na₂SO₄), filtered and the filtrate was concentrated in vacuo to a residue which was purified by flash column chromatography.

### Method J:

DIPEA (0.92 mmol) was added to the solution of appropriate acid (0.46 mmol), appropriate amine (0.69 mmol) and TFFH (0.69 mmol) in anhydrous pyridine (3 mL) and the reaction mixture was stirred at 60° C. overnight. Volatiles were removed and the residue was suspended in water, extracted by EtOAc and the organic phase was washed by water, brine and was dried over Na₂SO₄, solvent was removed and the residue was chromatographed to give the product.

### Method K:

DIPEA (0.92 mmol) was added to the solution of appropriate acid (4.0 mmol), appropriate amine (3.2 mmol) and TFFH (6.0 mmol) in anhydrous pyridine (10 mL) and the reaction mixture was stirred at 70° C. overnight. Volatiles were removed and the residue was dissolved in EtOAc and the organic phase was washed by water, Na₂CO₃ aqueous solution, brine and was dried over Na₂SO₄, solvent was removed and the residue was chromatographed to yield the product.

### Method L:

To a solution of acid (0.5 mmol), N-(3-Dimethylaminopropyl)-N′-ethylcarbodiimide hydrochloride (1.0 mmol), 1-hydroxybenzotriazole hydrate (1.0 mmol) in DMF (5 mL) and CH₂Cl₂ (5 mL) were added amine (0.75 mmol) and diisopropylethylamine (1.0 mmol). The mixture was stirred at 40° C. overnight before diluted with EtOAc, washed with brine, dried over Na₂SO₄ and concentrated. The residue was purified by column to give the amide.

### Method M:

The amine (1 eq) was added in one portion to a stirred solution of the acid (I eq), N-(3-Dimethylaminopropyl)-N′-ethylcarbodiimide hydrochloride (1 eq), 4-N,N-dimethylaminopyridine (1 eq) and Et₃N (2 eq) in CH₂Cl₂ (ca. 3 mL per 0.125 mmol) and the mixture stirred until completion of the reaction (typically left overnight). The mixture was diluted with more CH₂Cl₂ (30 mL) and washed with H₂O (1×20 mL), then dried (Na₂SO₄), filtered and concentrated under vacuum. The residue was purified by column chromatography on silica gel or preparative thin-layer chromatography.

### Compound 187

### 2-Methyl-N-quinolin-3-yl-4-((E)-3,3,3-trifluoro-propenyl)-benzamide

To a stirred solution of 4-((E)-3,3,3-trifluoroprop-1-enyl)-2-methylbenzoic acid (4.0 g, 0.017 mol) in CH₂Cl₂ (50 mL) and DMF (2 drops) at 0° C. was added oxalyl chloride (2.20 mL, 0.0261 mol). The mixture was stirred at 0° C. for 1 h and then warmed to room temperature for 2 h. The solvent was removed in vacuo.

The above acid chloride was reacted with 3-quinolinamine (2.50 g, 0.0174 mol) in CH₂Cl₂ (20 mL) and pyridine (10 mL) at room temperature overnight. The mixture was concentrated in vacuo, and the residue was treated with EtOAc and aq. NaHCO₃. The organic layer was separated, washed with brine, dried (Na₂SO₄), and evaporated. The residue was purified by column (EtOAc/CH₂Cl₂: 0-30%) to give a white solid (4.9 g, 81%). (d₆-DMSO) a 10.86 (s, 1H), 9.04 (d, 1H, J=2.4 Hz), 8.88 (d, 1H, J=2.4 Hz), 7.98 (d, 2H, J=8.8 Hz), 7.72-7.58 (m, 5H), 7.43-7.35 (m, 1H), 6.90 (dq, 1H, J=16.4, 7.2 Hz), 2.46 (s, 3H). MS (ESI): m/z 357 (M+H)⁺

### Compound 187

### Alternate Method

A well stirred mixture of methyl 4-bromo-2-methylbenzoate (50 g, 0.22 mol), Palladium Acetate (4.9 g, 0.02 mol), tri-o-Tolylphosphine (10 g, 0.04 mol), Tetra-N-butylammonium chloride (20 g, 0.06 mol) and Cesium Carbonate (71 g, 0.22 mol) in N,N-Dimethylacetamide (200 mL, 2 mol) was cooled to −78° C. in a 500 mL par pressure reactor equipped with a pressure gauze. 3,3,3-Trifluoroprop-1-ene was then pumped in until the desired amount (84 g, 0.87 mol) condensed into the reactor. The valve was securely closed and the flask was heated in an oil bath at 135° C. for 3 days.

After the completion of the reaction, the reactor was cooled down again to −78° C. before carefully opening the valve to air. The reactor was slowly allowed to warm to ambient temperature.

The solids were filtered through Celite®, concentrated at reduced pressure to half the volume, dissolved in EtOAc (400 mL), washed successively with water (2×400 mL) and brine, dried (MgSO₄) and concentrated to give a dark oil. LC/MS analysis indicated the presence of saponified product (˜25%) in addition to other non polar impurities which were not identified.

The dark oil was then dissolved in anhydrous THF (200 mL), cooled to 0° C. and treated with oxalyl chloride (30 mL, large excess). A few drops of DMF was added to initiate the reaction. After stirring for 1 hour at the same temperature, the mixture was concentrated to dryness, re-dissolved in MeOH (100 mL) and carefully treated with triethylamine (30 mL, large excess). After stirring for few hours, the mixture was concentrated to dryness, re-dissolved in hot EtOAc (500 mL) and washed twice with warm water. The organic layer was dried and concentrated to obtain the crude ester as a dark oil which was passed through a short column of silica gel using 30% EtOAc in Hexane. LC/MS analysis of the above product indicated ˜80% purity.

The crude ester was saponified as follows. The ester was treated with LiOH (10.45 g, 0.44 mol) in a 3:1 mixture (200 mL) of THF and water and the mixture was heated to reflux for 4 hours. The mixture was concentrated to half the volume, diluted with water (1.5 L) and cooled to 0° C. before being acidified to pH 2.0 with conc.HCl. The white precipitate was filtered, washed with water and vacuum dried to constant weight.

The crude product was repeatedly crystallized from EtOAc/hexane to −99% purity.

To a stirred solution of 4-((E)-3,3,3-trifluoroprop-1-enyl)-2-methylbenzoic acid (30 g, 0.13 mol) in CH₂Cl₂ (200 mL) and DMF (2 drops) at 0° C. was added oxalyl chloride (19.85 g, 0.16 mol). The mixture was stirred at 0° C. for 1 h and then warmed to ambient temperature and further stirred for 2 h. The mixture was concentrated to dryness and vacuum dried until constant weight to yield the acid chloride.

The above acid chloride was reacted with 3-quinolinamine (22.55 g, 0.16 mol) in THF (200 mL) and triethylamine (15.83 g, 0.16 mol) at ambient temperature overnight. The mixture was concentrated in vacuo, and the crude product was treated with EtOAc and aq. NaHCO₃. The organic layer was separated, washed with brine, dried (Na₂SO₄), and evaporated. The crude product was purified by repetitive crystallizations to obtain the title compound as a white solid.

### Compound 197

A mixture of 2-methyl-N-(2-methylbenzo[d]thiazol-5-yl)-4-(3,3-dimethylbut-1-ynyl)benzamide (50 mg, 0.14 mmol), selenium dioxide (46 mg, 0.41 mmol), and 1,4-dioxane (10 mL) was stirred under an atmosphere of nitrogen at 80° C. overnight. After cooling, the mixture was filtered through celite and the filtrate was treated with aq. NaHCO₃ and extracted with EtOAc. The organic layer was washed with brine, dried (Na₂SO₄), and concentrated under vacuum. The residue was dissolved in THF—H₂O (2:1) (10 mL) and NaBH₄ (50 mg) was added slowly. The mixture was stirred at rt for 2 h and then acidified with 1N HCl. After treated with aq. NaHCO₃, the mixture was extracted with EtOAc. The combined organic layers were washed with brine, dried (Na₂SO₄), and concentrated under vacuum. The residue was purified by preparative thin-layer chromatography to give N-(benzo[d]thiazol-5-yl)-2-methyl-4-(3,3-dimethylbut-1-ynyl)benzamide (compound 198-11 mg) as a light yellow solid and N-(2-(hydroxymethyl)benzo[d]thiazol-5-yl)-2-methyl-4-(3,3-dimethylbut-1-ynyl)benzamide (compound 197-27 mg) as a light yellow solid.

### Compound 225

(E)-4-(3,3,3-trifluoroprop-1-enyl)-2-methyl-N-(2-methylbenzo[d]thiazol-5-yl)benzamide (200 mg, 0.0005 mol) and selenium dioxide (177 mg, 0.00160 mol) were placed in 20 mL dioxane and the reaction was heated at 80° C. overnight under nitrogen. The reaction was cooled and filtered through celite. The filtrate was partitioned between EtOAc and NaHCO₃. The organic layer was separated, washed with water, brine, dried (Na₂SO₄) and concentrated under vacuum. The residue was dissolved in THF/H₂O (2:1; 20 mL) and NaBH₄ (200 mg, 5.3 mmol) was added in three batches. The mixture was stirred at room temperature for 2 h, then quenched by addition of 1N HCl. The mixture was basified by addition of sat'd NaHCO₃ and extracted with EtOAc. The organic layer was washed with water, brine, dried (Na₂SO₄) and concentrated under vacuum. The residue was purified by column chromatography on silica gel using EtOAc/hexane (0-100%) as eluent and then again using MeOH/CH₂Cl₂ (0-3%) as eluent to give the product (40 mg) as a solid. m/z=392.6 Further purification by preparative HPLC (water/acetonitrile) gave the product (35 mg) as a white solid. m/z=392.6.

### Compound 228

To a stirred solution of (E)-4-(3,3,3-trifluoroprop-1-enyl)-2-methylbenzoic acid (0.20 g, 0.87 mmol) in CH₂Cl₂ (50 mL) and DMF (2 drops) at 0° C. was added oxalyl chloride (0.11 mL, 1.3 mmol). The mixture was stirred at 0° C. for 1 h and then warmed to rt for 2 h. The solvent was removed in vacuo. The above acid chloride was added to a solution of (7-aminoquinolin-3-yl)methanol (76 mg, 0.43 mmol) in CH₂Cl₂ (5 mL) and pyridine (10 mL). The reaction mixture was stirred at rt overnight, and then concentrated in vacuo. The residue was treated with EtOAc and aq. NaHCO₃ solution. The organic layer was separated, washed with brine, dried (Na₂SO₄), and concentrated under vacuum. The residue was purified by column chromatography on silica gel using EtOAc/hexane (0-50%) as eluent to give the ester [95 mg, m/z: 599.2 (M+1)]. The ester was dissolved in MeOH (5 mL) and K₂CO₃ (200 mg) was added. The mixture was stirred at rt for 3 h, and then methanol was removed under vacuum. The residue was treated with water and EtOAc. The organic layer was separated, washed with brine, dried (Na₂SO₄), and concentrated under vacuum. The residue was purified by preparative thin-layer chromatography with acetone-CH₂Cl₂ (1:1) to give a white solid (43 mg, 240%). LC-MS: 2.29 min, 387.7 (M+1).

### Compound 229

To a stirred solution of 7,8-Dihydro-5H-pyrano[4,3-b]pyridin-3-ylamine (50 mg, 0.3 mmol) in anhydrous DMF (2 mL) was added a stirred solution of (E)-4-(3,3,3-trifluoroprop-1-enyl)-2-methylbenzoic acid (91.96 mg, 0.4 mmol), N-(3-dimethylaminopropyl)-N′-ethylcarbodiimide hydrochloride (76.59 mg, 0.4 mmol), HOBt (62.98 mg, 0.46 mmol), 4-N,N-dimethylaminopyridine (2 mg, 0.02 mmol) and DIPEA (139 μL, 0.8 mmol) in anhydrous DMF (3 mL). The reaction was stirred overnight at room temperature. The reaction mixture was poured into saturated NaHCO₃ solution (50 mL) and extracted with EtOAc (3×50 mL). The combined organics were washed with brine (3×50 mL), dried (MgSO₄), filtered and concentrated under vacuum. Purification by column chromatography on silica gel (0 to 5% MeOH in DCM over 60 minutes) gave the desired product (39 mg, 30%) as an off-white solid.

### Compound 301

### Preparation of (E)-7-(2-methyl-4-(3,3,2-trifluoroprop-1-enyl)benzamido)quinoline-3-carboxylic acid

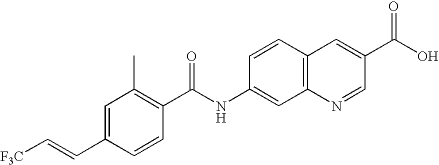

### a. (E)-Methyl 7-(2-methyl-4-(3,3,3-trifluoroprop-1-enyl)benzamido)quinoline-3-carboxylate

To a stirred solution of 4-((E)-3,3,3-trifluoroprop-1-enyl)-2-methylbenzoic acid (260 mg, 1.1 mmol) in CH₂Cl₂ (10 mL) and DMF (1 drops) at 0° C. was added oxalyl chloride (140 μL, 1.7 mmol). The mixture was stirred at 0° C. for 1 h and then warmed to rt and stirred for 3 h. The solvent was removed under vacuum and the obtained acid chloride was reacted with 7-amino-quinoline-3-carboxylic acid methyl ester (230 mg, 1.1 mmol) in CH₂Cl₂ (3 mL) and pyridine (2 mL) at rt overnight. The mixture was diluted with EtOAc (100 mL), washed with aq. NaHCO₃ and brine, dried (Na₂SO₄), and concentrated under vacuum. The residue was purified by column chromatography on silica gel using 0-40% EtOAc I hexane as eluent to give the product (280 mg, 59%) as a solid. m/z=415.2 (M+1); rt=3.45 min. ¹H NMR (400 MHz; d₆-DMSO) δ 10.96 (s, 1H), 9.28 (d, 1H, J=2.4 Hz), 8.93 (d, 1H, J=2.4 Hz), 8.67 (s, 1H), 8.19 (d, 1H, J=8.8 Hz), 7.96 (dd, 1H, J=8.8, 2.0 Hz), 7.71 (s, 1H), 7.68 (d, 1H, J=8.4 Hz), 7.62 (d, 1H, J=8.0 Hz), 7.42-7.35 (m, 1H), 6.88 (dq, 1H, J=16.4, 7.2 Hz), 3.95 (s, 3H), 2.45 (s, 3H).

### b. (E)-7-(2-methyl-4-(3,3,3-trifluoropropl-enyl)benzamido)quinoline-3-carboxylic acid

A mixture of (E)-methyl 7-(2-methyl-4-(3,3,3-trifluoroprop-1-enyl)benzamido)quinoline-3-carboxylate (110 mg, 0.26 mmol), lithium hydroxide (65 mg, 2.7 mmol), MeOH (10 mL), THF (10 mL), and water (5 mL) was stirred at 50° C. overnight. The mixture was concentrated under vacuum and the residue was acidified with 1N aq. HCl to pH 4-5 and extracted with EtOAc (100 mL×2). The combined organic layers were washed with brine, dried (Na₂SO₄), and concentrated under vacuum. The residue was washed with CH₂Cl₂ to give the product as a solid. m/z=399.2 (M−1); rt=2.95 min. ¹H NMR (400 MHz; d₆-DMSO) δ 13.39 (br s, 1H), 10.89 (s, 1H), 9.27 (d, 1H, J=2.4 Hz), 8.88 (d, 1H, J=2.0 Hz), 8.66 (s, 1H), 8.16 (d, 1H, J=8.8 Hz), 7.95 (dd, 1H, J=8.8, 2.0 Hz), 7.71 (s, 1H), 7.68 (d, 1H, J=8.0 Hz), 7.62 (d, 1H, J=8.0 Hz), 7.42-7.35 (m, 1H), 6.89 (dq, 1H, J=16.4, 7.2 Hz), 2.45 (s, 3H).

### Compound 302

### Preparation of (E)-N-(7-hydroxynaphthalen-1-yl)-2-methyl-4-(3,3,3-trifluoroprop-1-enyl)benzamide

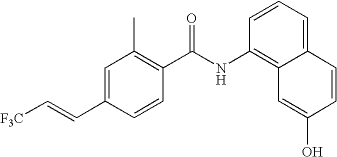

To a stirred solution of 8-iminonaphthalen-2-ol (130 mg, 0.82 mmol) in anhydrous toluene (7 mL) was added a solution of trimethylaluminium in hexanes (IM; 0.82 mL, 0.82 mmol), drop wise over 5 minutes. The reaction was stirred at room temperature for 16 hours, then a solution of 2-methyl-4-((E)-3,3,3-trifluoropropenyl)benzoic acid methyl ester (100 mg, 0.4 mmol) in anhydrous toluene (3 mL) was added, and the reaction was heated at reflux for 3 hours. After cooling, the reaction mixture was poured into saturated NaHCO₃ solution (50 mL) and extracted with EtOAc (3×50 mL). The combined organic extracts were washed with brine (3×50 mL), dried (MgSO₄), filtered and concentrated under vacuum. Purification by column chromatography on silica gel using 0 to 30% EtOAc/hexane as eluent gave the title compound (44 mg, 30%) as a solid. m/z=372 (M+1); r.t.=3.32 min. ¹H NMR (400 MHz; d₆-DMSO) δ 10.21 (s, 1H), 9.84 (s, 1H), 7.82 (d, 1H), 7.73-7.68 (m, 4H), 7.51 (d, 11H), 7.39 (d, 11H), 7.32-7.29 (m, 2H), 7.11 (dd, 11H), 6.93-6.84 (m, 1H), 2.51 (s, 3H).

### Compound 303

### Preparation of (E)-N-(3-(2-Hydroxypropan-2-yl)quinolin-7-yl)-2-methyl-4-(3,3,3-trifluoroprop-1-enyl)benzamide

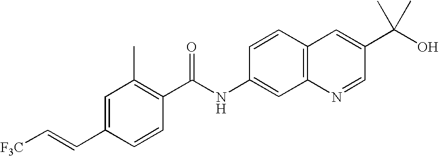

To a stirred solution of 4-((E)-3,3,3-trifluoroprop-1-enyl)-2-methylbenzoic acid (62 mg, 0.27 mmol) in CH₂Cl₂ (5 mL) and DMF (1 drop) at 0° C. was added oxalyl chloride (34 μL, 0.41 mmol). The mixture was stirred at 0° C. for 1 h and then warmed to rt and stirred for 3 h. The solvent was removed under vacuum and the obtained acid chloride was reacted with 2-(7-aminoquinolin-3-yl)propan-2-ol (55 mg, 0.27 mol) in CH₂Cl₂ (3 ml) and pyridine (2 mL) at rt overnight. The mixture was diluted with EtOAc (100 mL), washed with aq. NaHCO₃ and brine, dried (Na₂SO₄), and concentrated under vacuum. The residue was purified by column chromatography on silica gel using 30-100% EtOAc/hexane as eluent to give the product (95 mg, 82%) as a solid. m/z=415.2 (M+1); rt=2.33 min. ¹H NMR (400 MHz; d₆-DMSO) δ10.69 (s, 1H), 9.02 (d, 1H, J=2.4 Hz), 8.55 (s, 1H), 8.26 (d, 1H, J=2.4 Hz), 7.94 (d, 1H, J=8.8 Hz), 7.84 (dd, 1H, J=8.8, 1.6 Hz), 7.70 (s, 1H), 7.66 (d, 1H, J=8.0 Hz), 7.60 (d, 1H, J=8.0 Hz), 7.42-7.35 (m, 1H), 6.89 (dq, 1H, J=16.4, 7.2 Hz), 5.34 (s, 1H), 2.45 (s, 3H), 1.56 (s, 6H).

### Compound 304

### Preparation of (E)-N-(3-(1-hydroxyethyl)quinolin-7-yl)-2-methyl-4-(3,3,3-trifluoroprop-1-enyl)benzamide

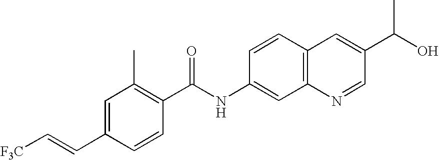

To a stirred solution of 4-((E)-3,3,3-trifluoroprop-1-enyl)-2-methylbenzoic acid (115 mg, 0.5 mmol) in CH₂Cl₂ (10 mL) and DMF (1 drop) at 0° C. was added oxalyl chloride (63 μL, 0.75 mmol). The mixture was stirred at 0° C. for 1 h and then warmed to rt and stirred for 3 h. The solvent was removed under vacuum and the obtained acid chloride was reacted with 1-(7-aminoquinolin-3-yl)ethanol (94.0 mg, 0.5 mmol) in CH₂Cl₂ (3 mL) and pyridine (2 mL) at rt overnight. The mixture was diluted with EtOAc (100 mL), washed with aq. NaHCO₃ and brine, dried (Na₂SO₄), and concentrated under vacuum. The residue was purified by column chromatography on silica gel using EtOAc as eluent gave the product (170 mg, 85%) as a solid. m/z=401.3 (M+1); rt=2.32 min. ¹H NMR (400 MHz; d₆-DMSO) δ 10.69 (s, 1H), 8.87 (d, 1H, J=2.0 Hz), 8.55 (s, 1H), 8.17 (d, 1H, J=2.0 Hz), 7.94 (d, 1H, J=8.8 Hz), 7.85 (dd, 1H, J=8.8, 2.0 Hz), 7.70 (s, 1H), 7.66 (d, 1H, J=8.0 Hz), 7.60 (d, 1H, J=8.0 Hz), 7.42-7.35 (m, 1H), 6.89 (dq, 1H, J=16.4, 7.2 Hz), 5.45 (d, 1H, J=4.4 Hz), 4.95 (m, 1H), 2.45 (s, 3H), 1.46 (d, 3H, J=6.4 Hz).

### Compound 305

### Preparation of (E)-N-(3-((2-Hydroxyethoxy)methyl)quinolin-7-yl)-2-methyl-4-(3,3,3-trifluoroprop-1-enyl)benzamide

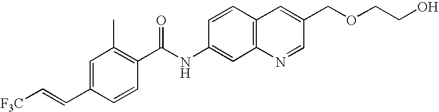

To a stirred solution of 4-((E)-3,3,3-trifluoroprop-1-enyl)-2-methylbenzoic acid (46 mg, 0.20 mmol) in CH₂Cl₂ (5 mL) and DMF (1 drop) at 0° C. was added oxalyl chloride (25 μL, 0.30 mmol). The mixture was stirred at 0° C. for 1 h and then warmed to rt and stirred for 3 h. The solvent was removed under vacuum and the obtained acid chloride was reacted with 3-((2-(tert-butyldimethylsilyloxy)ethoxy)methyl)quinolin-7-amine (66 mg, 0.20 mmol) in CH₂Cl₂ (3 mL) and pyridine (3 mL) at rt overnight. The mixture was concentrated under vacuum and the residue was purified by column chromatography on silica gel to give the intermediate as a white solid (100 mg; m/z=545.0 (M+1); rt 3.88 min). The intermediate was dissolved in MeOH (10 mL) and conc. HCl (1 mL) was added. The mixture was stirred at rt overnight and then concentrated under vacuum. The residue was treated with aq. NaHCO₃ (10 mL) and extracted with EtOAc (3×20 mL). The combined organic layers were washed with brine, dried (Na₂SO₄), and concentrated. The residue was purified by column chromatography on silica gel using 0-2% MeOH/EtOAc as eluent to give the product (67 mg, 75%) as a solid. m/z=431.1 (M+1); rt=2.33 min. ¹H NMR (400 MHz; d₆-DMSO) δ 10.73 (s, 1H), 8.84 (d, 1H, J=2.4 Hz), 8.57 (s, 1H), 8.23 (d, 1H, J=1.2 Hz), 7.96 (d, 1H, J=8.8 Hz), 7.87 (dd, 1H, J=8.8, 1.6 Hz), 7.70 (s, 1H), 7.67 (d, 1H, J=8.0 Hz), 7.60 (d, 1H, J=8.0 Hz), 7.42-7.35 (m, 1H), 6.89 (dq, 1H, J=16.4, 7.2 Hz), 4.70 (s, 3H), 3.61-3.52 (m, 4H), 2.45 (s, 3H).

### Compound 306

### Preparation of (E)-2-methyl-N-(8-oxo-5,6-7,8-tetrahydronaphthylen-2-yl)-4(3,3,3-trifluoroprop-1-enyl)benzamide

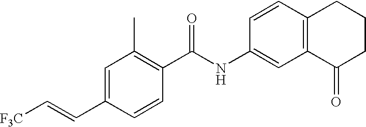

To a stirred solution of 7-Amino-3,4-dihydro-2H-naphthalen-1-one (52 mg, 0.32 mmol) in anhydrous DMF (2 mL) was added a stirred solution of 4-((E)-3,3,3-trifluoroprop-1-enyl)-2-methylbenzoic acid (75 mg, 0.32 mmol), N-(3-dimethylaminopropyl)-N′-ethylcarbodiimide hydrochloride (75 mg, 0.39 mmol), 0.5M 1-hydroxy-7-azabenzotriazole in DMF (0.8 mL, 0.4 mmol) and N,N-diisopropylethylamine (0.23 mL, 1.3 mmol) in DMF (2 mL). The mixture was stirred for 16 hours at room temperature then poured into saturated NaHCO₃ solution (50 mL) and extracted with EtOAc (3×50 mL). The combined organic extracts were washed with brine (3×50 mL), dried (MgSO₄), filtered and concentrated under vacuum. Purification by column chromatography on silica gel using 0-30% EtOAc/hexane gave the product (43 mg, 35%) as a solid. m/z=374 (M+1); r.t.=1.75 min. ¹H NMR (400 MHz; CDCl₃) δ 8.19 (d, 1H), 7.85 (d, 1H), 7.57 (s, 1H), 7.52 (d, 1H), 7.37-7.35 (m, 2H), 7.32 (d, 1H), 7.14 (dd, 1H), 6.31-6.22 (m, 1H), 2.96 (t, 2H), 2.67 (t, 2H), 2.53 (s, 3H), 2.18-2.12 (m, 2H).

### Compound 307

### Preparation of (E)-N-(8-hydroxy-5,6,7,8-tetrahydronaphthylen-2-yl)-2-methyl-4-(3,3,3-trifluoroprop-1-enyl)benzamide

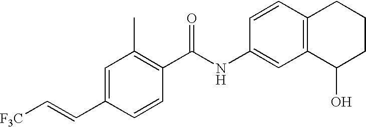

To a stirred solution of (E)-2-Methyl-N-(8-oxo-5,6,7,8-tetrahydronaphthylen-2-yl)-4-(3,3,3-trifluoroprop-1-enyl)benzamide (35 mg, 0.09 mmol) in EtOH (2 mL) was added sodium borohydride (10.6 mg, 0.28 mmol). The mixture was stirred for 16 hours at room temperature then poured into saturated NaHCO₃ solution (50 mL) and extracted with EtOAc (3×50 mL). The combined organic extracts were washed with brine (3×50 mL), dried (MgSO₄), filtered and concentrated under vacuum to give the product (8.5 mg, 24%) as a solid. m/z=376 (M+1); r.t.=3.33 min. ¹H NMR (400 MHz; d₆-DMSO) δ 10.17 (s, 1H), 7.81 (d, 1H), 7.63 (d, 1H), 7.61 (d, 1H), 7.49-7.46 (m, 2H), 7.38-7.33 (dd, 1H), 7.01 (d, 1H), 6.93-6.84 (m, 1H), 5.13 (d, 1H), 4.56-4.51 (m, 1H), 2.71-2.59 (m, 2H), 2.39 (s, 3H), 1.91-1.85 (m, 2H), 1.69-1.63 (m, 2H).

### Compound 308

### Preparation of (E)-N-(3-(1,2-dihydroxyethyl)quinolin-7-yl)-2-methyl-4-(3,3,3-trifluoroprop-1-enyl)benzamide

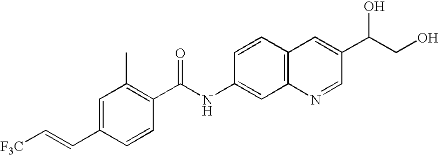

To a stirred solution of 4-((E)-3,3,3-trifluoroprop-1-enyl)-2-methylbenzoic acid (170 mg, 0.73 mmol) in CH₂Cl₂ (10 mL) and DMF (1 drop) at 0° C. was added oxalyl chloride (93 μL, 1.1 mmol). The mixture was stirred at 0° C. for 1 h and then warmed to rt and stirred for 3 h. The solvent was removed under vacuum and the obtained acid chloride was dissolved in CH₂Cl₂ (5 mL) and added to a solution of 1-(7-aminoquinolin-3-yl)ethane-1,2-diol (150 mg, 0.73 mol) in CH₂Cl₂ (5 mL) and pyridine (10 mL) at −30° C. The mixture was warmed to rt, stirred overnight, then diluted with EtOAc (150 mL). The organic layer was washed with aq. NaHCO₃ and brine, dried (Na₂SO₄), and concentrated under vacuum. The residue was purified by column chromatography on silica gel using 0-15% MeOH/EtOAc as eluent to the product (210 mg, 69%) as a solid. m/z=417.4 (M+1); rt=2.12 min. ¹H NMR (400 MHz; d₆-DMSO) δ 10.69 (s, 1H), 8.84 (d, 1H, J=2.0 Hz), 8.55 (s, 1H), 8.18 (d, 1H, J=1.6 Hz), 7.94 (d, 1H, J=8.8 Hz), 7.85 (dd, 1H, J=8.8, 1.6 Hz), 7.70 (s, 1H), 7.66 (d, 1H, J=8.4 Hz), 7.60 (d, 1H, J=7.6 Hz), 7.42-7.35 (m, 11H), 6.88 (dq, 1H, J=16.4, 7.2 Hz), 5.54 (d, 1H, J=4.4 Hz), 4.86 (t, 1H, J=5.6 Hz), 4.75 (dd, 1H, J=10.0, 5.6 Hz), 3.65-3.52 (m, 2H), 2.45 (s, 3H).

### Compound 309

### Preparation of (E)-N-(7-hydroxy-5,6,7,8-tetrahydronaphthylen-1-yl)-2-methyl-4-(3,3,3-trifluoroprop-1-enyl)benzamide

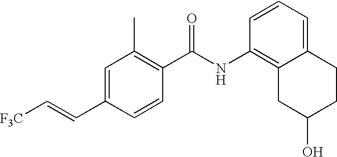

To a stirred solution of 8-Amino-1,2,3,4-tetrahydronaphthylen-2-ol (116 mg, 0.71 mmol) in anhydrous DMF (6 mL) was added a solution containing 4-((E)-3,3,3-trifluoroprop-1-enyl)-2-methylbenzoic acid (196 mg, 0.85 mmol), N-(3-dimethylaminopropyl)-N′-ethylcarbodiimide hydrochloride (164 mg, 0.85 mmol), 0.5M 1-hydroxy-7-azabenzotriazole in DMF (1.7 mL, 0.85 mmol), 4-N,N-dimethylaminopyridine (4 mg, 0.04 mmol) and N,N-diisopropylethylamine (0.30 mL, 1.7 mmol) in anhydrous DMF (4 mL). The reaction was stirred for 16 hours at room temperature then poured into saturated NaHCO₃ solution (50 mL) and extracted with EtOAc (3×50 mL). The combined organic extracts were washed with brine (3×50 mL), dried (MgSO₄), filtered and concentrated under vacuum. Purification by column chromatography on silica gel using 0-4% MeOH/CH₂Cl₂ as eluent gave the title compound (88 mg, 33%) as a solid. m/z=376 (M+1); r.t.=3.13 min. ¹H NMR (400 MHz; d₆-DMSO) δ 9.72 (s, 1H), 7.65-7.53 (m, 2H), 7.54 (d, 1H), 7.36 (d, 1H), 7.20 (d, 1H), 7.12 (t, 1H), 7.00 (d, 1H), 6.89-6.81 (m, 1H), 4.83 (d, 1H), 3.90 (br. s, 1H), 2.98-2.92 (m, 2H), 2.91-2.87 (m, 1H), 2.45 (s, 3H), 1.90-1.88 (m, 1H), 1.62-1.59 (m, 1H).

### Compound 310

### Preparation of (E)-N-(7-hydroxymethyl-7,8-dihydro-5H-pyrano[4,3-b]pyridin-3-yl)-2-methyl-4-(3,3,3-trifluoroprop-1-enyl)benzamide

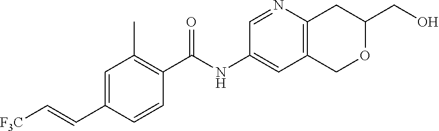

To a stirred solution of (3-amino-7,8-dihydro-5H-pyrano[4,3-b]pyridin-7-yl)methanol (35 mg, 0.19 mmol) in anhydrous DMF (2 mL) was added a solution containing 4-((E)-3,3,3-trifluoroprop-1-enyl)-2-methylbenzoic acid (54 mg, 0.23 mmol), N-(3-dimethylaminopropyl)-N′-ethylcarbodiimide hydrochloride (45 mg, 0.23 mmol), 0.5M 1-hydroxy-7-azabenzotriazole in DMF (0.5 mL, 0.23 mmol), 4-N,N-dimethylaminopyridine (1 mg, 0.008 mmol) and N,N-diisopropylethylamine (0.14 mL, 0.78 mmol) in anhydrous DMF (2 mL). The mixture was stirred for 16 hours at room temperature then it was poured into saturated NaHCO₃ solution (50 mL) and extracted with EtOAc (3×50 mL). The combined organic extracts were washed with brine (3×50 mL), dried (MgSO₄), filtered and concentrated under vacuum. Purification by column chromatography on silica gel using 0-5% MeOH/CH₂Cl₂ as eluent gave the product (19 mg, 23%) as a solid. m/z=393 (M+1); r.t.=2.29 min. ¹H NMR (400 MHz; CDCl₃) δ 8.37 (s, 1H), 8.18 (s, 1H), 7.60 (br. s, 1H), 7.54 (d, 1H), 7.37 (m, 2H), 7.14 (d, 1H), 6.31-6.23 (m, 1H), 4.91 (q, 2H), 3.96-3.91 (m, 11H), 3.85 (dd, 11H), 3.77-3.72 (m, 11H), 2.94-2.80 (m, 2H), 2.52 (s, 3H).

### Compound 311

### Preparation of (E)-N-(7-hydroxy-1,8-naphthyridin-2-yl)-2-methyl-4-(3,3,3-trifluoroprop-1-en-1)benzamide

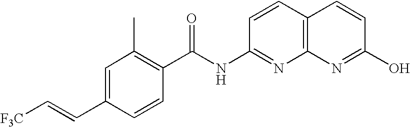

To a stirred solution of 4-((E)-3,3,3-trifluoroprop-1-enyl)-2-methylbenzoic acid (90 mg, 0.4 mmol) in CH₂Cl₂ (5 mL) and DMF (1 drop) at 0° C. was added oxalyl chloride (50 μL, 0.6 mmol). The mixture was stirred at 0° C. for 1 h and then warmed to rt and stirred for 3 h. The solvent was removed under vacuum and the obtained acid chloride was reacted with 7-amino-1,8-naphthyridin-2-ol (63 mg, 0.4 mmol) (prepared according to Stuk. T. L. et al, Org. Process Re. Dev. 2003, 7, 851) in pyridine (5 mL) at 110° C. overnight. The mixture was concentrated under vacuum and the residue was treated with aq. NaHCO₃ solution and filtered. The solid was washed with water, EtOAc, MeOH and dried under vacuum to give the product (65 mg) as a solid. m/z=374.0 (M+1); rt=3.03 min. ¹H NMR (400 MHz; d₆-DMSO) δ 11.92 (s, 1H), 11.04 (s, 11H), 8.14 (d, 1H, J=8.4 Hz), 8.03 (d, 1H, J=8.4 Hz), 7.89 (d, 1H, J=9.6 Hz), 7.64 (s, 1H), 7.60 (d, 1H, J=8.0 Hz), 7.53 (d, 1H, J=8.0 Hz), 7.40-7.32 (m, 11H), 6.86 (dq, 1H, J=16.4, 7.2 Hz), 6.46 (dd, 1H, J=9.6, 1.6 Hz), 2.40 (s, 3H).

### Compound 312

### Preparation of (E)-2-methyl-N-(5,6,7,8-tetrahydroquinolin-3-yl-4-(3,3,3-trifluoroprop-1-enyl)benzamide

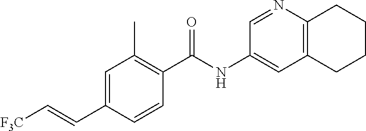

To a stirred solution of 4-((E)-3,3,3-trifluoroprop-1-enyl)-2-methylbenzoic acid (230 mg, 1.0 mmol) in CH₂Cl₂ (10 mL) and DMF (1 drop) at 0° C. was added oxalyl chloride (130 μL, 1.5 mmol). The mixture was stirred at 0° C. for 1 h and then warmed to rt and stirred for 3 h. The solvent was removed under vacuum and the obtained acid chloride was dissolved in CH₂Cl₂ (2 mL) and added to a solution of 5; 6,7,8-tetrahydroquinolin-3-amine (150 mg, 1.0 mmol) (prepared according to Skupinska, K. A. et al, J. Org. Chem. 2002, 67, 7890) in CH₂Cl₂ (5 mL) and pyridine (5 mL). The mixture was stirred at rt overnight, and then diluted with EtOAc (100 mL). The organic layer was washed with aq. NaHCO₃ and brine, dried (Na₂SO₄), and concentrated under vacuum. The residue was purified by column chromatography on silica gel to give the product (240 mg, 65%) as a solid. m/z=361.8 (M+1); rt=2.24 min. ¹H NMR (400 MHz; d₆-DMSO) δ 10.41 (s, 1H), 8.55 (d, 1H, J=2.0 Hz), 7.88 (d, 1H, J=2.0 Hz), 7.67 (s, 1H), 7.63 (d, 1H, J=7.6 Hz), 7.53 (d, 1H, J=7.6 Hz), 7.39-7.32 (m, 1H), 6.87 (dq, 1H, J=16.4, 7.2 Hz), 2.80-2.72 (m, 4H), 2.40 (s, 3H), 1.86-1.70 (m, 4H).

### Compound 313

### Preparation of (E)-N—((S)-7-hydroxy-5,6,7,8-tetrahydronaphthylen-1-yl)-2-methyl-4-(3,3,3-trifluoroprop-1-enyl)benzamide

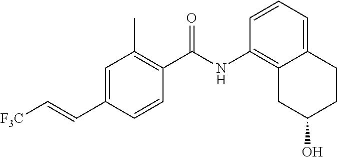

A sample of racemic (E)-N-(7-hydroxy-5,6,7,8-tetrahydronaphthylen-1-yl)-2-methyl-4-(3,3,3-trifluoroprop-1-enyl)benzamide (520 mg) was purified using chiral HPLC, giving the product (250 mg) which was arbitrarily assigned (S) stereochemistry (i.e. the stereochemistry has not been unambiguously assigned). m/z=376 (M+1); r.t.=3.13 min. ¹H NMR (400 MHz; CDCl₃) δ 7.76 (d, 1H), 7.55 (d, 1H), 7.37-7.35 (m, 2H), 7.24-7.20 (m, 2H), 7.15 (dd, 1H), 7.02 (d, 1H), 6.31-6.22 (m, 1H), 4.24-4.18 (m, 1H), 3.10-2.97 (m, 2H), 2.93-2.86 (m, 1H), 2.62-2.54 (m, 1H), 2.56 (s, 3H), 2.10-2.04 (m, 1H), 1.87-1.79 (m, 1H).

### Compound 314

### Preparation of (E)-N—((R)-7-hydroxy-5,6,7,8-tetrahydronaphthylen-1-yl)-2-methyl-4-(3,3,3-trifluoroprop-1-enyl)benzamide

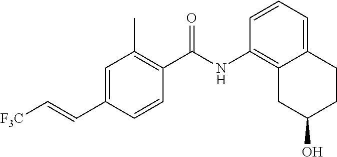

A sample of racemic (EN-(7-hydroxy-5,6,7,8-tetrahydronaphthylen-1-yl)-2-methyl-4-(3,3,3-trifluoroprop-1-enyl)benzamide (520 mg) was purified using chiral HPLC, giving the product (250 mg) which was arbitrarily assigned (R) stereochemistry (i.e. the stereochemistry has not been unambiguously assigned). m/z=376 (M+1); r.t.=3.13 min. ¹H NMR (400 MHz; CDCl₃) δ 7.76 (d, 11H), 7.55 (d, 11H), 7.37-7.35 (m, 2H), 7.24-7.21 (m, 2H), 7.15 (dd, 1H), 7.02 (d, 11H), 6.31-6.23 (m, 1H), 4.24-4.18 (m, 1H), 3.11-2.98 (m, 2H), 2.92-2.84 (m, 11H), 2.62-2.58 (m, 1H), 2.55 (s, 3H), 2.11-2.04 (m, 1H), 1.83-1.78 (m, 1H).

### Compound 315

### Preparation of (E)-N-(6-hydroxy-5,6,7,8-tetrahydroquinolol-3-yl)-2-methyl-4-(3,3,3-trifluoroprop-1-enyl)benzamide

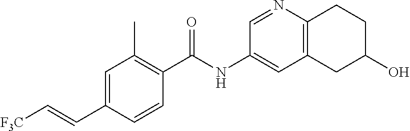

To a stirred solution of 3-amino-5,6,7,8-tetrahydroquinolin-6-ol (160 mg, 0.97 mmol) in anhydrous acetonitrile (2 mL) was added a solution containing 4-((E)-3,3,3-trifluoroprop-1-enyl)-2-methylbenzoic acid (236 mg, 1.0 mmol), N-(3-dimethylaminopropyl)-N′-ethylcarbodiimide hydrochloride (224 mg, 1.17 mmol), 0.5M 1-hydroxy-7-azabenzotriazole in DMF (2.34 mL, 1.17 mmol), 4-N,N-dimethylaminopyridine (6 mg, 0.05 mmol) and N,N-diisopropylethylamine (0.41 mL, 2.3 mmol) in anhydrous acetonitrile (2 mL). The mixture was stirred for 16 hours at room temperature then poured into saturated NaHCO₃ solution (50 mL) and extracted with EtOAc (3×50 mL). The combined organic extracts were washed with brine (3×50 mL), dried (MgSO₄), filtered and concentrated under vacuum. Purification by column chromatography on silica gel using 0-5% MeOH/CH₂Cl₂ as eluent gave the product (55 mg, 14%) as a solid. m/z=377 (M+1); r.t.=1.97 min. ¹H NMR (400 MHz; d₆-DMSO) δ 10.41 (s, 1H), 8.55 (d, 1H), 7.86 (d, 1H), 7.67 (s, 1H), 7.63 (d, 11H), 7.53 (d, 1H), 7.36 (dd, 1H), 6.92-6.82 (m, 11H), 4.86 (d, 1H), 4.01-3.98 (m, 1H), 2.97-2.86 (m, 2H), 2.80-2.72 (m, 1H), 2.68-2.62 (m, 1H), 2.40 (s, 3H), 1.96-1.92 (m, 1H), 1.82-1.76 (m, 11H).

### Compound 316

### Preparation of (Q-2-methyl-N-(quinolin-3-yl)-4-(3,3,3-trifluoro-2-methylprop-1-enylbenzamide

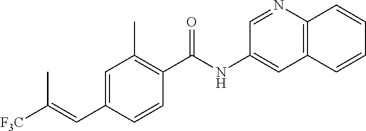

To a stirred solution of (E)-2-methyl-4-(3,3,3-trifluoro-2-methylprop-1-enyl)benzoic acid (90 mg, 0.4 mmol) in CH₂Cl₂ (10 mL) and DMF (1 drop) at 0° C. was added oxalyl chloride (47 μL, 0.6 mmol). The mixture was stirred at 0° C. for 1 h and then warmed to rt and stirred for 3 h. The solvent was removed under vacuum and the obtained acid chloride in CH₂Cl₂ (1 mL) was added to a solution of 3-quinolinamine (53 mg, 0.4 mmol) in CH₂Cl₂ (2 mL) and pyridine (2 mL). The mixture was stirred at rt overnight, and then diluted with EtOAc (100 mL). The organic layer was washed with aq. NaHCO₃ and brine, dried (Na₂SO₄), and concentrated under vacuum. The residue was purified by column chromatography on silica gel to give the product (125 mg, 90%) as a solid. m/z=371.1 (M+1); rt=3.49 min. ¹H NMR (400 MHz; d₆-DMSO) δ 10.86 (s, 1H), 9.04 (d, 1H, J=2.4 Hz), 8.89 (d, 1H, J=2.4 Hz), 7.98 (d, 2H, J=8.4 Hz), 7.70-7.57 (m, 3H), 7.45 (s, 1H), 7.44 (d, 1H, J=6.4 Hz), 7.22 (s, 1H), 2.47 (s, 3H), 2.05 (s, 3H).

### Compound 317

### Preparation of (E)-N-(7-(Hydroxymethyl)-1,5-naphthyridin-3-yl)-2-methyl-4-(3,33-trifluoroprop-1-enyl)benzamide

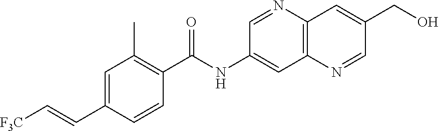

To a stirred solution of 4-((E)-3,3,3-trifluoroprop-1-enyl)-2-methylbenzoic acid (98 mg, 0.43 mmol) in CH₂Cl₂ (5 mL) and DMF (1 drop) at 0° C. was added oxalyl chloride (87 mg, 0.7 mmol). The mixture was stirred at 0° C. for 1 h and then warmed to rt and stirred for 2 h. The solvent was removed under vacuum and the resulting acid chloride was reacted with (7-amino-1,5-naphthyridin-3-yl)methanol (30 mg, 0.17 mmol) in CH₂Cl₂ (2 mL) and pyridine (2 mL) at 50° C. overnight. The mixture was concentrated under vacuum and the residue was dissolved in MeOH (10 mL) and potassium carbonate (250 mg, 1.8 mmol) was added. The mixture was stirred at rt for 4 h then concentrated under vacuum. The residue was treated with water (10 mL) and EtOAc (100 mL). The aqueous and organic layers were partitioned and the organic layer was washed with brine, dried (Na₂SO₄), and concentrated under vacuum. The residue was purified by column chromatography to give the product (32 mg) as a solid. m/z=388.1 (M+1); rt=2.69 min. ¹H NMR (400 MHz; d₆-DMSO) δ 10.99 (s, 1H), 9.17 (d, 1H, J=2.4 Hz), 8.94 (d, 1H, J=1.6 Hz), 8.91 (d, 1H, J=2.4 Hz), 8.24 (s, 1H), 7.71 (s, 1H), 7.70-7.65 (m, 2H), 7.43-7.36 (m, 1H), 6.89 (dq, 1H, J=16.4, 6.8 Hz), 5.55 (t, 1H, J=5.6 Hz), 4.77 (d, 2H, J=5.6 Hz), 2.46 (s, 3H).

### Compound 318

### Preparation of (E)-2-methyl-N-(1,5-naphthyridin-3-yl)-4-(3,3,3-trifluoroprop-1-enyl)benzamide

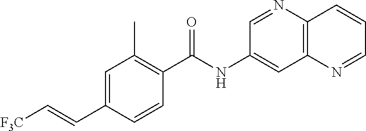

To a stirred solution of 4-((E)-3,3,3-trifluoroprop-1-enyl)-2-methylbenzoic acid (120 mg, 0.52 mmol) in CH₂Cl₂ (10 mL) and DMF (1 drop) at 0° C. was added oxalyl chloride (66 μL, 0.77 mmol). The mixture was stirred at 0° C. for 1 h and then warmed to rt and stirred for 3 h. The solvent was removed under vacuum and the resulting acid chloride in CH₂Cl₂ (1 mL) was added to a solution of 1,5-naphthyridin-3-amine (75 mg, 0.52 mmol) in CH₂Cl₂ (2 mL) and pyridine (2 mL). The mixture was stirred at rt overnight, and then diluted with EtOAc (150 mL). The organic layer was washed with aq. NaHCO₃ and brine, dried (Na₂SO₄), and concentrated under vacuum. The residue was purified by column chromatography on silica gel to give the product (110 mg, 58%) as a solid. m/z=358.0 (M+1); rt=2.96 min. ¹H NMR (400 MHz; d₆-DMSO) δ 11.02 (s, 1H), 9.19 (d, 1H, J=2.4 Hz), 8.98 (dd, 1H, J=4.4, 1.6 Hz), 8.92 (d, 1H, J=2.0 Hz), 8.39 (dq, 1H, J=8.0, 0.8 Hz), 7.72-7.65 (m, 4H), 7.43-7.36 (m, 1H), 6.90 (dq, 1H, J=16.0, 6.8 Hz), 2.47 (s, 3H).

### Compound 319

### Preparation of (E)-2-methyl-N-(1,8-naphthyridin-2-yl)-4-(3,3,3-trifluoroprop-1-enyl)benzamide

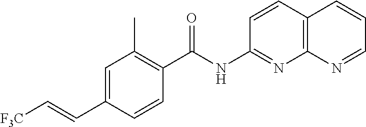

To a stirred solution of 4-((E)-3,3,3-trifluoroprop-1-enyl)-2-methylbenzoic acid (160 mg, 0.7 mmol) in CH₂Cl₂ (10 mL) and DMF (1 drop) at 0° C. was added oxalyl chloride (87 μL, 1.0 mol). The mixture was stirred at 0° C. for 1 h and then warmed to rt and stirred for 3 h. The solvent was removed under vacuum and the resulting acid chloride in CH₂Cl₂ (1 mL) was added to a solution of 1,8-naphthyridin-2-amine (100 mg, 0.7 mmol) in CH₂Cl₂ (5 mL) and pyridine (5 mL). The mixture was stirred at rt overnight, and then diluted with EtOAc (150 mL). The organic layer was washed with aq. NaHCO₃ and brine, dried (Na₂SO₄), and concentrated under vacuum. The residue was purified by column chromatography on silica gel to give the product (55 mg, 22%) as a solid. m/z=358.0 (M+1); rt=2.90 min. ¹H NMR (400 MHz; d₆-DMSO) δ 11.50 (s, 1H), 9.02 (dd, 1H, J=4.4, 2.0 Hz), 8.51 (s, 2H), 8.44 (dd, 1H, J=8.0, 2.0 Hz), 7.66 (s, 11H), 7.62 (s, 2H), 7.56 (dd, 1H, J=8.0, 4.4 Hz), 7.41-7.34 (m, 1H), 6.89 (dq, 1H, J=16.4, 6.8 Hz), 2.46 (s, 3H).

### Compound 320

### Preparation of (E)-N-(7-(1-hydroxyethyl)-1,5-naphthyridin-3-yl)-2-methyl-4-(3,33-trifluoroprop-1-enyl)benzamide

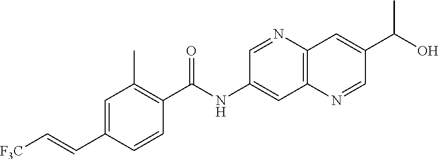

To a stirred solution of 4-((E)-3,3,3-trifluoroprop-1-enyl)-2-methylbenzoic acid (130 mg, 0.58 mmol) in CH₂Cl₂ (5 mL) and DMF (1 drop) at 0° C. was added oxalyl chloride (74 μL, 0.87 mmol). The mixture was stirred at 0° C. for 1 h and then warmed to rt and stirred for 3 h. The solvent was removed under vacuum and the resulting acid chloride in CH₂Cl₂ (1 mL) was added to a solution of 1-(7-amino-1,5-naphthyridin-3-yl)ethanol (110 mg, 0.58 mmol) in CH₂Cl₂ (2 mL) and pyridine (2 mL). The mixture was stirred at rt overnight, and then diluted with EtOAc (150 mL). The organic layer was washed with aq. NaHCO₃ and brine, dried (Na₂SO₄), and concentrated under vacuum. The residue was purified by column chromatography on silica gel to give the product (120 mg, 51%) as a solid. m/z=401.8 (M+1); rt=2.77 min. ¹H NMR (400 MHz; d₆-DMSO) δ 10.99 (s, 1H), 9.17 (d, 1H, J=2.4 Hz), 8.99 (d, 1H, J=2.0 Hz), 8.90 (d, 1H, J=1.2 Hz), 8.24 (t, 1H, J=1.2 Hz), 7.72 (s, 1H), 7.70-7.64 (m, 2H), 7.43-7.35 (m, 11H), 6.89 (dq, 1H, J=16.0, 4.8 Hz), 5.56 (d, 1H, J=4.4 Hz), 5.02 (m, 1H), 2.46 (s, 3H), 1.49 (d, 3H, J=6.4 Hz).

### Compound 321

### Preparation of (E)-2-methyl-N-(1,8-naphthyridin-3-yl)-4-(3,3,3-trifluoroprop-1-enyl)benzamide

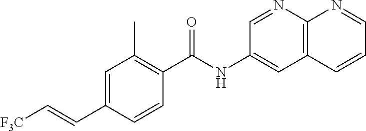

To a stirred solution of 4-((E)-3,3,3-trifluoroprop-1-enyl)-2-methylbenzoic acid (160 mg, 0.69 mmol) in CH₂Cl₂ (5 mL) and DMF (1 drop) at 0° C. was added oxalyl chloride (87 μL, 1.0 mmol). The mixture was stirred at 0° C. for 1 h and then warmed to rt and stirred for 3 h. The solvent was removed under vacuum and the resulting acid chloride in CH₂Cl₂ (1 mL) was added to a solution of 1,8-naphthyridin-3-amine (100 mg, 0.69 mmol) in CH₂Cl₂ (2 mL) and pyridine (2 mL). The mixture was stirred at rt overnight, and then diluted with EtOAc (150 mL). The organic layer was washed with aq. NaHCO₃ and brine, dried (Na₂SO₄), and concentrated under vacuum. The residue was purified by column chromatography to give the product (15 mg) as a solid. m/z=358.0 (M+1); rt=2.88 min. ¹H NMR (400 MHz; d₆-DMSO) δ 11.00 (s, 1H), 9.18 (d, 1H, J=2.8 Hz), 9.01 (d, 1H, J=2.8 Hz), 8.99 (dd, 1H, J=4.0, 1.6 Hz), 8.52 (dd, 1H, J=8.0, 1.6 Hz), 7.72 (s, 11H), 7.69 (AB, 1H, J=8.0 Hz), 7.66 (AB, 1H, J=8.0 Hz), 7.63 (dd, 1H, J=8.0, 4.4 Hz), 7.42-7.35 (m, 11H), 6.90 (dq, 1H, J=16.4, 7.2 Hz), 2.46 (s, 3H).

### Compound 322

### Preparation of (E)-N-(1-acetyl-1,2,3,4-tetrahydroquinolin-7-yl)-2-methyl-4-(3,3,3-trifluoroprop-1-enyl)benzamide

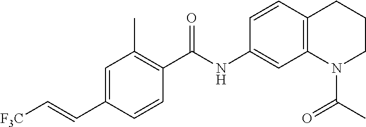

A mixture of 4-((E)-3,3,3-trifluoroprop-1-enyl)-2-methylbenzoic acid (150 mg, 0.65 mmol), 1-hydroxybenzotriazole hydrate (100 mg, 0.65 mol), N-(3-dimethylaminopropyl)-N′-ethylcarbodiimide hydrochloride (250 mg, 1.3 mmol), 1-(7-amino-3,4-dihydroquinolin-1(2H)-yl)ethanone (120 mg, 0.65 mmol), N,N-diisopropylethylamine (230 μL, 1.3 mol) in CH₂Cl₂ (5 mL) was stirred at rt over the weekend. The mixture was diluted with EtOAc (100 mL), washed with brine, dried (Na₂SO₄), and concentrated under vacuum. The residue was purified by column chromatography on silica gel to give the product (160 mg, 60%) as a solid. m/z=403.1 (M+1); rt=3.03 min. ¹H NMR (400 MHz; d₆-DMSO) δ 10.33 (s, 1H), 7.79 (brs, 1H), 7.66 (s, 1H), 7.62 (d, 1H, J=8.0 Hz), 7.51 (d, 1H, J=8.0 Hz), 7.48 (dd, 1H, J=8.4, 2.0 Hz), 7.39-7.32 (m, 1H), 7.14 (d, 1H, J=8.4 Hz), 6.86 (dq, 1H, J=16.4, 6.8 Hz), 3.67 (t, 2H, J=6.4 Hz), 2.67 (t, 2H, J=6.4 Hz), 2.39 (s, 3H), 2.18 (s, 3H), 1.86 (m, 2H).

### Compound 324

### Preparation of (E)-N-(7-acetyl-1,5-naphthyridin-3-yl-2-methyl-4-(3,3,3-trifluoroprop-1-enyl)benzamide

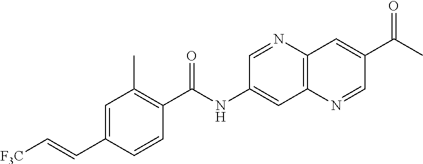

A mixture of (E)-N-(7-(1-hydroxyethyl)-1,5-naphthyridin-3-yl)-2-methyl-4-(3,3,3-trifluoroprop-1-enyl)benzamide (45 mg, 0.11 mmol), 15 wt % Dess-Martin periodinane (950 mg, 0.34 mol) solution in CH₂Cl₂, and CH₂Cl₂ (5 mL) was stirred at rt for 3 h. The solvent was removed under vacuum and the residue was treated with aq. NaHCO₃ solution and extracted with EtOAc (50 mL×2). The combined organic layers were washed with brine, dried (Na₂SO₄), and concentrated under vacuum. The residue was purified by column chromatography on silica gel using 0-50% THF/CH₂Cl₂ as eluent to give the product (40 mg) as a solid. m/z=400.0 (M+1); rt=3.14 min. ¹H NMR (400 MHz; d₆-DMSO) δ 11.18 (s, 1H), 9.39 (d, 1H, J=2.4 Hz), 9.29 (d, 1H, J=2.4 Hz), 8.99 (d, 1H, J=2.0 Hz), 8.91 (d, 1H, J=2.0 Hz), 7.73 (s, 1H), 7.69 (s, 2H), 7.43-7.36 (m, 1H), 6.91 (dq, 1H, J=16.4, 6.8 Hz), 2.78 (s, 3H), 2.47 (s, 3H).

### Compound 325

### Preparation of (E)-2-methyl-N-(Quinoxalin-6-yl)-4-(3,3,3-trifluoroprop-1-enyl)benzamide

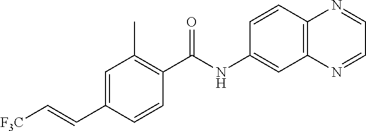

Oxalyl chloride (60 μL, 0.7 mmol) was added to a stirred solution of 4-((E)-3,3,3-trifluoroprop-1-enyl)-2-methylbenzoic acid (80 mg, 0.3 mmol) and DMF (1 drop) in THF (3 mL) at room temperature. The mixture was stirred at rt for 90 min then the solvent was removed under vacuum. The residue (acid chloride) was dissolved in CH₂Cl₂ and quinoxalin-6-amine (66 mg, 0.45 mmol) was added, followed by Et₃N (100 μL, 1.0 mmol) and 4-N,N-dimethylaminopyridine (cat. quantity). The mixture was stirred at rt over the weekend then the solvent was removed under vacuum. The residue was purified by column chromatography on silica gel using 1-5% MeOH/CH₂Cl₂ as eluent to give a solid (62 mg). The solid was triturated with hexane to give the product. m/z=358.3 (M+1). ¹H NMR (400 MHz; d₆-DMSO) δ 10.89 (s, 1H), 8.91 (d, 1H), 8.85 (d, 1H), 8.67 (s, 1H), 8.08 (s, 2H), 7.61-7.71 (m, 3H), 7.38 (dd, 1H), 6.86-6.92 (m, 1H), 2.48 (s, 3H).

### Compound 326

### Preparation of (E)-N-(7-(2-hydroxypropan-2-yl)-1,5-naphthyridin-3-yl)-2-methyl-4-(3,3,3-trifluoroprop-1-enyl)benzamide

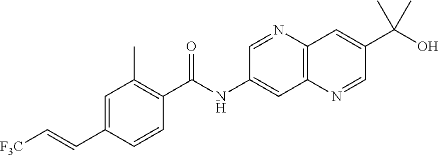

To a stirred solution of (E)-N-(7-acetyl-1,5-naphthyridin-3-yl)-2-methyl-4-(3,3,3-trifluoroprop-1-enyl)benzamide (30 mg) in THF (15 mL) at −78° C. under N₂ was added a solution of MeLi in THF (1.6 M; 0.3 mL). The mixture was stirred at −78° C. for 30 min and then quenched by addition of sat. aq. NH₄Cl solution. The mixture was extracted with EtOAc (50 mL×2) and the combined organic layers were washed with brine, dried Na₂SO₄), and concentrated under vacuum. The residue was purified by column chromatography on silica gel to give the product (14 mg) as a solid. m/z=416.1 (M+1); rt=2.79 min. ¹H NMR (400 MHz; d₆-DMSO) δ 10.98 (s, 1H), 9.17 (d, 1H, J=2.4 Hz), 9.13 (d, 1H, J=2.0 Hz), 8.89 (d, 1H, J=2.0 Hz), 8.32 (d, 1H, J=2.4 Hz), 7.72 (s, 1H), 7.70-7.65 (m, 2H), 7.42-7.35 (m, 1H), 6.91 (dq, 1H, J=16.4, 6.8 Hz), 5.46 (s, 1H), 2.46 (s, 3H), 1.58 (s, 6H).

### Compound 327

### Preparation of (E)-2-methyl-N-(1,7-naphthyridin-8-yl)-4-(3,3,3-trifluoroprop-1-enyl)benzamide

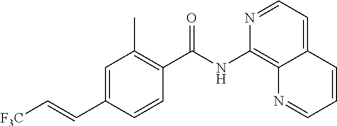

Oxalyl chloride (93 μL, 1.1 mmol) was added to a stirred suspension of 4-((E)-3,3,3-trifluoroprop-1-enyl)-2-methylbenzoic acid (230 mg, 1.0 mmol) in CH₂Cl₂ (5 mL) at 0° C. The mixture was stirred at 0° C. for 15 min then allowed to warm to rt and stirred for 2 h, after which further oxalyl chloride (50 μL, ca. 0.5 mmol) was added and the mixture was stirred for an additional 30 min. The solvent was removed under vacuum and the residue was re-dissolved in CH₂Cl₂ (5 mL) and cooled to 0° C. Et₃N (280 μL, 2.0 mmol) was added followed by 4-N,N-dimethylaminopyridine (cat. quantity) and 1,7-naphthyridin-8-amine (160 mg, 1.1 mmol). The mixture was stirred at 0° C. for 1 h then allowed to warm to rt and stirred overnight. CH₂Cl₂ (30 mL) was added and the mixture was washed with H₂O (1×20 mL). The aqueous layer was extracted with CH₂Cl₂ (1×20 mL) and the combined organic extracts were dried (MgSO₄), filtered and concentrated under vacuum to leave a crude solid. The solid was purified by column chromatography on silica gel using 20-50% EtOAc/hexane as eluent to give a solid (2^{nd} product from column) which was purified further by recrystallization from a MeOH/H₂O mixture to give the product (100 mg, 30%) as a solid. m/z=359.1 (M+2); rt=2.79 min. ¹H NMR (400 MHz; CDCl₃) δ 10.40 (1H, br. s), 8.85-8.88 (1H, m), 8.49 (1H, d), 8.17 (1H, d), 7.72 (1H, d), 7.67 (1H, m), 7.39-7.44 (3H, m), 7.16 (1H, d), 6.25-6.34 (1H, m), 2.65 (3H, s).

### Compound 401

### Preparation of (E)-N-(1-methanesulfonyl-2,3-dihydro-1H-indol-6-yl]-2-methyl-4-(3,3,3-trifluoroprop-1-enyl)benzamide

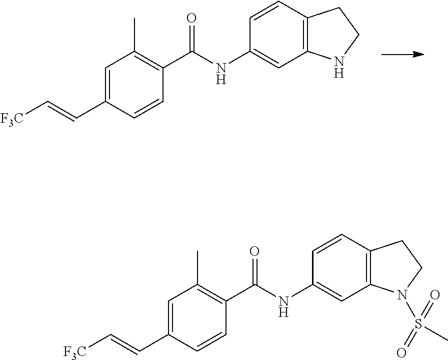

4-((E)-3,3,3,-Trifluoroprop-1-enyl)-2-methylbenzoic acid (104 mg, 0.45 mmol), N-(3-dimethylaminopropyl)-N′-ethylcarbodiimide hydrochloride (260 mg, 1.4 mmol), 1-hydroxybenzotriazole hydrate (104 mg, 0.68 mmol) and 1-methanesulfonyl-2,3-dihydro-1H-indol-6-ylamine (190 mg, 0.90 mmol) were combined in DMF (40 mL) and stirred at room temperature overnight. The mixture was then partitioned between EtOAc and sat'd NaHCO₃ and the organic layer was washed with H₂O and brine, then dried (Na₂SO₄), filtered and the solvent removed under vacuum to leave a crude oil. The oil was purified by column chromatography on silica gel using 0-50% EtOAc/hexane as eluent to give the product (48 mg, 24%) as a solid. m/z=425.2 (M+1). ¹H NMR (400 MHz; d₆-DMSO) δ 10.45 (1H, s), 7.78 (1H, d), 7.67-7.6 (2H, m), 7.5 (1H, d), 7.44 (1H, dd), 7.35 (1H, dd), 7.25 (1H, d), 6.85 (1H, m), 3.95 (2H, m), 3.15 (2H, m), 3.0 (3H, s), 2.4 (3H, s).

### Compound 402

### Preparation of (E)-N-(1-cyclopropanecarbonyl-2,3-dihydro-1H-indol-6-yl)-2-methyl-4-(3,3,3-trifluoro-prop-1-enyl)benzamide

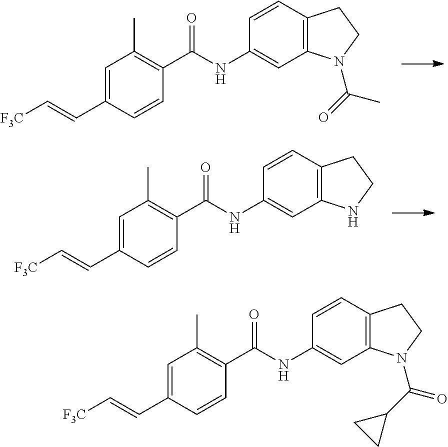

### a. 4-((E)-3,3,3-Trifluoroprop-1-enyl)-N-(indolin-6-yl)-2-methylbenzamide

(E)-N-(1-Acetylindolin-6-yl)-2-methyl-4-(3,3,3-trifluoroprop-1-enyl)benzamide (see WO; 200 mg, mmol) was placed in MeOH (40 mL) and 1N HCl (10 mL). The mixture was heated to reflux and stirred for 4 days (note: LC/MS analysis indicated deacetylation was not complete). The mixture was neutralized, then extracted with EtOAC. Concentration of the organic layer gave a solid (200 mg). The solid was dissolved in a solution of NaOMe in MeOH (25% wt/vol, 20 mL) and refluxed for 14 h. After allowing to cool to room temperature, the volume of the mixture was reduced to approximately half by concentration under vacuum. H₂O was added and the mixture was extracted with EtOAc. The organic extract was washed with brine, dried and concentrated under vacuum to give the product as a solid. The material was used without further purification in the next step. m/z=347.1 (M+1).

### b. (E)-N-(1-Cyclopropanecarbonyl-2,3-dihydro-1H-indol-6-yl)-2-methyl-4-(3,3,3-trifluoroprop-1-enyl)benzamide

Crude 4-((E)-3,3,3-trifluoroprop-1-enyl)-N-(indolin-6-yl)-2-methylbenzamide (200 mg, 0.58 mmol) was placed in CH₂Cl₂ (50 mL). N,N-Di-iso-propylethylamine (202 μL, 1.16 mmol) was added, followed by the addition of cyclopropanecarbonyl chloride (64 μL, 0.69 mmol). The mixture was stirred overnight at room temperature, then partitioned between H₂O and CH₂Cl₂. The organic layer was dried and concentrated under vacuum to leave a crude residue. The residue was purified by column chromatography on silica gel to give a solid (100 mg; this is ca. 20% diacetylated material and ca. 80% title compound by LC/MS). Further purification by preparative high-performance liquid chromatography gave the product (75 mg, 37%) as a solid. m/z=415.2 (M+1). ¹H NMR (400 MHz; d₆-DMSO) δ 10.3 (1H, s), 8.45 (1H, s), 7.7-7.55 (2H, m), 7.49 (1H, d), 7.42-7.32 (2H, m), 7.27 (1H, d), 6.85 (1H, m), 4.3 (2H, m), 3.15 (2H, m), 2.4 (3H, s), 1.9 (1H, m), 0.95-0.83 (4H, m).

### Compound 403

### Preparation of 4-((E)-(3,3,3-trifluoroprop-1-enyl)-N-(2-(1-hydroxyethyl)benzo[d]thiazol-5-yl)-2-methylbenzamide

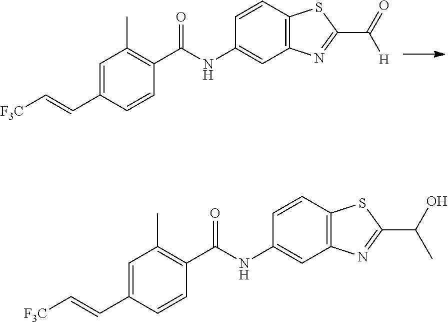

4-((E)-3,3,3-Trifluoroprop-1-enyl)-N-(2-formylbenzo[d]thiazol-5-yl)-2-methylbenzamide (300 mg, 0.8 mmol) was placed in THF (30 mL) and cooled to −78° C. under an atmosphere of nitrogen. A solution of MeLi in Et₂O (1.6 M; 1.6 mL, 2.6 mmol) was added and the mixture was warmed to room temperature and stirred overnight. The reaction was quenched by addition of aqueous NH₄Cl and the mixture was extracted with EtOAc. The organic layer was washed with water, brine, dried and concentrated under vacuum to leave a crude oil. Purification by column chromatography on silica gel using 0-50% EtOAc/hexane as eluent gave a solid (70 mg), which was purified further by preparative high-performance liquid chromatography to give the product (32 mg, 11%) as a solid (ca. 90% pure). m/z=407.1 (M+1). ¹H NMR (400 MHz; d₆-DMSO) δ 10.5 (1H, s), 8.5 (1H, s), 8.0 (1H, d), 7.73-7.55 (4H, m), 7.35 (1H, dd), 6.85 (1H, m), 6.33 (1H, d), 5.05 (1H, m), 2.4 (3H, s), 1.9 (3H, d).

### Compound 404

### Preparation of (E)-N-(2-(2-hydroxyethyl)-1,3-dioxoisoindolin-5-yl)-2-methyl-(3,3,3-trifluoroprop-1-enyl)benzamide

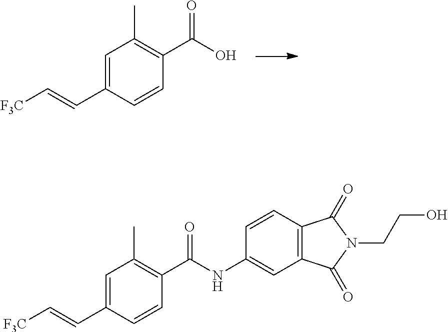

A mixture of 4-((E)-3,3,3,-trifluoroprop-1-enyl)-2-methylbenzoic acid (40 mg, 0.17 mmol), 5-amino-2-(2-hydroxyethyl)isoindoline-1,3-dione (43 mg, 0.21 mmol), N-(3-dimethylaminopropyl)-N′-ethylcarbodiimide hydrochloride (33 mg, 0.17 mmol), 4-N,N,-dimethylaminopyridine (21 mg, 0.17 mmol) and Et₃N (48 μL, 0.35 mmol) were combined in CH₂Cl₂ and stirred overnight. H₂O was added and the aqueous and organic layers were partitioned. The aqueous layer was extracted with CH₂Cl₂ (×3) and the combined organic extracts were washed with brine (×1), dried (Na₂SO₄), filtered and the solvent removed under vacuum to leave a crude residue. The residue was purified by column chromatography on silica gel using 0-30% EtOAc/hexanes as eluent gave a solid (17 mg). The solid was triturated with hexanes and filtered to give the product (17 mg, 23%) as a solid. m/z=419.5 (M+1). ¹H NMR (400 MHz; CDCl₃) δ 7.90 (1H, d), 7.61 (1H, d), 7.26-7.31 (2H, m), 7.12 (1H, dd), 7.03 (1H, d), 6.82 (1H, dd), 6.21-6.29 (1H, m), 4.51 (2H, t), 4.32 (1H, br. s), 4.05 (2H, t), 2.57 (3H, s).

### 405

### Preparation of (E)-N-[1-(2,2-dimethyl-propionyl)-2,3-dihydro-1H-indol-6-yl]-2-methyl-4-(3,3,3-trifluoro-prop-1-enyl)benzamide

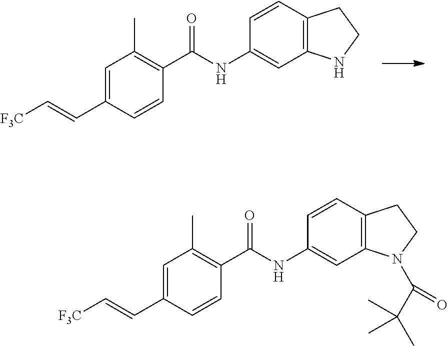

Crude 4-((E)-3,3,3-trifluoroprop-1-enyl)-N-(indolin-6-yl)-2-methylbenzamide (115 mg, 0.33 mmol; prepared by NaOMe/MeOH deacetylation of (E)-N-(1-acetylindolin-6-yl)-2-methyl-4-(3,3,3-trifluoroprop-1-enyl)benzamide as above) was placed in CH₂Cl₂ (50 mL). N,N-Di-Iso-propylethylamine (116 μL, 0.66 mmol) was added, followed by the addition of trimethylacetyl chloride (41 μL, 0.33 mmol). The mixture was stirred overnight at room temperature, then partitioned between H₂O and CH₂Cl₂. The organic layer was dried and concentrated under vacuum to leave a crude residue. The residue was purified by column chromatography on silica gel to give a solid (35 mg; containing ca. 20% diacetylated material). Further purification by preparative high-performance liquid chromatography gave the product (17 mg, 13%) as a solid. m/z=431.2 (M+1). ¹H NMR (400 MHz; d₆-DMSO) δ 9.5 (1H, s), 8.25 (1H, s), 7.5-7.45 (4H, m), 7.42-7.22 (2H, m), 6.5 (1H, m), 4.25 (2H, m), 3.05 (2H, m), 2.4 (3H, s), 1.8 (9H, s).

### Compound 406

### Preparation of (E)-2-methyl-N-(1-propionylindoline-6-yl]-4-(3,3,3-trifluoro-prop-1-enyl)benzamide

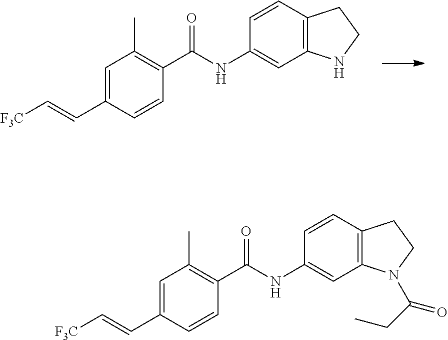

4-((E)-3,3,3-Trifluoroprop-1-enyl)-N-(indolin-6-yl)-2-methylbenzamide (113 mg, 0.32 mmol) was dissolved in CH₂Cl₂ (25 mL). N,N-Di-iso-propylethylamine (114 μL, 0.64 mmol) was added, followed by the addition of propanoyl chloride (34 μL, 0.39 mmol). The mixture was stirred overnight at room temperature, then partitioned between H₂O and CH₂Cl₂. The organic layer was dried (MgSO₄) and concentrated under vacuum to leave a crude residue. The residue was purified by column chromatography on silica gel to give a solid (62 mg). Further purification by preparative high-performance liquid chromatography gave the product (31 mg, 24%) as a solid. m/z=403.6 (M+1). ¹H NMR (400 MHz; d₆-DMSO) δ 10.25 (1H, s), 8.5 (1H, s), 7.68-7.48 (2H, m), 7.5 (1H, d), 7.45-7.32 (2H, m), 7.25 (1H, d), 6.85 (1H, m), 4.15 (2H, m), 3.15 (2H, m), 2.47 (2H, q), 2.4 (3H, s), 1.1 (3H, t).

### Compound 407

### Preparation of (E)-N-(1-(2-hydroxyacetyl)-indolin-yl)-2-methyl-4-(3,3,3-trifluoro-Prop-1-enyl)benzamide

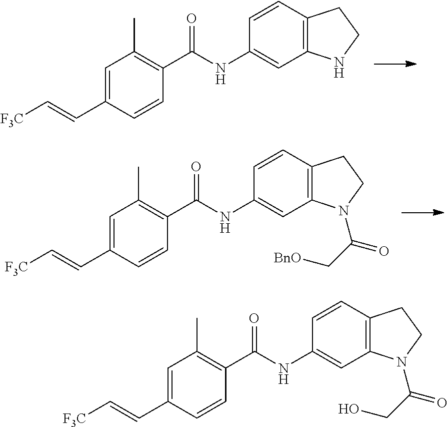

### a. (E)-N-(1-(2-(benzyloxy)acetyl)indolin-6-yl)-2-methyl-4-(3,3,3-trifluoroprop-1-enyl)benzamide

(E)-N-(indolin-6-yl)-2-methyl-4-(3,3,3-trifluoroprop-1-enyl)benzamide (100 mg, 0.3 mmol) was dissolved CH₂Cl₂ (25 mL). N,N-Di-iso-propylethylamine (114 μL, 0.64 mmol) was added, followed by the addition of benzyloxyacetyl chloride (49 μL, 0.31 mmol). The mixture was stirred overnight at room temperature, then partitioned between H₂O and CH₂Cl₂. The organic layer was dried (MgSO₄) and concentrated under vacuum to leave a crude residue. The residue was purified by column chromatography on silica gel to give a solid (85 mg). m/z=495.4 (M+1).

### b. (E)-N-(1-(2-hydroxyacetyl)-indolin-6-yl)-2-methyl-4-(3,3,3-trifluoro-prop-1-enyl)benzamide

(E)-N-(1-(2-(benzyloxy)acetyl)indolin-6-yl)-2-methyl-4-(3,3,3-trifluoroprop-1-enyl)benzamide (85 mg, 0.17 mmol) was dissolved in CH₂Cl₂ (20 mL) and cooled to −78° C. under an atmosphere of nitrogen. Boron tribromide (49 μL, 0.52 mmol) was added, and the mixture was warmed to room temperature over 6 h. The mixture was then partitioned between CH₂Cl₂ and NaHCO₃ and the organic extract dried and concentrated under vacuum to leave a crude oil. Purification by column chromatography on silica gel gave a product (32 mg), which was purified further by preparative high-performance liquid chromatography to give the title compound (8 mg, 10%) as solid. m/z=405.0 (M+1). ¹H NMR (400 MHz; d₆-DMSO) δ 10.25 (1H, s), 8.5 (1H, s), 7.68-7.6 (2H, m), 7.55-7.48 (2H, m), 7.35 (1H, dd), 7.2 (1H, d), 6.85 (1H, m), 4.35 (1H, t), 4.25 (2H, d), 4.15 (2H, m), 3.15 (2H, m), 2.4 (3H, s).

### Compound 408

### Preparation of (E)-N-(1-acetyl-1H-pyrrolo[2,3-b]pyridin-5-yl]-2-methyl-4-(3,3,3-trifluoroprop-1-enyl)benzamide

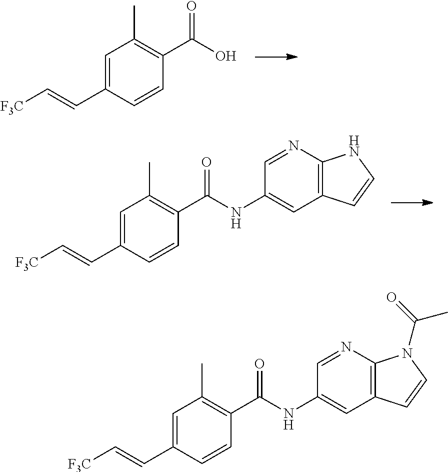

### a. (E)-2-methyl-N-(1H-pyrrolo[2,3-b]pyridin-5-yl]-4-(3,3,3-trifluoroprop-1-enyl)benzamide

4-((E)-3,3,3,-Trifluoroprop-1-enyl)-2-methylbenzoic acid (740 mg, 3.2 mmol), N-(3-dimethylaminopropyl)-N′-ethylcarbodiimide hydrochloride (1.8 g, 9.4 mmol), 1-hydroxybenzotriazole hydrate (740 mg, 4.8 mmol) and 1H-pyrrolo[2,3-b]pyridine-5-amine (0.85 g, 6.4 mmol) were combined in DMF (40 mL) and stirred at room temperature overnight. The mixture was then partitioned between EtOAc and sat'd NaHCO₃ and the organic layer was washed with H₂O and brine, then dried (Na₂SO₄), filtered and the solvent removed under vacuum to leave a crude oil. The oil was purified by column chromatography on silica gel using 0-10% MeOH/CH₂Cl₂ as eluent t give an oil. Further purification by column chromatography on silica gel using 0-50%. EtOAc/hexane as eluent gave a solid, which was purified further by preparative high-performance liquid chromatography to give the product (130 mg, 12%) as a solid. m/z=345.6 (M+1). ¹H NMR (400 MHz; MeOH-d₄) δ 8.48 (2H, d), 7.65-7.6 (3H, m), 7.48 (1H, d), 7.35 (1H, dd), 6.65 (1H, m), 6.5 (1H, d), 2.52 (3H, s).

### b. (E)-N-(1-acetyl-1H-pyrrolo[2,3-b]pyridin-5-yl]-2-methyl-4-(3,3,3-trifluoroprop-1-enyl)benzamide

Acetyl chloride (12 μL, 0.17 mmol) was added to a stirred solution of N,N-di-iso-propylethylamine (61 μL, 0.35 mmol) and (E)-2-methyl-N-(1H-pyrrolo[2,3-b]pyridin-5-yl)-4-(3,3,3-trifluoroprop-1-enyl)benzamide (60 mg, 0.17 mmol) in CH₂Cl₂ (15 mL) at room temperature under an atmosphere of nitrogen. The mixture was stirred overnight at room temperature, then partitioned between H₂O and CH₂Cl₂. The organic layer was dried (MgSO₄) and concentrated under vacuum to leave a crude residue. Purification by column chromatography on silica gel gave a product, which was purified further by preparative high-performance liquid chromatography to give the title compound (5 mg, 7%) as solid. m/z=388.2 (M+1). ¹H NMR (400 MHz; d₆-DMSO) δ 10.5 (1H, s), 8.5 (2H, d), 7.75-7.68 (4H, m), 7.4 (1H, d), 6.9 (1H, m), 6.65 (1H, d), 2.43 (3H, s), 2.35 (3H, s).

### Compound 409

### Preparation of (E)-N-(2-(2-hydroxypropan-2-yl)benzo[d]thiazol-5-yl)-2-methyl-4-(3,3,3-trifluoroprop-1-enyl)benzamide

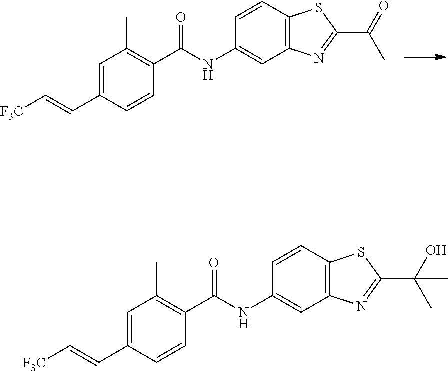

A solution of (E)-N-(2-acetylbenzo[d]thiazol-5-yl)-2-methyl-4-(3,3,3-trifluoroprop-1-enyl)benzamide (65 mg, 0.16 mmol) in THF (25 mL) was cooled to −78° C. A solution of MeLi in Et₂O (1.6 M; 200 μL, 0.32 mmol) was added, and the mixture was slowly warmed to room temperature. The mixture was then partioned between a solution of NH₄Cl and EtOAc. The organic layer was washed with water, brine, dried (Na₂SO₄), filtered and concentrated under vacuum to leave a crude residue. The residue was purified by column chromatography on silica gel to give the product (23 mg). m/z=420.8 (M+1). ¹H NMR (400 MHz; acetone-d₆) δ 9.6 (1H, s), 8.5 (1H, s), 7.9 (1H, d), 7.72 (1H, dd), 7.6-7.5 (3H, m), 7.38 (1H, dd), 6.65 (1H, m), 2.45 (3H, s), 1.62 (6H, s).

### Compound 410

### Preparation of (E)-N-(2-acetylbenzo[d]thiazol-5-yl)-2-methyl-4-(3,3,3-trifluoroprop-1-enyl)benzamide

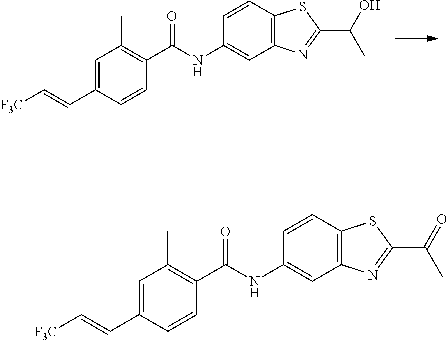

DMSO (142 μL, 2.0 mmol) was added to a solution of oxalyl chloride (86 μL, 1.0 mmol) in CH₂Cl₂ (10 mL) at −78° C. under nitrogen. The mixture was stirred for 10 min, then a solution of 4-((E)-3,3,3-trifluoroprop-1-enyl)-N-(2-(1-hydroxyethyl)benzo[d]thiazol-5-yl)-2-methylbenzamide (370 mg, 0.91 mmol) in CH₂Cl₂ (5 mL) was added. The mixture was stirred for 15 min at −78° C. then Et₃N (634 μL, 4.55 mmol) was added. The mixture was stirred for 30 min at −78° C. then allowed to warm to room temperature. The mixture was partitioned between CH₂Cl₂ and water and the organic layer was dried (MgSO₄), filtered and concentrated under vacuum to leave a crude oil. The oil was purified by preparative high-performance liquid chromatography to give the product (90 mg, 24%) as a solid (ca. 90% pure). m/z=404.8 (M+1). ¹H NMR (400 MHz; d₆-DMSO) δ 10.75 (1H, s), 8.75 (1H, s), 8.2 (1H, d), 7.9 (1H, dd), 7.73-7.55 (3H, m), 7.45 (1H, dd), 6.9 (1H, m), 2.75 (3H, s), 2.45 (3H, s).

### Compound 411

### Preparation of (E)-N-(2-(hydroxymethyl)benzo[d]oxazol-5-yl)-2-methyl-(3,3,3-trifluoroprop-1-enyl)benzamide

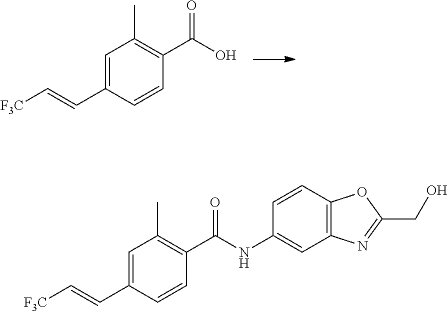

N,N-Di-iso-propylethylamine (80 μL, 0.6 mmol) was added to a mixture of 4-((E)-3,3,3,-trifluoroprop-1-enyl)-2-methylbenzoic acid (50 mg, 0.2 mmol), 1-hydroxybenzotriazole (35 mg, 0.26 mmol) and N-(3-dimethylaminopropyl)-N′-ethylcarbodiimide hydrochloride (50 mg, 0.26 mmol) in CH₂Cl₂ at room temperature. After stirring the mixture for 15 min (5-aminobenzo[d]oxazol-2-yl)methanol (43 mg, 0.26 mmol) was added and the mixture was stirred for 48 hr. The mixture was concentrated under vacuum and the residue was purified by column chromatography on silica gel using 0-40% EtOAc/hexane then 80% EtOAc/hexane as eluent to give the product (24 mg, 30%) as a solid. m/z=376.5 (M+1). ¹H NMR (400 MHz; CDCl₃) δ 7.61 (2H, s), 7.49 (1H, d), 7.36 (3H, t), 7.14 (1H, dd), 6.96 (1H, d), 6.83 (1H, dd), 6.30-6.34 (1H, m), 4.60 (2H, s), 2.52 (3H, s).

### Compound 412

### Preparation of (E)-2-methyl-N-(2-methylthiazolo[5,4-b]pyridin-6-yl)-4-(3,3,3-trifluoroprop-1-enyl)benzamide

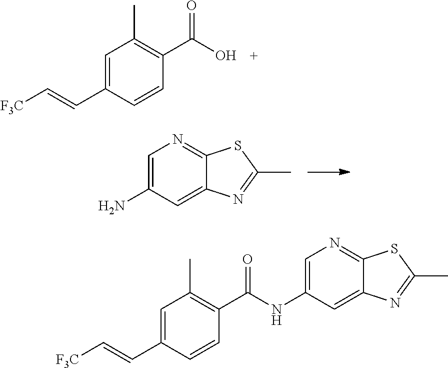

4-((E)-3,3,3-trifluoroprop-1-enyl)-2-methylbenzoic acid (700 mg, 3.0 mmol) was suspended in CH₂Cl₂ (25 mL). Oxalyl chloride (520 μL, 6.1 mmol) was added, followed by the addition of 1 drop of DMF. The mixture was stirred at room temperature for 2 h, then the volatiles were removed under vacuum. The residue was re-suspended in CH₂Cl₂ and triethylamine (1.26 mL, 9.0 mmol) was added, followed by the addition of a solution of 2-methylthiazolo[5,4-b]pyridin-6-amine (502 mg, 3.0 mmol) in CH₂Cl₂ (5 mL). The mixture was stirred at room temperature overnight and then partitioned between EtOAC and water. The organic layer was separated and dried, filtered and then concentrated under vacuum to an oil. Purification of the oil by column chromatography on silica gel using EtOAc/hexane as eluent (0-50%) gave a solid. Trituration with ether gave (E)-2-methyl-N-(2-methylthiazolo[5,4-b]pyridin-6-yl)-4-(3,3,3-trifluoroprop-1-enyl)benzamide (140 mg, 12%) as a solid. m/z=377.8 (M+1). ¹H NMR (400 MHz; d₆-DMSO) δ 11.5 (1H, s), 8.85 (1H, d), 8.71 (1H, d), 7.72-7.58 (3H, m), 7.47 (1H, dd), 6.90 (1H, m), 2.85 (3H, s), 2.45 (3H, s).

### Compound 413

### Preparation of (E)-N-(2-(hydroxymethyl)thiazolo[5,4-b]pyridin-6-yl)-2-methyl-4-(3,3,3-trifluoroprop-1-enyl)benzamide

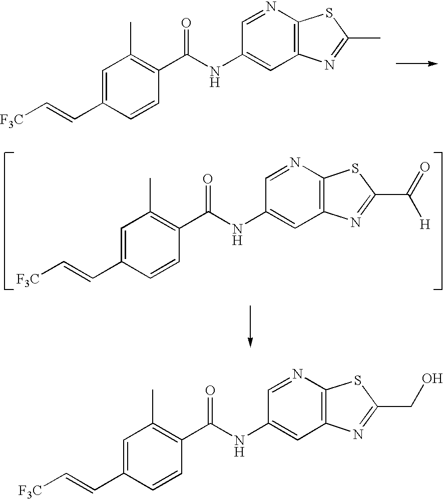

(E)-2-Mmethyl-N-(2-methylthiazolo[5,4-b]pyridin-6-yl)-4-(3,3,3-trifluoroprop-1-enyl)benzamide (60 mg, 0.2 mmol) was placed in dioxane (20 mL). Selenium dioxide (200 mg, 2.0 mmol) was added and the mixture was heated at 105° C. for 28 h. After allowing to cool to room temperature, the mixture was filtered and the and the filtrate was partitioned between EtOAc and aqueous NaHCO₃. The organic layer was washed with water then brine, dried, filtered and concentrated under vacuum to leave a crude oil. The oil was dissolved in THF (20 mL) and water (10 mL) then sodium tetrahydroborate (60 mg, 2.0 mmol) was added in two portions. The mixture was stirred at room temperature for 2 h (monitoring by LCMS), then cooled to 0° C. and adjusted to pH 1 with 1N HCl. The pH was then re-adjusted to pH 8 with sat'd aqueous NaHCO₃. The mixture was extracted with EtOAc and the organic layer was dried, filtered and concentrated under vacuum to leave a crude oil. Purification of the oil by preparative high-performance liquid chromatography gave (E)-N-(2-(hydroxymethyl)thiazolo[5,4-b]pyridin-6-yl)-2-methyl-4-(3,3,3-trifluoroprop-1-enyl)benzamide (13 mg, 20%) as a solid. m/z=394.0 (M+1). ¹H NMR (400 z; d₆-DMSO) δ 11.5 (1H, s), 8.85 (1H, d), 8.71 (1H, d), 7.72-7.58 (3H, m), 7.38 (1H, dd), 6.90 (1H, m), 6.4 (1H, bs), 4.88 (2H, s), 2.45 (3H, s).

### Alternative preparation of (E)-N-(2-(hydroxymethyl)thiazolo[5,4-b]pyridin-6-yl)-2-methyl-4-(3,3,3-trifluoroprop-1-enyl)benzamide

### Compound 413

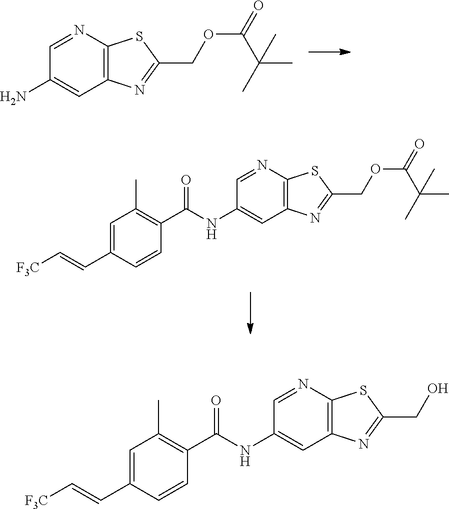

### a. (E)-(6-(2-methyl-4-(3,3,3-trifluoroprop-1-enyl)benzamido)thiazolo[5,4-b]pyridin-2-yl)methyl pivalate

Oxalyl chloride (236 μL, 2.8 mmol) was added in one portion to a stirred solution of (E)-2-methyl-4-(3,3,3-trifluoroprop-1-enyl)benzoic acid (321 mg, 1.39 mmol) in methylene chloride (10 mL) and DMF (5 drops) at 0° C. under nitrogen. The mixture was stirred at 0° C. for 15 min then allowed to warm to room temperature and stirred for ca. 45 min. TLC indicated a small amount of acid left so added further oxalyl chloride (120 μL) at room temperature and stirred for a further 20 min. The mixture was then concentrated under vacuum to leave a crude residue which was dissolved in THF (5 mL) and cooled to 0° C. under an atmosphere of nitrogen. Triethylamine (243 μL, 1.74 mmol) was added, followed by a solution of (6-aminothiazolo[5,4-b]pyridin-2-yl)methyl pivalate (370 mg, 1.4 mmol) in THF (10 mL). The mixture was stirred at 0° C. for 15 min, then allowed to warn to room temperature and stirred overnight. The solvent was removed under vacuum to leave a crude residue which was partitioned between EtOAc (100 mL) and H₂O (50 mL.) The organic layer was washed with H₂O (1×50 mL), sat'd NaHCO₃ (2×50 mL), brine (1×50 mL), then dried (MgSO₄), filtered and the solvent removed under vacuum to leave a crude solid (630 mg, 85%). The solid was used without further purification (NMR indicated it was approximately 90% pure).

### b. (E)-N-(2-(hydroxymethyl)thiazolo[5,4-b]pyridin-6-yl)-2-methyl-4-(3,3,3-trifluoroprop-1-enyl)benzamide

Sodium (200 mg, 8.7 mmol) was added in one portion to stirring methanol (25 mL) at room temperature under nitrogen. After complete dissolution of the sodium (reaction is exothermic), (E)-(6-(2-methyl-4-(3,3,3-trifluoroprop-1-enyl)benzamido)thiazolo[5,4-b]pyridin-2-yl)methyl pivalate (550 mg, 1.2 mmol) was added in one portion as a suspension in MeOH (20 mL) (a further 10 mL MeOH rinse was used to ensure all material deposited in the reaction mixture). The mixture was stirred at room temperature for ca. 15 mins then the mixture was concentrated under vacuum. The residue was partitioned between 1M NH₄Cl (100 mL) and EtOAc (150 mL). The organic layer was dried (MgSO₄), filtered and the solvent removed under vacuum to leave a solid. The solid was purified by trituration with MeOH (ca. 5-10 mL) to give the desired product (140 mg) as a solid. The filtrate was concentrated under vacuum and the residue was purified by column chromatography on silica gel using 50-80% EtOAc/hexane to give a solid. This solid was triturated with MeOH to give further desired product (50 mg) as a solid. The filtrate was again concentrated under vacuum and the residue purified by preparative thin-layer chromatography using 75% EtOAc/hexane to give the desired compound (120 mg) as a solid. The total yield of (E)-N-(2-(hydroxymethyl)thiazolo[5,4-b]pyridin-6-yl)-2-methyl-4-(3,3,3-trifluoroprop-1-enyl)benzamide from this reaction was 310 mg (67%). Analytical data was identical to that described above.

### Compound 414

### Preparation of (E)-ethyl (2-methyl-4-(3,33-trifluoroprop-1-enyl)benzamido)thiazolo[5,4-b]pyridin-2-carboxylate

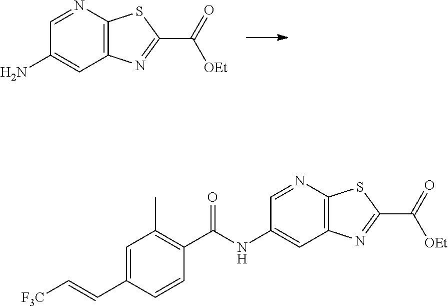

4-((E)-3,3,3-trifluoroprop-1-enyl)-2-methylbenzoic acid (103 mg, 0.45 mmol) was suspended in CH₂Cl₂. Oxalyl chloride (77 μL, 0.91 mmol) was added, followed by the addition of I drop of DMF. The mixture was stirred at room temperature for 1 h, then the volatiles were removed under vacuum. The residue was re-suspended in CH₂Cl₂ and triethylamine (187 μL, 1.35 mmol) was added, followed by the addition of a solution of ethyl-6-aminothiazolo[5,4-b]pyridin-2-carboxylate (100 mg, 0.4 mmol). The mixture was stirred at room temperature for 1 h then partitioned between EtOAC and water. The organic layer was separated and dried, filtered and then concentrated under vacuum to an oil. Purification of the oil by column chromatography on silica gel using 0.5% EtOAc/hexane as eluent gave a solid, which was purified further by trituration with Et₂O to give the product (15 mg, 7%) as a solid. m/z=436.1 (M+1). ¹H NMR (400 MHz; d₆-DMSO) δ 11.0, (1H, s), 9.05 (2H, dd), 7.8-7.6 (3H, m), 7.4 (1H, dd), 6.95 (1H, m), 4.45 (2H, q), 2.45 (3H, s), 1.45 (2H, t).

### Compound 415

### Preparation of (E)-2-methyl-N-(thiazolo[5,4-b]pyridin-6-yl)-4-(3,3,3-trifluoroprop-1-enyl)benzamide

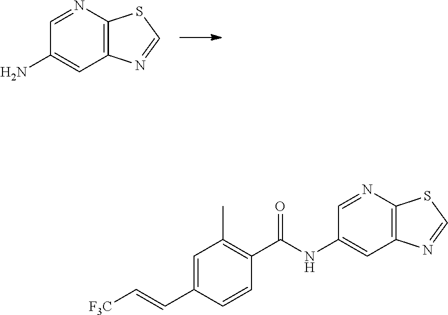

4-(E)-3,3,3-trifluoroprop-1-enyl)-2-methylbenzoic acid (456 mg, 1.98 mmol) was suspended in CH₂Cl₂. Oxalyl chloride (340 μL, 4.0 mmol) was added, followed by the addition of 1 drop of DMF. The mixture was stirred at room temperature for 1 h, then the volatiles were removed under vacuum. The residue was re-suspended in CH₂Cl₂ and triethylamine (830 mL, 6.0 mmol) was added, followed by the addition of a solution of thiazolo[5,4-b]pyridin-6-amine (300 mg, 1.98 mmol) in DMF (1 mL). The mixture was stirred at room temperature for 1 h then partitioned between EtOAC and water. The organic layer was separated and dried, filtered and then concentrated under vacuum to an oil. Purification of the oil by column chromatography on silica gel gave a solid (90 mg) which was purified further by preparative high-performance liquid chromatography to give the product (35 mg, 5%) as a solid. m/z=364.3 (M+1). ¹H NMR (400 MHz; acetone-d₆) δ 9.75, (1H, bs), 9.3 (1H, s), 8.85 (2H, dd), 7.55 (3H, m), 7.2 (1H, dd), 6.62 (1H, m), 2.4 (3H, s).

### General Method for Automated parallel LC-MS Purification of Libraries

The libraries were purified using a Perkin Elmer API100 mass spectrometer coupled to Shimadzu LC pumps. The chromatographic method employed was 10-100% gradient of acetonitrile to water over 8 minutes at a flow rate of 6 ml per minute. The column used was a 10×50 mm YMC C18 and the compounds were collected using a Gilson 204 fraction collector.

Following the methods described above and the appropriate reagents, starting materials and purification methods known to those skilled in the art, the amide compounds of this invention were or can be prepared.

The synthetic and biological examples presented herein are offered to illustrate this invention and are not to be construed in any way as limiting the scope of this invention. In the examples below, all temperatures are in degrees Celsius (unless otherwise indicated).

The compounds that have been prepared in accordance with the invention are presented in Table 1, below. The syntheses of these representative compounds were carried out in accordance with the methods set forth above, and activity of the compounds was measured by percent inhibition in a calcium uptake assay, the details of which are described below.

### Calcium Uptake Assay.

Functional activity of compounds against the VR1 receptor was determined by measuring changes in intracellular calcium in HEK 293 cells expressing hVR1. Compounds were examined for their ability to inhibit agonist-induced calcium influx. Dual wavelength ratiometric dye, Fura2, was used as an indicator of relative levels of [Ca²⁺] in a 96-well format using a Flex Station®, Molecular Devices.

### Cell Line and Culture Conditions:

hVR1 was cloned into a pcDNA5/TO vector from Invitrogen and stably transformed into T-REx HEK 293 cell line from Invitrogen. HEK 293 cells expressing hVR1 were grown to confluency (24 hour culture) on PDL-coated, plastic 96-well black-walled plates, in the presence of DMEM medium containing 5% PenStrep, 5% Glutamax, 200 μg/mL Hygromycin, 5 μg/mL Blasticidin and 10% heat inactivated FBS. Twenty-four hours prior to assay, cells were transferred to DMEM media containing 1 μg/mL doxycycline. Prior to the assay, cells were loaded with 5 μg/mL Fura-2 (Molecular Probes) in saline solution (130 mM NaCl, 3 mM KCl, 1 mM CaCl₂, 0.6 mM MgCl₂, 10 mM HEPES, 10 mM glucose and 50 mM sucrose pH 7.4) at 37° C. for 40 minutes. The dye was then aspirated and replaced with 100 μL saline before commencement of the assay in Flex Station®.

### Agonist Concentration and Compound Dilutions:

The agonist EC₅₀ was determined at the start of the assay and compound IC₅₀ experiments were run using an agonist concentration equal to its EC₅₀ as stimulus. The agonists used were capsaicin (EC₅₀=2.5 nM) and protons (saline solution plus 10 mM citric acid buffered to pH 5.7 with HCl). Compounds were tested at concentrations ranging from 10 nM to 3.3 μM.

The assay consists of two stages: a pre-treatment phase followed by a treatment phase. 50 μl of a compound solution was added to the cells (Pre-treatment). In some instances, following pre-treatment, 50 μl of the test compound in a saline solution at pH 5.1 was added (Treatment). Compounds were tested as follows: For the pre-treatment phase, 50 μL of 3× concentration of test compound in saline is added to cells containing 100 mL of saline to achieve a final concentration of x. For the treatment phase, at a determined time after pre-treatment, 50 μL of test compound plus agonist solution is added to cells at the relevant concentrations.

Recordings were made at 4 second intervals at wavelengths of 340 nM and 380 nM and the fluorescence ratio analyzed. Responses were measured as peak fluorescence ratio after compound-agonist addition minus baseline fluorescence ratio prior to treatment and were calculated using the SoftMaxPro software from Molecular Devices. Percent inhibition was calculated as follows and is depicted in Table 1:

$$Percentage inhibition = 1 - \frac{( \begin{matrix}Compound Response - \\ Control Response\end{matrix} )}{( \begin{matrix}Agonist Response - \\ Control Response\end{matrix} )} \times 100$$

TABLE 1
AMIDE COMPOUNDS

| | | | | | Low pH |
|---|---|---|---|---|---|
| | | | | | % |
| | | MS | Method | | Inhib. |
| | | observed | of | | @ 0.3 |
| ID | Strucuture | (calcd) | Synth. | H NMR | μM |
| 1 | 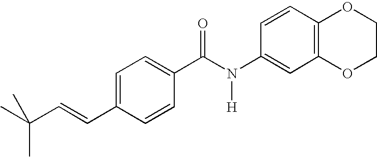 | 338.29 (337.42) | A | | 83 |
| 45 | 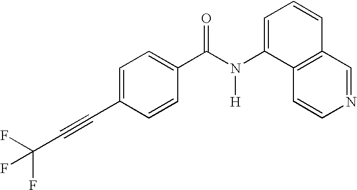 | 339.20 (340.31) | I | (d₆-DMSO) δ 10.73 (s, 1H) 9.38 (s, 1H) 8.53 (d, 1H) 8.19 (d, 2H) 8.08 (d, 1H) 7.98-7.85 (m, 4H) 7.75 (t, 1H) | 99 |
| 77 | 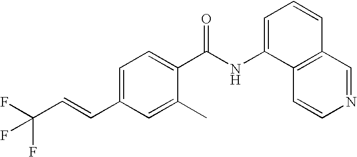 | 357.20 (356.35) | E | (d₆-DMSO) δ 10.53 (s, 1H), 9.36 (s, 1H), 8.55 (d, 1H, J = 5.6 Hz), 8.04 (d, 1H, J = 8.0 Hz), 8.02 (s, 1 H), 7.95 (d, 1H, J = 5.6 Hz), 7.80-7.65 (m, 4H), 7.42-7.35 (m, 1H), 6.88 (dq, 1H, J = 16.4, 6.8 Hz), 2.50 (s, 3H). | 90 |
| 96 | 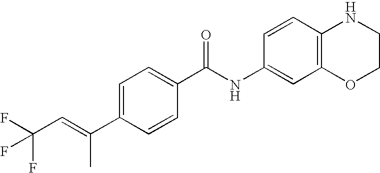 | 363.40 (362.35) | D | (d₆-DMSO) δ 9.94 (s, 1H), 7.95 (d, 2H, J = 8.4 Hz). 7.71 (d, 2H, J = 8.4 Hz), 7.15 (d, 1H, J = 2.4 Hz), 7.07 (dd, 1H, J = 8.8, 2.4 Hz), 6.52 (d, 1H, J = 8.8 Hz), 6.32 (q, 1H, J = 9.2 Hz), 5.61 (s, 1H), 4.12 (t, 2H, J = 4.4 Hz), 3.26 (m, 2H), 2.30 (m, 3H). | 99 |
| 118 | 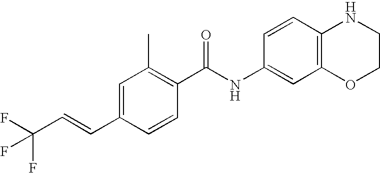 | 363.40 (362.35) | D | (d₆-DMSO) δ 9.93 (s, 1H), 7.63 (s, 1H), 7.59 (d, 1H, J = 8.0 Hz). 7.44 (d, 1H, J = 8.0 Hz), 7.37-7.31 (m, 1H), 7.11 (d, 1H, J = 2.4 Hz), 7.01 (dd, 1H, J = 8.8, 2.4 Hz), 6.82 (dq, 1H, J = 16.4, 7.2 Hz), 6.50 (d, 1H, J = 8.8 Hz), 5.59 (s, 1H), 4.11 (t, 2H, J = 4.4 Hz), 3.24 (m, 2H), 2.37 (s, 3H). | 103 |
| 119 | 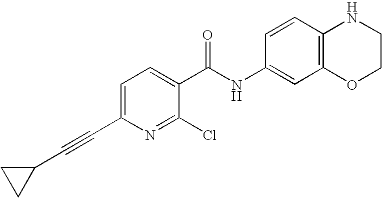 | 354.20 (353.81) | I | (d₆-DMSO) δ 10.25 (1H, s), 7.9 (1H, d), 7.55 (1H, d), 7.15 (1H, s), 6.95 (1H, d), 6.55 (1H, d), 5.78 (1H, s), 4.25 (2H, m), 3.35 (2H, m), 1.85 (1H, m), 1.05 (2H, m), 0.95 (2H, m) | 0 |
| 120 | 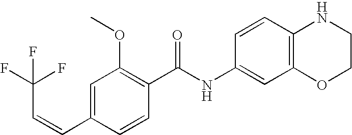 | 379.20 (378.35) | D | (d₆-DMSO) δ 9.77 (s, 1H), 7.64 (d, 1H, J = 8.0 Hz), 7.24 (d, 1H, J = 12.8 Hz), 7.15 (s, 1H), 7.13 (d, 1H, J = 2.0 Hz). 7.08 (d, 1H, J = 8.0 Hz), 7.01 (dd, 1H, J = 8.4, 2.4 Hz), 6.50 (d, 1H, J = 8.4 Hz), 6.18 (dq, 1H, J = 12.8, 9.6 Hz), 5.59 (s, 1H), 4.11 (1, 2H, J = 4.4 Hz), 3.91 (s, 3H), 3.25 (m, 2H). | 19 |
| 121 | 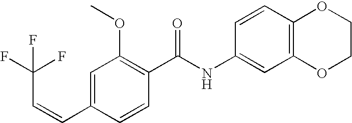 | 380.20 (379.34) | D | (d₆-DMSO) δ 9.78 (s, 1H), 7.62 (d, 1H, J = 8.0 Hz), 7.36 (d, 1H, J = 2.4 Hz), 7.24 (d, 1H, J = 12.4 Hz), 7.15 (s, 1H), 7.13 (dd, 1H, J = 8.8, 2.4 Hz), 7.08 (d, 1H, J = 8.0 Hz), 6.80 (d, 1H, J = 9.2 Hz), 6.19 (dq, 1H, J = 12.4, 9.2 Hz), 4.22 (m, 4H), 3.88 (s, 3H). | 19 |
| 122 | 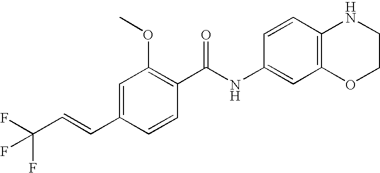 | 379.20 (378.35) | F | (d₆-DMSO) δ 9.76 (s, 1H), 7.65 (d, 1H, J = 8.0 Hz), 7.49 (s, 1H), 7.42-7.33 (m, 2H), 7.13 (d, 1H, J = 2.4 Hz). 7.00 (dd, 1H, J = 8.4, 2.4 Hz), 6.94 (dq, 1H, J = 16.4, 7.2 Hz), 6.50 (d, 1H, J = 8.8 Hz), 5.59 (s, 1H), 4.11 (t, 2H, J = 4.4 Hz), 3.94 (s, 3H), 3.25 (m, 2H). | 19 |
| 123 | 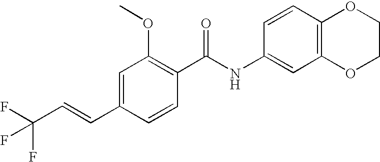 | 380.10 (379.34) | F | (d₆-DMSO) δ 9.97 (s, 1H), 7.64 (d, 1H, J = 8.0 Hz), 7.50 (s, 1H), 7.43-7.34 (m, 3H), 7.12 (dd, 1H, J = 8.8, 2.4 Hz), 6.95 (dq, 1H, J = 16.4, 7.2 Hz), 6.80 (d, 1H, J = 8.8 Hz), 4.22 (m, 4H), 3.93 (s, 3H). | 19 |
| 124 | 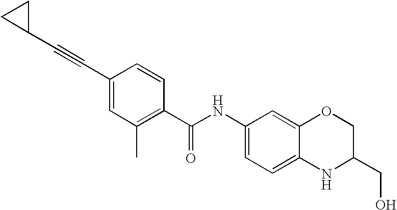 | 363.0 (362.43) | B | | 112 |
| 125 | 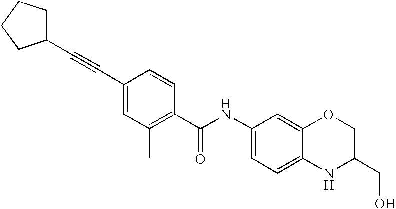 | 391.0 (390.49) | B | | 112 |
| 126 | 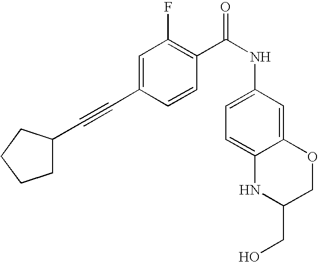 | 395.30 (394.45) | B | | 96 |
| 127 | 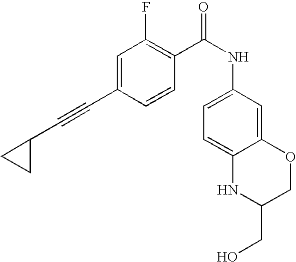 | 367.30 (366.40) | B | (d₆-DMSO) δ 10.00 (s, 1H) 7.55 (t 1H) 7.32 (dd, 1H) 7.27 (dd, 1H) 7.09 (d, 1H) 6.99 (dd, 1H) 6.54 (d, 1H) 5.7 (s, 1H) 4.87 (t, 1H) 4.11 (dd 1H) 3.92 (dd 1H) 3.45-3.27 (m, 2H) 1.63-1.54 (m, 1H) 0.96-0.896 (m, 2H) 0.81-0.75 (m, 2H) | 100 |
| 128 | 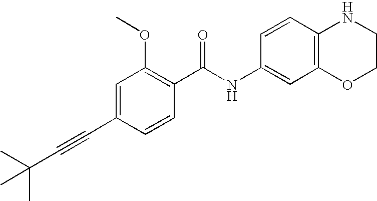 | 365.50 (364.45) | D | (d₆-DMSO) δ 9.71 (s, 1H), 7.61 (d, 1H, J = 8.0 Hz), 7.12 (d, 1H, J = 2.4 Hz), 7.06 (d, 1H, J = 1.6 Hz), 7.02 (dd, 1H, J = 8.0, 1.6 Hz), 6.99 (dd, 1H, J = 8.4, 2.4 Hz), 6.50 (d, 1H, J = 8.4 Hz). 5.59 (s, 1H), 4.11 (t, 2H, J = 4.4 Hz), 3.91 (s, 3H), 3.25 (m, 2H), 1.31 (s, 9H). | 12 |
| 129 | 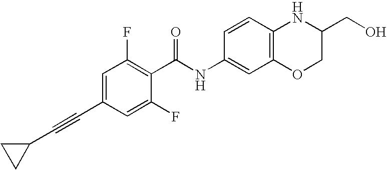 | 385.40 (384.39) | D | (d₆-DMSO) δ 10.36 (s, 1H), 7.23 (d, 2H, J = 8.0 Hz), 7.06 (d, 1H, J = 2.4 Hz), 6.95 (dd, 1H, J = 8.4, 2.4 Hz), 6.55 (d, 1H, J = 8.4 Hz), 5.73 (s, 1H), 4.86 (t, 1H, J = 5.2 Hz), 4.11 (dd, 1H, J = 10.4, 2.0 Hz), 3.92 (dd, 1H, J = 10.4, 6.0 Hz), 3.43-3.32 (m, 2H), 3.30 (m, 1H), 1.59 (m, 1H), 0.96-0.91 (m, 2H), 0.81-0.77 (m, 2H). | 104 |
| 130 | 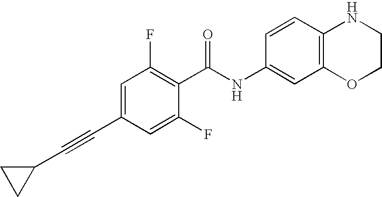 | 355.20 (354.36) | D | (d₆-DMSO) δ 10.36 (s, 1H), 7.23 (d, 2H, J = 8.0 Hz), 7.05 (d, 1H, J = 2.4 Hz), 6.95 (dd, 1H, J = 8.4, 2.4 Hz), 6.51 (d, 1H, J = 8.4 Hz), 5.67 (s, 1H), 4.11 (t, 2H, J = 4.4 Hz). 3.25 (m, 2H), 1.59 (m, 1H), 0.96-0.91 (m, 2H), 0.81-0.76 (m, 2H). | 104 |
| 131 | 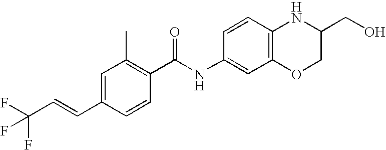 | 393.50 (392.38) | D | (d₆-DMSO) δ 9.93 (s, 1H), 7.63 (s, 1H), 7.59 (d, 1H, J = 8.0 Hz), 7.44 (d, 1H, J = 8.0 Hz), 7.34 (m, 1H), 7.13 (s, 1H, J = 2.4 Hz), 7.02 (dd, 1H, J = 8.4, 2.4 Hz), 6.83 (dq, 1H, J = 16.4, 7.2 Hz), 6.54 (d, 1H, J = 8.4 Hz), 5.64 (s, 1H), 4.86 (t, 1H, J = 5.2 Hz), 4.11 (dd, 1H, J = 10.4, 2.0 Hz), 3.92 (dd, 1H, J = 10.4, 5.6 Hz), 3.44-3.32 (m, 2H), 3.30 (m, 1H), 2.37 (s, 3H). | 104 |
| 132 | 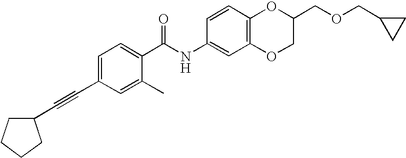 | 446.50 (445.56) | I | (d₆-DMSO) δ 10.25 (1H, s), 7.45 (2H, m), 7.35 (2H, m), 7.15 (1H, d), 6.85 (1H, d), 4.25 (2H, m), 4.05 (1H, m), 3.65 (2H, m), 3.55 (2H, m), 2.65 (1H, m) 2.35 (3H, s), 2.1-1.95 (2H, m), 1.85-1.5 (6H, m), 1.5 (1H, m), 1.45 (2H, m), 1.25 (2H, m) | |
| 133 | 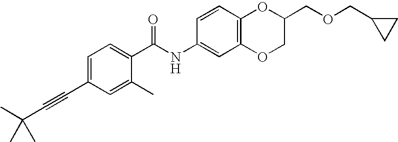 | 434.30 (433.55) | I | (d₆-DMSO) δ 10.25 (1H, s), 7.45 (2H, m), 7.35 (2H, m), 7.15 (1H, d), 6.85 (1H, d), 4.25 (2H, m), 4.05 (1H, m), 3.65 (2H, m), 3.55 (2H, m), 2.45 (3H, s) 1.85 (9H, s), 1.5 (1H, m), 1.45 (2H, m), 1.25 (2H, m) | |
| 134 | 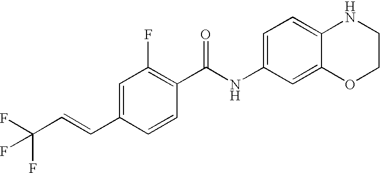 | 367.80 (366.32) | D | (d₆-DMSO) δ 10.04 (s, 1H), 7.74 (d, 1H, J = 11.2 Hz), 7.66 (t, 1H, J = 8.0 Hz), 7.61 (d, 1H, J = 8.0 Hz), 7.44-7.37 (m, 1H), J = 8.4, 2.4 Hz), 6.96 (m, 1H), 6.51 (d, 1H, J = 8.4 Hz), 5.63 (s, 1H), 4.11 (t, 2H, J = 4.4 Hz), 3.25 (m, 2H). | 108 |
| 135 | 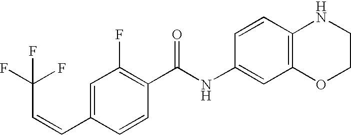 | 367.20 (366.32) | D | (d₆-DMSO) δ 10.06 (s, 1H), 7.67 (t, 1H, J = 7.6 Hz), 7.34-7.29 (m, 2H), 7.25 (d, 1H, J = 12.8 Hz), 7.10 (d, 1H, J = 2.4 Hz), 7.01 (dd, 1H, J = 8.4. 2.4 Hz), 6.51 (d, 1H, J = 8.4 Hz), 6.25 (dq, 1H, J = 12.4, 9.2 Hz), 5.63 (s, 1H), 4.10 (t, 2H, J = 4.4 Hz), 3.25 (m, 2H). | 29 |
| 137 | 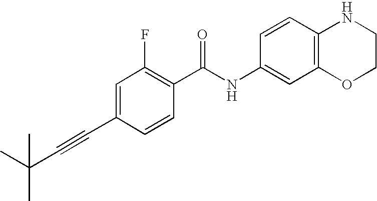 | 352.70 (352.41) | C | (d₆-DMSO) δ 9.98 (1H, s), 7.57 (1H, t), 7.32-7.25 (2H, m), 7.08 (1H, d), 6.99 (1H, dd), 6.51 (1H, d), 5.62 (1H, brs), 4.11 (2H, t), 3.24 (2H, t), 1.30 (9H, s). | 103 |
| 138 | 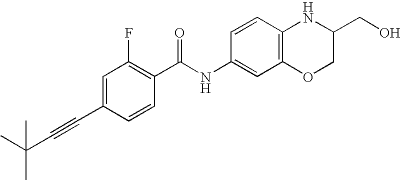 | 382.70 (382.44) | C | (d₆-DMSO) δ 9.99 (1H, s), 7.57 (1H, t), 7.32-7.25 (2H, m), 7.09 (1H, d), 6.99 (1H, dd), 6.54 (1H, d), 5.69 (1H, brs), 4.86 (1H, t), 4.12 (1H, dd), 3.91 (1H, dd), 3.43-3.28 (3H, m), 1.30 (9H, s). | 107 |
| 139 | 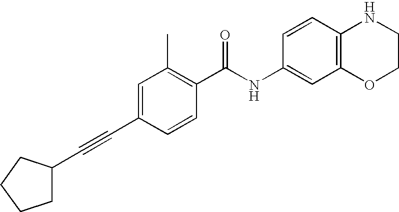 | 360.90 (360.46) | C | (d₆-DMSO) δ 9.88 (1H, s), 7.34 (1H, d), 7.28 (1H, s), 7.25-7.23 (1H, m), 7.09 (1H, d), 7.00 (1H, dd), 6.49 (1H, d), 5.57 (1H, brs), 4.20 (2H, t), 3.26-3.23 (2H, m), 2.89-2.73 (1H, m), 2.32 (3H, s), 1.99-1.97 (2H, m), 1.73-1.70 (2H, m), 1.63-1.56 (4H, m). | 95 |
| 140 | 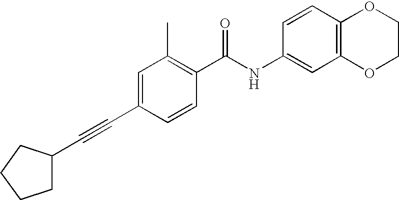 | 361.90 (361.44) | C | (d₆-DMSO) δ10.12 (1H, s), 7.38-7.25 (4H, m), 7.12 (1H, dd), 6.79 (1H, d), 4.23-4.20 (4H, m), 2.89-2.85 (1H, m), 2.33 (3H, s), 2.02-1.95 (2H, m), 1.73-1.68 (2H, m), 1.65-1.55 (4H, m). | 33 |
| 141 | 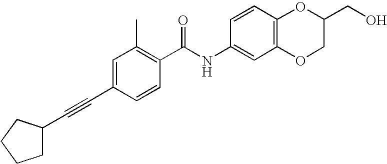 | 392.00 (391.47) | C | (d₆-DMSO) δ 10.12 (1H, s), 7.38-7.35 (2H, m), 7.30 (1H, s), 7.26 (1H, d), 7.13 (1H, dd), 6.83-6.81 (1H, m), 5.06-5.03 (1H, m), 4.31 (1H, dd), 4.12-4.10 (1H, m), 4.01-3.96 (1H, m), 3.66-3.58 (2H, m), 2.89-2.85 (2H, m), 2.33 (3H, s), 2.02-1.95 (2H, m), 1.73-1.67 (2H, m), 1.65-1.55 (4H, m). | 87 |
| 142 | 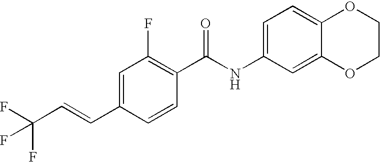 | 368.10 (367.30) | D | (d₆-DMSO) δ 10.27 (s, 1H), 7.76 (d, 1H, J = 10.4 Hz), 7.69 (t, 1H, J = 8.0 Hz), 7.63 (dd, 1H, J = 8.0, 1.2 Hz), 7.45-7.37 (m, 1H), 7.33 (d, 1H, J = 2.8 Hz), 7.12 (dd, 1H, J = 8.8, 2.8 Hz), 6.98 (dq, 1H, J = 16.4, 7.2 Hz), 6.82 (d, 1H, J = 8.8 Hz), 4.23 (m, 4H). | 70 |
| 143 | 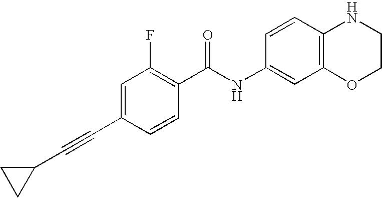 | 337.80 (336.37) | D | (d₆-DMSO) δ 9.98 (s, 1H), 7.55 (t, 1H, J = 7.6 Hz), 7.31 (dd, 1H, J = 11.2, 1.6 Hz), 7.26 (dd, 1H, J = 8.0, 1.6 Hz), 7.08 (d, 1H, J = 2.4 Hz), 6.99 (dd, 1H, J = 8.4, 2.4 Hz), 6.50 (d, 1H, J = 8.4 Hz), 5.62 (s, 1H), 4.11 (t, 2H, J = 4.4 Hz), 3.25 (m, 2H), 1.58 (m, 1H), 0.96-0.90 (m, 2H), 0.80-0.75 (m, 2H). | 100 |
| 144 | 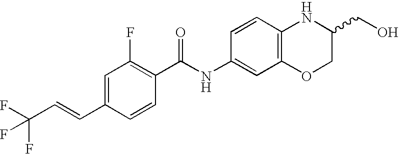 | 397.20 (396.34) | D | (d₆-DMSO) δ 10.04 (s, 1H), 7.74 (d, 1H, J = 11.2 Hz), 7.66 (t, 1H, J = 8.0 Hz), 7.61 (d, 1H, J = 8.0 Hz), 7.41 (m, 1H), 7.11 (d, 1H, J = 2.4 Hz), 7.01 (dd, 1H, J = 8.4, 2.4 Hz), 6.97 (m, 1H), 6.55 (d, 1H, J = 8.4 Hz), 5.69 (s, 1H), 4.86 (t, 1H, J = 5.2 Hz), 4.12 (dd, 1H, J = 10.4, 2.4 Hz), 3.92 (dd, 1H, J = 10.4, 5.6 Hz), 3.45-3.32 (m, 2H), 3.30 (m, 1H). | 105 |
| 145 | 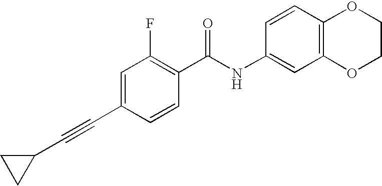 | 338.40 (337.35) | D | (d₆-DMSO) δ 10.22 (s, 1H), 7.58 (t, 1H, J = 7.6 Hz), 7.34 (dd, 1H, J = 11.2, 1.6 Hz), 7.31 (d, 1H, J = 2.4 Hz), 7.28 (dd, 1H, J = 8.0, 1.6 Hz), 7.10 (dd, 1H, J = 8.8, 2.4 Hz), 6.81 (d, 1H, J = 8.8 Hz), 4.22 (m, 4H), 1.58 (m, 1H), 0.96-0.90 (m, 2H), 0.80-0.76 (m, 2H). | 41 |
| 146 | 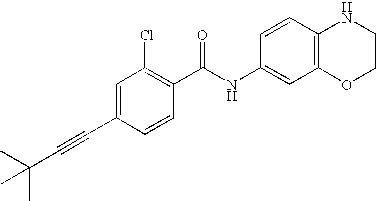 | 369.20 (368.87) | D | (d₆-DMSO) δ 10.09 (s, 1H), 7.49 (d, 1H, J = 1.6 Hz), 7.47 (d, 1H, J = 8.0 Hz), 7.38 (dd, 1H, J = 8.0. 1.6 Hz), 7.08 (d, 1H, J = 2.4 Hz), 6.98 (dd, 1H, J = 8.4, 2.4 Hz), 6.50 (d, 1H, J = 8.4 Hz), 5.62 (s, 1H), 4.11 (t, 2H, J = 4.4 Hz), 3.25 (m, 2H), 1.30 (s, 9H). | 93 |
| 147 | 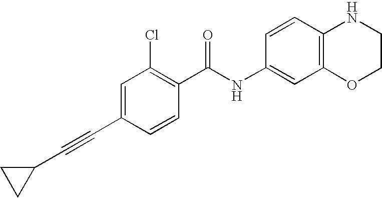 | 352.80 (352.82) | D | (d₆-DMSO) δ 10.08 (s, 1H), 7.51 (d, 1H, J = 1.6 Hz), 7.46 (d, 1H, J = 7.6 Hz), 7.38 (dd, 1H, J = 7.6, 1.6 Hz), 7.07 (d, 1H, J = 2.4 Hz), 6.98 (dd, 1H, J = 8.4, 2.4 Hz), 6.50 (d, 1H, J = 8.4 Hz), 5.61 (s, 1H), 4.10 (t, 2H, J = 4.4 Hz), 3.25 (m, 2H), 1.57 (m, 1H), 0.95-0.88 (m, 2H), 0.80-0.75 (m, 2H). | 88 |
| 148 | 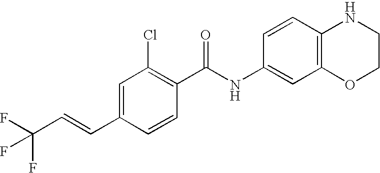 | 382.90 (382.77) | D | (d₆-DMSO) δ 10.12 (s, 1H), 7.93 (d, 1H, J = 1.2 Hz), 7.75 (dd, 1H, J = 8.0, 1.2 Hz), 7.58 (d, 1H, J = 8.0 Hz), 7.43-7.37 (m, 1H), 7.09 (d, 1H, J = 2.4 Hz), 6.99 (dd, 1H, J = 8.8, 2.4 Hz), 6.96 (m, 1H), 6.51 (d, 1H, J = 8.8 Hz), 5.62 (s, 1H), 4.11 (t, 2H, J = 4.4 Hz), 3.25 (m, 2H). | 104 |
| 149 | 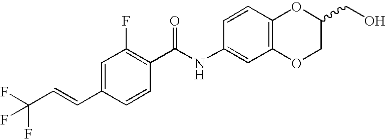 | 398.20 (397.33) | F | (d₆-DMSO) δ 10.28 (s, 1H), 7.76 (d, 1H, J = 11.2 Hz), 7.69 (t, 1H, J = 7.6 Hz), 7.63 (d, 1H, J = 8.4 Hz), 7.42 (m, 1H), 7.34 (d, 1H, J = 2.4 Hz), 7.13 (dd, 1H, J = 8.4, 2.4 Hz), 6.98 (dq, 1H, J = 16.4, 7.2 Hz), 6.84 (d, 1H, J = 8.4 Hz), 5.06 (t, 1H, J = 5.6 Hz), 4.32 (dd, 1H, J = 11.6, 2.0 Hz), 4.13 (m, 1H), 4.00 (dd, 1H, J = 11.6, 8.0 Hz), 3.68-3.56 (m, 2H). | 71 |
| 150 | 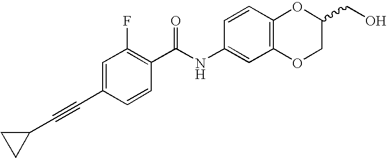 | 368.20 (367.38) | D | (d₆-DMSO) δ 10.22 (s, 1H), 7.58 (t, 1H, J = 8.0 Hz), 7.36-7.31 (m, 2H), 7.28 (dd, 1H, J = 8.0, 1.6 Hz), 7.12 (dd, 1H, J = 8.4, 2.4 Hz), 6.83 (d, 1H, J = 8.4 Hz), 5.05 (t, 1H, J = 5.6 Hz), 4.32 (dd, 1H, J = 11.2, 2.0 Hz), 4.13 (m, 1H), 3.99 (dd, 1H, J = 11.2, 7.6 Hz), 3.68-3.56 (m, 2H), 1.58 (m, 1H), 0.96-0.90 (m, 2H), 0.81-0.76 (m, 2H). | 75 |
| 151 | 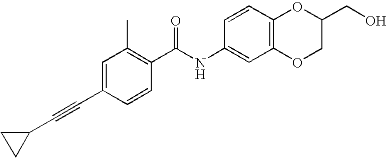 | 363.90 (363.42) | C | (d₆-DMSO) δ 10.12 (1H, s), 7.37-7.34 (2H, m), 7.29 (1H, s), 7.26 (1H, d), 7.13 (1H, dd), 6.81 (1H, d), 5.06-5.03 (1H, m), 4.31 (1H, dd), 4.14-4.10 (1H, m), 4.01-3.96 (1H, m), 3.66-3.57 (2H, m), 2.32 (3H, s), 1.58-1.54 (1H, m), 0.92-0.88 (2H, m), 0.76-0.72 (2H, m). | 108 |
| 152 | 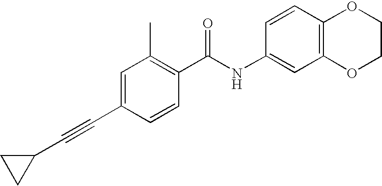 | 334.00 (333.39) | C | (d₆-DMSO) δ 10.11 (1H, s), 7.37-7.33 (2H, m), 7.29-7.24 (2H, m), 7.12 (1H, dd), 6.80 (1H, d), 4.23-4.20 (4H, m), 2.32 (3H, s), 1.58-1.54 (1H, m), 0.92-0.88 (2H, m), 0.76-0.72 (2H, m). | 73 |
| 153 | 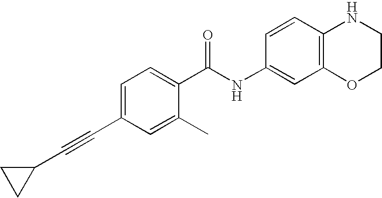 | 332.80 (332.41) | C | (d₆-DMSO) δ 9.88 (1H, s), 7.33 (1H, d), 7.27 (1H, s), 7.24 (1H, d), 7.09 (1H, d), 7.00 (1H, dd), 6.49 (1 H, d), 5.57 (1H, brs), 4.30 (2H, t), 3.29-3.23 (2H, m), 2.33 (3H, s), 1.57-1.53 (1H, m), 0.92-0.87 (2H, m), 0.76-0.72 (2H, m). | 104 |
| 154 | 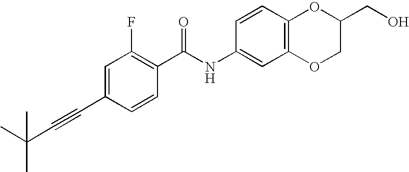 | 383.70 (332.42) | C | (d₆-DMSO) δ 10.23 (1H, s), 7.59 (1H, t), 7.34-7.27 (3H, m), 7.12 (1H, dd), 6.83 (1H, d), 5.05 (1H, t), 4.32 (1H, dd), 4.13-4.11 (1H, m), 4.02-3.97 (1H, m), 3.64-3.58 (2H, m), 1.30 (9H, s). | 95 |
| 155 | 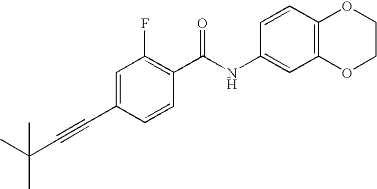 | 353.70 (353.40) | C | (d₆-DMSO) δ 10.22 (1H, s), 7.59 (1H, s), 7.34-7.27 (3H, m), 7.11 (1H, dd), 6.82 (1H, d), 4.24-4.20 (4H, m), 1.30 (9H, s). | 95 |
| 156 | 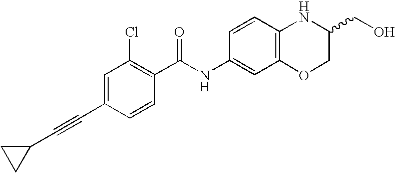 | 383.10 (382.85) | D | (d₆-DMSO) δ 10.08 (s, 1H), 7.51 (d, 1H, J = 1.6 Hz), 7.46 (d, 1H, J = 8.0 Hz), 7.39 (dd, 1H, J = 8.0. 1.6 Hz), 7.08 (d, 1H, J = 2.0 Hz), 6.98 (dd, 1H, J = 8.4, 2.4 Hz), 6.54 (d, 1H, J = 8.4 Hz), 5.67 (s, 1H), 4.86 (t, 1H, J = 5.6 Hz), 4.11 (dd, 1H, J = 10.8, 2.4 Hz), 3.92 (dd, 1H, J = 10.8, 5.6 Hz), 3.43-3.33 (m, 2H), 3.30 (m, 1H), 1.58 (m, 1H), 0.96-0.89 (m, 2H), 0.81-0.76 (m, 2H). | 102 |
| 157 | 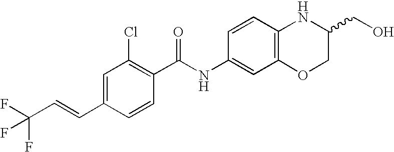 | 413.30 (412.80) | D | (d₆-DMSO) δ 10.12 (s, 1H), 7.93 (d, 1H, J = 1.2 Hz), 7.75 (dd, 1H, J = 8.0, 1.6 Hz), 7.58 (d, 1H, J = 8.0 Hz), 7.40 (m, 1H), 7.10 (d, 1H, J = 2.4 Hz), 7.00 (dd, 1H, J = 8.8, 2.4 Hz), 6.97 (m, 1H), 6.54 (d, 1H, J = 8.8 Hz), 5.68 (s, 1H), 4.86 (t, 1H, J = 5.6 Hz), 4.11 (dd, 1H, J = 10.8, 2.4 Hz), 3.92 (dd, 1H, J = 10.8, 5.6 Hz), 3.44-3.32 (m, 2H), 3.30 (m, 1H). | 107 |
| 158 | 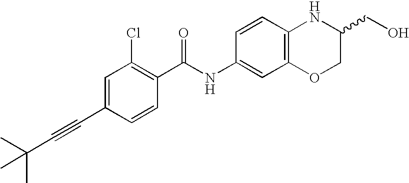 | 399.00 (398.89) | F | (d₆-DMSO) δ 10.09 (s, 1H), 7.49 (d, 1H, J = 1.6 Hz), 7.47 (d, 1H, J = 8.0 Hz), 7.38 (dd, 1H, J = 8.0, 1.6 Hz), 7.09 (d, 1H, J = 2.4 Hz), 6.99 (dd, 1H, J = 8.4, 2.4 Hz), 6.54 (d, 1H, J = 8.4 Hz), 5.68 (s, 1H), 4.86 (t, 1H, J = 5.6 Hz), 4.12 (dd, 1H, J = 10.8, 2.0 Hz), 3.92 (dd, 1H, J = 10.8, 5.6 Hz), 3.44-3.32 (m, 2H), 3.30 (m, 1H), 1.30 (s, 9H). | 107 |
| 159 | 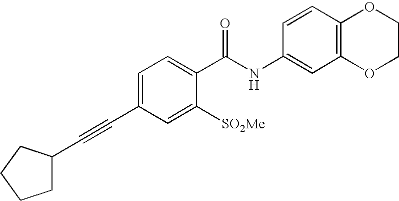 | 426.30 (425.51) | I | (d₆-DMSO) δ 10.5 (1H, s), 7.9 (1H, s), 7.85 (1H, d), 7.75 (1H, d), 7.25 (1H, s), 7.15 (1H, d), 6.85 (1H, d), 4.25 (4H, m), 3.95 (3H, s), 2.85 (1H, m), 2.2-1.95 (2H, m), 1.85-1.5 (6H, m) | 0 |
| 160 | 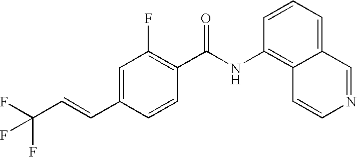 | 361.10 (360.31) | E | (d₆-DMSO) δ 10.61 (s, 1H), 9.37 (s, 1H), 8.57 (d, 1H, J = 6.0 Hz), 8.10-8.00 (m, 2H), 7.94 (4, 1H, J = 5.6 Hz), 7.90-7.81 (m, 2H), 7.76-7.67 (m, 2H), 7.50-7.40 (m, 1H), 7.01 (m, 1H). | 96 |
| 161 | 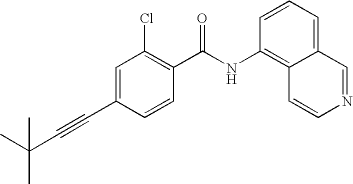 | 363.10 (362.86) | J | (CDCl₃) δ 9.30 (1H, s), 8.60 (1H, dd), 8.52 (1H, s), 8.41 (1H, d), 7.88 (2H, d), 7.75 (1H, d), 7.68 (1H, t), 7.54 (1H, s), 7.43 (1H, d), 1.32 (9H, s). | 95 |
| 162 | 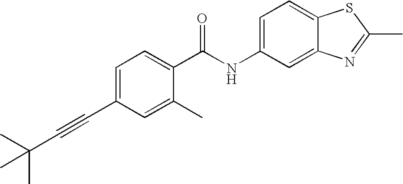 | 363.20 (362.50) | I | | 89 |
| 163 | 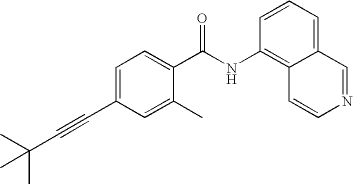 | 343.10 (342.44) | I | (d₆-DMSO) δ 10.5 (1H, s), 9.4 (1H, s), 8.6 (1H, d), 8.15 (2H, m), 7.85 (1H, d), 7.75 (1H, t), 7.35 (1H, d), 7.55 (2H, m), 2.45 (3H, s), 1.8 (9H, s) | 98 |
| 164 | 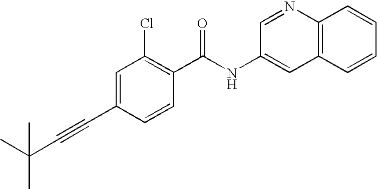 | 362.90 (362.86) | J | (CDCl₃) δ 8.91 (1H, dd), 8.83 (1H, dd), 8.30 (1H, s), 8.08 (1H, d), 7.86 (1H, d), 7.81 (1H, d), 7.69-7.65 (1H, m), 7.59-7.51 (1H, m), 7.42 (1H, dd), 7.41 (1H, dd), 1.30 (9H, s). | 105 |
| 165 | 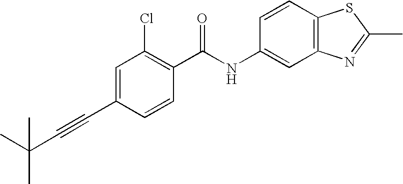 | 383.20 (382.92) | J | (CDCl₃) δ 8.18 (1H, d), 8.09 (1H, s), 7.81-7.72 (3H, m), 7.48 (1H, d), 7.37 (1H, dd), 2.84 (3H, s), 1.31 (9H, s). | 107 |
| 166 | 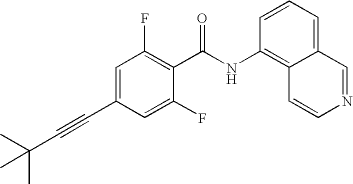 | 365.10 (364.40) | I | (d₆-DMSO) δ 11.0 (1H, s), 9.4 (1H, s), 8.6 (1H, d), 8.15 (2H, m), 7.85 (1H, d), 7.75 (1H, t), 7.55 (2H, m), 1.8 (9H, s) | 99 |
| 167 | 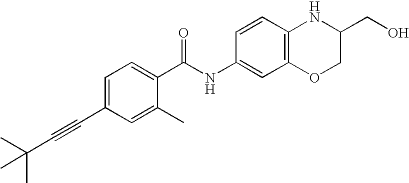 | 379.10 (378.48) | I | (d₆-DMSO) δ 9.8 (1H, s), 7.45-7.25 (3H, m), 7.15 (1H, s), 6.98 (1H, d), 6.52 (1H, d), 5, 75 (1H, s), 4.80 (3H, t), 4.2 (1H, m), 3.85 (1H, m), 3.65 (2H, m), 1.9 (9H, s) | 107 |
| 168 | 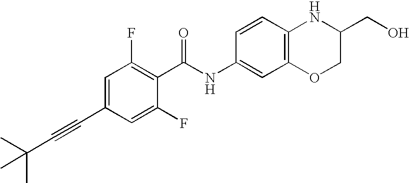 | 400.90 (400.43) | I | (d₆-DMSO) δ 10.4 (1H, s), 7.25 (2H, d), 7.15 (1H, s), 6.91 (1H, d), 6.52 (1H, d), 5.75 (1H, s), 4.80 (1H, t), 4.2 (1H, m), 3.85 (1H, m), 3.65 (2H, m), 1.9 (9H, s) | 104 |
| 169 | 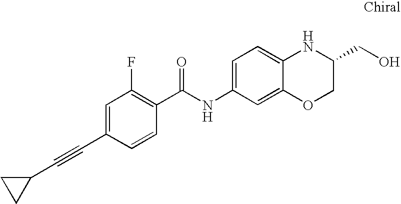 | 367.10 (366.40) | D | (d₆-DMSO) δ 10.00 (s, 1H) 7.55 (t 1H) 7.32 (dd, 1H) 7.27 (dd, 1H) 7.09 (d, 1H) 6.99 (dd, 1H) 6.54 (d, 1H) 5.7 (s, 1H) 4.87 (t, 1H) 4.11 (dd 1H) 3.92 (dd 1H) 3.45-3.27 (m, 2H) 1.63-1.54 (m, 1H) 0.96-0.896 (m, 2H) 0.81-0.75 (m, 2H) | 112 |
| 170 | 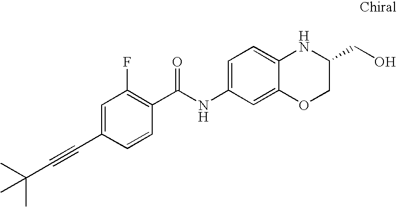 | 383.20 (382.44) | D | (d₆-DMSO) δ 9.99 (1H, s), 7.57 (1H, t), 7.32-7.25 (2H, m), 7.09 (1H, d), 6.99 (1H, dd), 6.54 (1H, d), 5.69 (1H, brs), 4.86 (1H, t), 4.12 (1H, dd), 3.91 (1H, dd), 3.43-3.28 (3H, m), 1.30 (9H, s). | 108 |
| 171 | 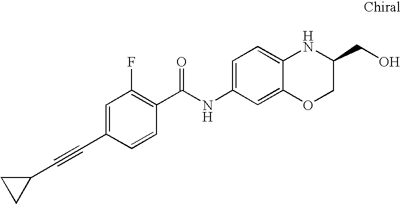 | 367.30 (366.40) | D | (d₆-DMSO) δ 10.00 (s, 1H) 7.55 (t 1H) 7.32 (dd, 1H) 7.27 (dd, 1H) 7.09 (d, 1H) 6.99 (dd, 1H) 6.54 (d, 1H) 5.7 (s, 1H) 4.87 (t, 1H) 4.11 (dd 1H) 3.92 (dd 1H) 3.45-3.27 (m, 2H) 1.63-1.54 (m, 1H) 0.96-0.896 (m, 2H) 0.81-0.75 (m, 2H) | 112 |
| 172 | 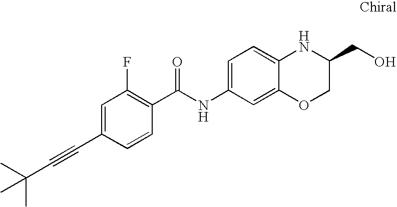 | 383.20 (382.44) | D | (d₆-DMSO) δ 9.99 (1H, s), 7.57 (1H, t), 7.32-7.25 (2H, m), 7.09 (1H, d), 6.99 (1H, dd), 6.54 (1H, d), 5.69 (1H, brs), 4.86 (1H, t), 4.12 (1H, dd), 3.91 (1H, dd), 3.43-3.28 (3H, m), 1.30 (9H, s). | 109 |
| 173 | 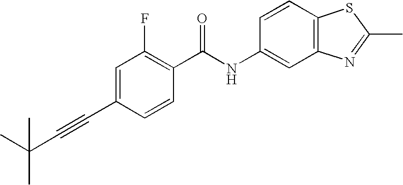 | 367.20 (366.46) | A | | 109 |
| 174 | 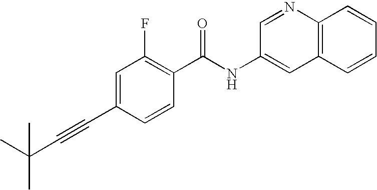 | 347.20 (346.41) | A | | 97 |
| 175 | 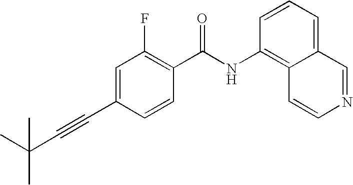 | 347.30 (346.41) | A | | 95 |
| 176 | 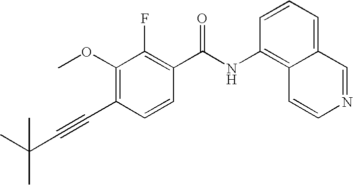 | 376.70 (376.43) | J | (CDCl₃) δ 9.30 (1H, s), 8.91-8.86 (1H, m), 8.63 (1H, d), 8.49 (1H, d), 7.88-7.83 (2H, m), 7.74-7.66 (2H, m), 7.32-7.26 (1H, m), 4.09 (3H, s), 1.38 (9H, s). | 89 |
| 177 | 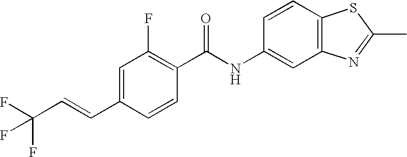 | 380.90 (380.37) | F | (d₆-DMSO) δ 10.67 (s, 1H), 8.40 (d, 1H, J = 2.0 Hz), 7.99 (d, 1H, J = 8.4 Hz), 7.80 (d, 1H, J = 11.6 Hz), 7.76 (d, 1H, J = 7.6 Hz), 7.70-7.65 (m, 2H), 7.47-7.41 (m, 1H), 7.01 (dq, 1H, J = 16.4, 7.2 Hz), 2.80 (s, 3H). | 85 |
| 178 | 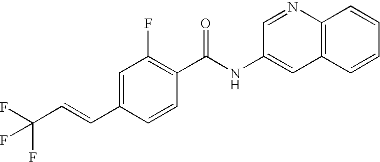 | 361.20 (360.31) | E | (d₆-DMSO) δ 10.97 (s, 1H), 9.04 (d, 1H, J = 2.4 Hz), 8.85 (d, 1H, J = 2.0 Hz), 7.99 (d, 2H, J = 8.4 Hz), 7.83 (m, 2H), 7.69 (m, 2H), 7.61 (t, 1H, J = 8.0 Hz), 7.49-7.42 (m, 1H), 7.03 (dq, 1H, J = 16.4, 7.2 Hz). | 9 |
| 179 | 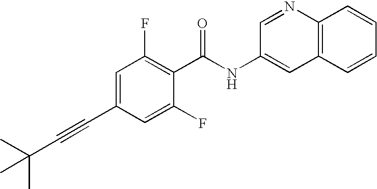 | 365.10 (364.40) | I | (d₆-DMSO) δ 11.5 (1H, s), 9.1 (1H, s), 8.8 (1H, s), 7.9 (2H, d), 7.75-7.50 (2H, m), 7.48-7.35 (2H, m), 1.8 (9H, s) | 100 |
| 180 | 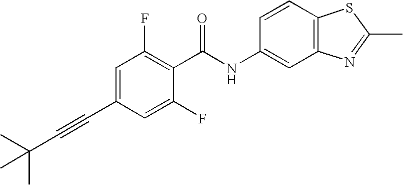 | 384.90 (384.45) | G | (d₆-DMSO) δ 11.0 (1H, s), 8.45 (1H, s), 8.0 (1H, d), 7.65 (1H, d), 7.35 (2H, m), 2.75 (3H, s), 2.8 (9H, s) | 99 |
| 181 | 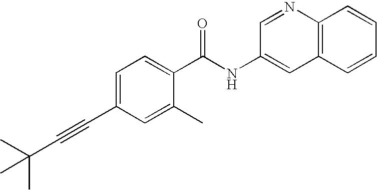 | 343.10 (342.44) | I | (d₆-DMSO) δ 10.8 (1H, s), 9.1 (1H, s), 8.8 (1H, s), 7.9 (2H, d), 7.75-7.50 (3H, m), 7.48-7.35 (2H, m), 2.38 (3H, s), 1.8 (9H, s) | 102 |
| 182 | 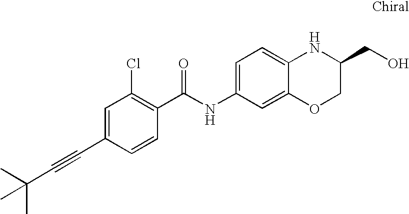 | 399.20 (398.89) | D | (d₆-DMSO) δ 10.09 (s, 1H), 7.49 (d, 1H, J = 1.6 Hz), 7.47 (d, 1H, J = 8.0 Hz), 7.38 (dd, 1H, J = 8.0, 1.6 Hz), 7.09 (d, 1H, J = 2.4 Hz), 6.99 (dd, 1H, J = 8.4, 2.4 Hz), 6.54 (d, 1H, J = 8.4 Hz), 5.68 (s, 1H), 4.86 (t, 1H, J = 5.6 Hz), 4.12 (dd, 1H, J = 10.8, 2.0 Hz), 3.92 (dd, 1H, J = 10.8, 5.6 Hz), 3.44-3.32 (m, 2H), 3.30 (m, 2H), 2.30 (s, 9H). | 103 |
| 183 | 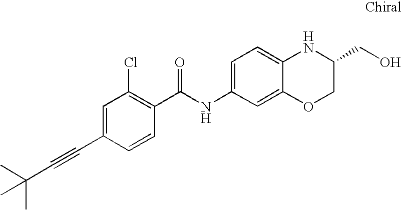 | 399.30 (398.89) | D | (d₆-DMSO) δ 10.09 (s, 1H), 7.49 (d, 1H, J = 1.6 Hz), 7.47 (d, 1H, J = 8.0 Hz), 7.38 (dd, 1H, J = 8.0, 1.6 Hz), 7.09 (d, 1H, J = 2.4 Hz), 6.99 (dd, 1H, J = 8.4, 2.4 Hz), 6.54 (d, 1H, J = 8.4 Hz), 5.68 (s, 1H), 4.86 (t, 1H, J = 5.6 Hz), 4.22 (dd, 1H, J = 10.8, 2.0 Hz), 3.92 (dd, 1H, J = 10.8, 5.6 Hz), 3.44-3.32 (m, 2H), 3.30 (m, 1H), 2.30 (s, 9H). | 98 |
| 184 | 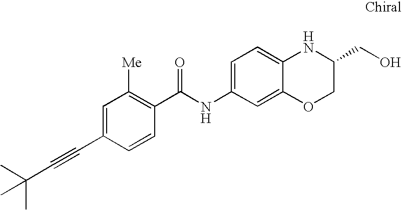 | 379.20 (378.48) | D | (d₆-DMSO) δ 9.8 (1H, s), 7.45-7.25 (3H, m), 7.15 (1H, s), 6.98 (1H, d), 6.52 (1H, d), 5.75 (1H, s), 4.80 (1H, t), 4.2 (1H, m), 3.85 (1H, m), 3.65 (2H, m), 1.9 (9H, s) | 102 |
| 185 | 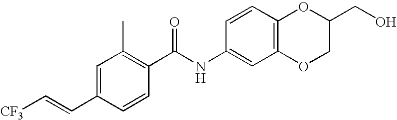 | 394.30 (393.37) | C | (d₆-DMSO) δ 10.18 (1H, s), 7.65 (1H, s), 7.61 (1H, d), 7.47 (1H, d), 7.37 (1H, d), 7.33 (1 H, d), 7.15 (1H, dd), 6.88-6.81 (2H, m), 5.07 (1H, t), 4.34-4.31 (1H, m), 4.15-4.10 (1H, m), 3.68-3.57 (2H, m), 3.41-3.29 (1H, m), 2.36 (3H, s). | 92 |
| 186 | 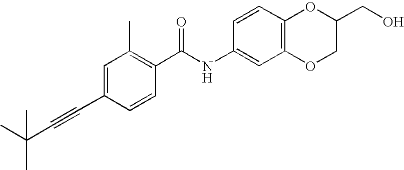 | 380.20 (379.46) | C | (d₆-DMSO) δ 10.15 (1H, s), 7.40-7.36 (2H, m), 7.30 (1H, s), 7.25 (1H, d), 7.15 (1H, dd), 6.83 (1H, d), 5.07 (1H, t), 4.32 (1H, dd), 4.13-4.09 (1H, m), 4.02-3.97 (1H, m), 3.65-3.60 (2H, m), 2.34 (3H, s), 1.30 (9H, s). | 91 |
| 187 | 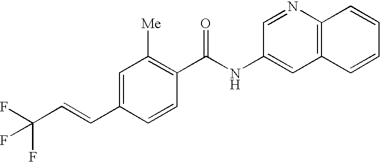 | 357.10 (356.35) | See “Prepn. of amides” Compd. 187 | (d₆-DMSO) δ 10.86 (s, 1H), 9.04 (d, 1H, J = 2.4 Hz), 8.88 (d, 1H, J = 2.4 Hz), 7.98 (d, 2H, J = 8.8 Hz), 7.72-7.58 (m, 5H), 7.43-7.35 (m, 1H), 6.99 (dq, 1H, J = 16.4, 7.2 Hz), 2.46 (s, 3H). | 98 |
| 188 | 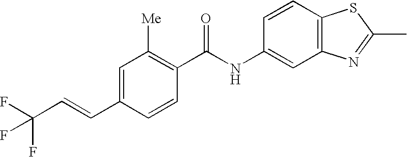 | 377.00 (376.40) | F | (d₆-DMSO) δ 10.56 (s, 1H), 8.42 (d, 1H, J = 2.0 Hz), 7.97 (d, 1H, J = 8.4 Hz), 7.71 (dd, 1H, J = 8.4, 2.0 Hz), 7.68 (s, 1H), 7.65 (d, 1H, J = 8.0 Hz), 7.57 (d, 1H, J = 8.0 Hz), 7.41-7.34 (m, 2H), 6.88 (dq, 1H, J = 16.4, 7.2 Hz), 2.80 (s, 3H), 2.43 (s, 3H). | 83 |
| 189 | 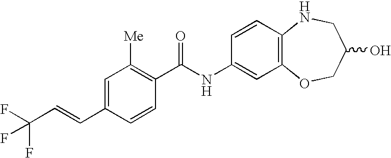 | 393.30 (392.38) | D | (d₆-DMSO) δ 10.03 (s, 1H), 7.64 (s, 1H), 7.60 (d, 1H, J = 8.0 Hz), 7.45 (d, 1H, J = 8.0 Hz), 7.39-7.31 (m, 1H), 7.22 (d, 1H, J = 2.4 Hz), 7.13 (dd, 1H, J = 8.4, 2.4 Hz), 6.84 (dq, 1H, J = 16.4, 7.2 Hz), 6.66 (d, 1H, J = 8.4 Hz), /5.25 (s, 1H), 4.99 (d, 1H, J = 5.2 Hz), 4.19 (dd, 1H, J = 12.0, 3.6 Hz), 3.87 (m, 1H), 3.79 (dd, 1H, J = 12.0, 5.2 Hz), 3.28 (m, 1H), 2.96 (m, 1H), 2.37 (s, 3H). | 87 |
| 190 | 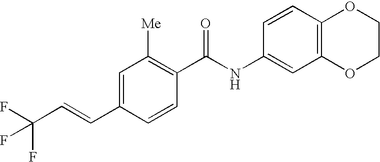 | 364.40 (363.34) | D | (d₆-DMSO) δ 10.18 (s, 1H), 7.65 (s, 1H), 7.61 (d, 1H, J = 8.0 Hz), 7.47 (d, 1H, J = 8.0 Hz), 7.38-7.32 (m, 2H), 7.13 (dd, 1H, J = 8.8, 2.8 Hz), 6.86 (dq, 1H, J = 16.4, 7.2. Hz), 6.81 (d, 1H, J = 8.8 Hz), 4.22 (m, 4H), 2.38 (s, 3H). | 87 |
| 191 | 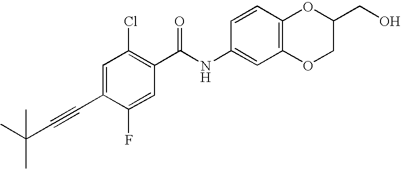 | 418.50 (417.87) | C | (d₆-DMSO) δ 10.40 (1H, s), 7.65 (1H, d), 7.59 (1H, d), 7.32 (1H, d), 7.10 (1H, dd), 6.85 (1H, d), 5.07 (1H, t), 4.33 (1H, dd), 4.14-4.08 (1H, m), 4.02-3.98 (1H, m), 3.65-3.60 (2H, m), 1.31 (9H, s). | 83 |
| 192 | 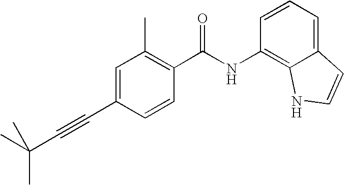 | 329.30 (330.43) | M | (CDCl₃) δ 10.05 (1H, br. s), 7.15 (1H, m), 7.53 (2H, dd), 7.35-7.28 (3H, m), 7.05 (1H, app. t), 6.77 (1H, dd), 6.59 (1H, m), 2.52 (3H, s), 1.34 (9H, s) | |
| 193 | 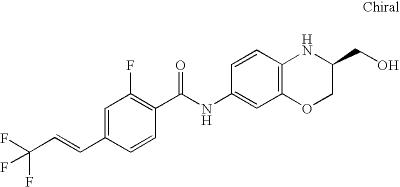 | 397.10 (396.34) | D | (d₆-DMSO) δ 10.04 (s, 1H), 7.74 (d, 1H, J = 11.2 Hz), 7.66 (s, 1H, J = 8.0 Hz), 7.61 (d, 1H, J = 8.0 Hz), 7.41 (m, 1H), 7.11 (d, 1H, J = 2.4 Hz), 7.01 (dd, 1H, J = 8.4, 2.4 Hz), 6.97 (m, 1H), 6.55 (d, 1H, J = 8.4 Hz), 5.69 (s, 1H), 4.86 (t, 1H, J = 5.2 Hz), 4.12 (dd, 1H, J = 10.4, 2.4 Hz), 3.92 (dd, 1H, J = 10.4, 5.6 Hz), 3.45-3.32 (m, 2H), 3.30 (m, 1H). | 103 |
| 194 | 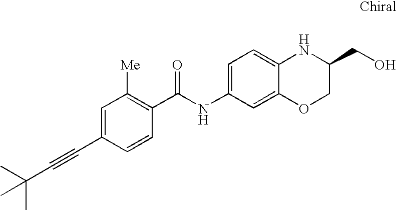 | 379.20 (378.48) | D | (d₆-DMSO) δ 9.8 (1H, s), 7.45-7.25 (3H, m), 7.15 (1H, s), 6.98 (1H, d), 6.52 (1H, d), 5.75 (1H, s), 4.80 (1H, t), 4.2 (1H, m), 3.85 (1H, m), 3.65 (2H, m), 1.9 (9H, s) | 102 |
| 195 | 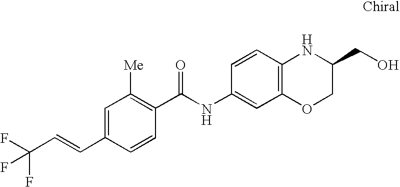 | 393.20 (392.38) | D | (d₆-DMSO) δ 9.93 (s, 1H), 7.63 (s, 1H), 7.59 (d, 1H, J = 8.0 Hz), 7.44 (d, 1H, J = 8.0 Hz), 7.34 (m, 1H), 7.13 (d, 1H, J = 2.4 Hz), 7.02 (dd, 1H, J = 8.4, 2.4 Hz), 6.83 (dq, 1H, J = 16.4, 7.2 Hz), 6.54 (d, 1H, J = 8.4 Hz), 5.64 (s, 1H), 4.86 (t, 1H, J = 5.2 Hz), 4.11 (dd, 1H, J = 10.4, 2.0 Hz), 3.92 (dd, 1H, J = 10.4, 5.6 Hz), 3.44-3.32 (m, 2H), 3.30 (m, 1H), 2.37 (s, 3H). | 60 |
| 196 | 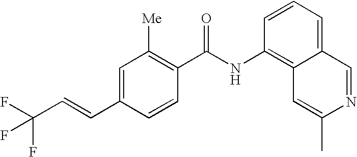 | 371.20 (370.38) | E | (d₆-DMSO) δ 10.44 (s, 1H), 9.26 (s, 1H), 7.98 (m, 2H), 7.79 (s, 1H), 7.76-7.60 (m, 5H), 7.40 (d, 1H, J = 16.0 Hz), 6.89 (m, 1H), 2.64 (s, 3H), 2.50 (s, 3H). | 86 |
| 197 | 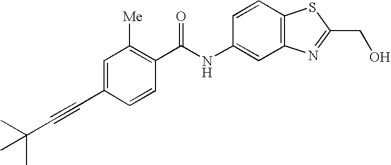 | 379.10 (378.50) | See “Prepn. of amides” Compd. 197 | (d₆-DMSO) δ 10.52 (s, 1H), 8.41 (d, 1H, J = 2.0 Hz), 8.02 (d, 1H J = 8.8 Hz), 7.72 (dd, 1H, J = 8.8, 2.0 Hz), 7.47 (d, 1H, J = 8.4 Hz), 7.33 (s, 1H), 7.29 (d, 1H, J = 8.4 Hz), 6.26 (t, 1H, J = 6.0 Hz), 4.86 (d, 2H, J = 6.0 Hz), 2.38 (s, 3H), 1.31 (s, 9H). | 107 |
| 198 | 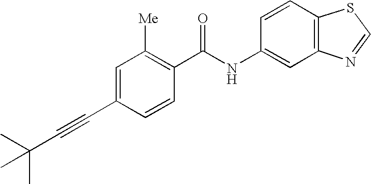 | 349.20 (348.47) | See “Prepn. of amides” Compd. 197 | (CDCl₃); δ 9.03 (s, 1H), 8.36 (s, 1H), 7.94 (d, 1H, J = 8.4 Hz), 7.81 (d, 1H, J = 8.0 Hz), 7.65 (s, 1H), 7.46 (d, 1H, J = 8.0 Hz), 7.32 (s, 1H), 7.29 (d, 1H, J = 8.0 Hz), 2.50 (s, 3H), 1.33 (s, 9H). | 101 |
| 199 | 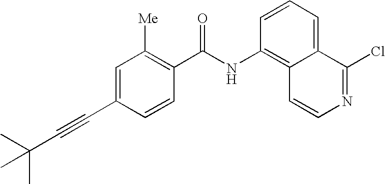 | 377.10 (376.89) | E | (d₆-DMSO) δ 10.62 (s, 1H), 8.36 (d, 1H, J = 6.0 Hz), 8.22 (d, 1H, J = 8.4 Hz), 8.09 (d, 1H, J = 7.2 Hz), 7.99 (d, 1H, J = 6.0 Hz), 7.87 (t, 1H, J = 8.0 Hz), 7.66 (d, 1H, J = 7.6 Hz), 7.35 (s, 1H), 7.32 (d, 1H, J = 8.0 Hz), 2.45 (s, 3H), 1.32 (s, 9H). | 67 |
| 200 | 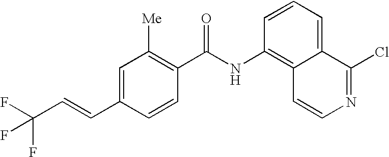 | 391.30 (390.80) | E | (d₆-DMSO) δ 10.65 (s, 1H), 8.41 (d, 1H, J = 6.0 Hz), 8.22 (d, 1H, J = 8.4 Hz), 8.13 (d, 1H, J = 6.8 Hz), 8.01 (d, 1H, J = 6.0 Hz), 7.88 (t, 1H, J = 8.0 Hz), 7.77-7.66 (m, 3H), 7.43-7.36 (m, 1H), 6.89 (dq, 1H, J = 16.0, 6.8 Hz), 2.50 (s, 3H). | 55 |
| 210 | 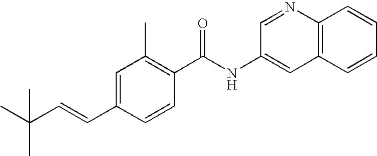 | 345.20 (344.46) | H | | 106 |
| 212 | 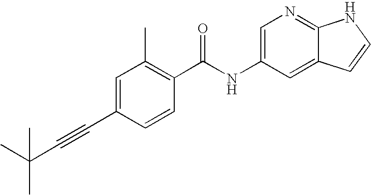 | 330.30 (331.42) | H | (d₆-DMSO) δ 10.95 (1H, br. s), 9.75 (1H, s), 8.82 (2H, m), 7.86-7.78 (2H, m), 7.61-7.55 (2H, m), 6.80 (1H, m). 2.78 (3H, s), 1.64 (9H, s) | 97 |
| 213 | 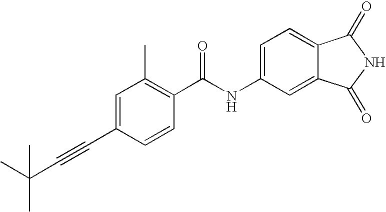 | 361.20 (360.42) | F | | 88 |
| 214 | 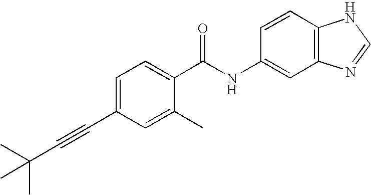 | 332.30 (331.42) | F | | 74 |
| 215 | 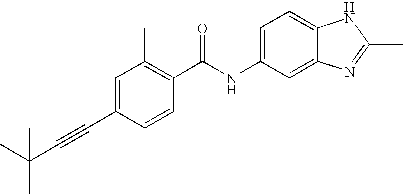 | 346.20 (345.45) | F | | 86 |
| 216 | 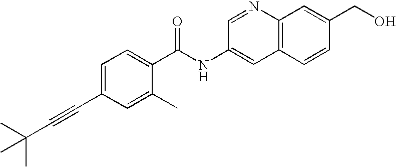 | 373.10 (372.47) | I | (d₄-MeOD) δ 8.88 (1H, d), 8.72 (1H, d), 7.88 (1H, s), 7.82 (1H, d), 7.55 (1H, dd), 7.4 (1H, d), 7.21 (1H, s), 7.2 (1H, d), 5.45 (2H, s), 2.38 (3H, s), 1.25 (9H, s) | 31 |
| 217 | 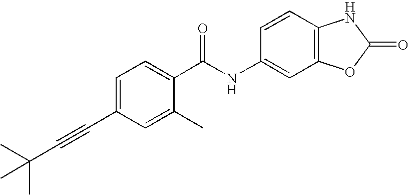 | 348.80 (348.41) | I | (d₆-DMSO) δ 11.5 (1H, s), 10.4 (1H, s), 7.79 (1H, d), 7.42-7.38 (2H, m), 7.31 (1H, s), 7.25 (1H, d), 7.05 (1H, d), 5.45 (2H, s), 2.35 (3H, s), 1.25 (9H, s) | 59 |
| 218 | 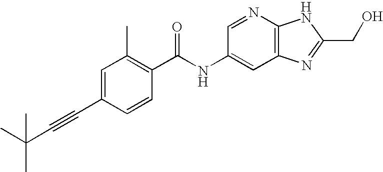 | 363.30 (362.44) | H | (d₆-MeOH) δ 8.48 (1H, s), 8.38 (1H, s), 7.36 (1H, d), 7.20-7.16 (2H, m), 3.28 (2H, m), 2.36 (3H, s), 1.23 (9H, s) | 6 |
| 221 | 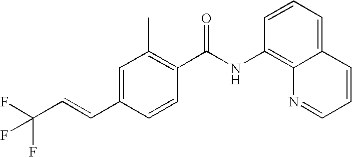 | 358.00 (356.35) | E | (d₆-DMSO) δ 10.27 (s, 1H), 8.92 (dd, 1H, J = 4.4, 1.6 Hz). 8, 72 (d, 1H, J = 7.6 Hz), 8.46 (dd, 1H, J = 8.4, 1.2 Hz), 7.80-7.64 (m, 6H), 7.42-7.37 (m, 1H), 6.95-6.85 (m, 1H), 2.52 (s, 3H). | |
| 222 | 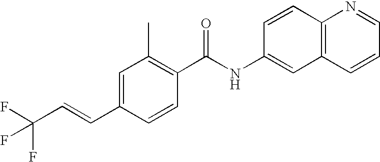 | 357.60 (356.35) | E | (d₆-DMSO) δ 10.71 (s, 1H), 8.81 (dd, 1H, J = 4.4, 1.6 Hz), 8.57 (d, 1H, J = 2.0 Hz), 8.34 (dd, 1H, J = 8.4, 1.2 Hz), 8.00 (d, 1H, J = 9.2 Hz), 7.92 (dd, 1H, J = 9.2, 2.4 Hz), 7.70 (s, 1H), 7.66 (d, 1H, J = 7.6 Hz), 7.60 (d, 1H, J = 8.0 Hz), 7.51 (dd, 1H, J = 8.4, 4.0 Hz), 7.41-7.35 (m, 1H), 6.94-6.84 (m, 1H), 2.44 (s, 3H). | 82 |
| 223 | 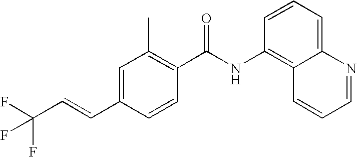 | 357.80 (356.35) | E | (d₆-DMSO) δ 10.55 (s, 1H), 8.94 (dd, 1H, J = 4.0, 1.6 Hz), 8.49 (d, 1H, J = 8.0 Hz), 7.95 (t, 1H, J = 4.8 Hz), 7.85-7.65 (m, 5H), 7.59 (dd, 1H, J = 8.4, 4.0 Hz), 7.42-7.36 (m, 1H), 6.95-6.84 (m, 1H), 2.50 (s, 3H). | |
| 224 | 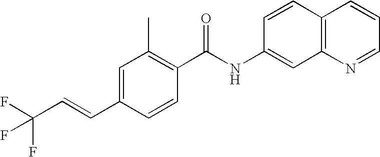 | 357.80 (356.35) | E | (d₆-DMSO) δ 10.72 (s, 1 H), 8.86 (dd, 1H, J = 4.0, 1.6 Hz). 8.57 (s, 1H), 8.30 (dd, 1H, J = 8.4, 1.2 Hz), 7.95 (d, 1H, J = 8.4 Hz), 7.87 (dd, 1H, J = 8.8, 1.6 Hz), 7.70 (s, 1H), 7.67 (d, 1H, J = 8.0 Hz), 7.60 (d, 1H, J = 7.6 Hz), 7.44 (dd, 1H, J = 8.0, 4.0 Hz), 7.42-7.35 (m, 1H), 6.88 (dq, 1H, J = 16.4, 7.2 Hz), 2.45 (s, 3H). | 82 |
| 225 | 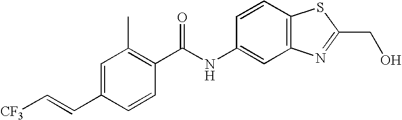 | 392.40 (393.00) | See “Prepn. of amides” Compd. 225 | (d₆-DMSO) δ 10.5 (1H, s), 8.45 (1H, d), 8.05 (1H, d), 7.75-7.62 (3H, m), 7.55 (1H, d), 7.35 (1H, dd), 6.85 (1H, m), 6.25 (1H, t), 4.85 (2H, d), 2.38 (3H, s) | 110 |
| 228 | 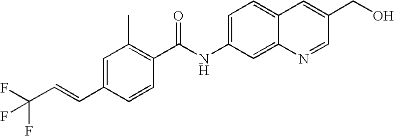 | 387.70 (386.38) | See “Prepn. of amides” Compd. 228 | (d₆-DMSO) δ 10.70 (s, 1H), 8.82 (d, 1H, J = 2.4 Hz). 8.56 (s, 1H), 8.17 (s, 1H), 7.94 (d, 1H, J = 8.8 Hz), 7.86 (dd, 1H, J = 8.8, 2.0 Hz), 7.70 (s, 1H), 7.66 (d, 1H, J = 8.0 Hz), 7.60 (d, 1H, J = 8.0 Hz), 7.42-7.35 (m, 1H), 6.94-6.84 (m, 1H), 5.44 (t, 1H, J = 5.6 Hz), 4.70 (d, 2H, J = 5.6 Hz), 2.45 (s, 3H). | 100 |
| 229 | 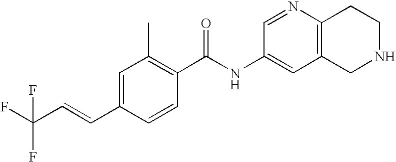 | 361.70 (361.37) | See “Prepn. of amides” Compd. 229 | (d₆-DMSO) δ 10.79 (1H, s), 9.60 (2H, br. s), 8.75 (1H, m), 8.22 (1H, s), 7.70-7.65 (2H, m), 7.59 (1H, d), 7.39 (1H, app. d), 6.90 (1H, m), 4.38 (2H, app. t), 3.48 (2H, m), 3.11 (2H, app. t), 2.43 (3H, s) | 54 |
| 230 | 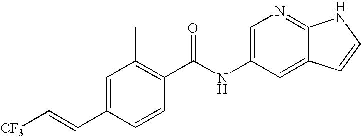 | 346.10 (345.33) | I | (d₄-MeOD) δ 8.45 (2H, dd), 7.65-7.55 (3H, m), 7.48 (1H, d), 7.35 (1H, dd), 6.65 (1H, m), 6.55 (1H, d), 3.85 (3H, s), 2.58 (3H, s) | 99 |
| 231 | 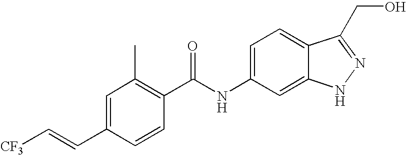 | 376.60 (375.35) | I | (d₄-MeOD) δ 7.75 (1H, d), 7.68 (1H, d), 7.58-7.48 (3H, m), 7.35 (1H, dd), 6.88 (1H, dd), 6.65 (1H, m), 4.78 (2H, s), 2.38 (3H, s) | 0 |
| 232 | 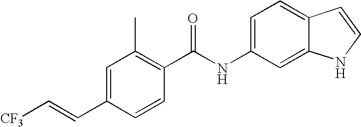 | 345.60 (344.34) | I | (d₄-MeOD) δ 8.05 (1H, d), 7.62-7.55 (4H, m), 7.35 (1H, dd) 7.25 (1H, d), 7.15 (1H, dd), 6.65 (1H, m), 6.49 (1H, d), 2.38 (3H, s) | |
| 233 | 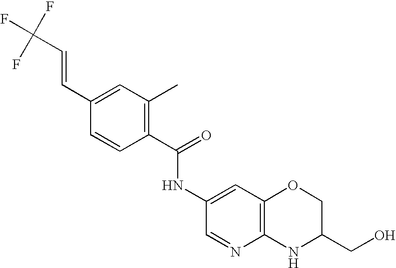 | 393.60 (393.37) | G | (d₆-DMSO) δ 9.93 (s, 1H) 8.59 (d, 1H) 7.95 (s, 1H) 7.62 (dd, 1H) 7.59 (s, 1H) 6.83-6.79 (m, 1H) 6.81 (d, 1H) 6.58 (d, 1H) 5.01 (t, 1H) 4.39-4.07 (m, 2H) 3.65-3.57 (m, 1H) 3.56-3.48 (m, 1H) 3.44-3.36 (m, 1H) 2.32 (s, 3H) | 100 |
| 235 | 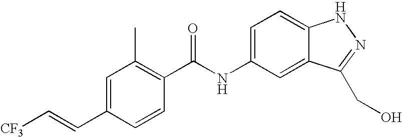 | 377.80 (375.35) | F | (d₄-MeOH) δ 8.45 (1H, s), 7.65-7.45 (5H, m), 7.25 (1H, dd), 6.55 (1H, m), 4.95 (2H, s), 2.45 (3H, s) | |
| 236 | 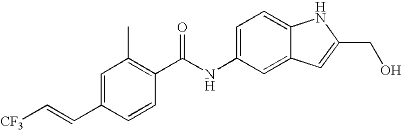 | 375.20 (374.37) | F | (d₆-DMSO) δ 10.95 (1H, s), 10.10 (1H, s), 7.95 (1H, s), 7.65 (1H, s), 7.62 (1H, d), 7.51 (1H, d), 7.35 (1H, dd), 7.29-7.25 (2H, m), 6.85 (1H, m), 6.25 (1H, s), 5.25 (1H, t), 4.55 (2H, d), 2.41 (3H, s) | |
| 237 | 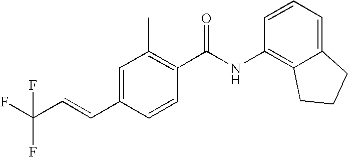 | 345.80 (345.37) | M | (CDCl₃) δ 7.89 (3H, d), 7.53 (1H, d), 7.35-7.30 (2H, m), 7.21 (1H, t), 7.16 (1H, app. t), 7.09 (1H, d), 6.26 (1H, m), 2.98 (2H, t), 2.84 (2H, t), 2.54 (3H, s), 2.13 (2H, quintet) | |
| 238 | 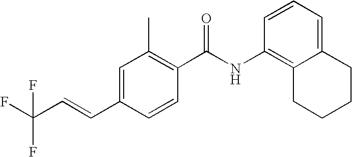 | 360.00 (359.39) | M | (CDCl₃) δ 7.80 (1H, d), 7.53 (1H, d), 7.36-7.12 (4H, m), 6.98 (1H, d), 6.26 (1H, m), 2.80 (2H, t), 2.63 (2H, t), 2.55 (3H, s), 1.89-1.82 (2H, m), 1.82-1.74 (2H, m) | |
| 239 | 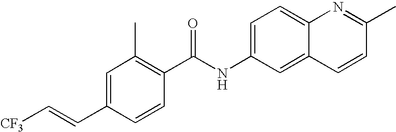 | 370.60 (370.38) | F | (d₆-DMSO) δ 10.55 (1H, s), 8.5 (1H, s), 8.22 (1H, d), 7.90-7.85 (2H, m), 7.69-7.64 (2H, m), 7.59 (1H, d), 7.44-7.35 (2H, m), 6.87 (1H, m), 2.65 (3H, s), 2.41 (3H, s) | |
| 240 | 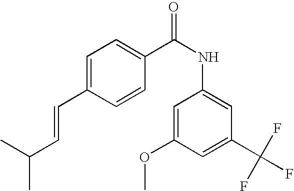 | 364.29 (363.38) | | | |

TABLE 2
AMIDE COMPOUNDS

| | | | Low pH % |
|---|---|---|---|
| | | Mw | Inhib. @ 0.3 |
| ID | Structure | (calcd) | μM |
| 301 | 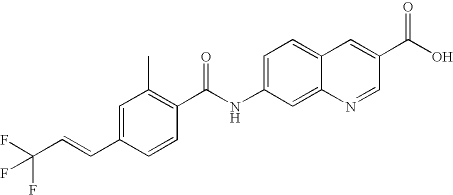 | 400.35 | 43 |
| 302 | 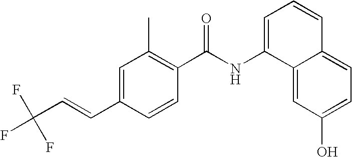 | 371.36 | |
| 303 | 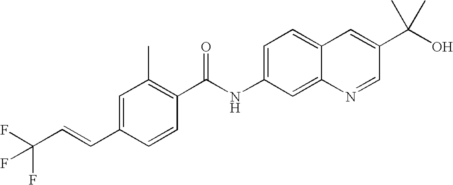 | 414.42 | 90 |
| 304 | 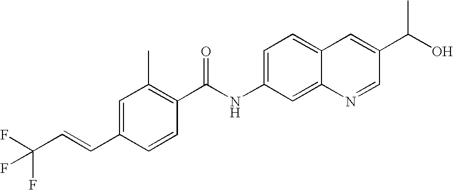 | 400.40 | 98 |
| 305 | 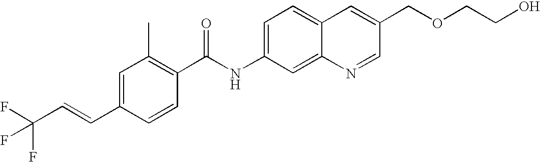 | 430.42 | 105 |
| 306 | 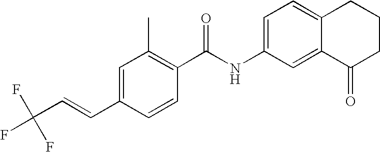 | 373.37 | 51 |
| 307 | 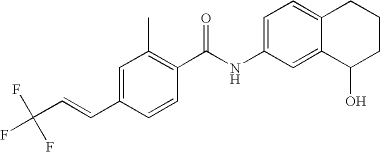 | 375.39 | 75 |
| 308 | 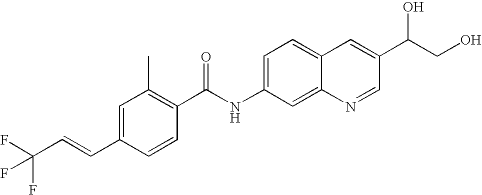 | 416.40 | 101 |
| 309 | 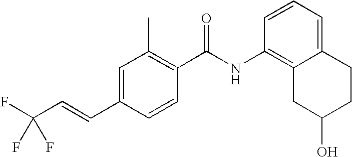 | 375.39 | 107 |
| 310 | 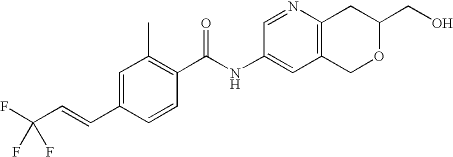 | 392.38 | 71 |
| 311 | 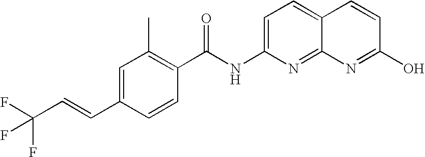 | 373.33 | 20 |
| 312 | 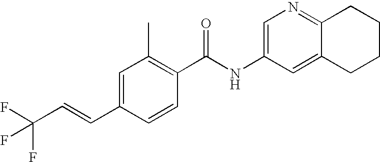 | 360.38 | 97 |
| 313 | 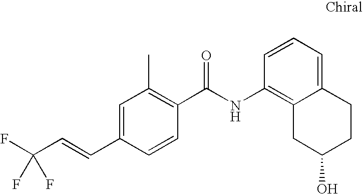 | 375.39 | 105.89 |
| 314 | 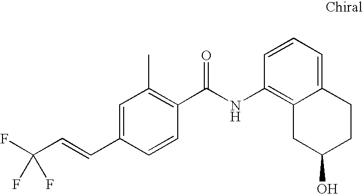 | 375.36 | 121.45 |
| 315 | 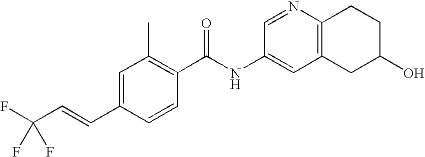 | 376.38 | 51.3 |
| 316 | 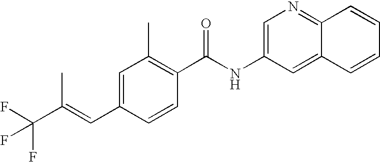 | 370.37 | 52 |
| 317 | 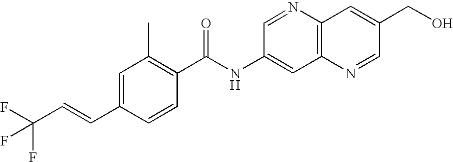 | 387.36 | 100 |
| 318 | 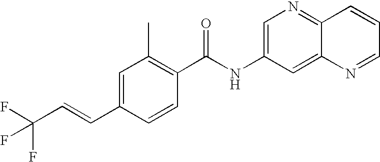 | 357.33 | 104 |
| 319 | 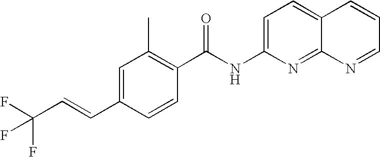 | 357.33 | |
| 320 | 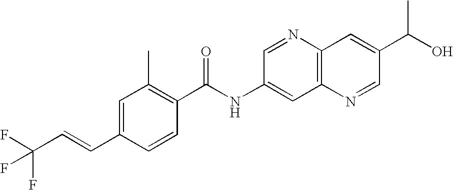 | 401.39 | 47 |
| 321 | 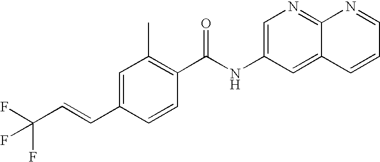 | 357.33 | 41.45 |
| 322 | 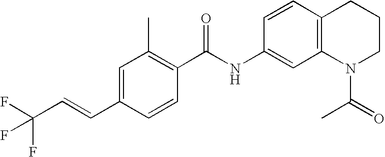 | 402.41 | 42.69 |
| 324 | 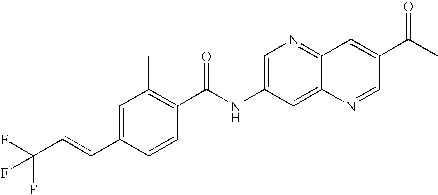 | 399.37 | 0.74 |
| 325 | 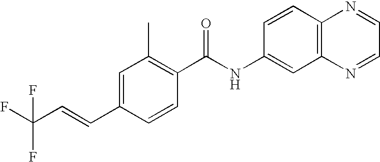 | 357.33 | 72.3 |
| 326 | 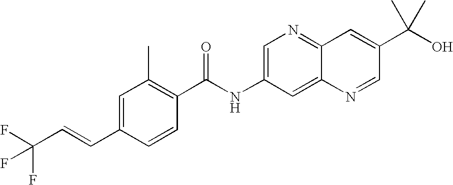 | 415.41 | 6.76 |
| 327 | 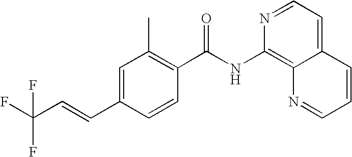 | 357.33 | |

TABLE 3
AMIDE COMPOUNDS

| | | | Low pH % |
|---|---|---|---|
| | | Mw | Inhib. @ 0.3 |
| ID | Structure | (calcd) | μM |
| 401 | 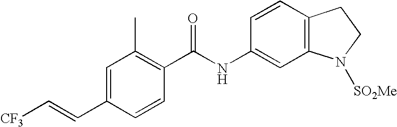 | 424.44 | 88 |
| 402 | 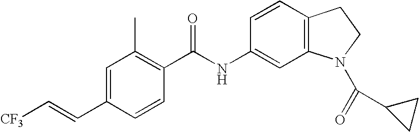 | 414.42 | 0 |
| 403 | 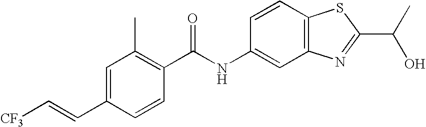 | 406.43 | 81 |
| 404 | 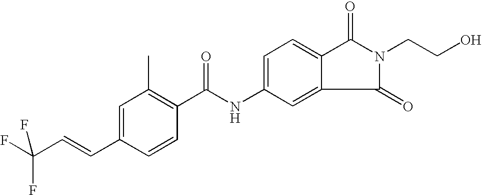 | 418.37 | 10 |
| 405 | 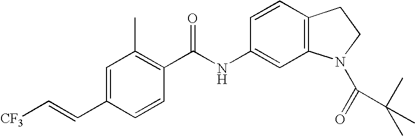 | 430.47 | 14 |
| 406 | 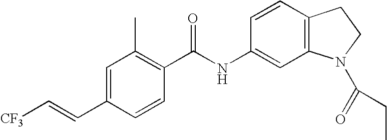 | 402.41 | 45 |
| 407 | 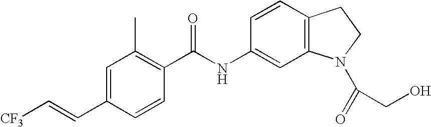 | 404.39 | 95 |
| 408 | 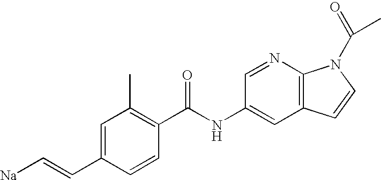 | 387.36 | 91 |
| 409 | 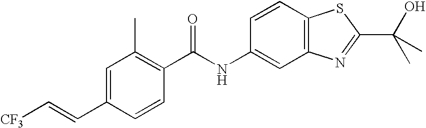 | 420.45 | 103 |
| 410 | 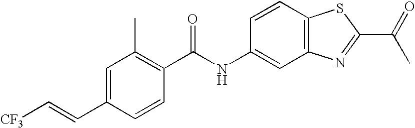 | 404.41 | 12 |
| 411 | 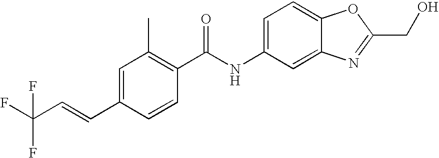 | 376.33 | 102 |
| 412 | 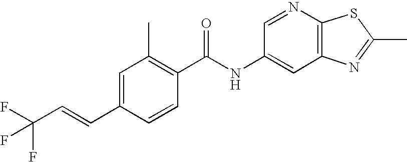 | 377.39 | 105 |
| 413 | 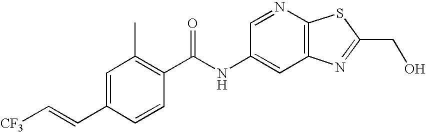 | 393.39 | 110 |
| 414 | 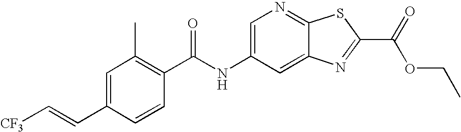 | 435.42 | |
| 415 | 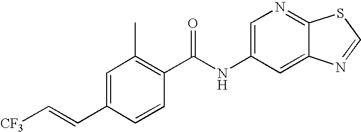 | 363.36 | 130 |

Acid Stimulation Assay:

The Acid-induced changes in the intracellular calcium concentration were monitored using FDSS 6000 (Hamamatsu Photonics, Japan), a fluorometric imaging system. The cell suspension in resting buffer (HBSS supplemented with 10 mM HEPES, pH 7.4) was pre-incubated with varying concentrations of the test compounds or resting buffer (buffer control) for 15 minutes at room temperature under dark conditions. The cells were automatically added the stimulating solution (HBSS supplemented with MES, final assay buffer pH5.8) by the FDSS 6000. The IC₅₀ values of VR1 antagonists were determined from the half of the increase demonstrated by buffer control samples after acidic stimulation, and the results obtained with selected compounds of the invention are set forth in Table 4, below.

TABLE 4
IC₅₀ Data for Selected Amido Compounds

| | IC₅₀ |
|---|---|
| ID | (nM) |
| 118 | 5.00 |
| 127 | 5.00 |
| 138 | 3.00 |
| 166 | 8.00 |
| 167 | 7.00 |
| 170 | 3.00 |
| 172 | 3.00 |
| 176 | 4.00 |
| 187 | 3.00 |
| 193 | 3.00 |
| 194 | 3.00 |
| 195 | 3.00 |
| 197 | 3.00 |
| 198 | 3.00 |
| 210 | 3.00 |
| 214 | 3.00 |
| 225 | 0.90 |
| 233 | 3.00 |
| 302 | 1 |
| 303 | 37 |
| 304 | 4 |
| 305 | 12 |
| 307 | 118 |
| 308 | 11 |
| 309 | 2 |
| 310 | 109 |
| 312 | 103 |
| 313 | 14 |
| 314 | 23 |
| 317 | 11 |
| 318 | 3 |
| 325 | 147 |
| 401 | 94 |
| 402 | 1000 |
| 403 | 147 |
| 404 | 1000 |
| 405 | 1000 |
| 406 | 323 |
| 407 | 32 |
| 408 | 61 |
| 409 | 59 |
| 410 | 707 |
| 411 | 28 |
| 412 | 20 |
| 413 | 4 |
| 415 | 8 |

Half-life in Human Liver Microsomes (HLM)

Test compounds (1 μM) are incubated with 3.3 mM MgCl₂ and 0.78 mg/mL HLM (HL101) in 100 mM potassium phosphate buffer (pH 7.4) at 37° C. on the 96-deep well plate. The reaction mixture is split into two groups, a non-P450 and a P450 group. NADPH is only added to the reaction mixture of the P450 group. An aliquot of samples of P450 group is collected at 0, 10, 30, and 60 min time point, where 0 min time point indicated the time when NADPH is added into the reaction mixture of P450 group. An aliquot of samples of non-P450 group is collected at −10 and 65 min time point. Collected aliquots are extracted with acetonitrile solution containing an internal standard. The precipitated protein is spun down in centrifuge (2000 rpm, 15 min). The compound concentration in supernatant is measured by LC/MS/MS system.

The half-life value is obtained by plotting the natural logarithm of the peak area ratio of compounds/internal standard versus time. The slope of the line of best fit through the points yields the rate of metabolism (k). This is converted to a half-life value using following equations:

Half-life=ln 2/k

The results of the tests and corresponding T_{1/2} values are set forth in Table 3, below.

TABLE 5
T-Half Life In Hours For Exemplary Compounds

| | Half Life |
|---|---|
| ID | (hr) |
| 118 | 1.03 |
| 122 | 1.25 |
| 123 | 1.88 |
| 124 | 1.01 |
| 125 | 0.67 |
| 126 | 1.86 |
| 127 | 1.37 |
| 129 | 1.72 |
| 131 | 1.97 |
| 134 | 1.56 |
| 144 | 1.18 |
| 157 | 1.37 |
| 158 | 1.43 |
| 160 | 0.58 |
| 162 | 1.43 |
| 163 | 1.16 |
| 164 | 2.03 |
| 172 | 1.24 |
| 181 | 1.02 |
| 184 | 0.64 |
| 187 | 9.47 |
| 188 | 1.34 |
| 207 | 6.63 |
| 225 | 3.26 |
| 228 | 1.27 |
| 302 | 0.61 |
| 303 | 0.96 |
| 304 | 0.85 |
| 305 | 0.28 |
| 308 | 1.05 |
| 309 | 2.25 |
| 317 | 0.62 |
| 318 | 2.96 |
| 412 | 0.81 |
| 413 | 3.22 |
| 415 | 4.82 |

Pharmacokinetic Evaluation of Compounds Following Intravenous and Oral Administration in Rats.

Male Sprague-Dawley rats are acclimatized for at least 24 hours prior to experiment initiation. During acclimation period, all animals receive food and water ad libitum. However, food but not water is removed from the animal's cages at least 12 hours before initiation of the experiment. During the first 3 hours of experimentation, the animals receive only water ad libitum. At least three animal each are tested for intravenous and oral dosage. For intravenous formulation, compounds were dissolved (0.25 to 1 mg/mL) in a mixture of 3% dimethyl sulfoxide, 40% PEG 400 and the rest percentage of 40% Captisol in water (w/v). For oral formulation, compounds of this invention are dissolved (2 mg/mL) in a mixture of 5% of 10% Tween 80 in water (v/v) and 95% of 0.5% methyl cellulose in water (w/v). The animals are weighed before dosing. The determined body weight is used to calculate the dose volume for each animal.

Dose volume(mL/kg)=1 mg/kg/formulation concentration(mg/mL)

In instances where the formulation concentrations were less than 0.5 mg/mL, the dosing volume is about 2 mL/kg. PO rats are typically dosed through oral gavage at 2.5 mL/kg to achieve a dose level of 5 mg/kg. For IV dosing, blood samples are collected (using a pre-heparinized syringe) via the jugular vein catheter at 2, 5, 15, 30, 60, 120, 180, 300, 480, and 1440 minutes post dosing. For PO dosing, blood samples are collected (using a pre-heparinized syringe) via the jugular vein catheter before dosing and at 5, 15, 30, 60, 120, 180, 300, 480, and 1440 minutes post dosing. About 250 uL of blood is obtained at each time point from the animal. Equal volumes of 0.9% normal saline are replaced to prevent dehydration. The whole blood samples are maintained on ice until centrifugation. Blood samples are then centrifuged at 14,000 rpm for 10 minutes at 4° C. and the upper plasma layer transferred into a clean vial and stored at −80° C. The resulting plasma samples are then analyzed by liquid chromatography-tandem mass spectrometry. Following the measurement of plasma samples and dosing solutions, plasma concentration-time curve is plotted. Plasma exposure is calculated as the area under the concentration-time curve extrapolated to time infinite (AUC_{inf}). The AUC_{inf} is averaged and the oral bioavailability (% F) for individual animal is calculated as:

AUC_{inf} (PO)/AUC_{inf} (IV), normalized to their respective dose levels. The % F is reported as the mean % F of all animals dosed orally with the compound of the invention at the specified level (Table 6).

In vivo clearance in the rat was calculated by conducting a non-compartmental analysis of the pharmacokinetic profile using WinNonlin software.

TABLE 6
Oral Bioavailability of Exemplary Compounds

| | Oral |
|---|---|
| | Bioavailability |
| ID | F (%) |
| 124 | 67 |
| 125 | 17 |
| 126 | 35 |
| 127 | 66 |
| 129 | 68 |
| 131 | >100 |
| 134 | 63 |
| 138 | 36 |
| 144 | 68 |
| 157 | 83 |
| 158 | 42 |
| 160 | 22 |
| 162 | 2 |
| 163 | 53 |
| 164 | 1 |
| 172 | 76 |
| 181 | 1 |
| 184 | 17 |
| 187 | 43 |
| 188 | 8 |
| 224 | >100 |
| 228 | 39 |
| 230 | 17 |
| 233 | 54 |
| 302 | 2 |
| 303 | >100 |
| 304 | >100 |
| 305 | >100 |
| 308 | 71 |
| 309 | 29 |
| 317 | 47 |
| 318 | 59 |
| 412 | 11 |
| 413 | >100 |
| 415 | 45 |

In vivo clearance in the rat was calculated by conducting a non-compartmental analysis of the pharmacokinetic profile using WinNonlin software.

TABLE 7
In vivo clearance of Exemplary Compounds

| | In vivo Clearance |
|---|---|
| ID | (L/hr/kg) |
| 118 | 1.061 |
| 122 | 3.662 |
| 123 | 1.917 |
| 124 | 1.242 |
| 125 | 2.021 |
| 126 | 2.695 |
| 127 | 1.853 |
| 129 | 0.113 |
| 131 | 1.296 |
| 134 | 5.106 |
| 138 | 2.322 |
| 144 | 1.890 |
| 157 | 0.936 |
| 158 | 0.781 |
| 160 | 5.121 |
| 162 | 2.660 |
| 163 | 6.618 |
| 164 | 2.010 |
| 172 | 2.474 |
| 181 | 2.507 |
| 184 | 2.114 |
| 187 | 0.110 |
| 188 | 0.462 |
| 224 | 0.598 |
| 225 | 0.091 |
| 228 | 2.243 |
| 230 | 0.916 |
| 233 | 2.000 |
| 302 | 4.039 |
| 303 | 0.677 |
| 304 | 1.238 |
| 305 | 0.674 |
| 308 | 0.734 |
| 309 | 0.461 |
| 317 | 0.990 |
| 318 | 0.080 |
| 412 | 0.430 |
| 413 | 0.230 |
| 415 | 0.050 |

### Parallel Artificial Membrane Permeation Assay (PAMPA)

Experiments were performed in 96-well acceptor and donor plates. Such 96-well system was described in Journal of Medicinal Chemistry, 1998, vol. 41, No. 7, 1007-1010.4% phosphatidylcholine and 1% stearic acid in dodecane were used as artificial membrane material. The acceptor plate (96 well hydrophobic filter plate (MAIP N45, Millipore)) was prepared by adding 5 μL of artificial membrane material on the top of the filter and the plate was filled with 250 μL of 2-(N-morpholino)ethanesulfonic acid (MES) buffered Hank's balanced salt solution (HBSS) (pH 6.5). The donor plate (Transport Receiver plate (MATRNPS50, Millipore)) was filled with 300 μL of MES buffered HBSS (pH 6.5) containing 10 μM of the test compounds. The acceptor plate was placed onto the donor plate to form a “sandwich” and was incubated at 30° C. for 2.5 hours. After the incubation period, acceptor, donor and initial donor solution (reference) were analyzed via LC-MS/MS. Data were reported as the effective permeability value in cm×10⁻⁶/sec and the membrane retention value.

### Intrinsic Clearance

Test compounds (1 μM) were incubated with 1 mM MgCl₂, 1 mM NADP+, 5 mM isocitric acid, 1 U/mL isocitric dehydrogenase and 0.8 mg/mL HLM(human liver microsomes) in 100 mM potassium phosphate buffer (pH 7.4) at 37° C. on a number of 384-well plates. At several time points, a plate was removed from the incubator and the reaction was terminated with two incubation volumes of acetonitrile. The compound concentration in supernatant was measured by LC/MS/MS system. The intrinsic clearance value (Clᵢₙₜ) was calculated using following equations:

Clᵢₙₜ(μl/min/mg protein)=(k×incubation volume)/Protein concentration

k(min⁻¹)=−slope of ln(concentration vs. time)

### Example 1

Calcium Imaging Assay

VR1 protein is a heat-gated cation channel that exchanges approximately ten calcium ions for every sodium ion resulting in neuronal membrane depolarization and elevated intracellular calcium levels. Therefore the functional activity of compounds at the VR1 receptor may be determined by measuring changes in intracellular calcium levels in neurons such as the dorsal root ganglion.

DRG neurons were grown on PDL coated 96-well black-walled plates, in the presence of DMEM medium containing 5% Penstrep, 5% Glutamax, 200 μg/ml hygromycin, 5 μg/ml blasticide and 10% heat inactivated FBS. Prior to assay, cells were loaded with 5 μg/ml Fura2 in normal saline solution at 37° C. for 40 minutes. Cells were then washed with normal saline to remove dye before commencement of the experiment.

The plated neurons were transferred into a chamber on the stage of a Nikon eclipse TE300 microscope after which neurons were allowed to attain a stable fluorescence for about 10 minutes before beginning the experiment. The assay consists of two stages, a pretreatment phase followed by a treatment phase. First, a solution of the test compound was added from a multivalve perfusion system to the cells for 1 minute (pretreatment). Immediately following, capsaicin (250 nM) was added in the presence of the test compound (treatment) for a specific period between 20 and 60 seconds.

Fura2 was excited at 340 and 380 nM to indicate relative calcium ion concentration. Changes in wavelength measurements were made throughout the course of the experiment. The fluorescence ratio was calculated by dividing fluorescence measured at 340 nM by that at 380 nM. Data were collected using Intelligent Imaging's Slidebook software. All compounds that inhibited capsaicin induced calcium influx greater than 75% were considered positives.

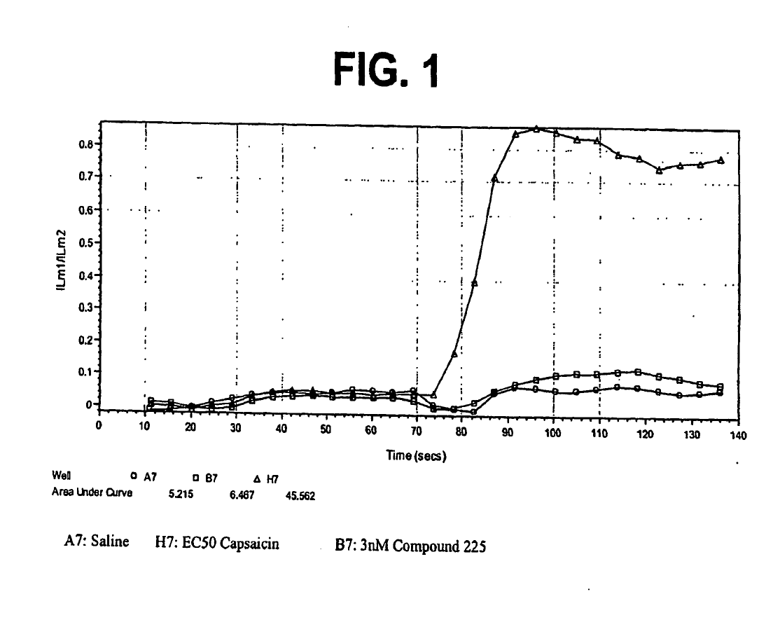

Table 8 provides the data obtained. FIG. 1 demonstrates results obtained when compound 225 is administered with capsaicin. Fluorescence reflecting calcium ion influx is reduced.

TABLE 8

| | | | % inhibition of |
|---|---|---|---|
| | | Treatment time | capsaicin induced |
| Compound ID | Concentration | (sec) | calcium influx |
| 225 | 3 nM | 20 | >75 |

### Example 2

High Throughput Analysis of VR1 Antagonists for Determination of In Vitro Efficacy Using a Calcium Imaging Assay

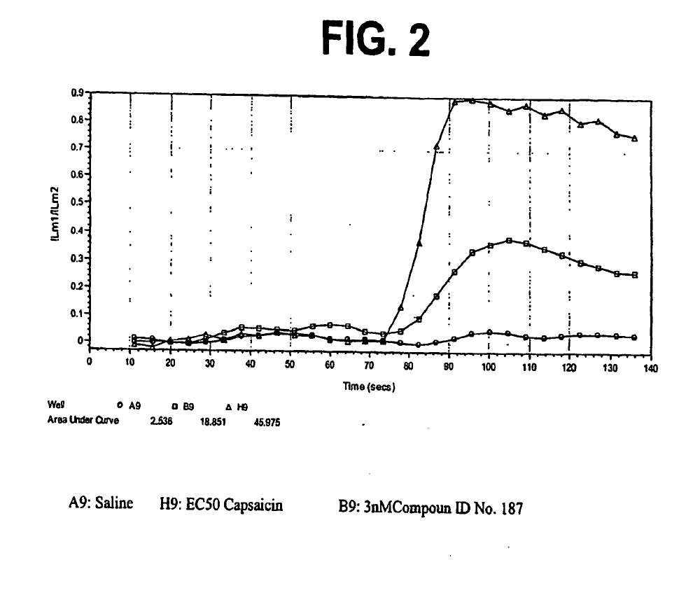

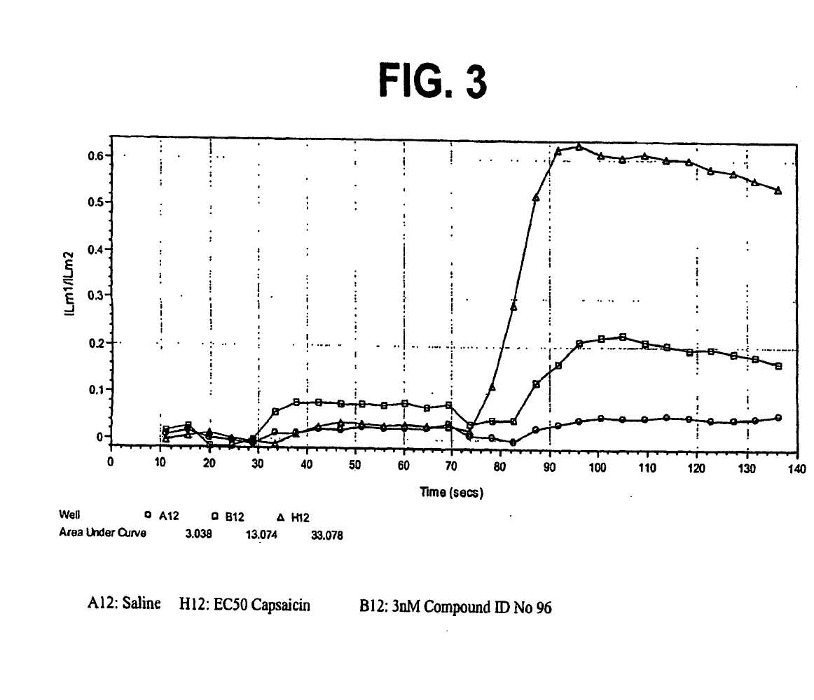

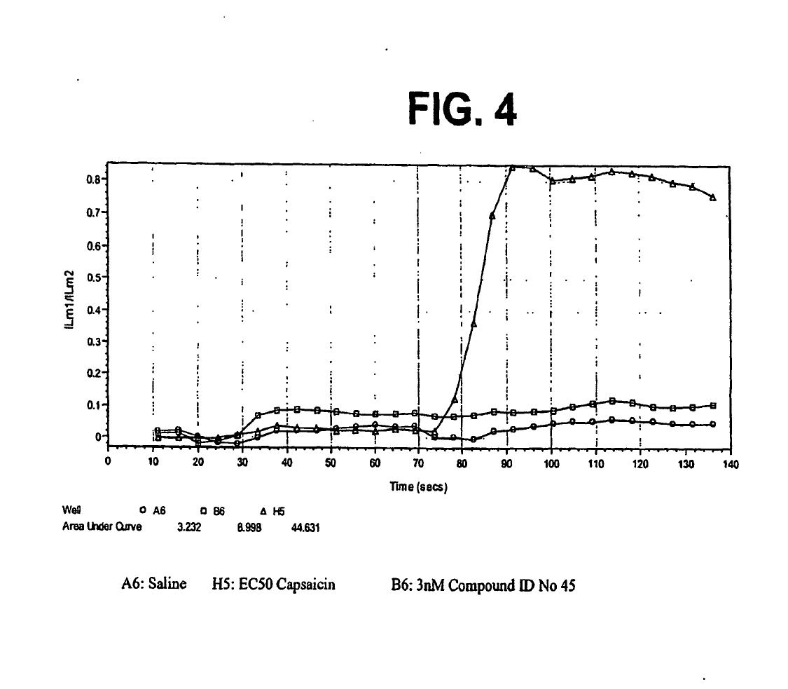

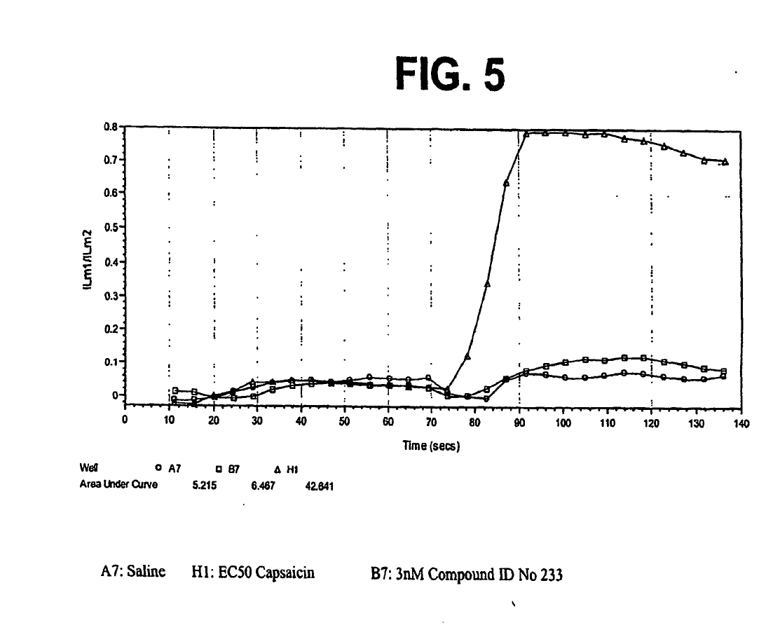

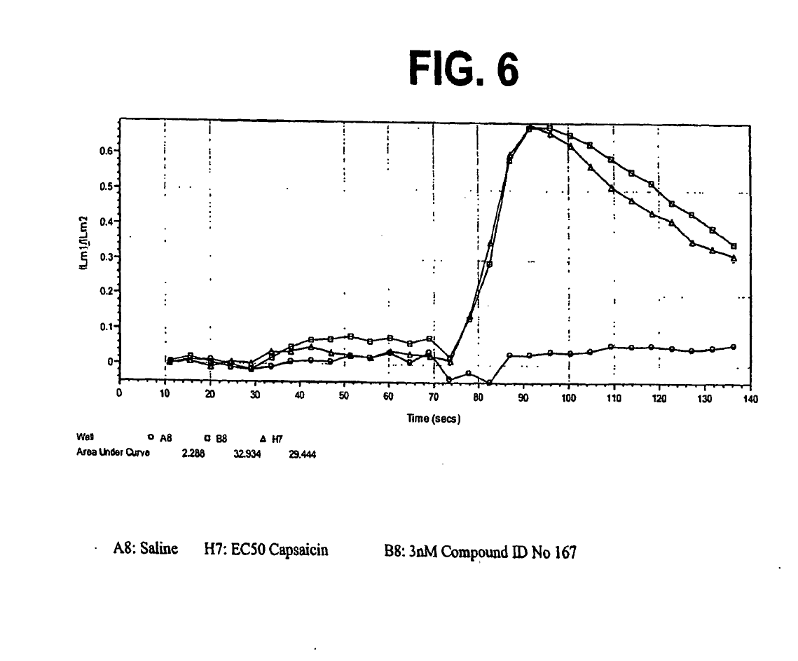

Inhibition of the capsacin response in the presence and absence of the test compound was measured and assessed, using the method for calcium uptake assay, described hereinabove with respect to the data presented in Table 1. Such data is also graphically depicted in FIGS. 2-6, where significant reduction of the capsaicin response is observed in the presence of the representative test compound. No such reduction in response is observed in the absence of the test compound.

### Example 3

Whole-Cell Patch Clamp Electrophysiology

Dorsal root ganglion (DRG) neurons were recovered from either neonatal or adult rats and plated onto poly-D-lysine coated glass coverslips. The plated neurons were transferred into a chamber to allow drug solutions to be added to the cells using a computer-controlled solenoid-valve based perfusion system. The cells were imaged using standard DIC optics. Cells were patched using finely-pulled glass electrodes. Voltage-clamp electrophysiology experiments were carried out using an Axon Instruments Multiclamp amplified controlled by pCLAMP8 software.

The cells were placed into a whole-cell voltage clamp and held at a voltage of −80 mV while monitoring the membrane current in gap-free recording mode. 500 nM capsaicin was added for 30 seconds as a control. Test compounds at various concentrations were added to the cells for 1 minute prior to a 30 second capsaicin application. Differences between control experiments and drug positive capsaicin experiments were used to determine the efficacy of each test compound. All compounds that inhibited capsaicin induced current greater than 50% were considered positives. The data obtained for compound 240 is set forth in Table 9.

TABLE 9

| | | | % inhibition of |
|---|---|---|---|
| | | Treatment time | capsaicin induced |
| Compound ID | Concentration | (seconds) | current |
| 240 | 100 nM | 20 | 50 |

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All such modifications coming within the scope of the appended claims are intended to be included therein.

1.-84. (canceled)

85. A compound according to formula: 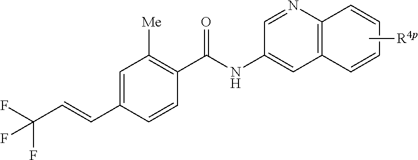
or a pharmaceutically acceptable salt, a solvate, a stereoisomer, an isotopic variant, or a tautomer thereof, and wherein R^{4p} is independently H, C₁-C₆ alkyl, halo, hydroxyl, carbalkoxy [C(O)(C₁-C₆ alkoxy)], acyl [C(O)(C₁-C₆ alkyl)] or hydroxy C₁-C₆ alkyl.

86. A compound according to claim 85 wherein R^{4p} is H.

87. A compound according to formula: 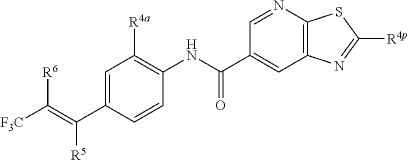
or a pharmaceutically acceptable salt, a solvate, a stereoisomer, an isotopic variant, or a tautomer thereof, wherein:
R^{4a} is C₁-C₆ alkyl, halo C₁-C₆ alkyl, C₁-C₆ alkoxy, sulfone [S(O)₂(C₁-C₆ alkyl)] or halo;
R^{4p} is independently H, C₁-C₆ alkyl, halo, hydroxyl, carbalkoxy [C(O)(C₁-C₆ alkoxy)], acyl [C(O)(C₁-C₆ alkyl)] or hydroxy C₁-C₆ alkyl; and each of R⁵ and R⁶ is independently H, or C₁-C₆ alkyl.

88. A compound according to formula: 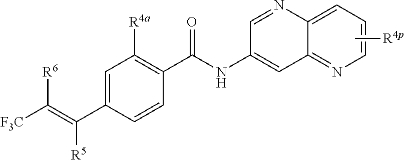
or a pharmaceutically acceptable salt, a solvate, a stereoisomer, an isotopic variant, or a tautomer thereof, wherein:
R^{4a} is C₁-C₆ alkyl, halo C₁-C₆ alkyl, C₁-C₆ alkoxy, sulfone [S(O)₂(C₁-C₆ alkyl)] or halo;
R^{4p} is independently H, C₁-C₆ alkyl, halo, hydroxyl, carbalkoxy [C(O)(C₁-C₆ alkoxy)], acyl [C(O)(C₁-C₆ alkyl)] or hydroxy C₁-C₆ alkyl; and each of R⁵ and R⁶ is independently H, or C₁-C₆ alkyl.

89. A compound according to claim 87 or 88 wherein R^{4a} is Me and each of R⁵ and R⁶ is H.

90. A compound according to claim 87 or 88 wherein R^{4p} is H.

91. A compound according to claim 87 or 88 wherein R^{4p} is CH₂OH.

92.-103. (canceled)

104. A compound selected from the group consisting of: 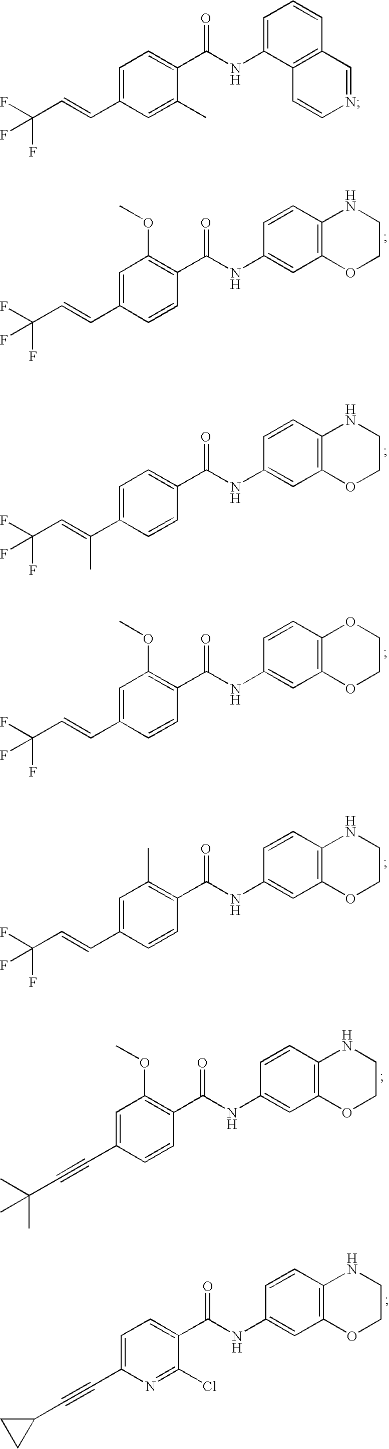 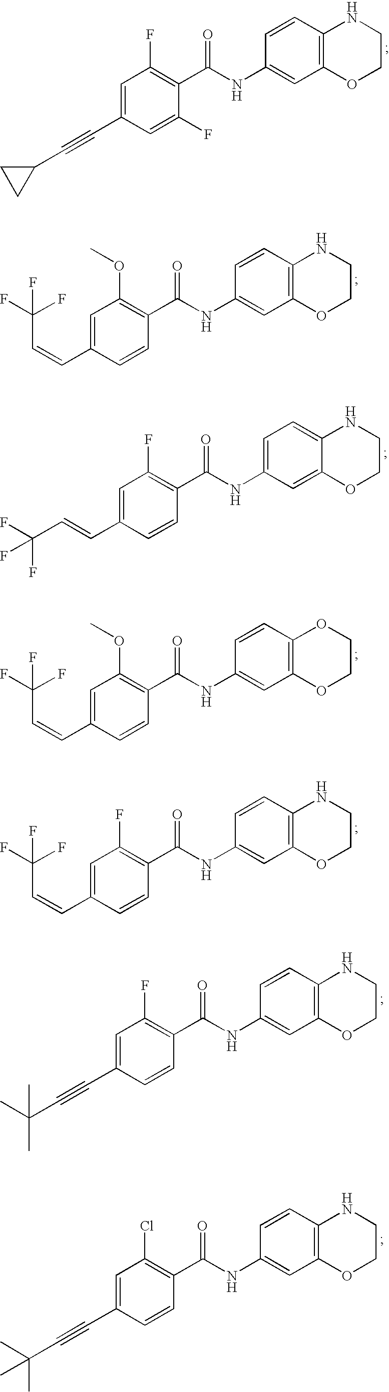 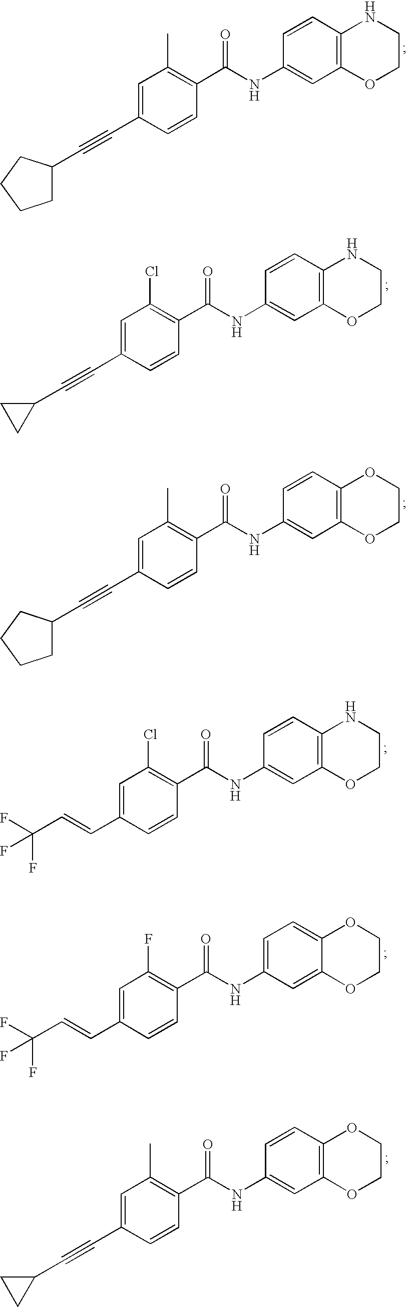 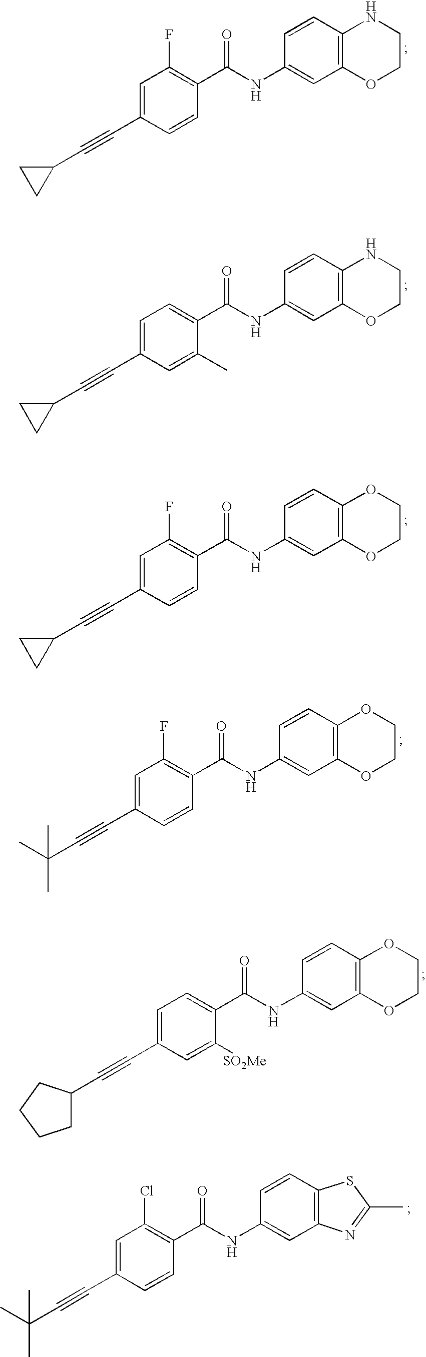 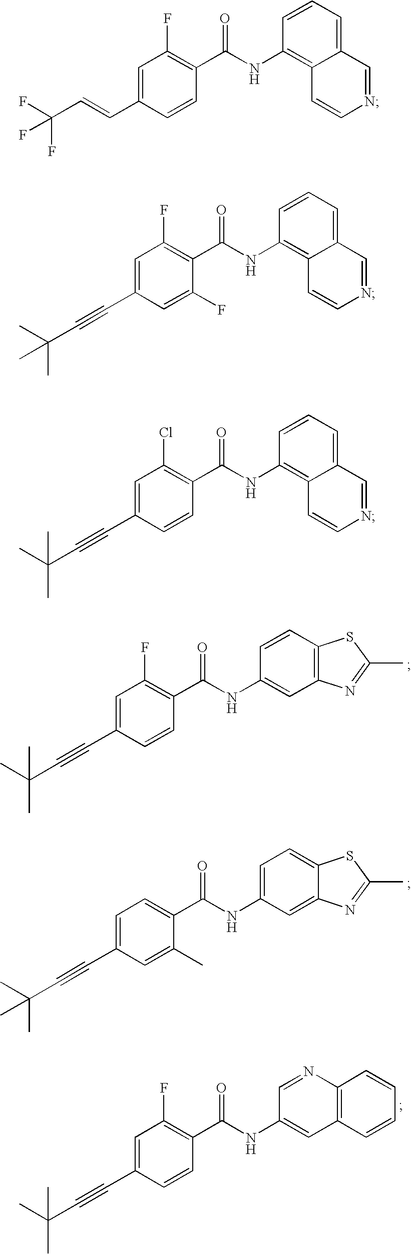 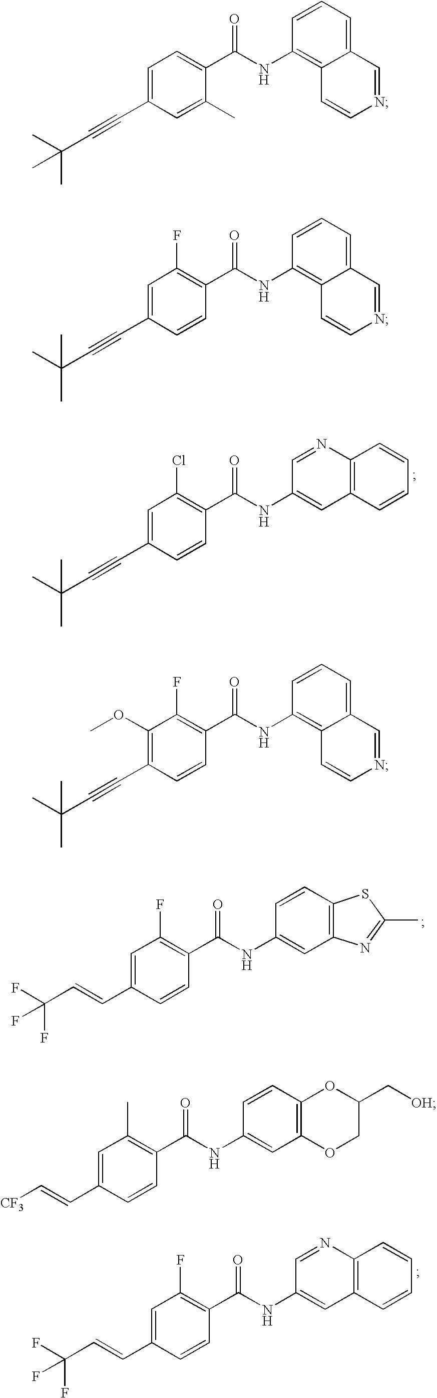 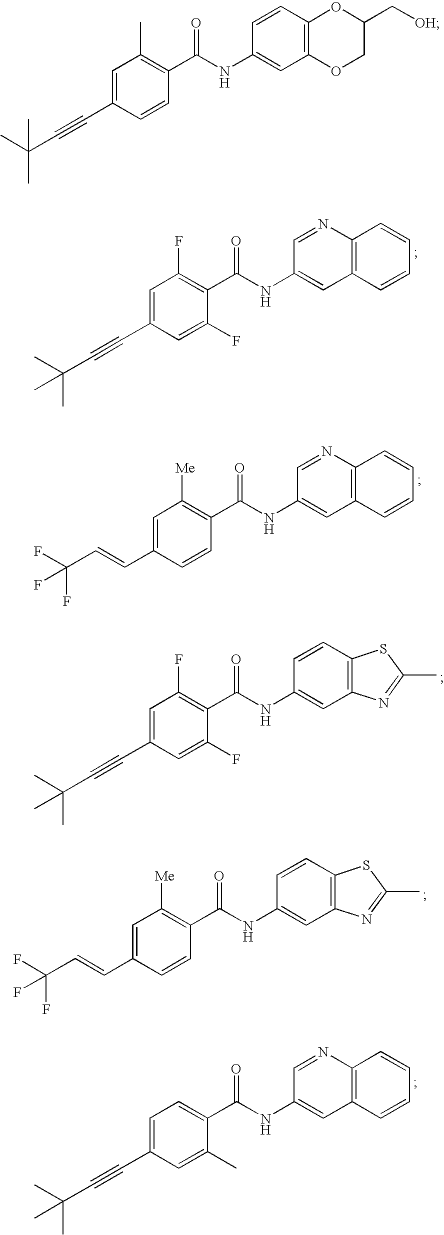 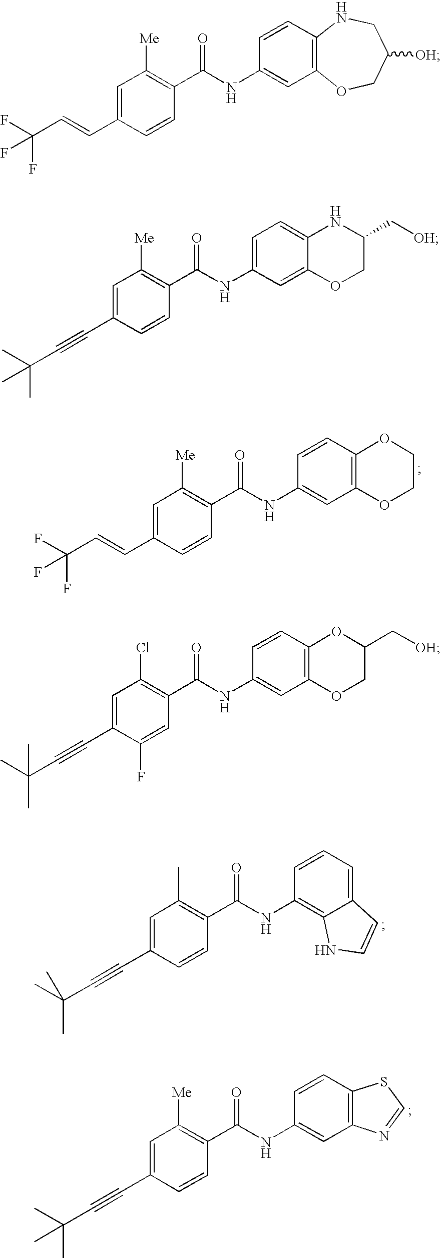 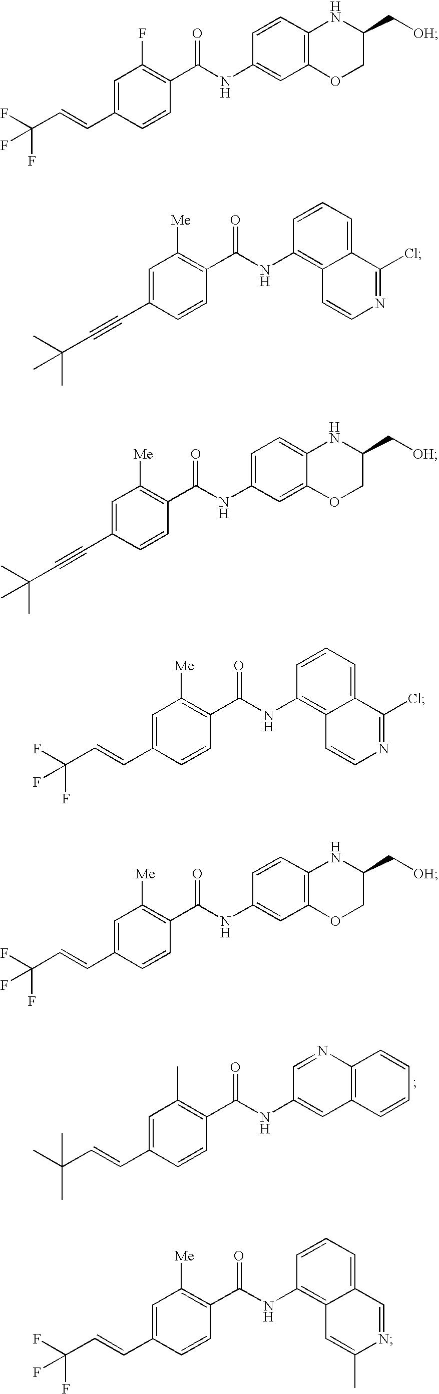 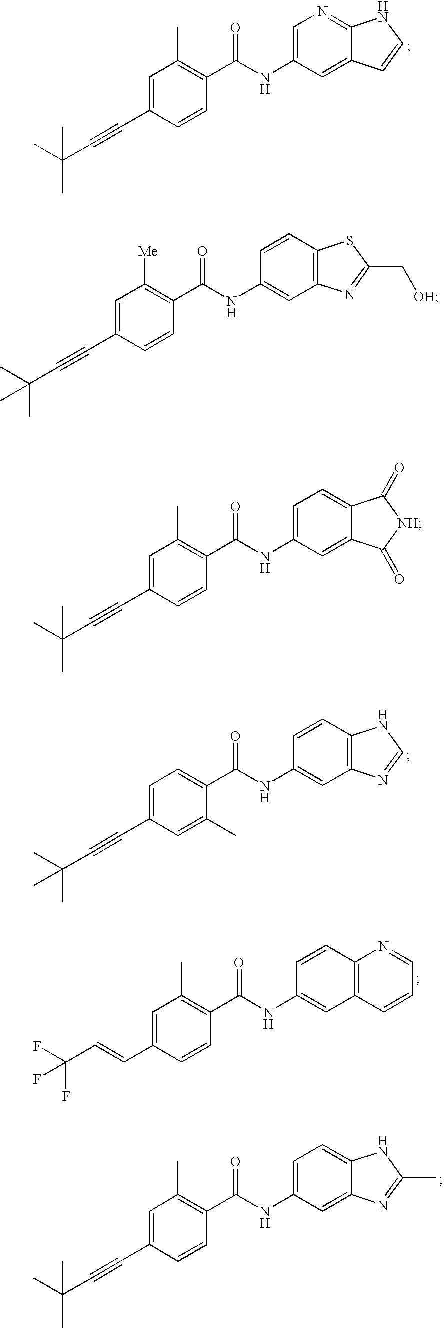 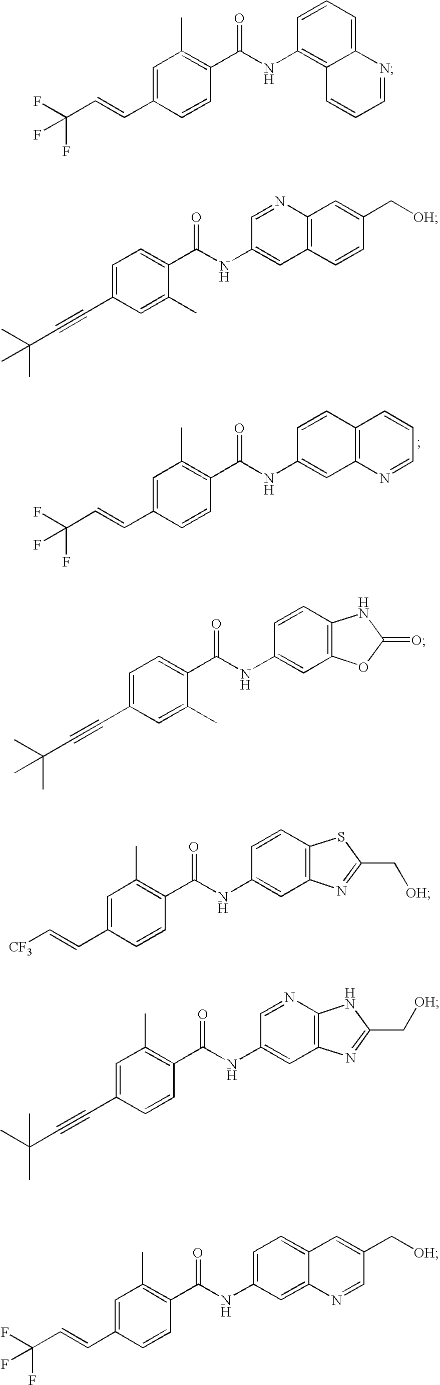 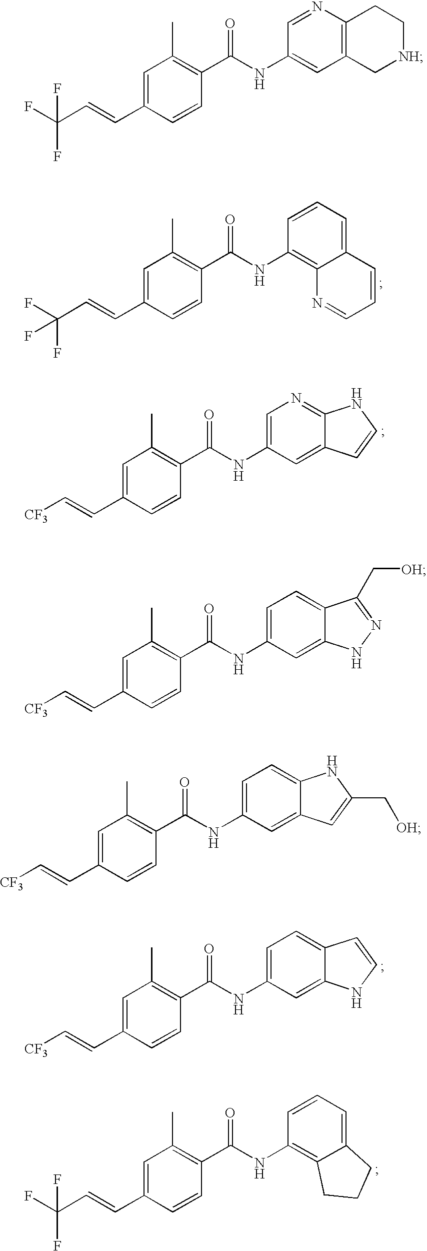 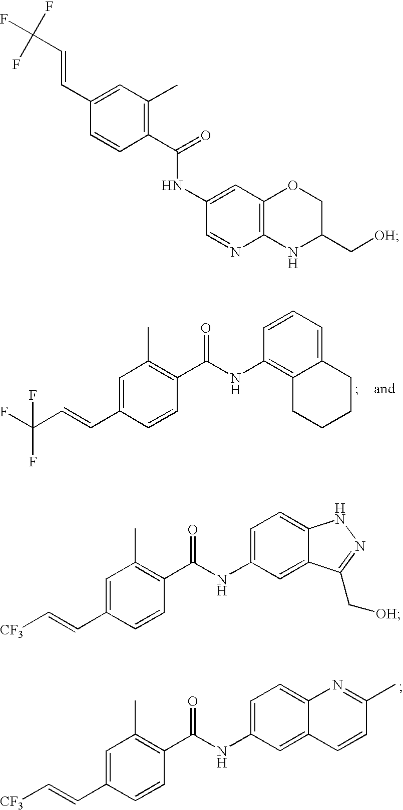
or a pharmaceutically acceptable salt, a solvate, a stereoisomer, an isotopic variant, or a tautomer thereof.

105. A compound selected from the group consisting of:
(E)-N-(isoquinolin-5-yl)-2-methyl-4-(3,3,3-trifluoroprop-1-enyl)benzamide;
(E)-N-(3,4-dihydro-2H-benzo[b][1,4]oxazin-7-yl)-4-(4,4,4-trifluorobut-2-en-2-yl)benzamide;
(E)-N-(3,4-dihydro-2H-benzo[b][1,4]oxazin-7-yl)-2-methyl-4-(3,3,3-trifluoroprop-1-enyl)benzamide;
2-chloro-6-(cyclopropylethynyl)-N-(3,4-dihydro-2H-benzo[b][1,4]oxazin-7-yl)nicotinamide;
(Z)-N-(3,4-dihydro-2H-benzo[b][1,4]oxazin-7-yl)-2-methoxy-4-(3,3,3-trifluoroprop-1-enyl)benzamide;
(Z)-N-(2,3-dihydrobenzo[b][1,4]dioxin-6-yl)-2-methoxy-4-(3,3,3-trifluoroprop-1-enyl)benzamide;
(E)-N-(3,4-dihydro-2H-benzo[b][1,4]oxazin-7-yl)-2-methoxy-4-(3,3,3-trifluoroprop-1-enyl)benzamide;
(E)-N-(2,3-dihydrobenzo[b][1,4]dioxin-6-yl)-2-methoxy-4-(3,3,3-trifluoroprop-1-enyl)benzamide;
N-(3,4-dihydro-2H-benzo[b][1,4]oxazin-7-yl)-4-(3,3-dimethylbut-1-ynyl)-2-methoxybenzamide;
4-(cyclopropylethynyl)-N-(3,4-dihydro-2H-benzo[b][1,4]oxazin-7-yl)-2,6-difluorobenzamide;
(E)-N-(3,4-dihydro-2H-benzo[b][1,4]oxazin-7-yl)-2-fluoro-4-(3,3,3-trifluoroprop-1-enyl)benzamide;
(Z)-N-(3,4-dihydro-2H-benzo[b][1,4]oxazin-7-yl)-2-fluoro-4-(3,3,3-trifluoroprop-1-enyl)benzamide;
N-(3,4-dihydro-2H-benzo[b][1,4]oxazin-7-yl)-4-(3,3-dimethylbut-1-ynyl)-2-fluorobenzamide;
4-(cyclopentylethynyl)-N-(3,4-dihydro-2H-benzo[b][1,4]oxazin-7-yl)-2-methylbenzamide;
4-(cyclopentylethynyl)-N-(2,3-dihydrobenzo[b][1,4]dioxin-6-yl)-2-methylbenzamide;
(E)-N-(2,3-dihydrobenzo[b][1,4]dioxin-6-yl)-2-fluoro-4-(3,3,3-trifluoroprop-1-enyl)benzamide;
4-(cyclopropylethynyl)-N-(3,4-dihydro-2H-benzo[b][1,4]oxazin-7-yl)-2-fluorobenzamide;
4-(cyclopropylethynyl)-N-(2,3-dihydrobenzo[b][1,4]dioxin-6-yl)-2-fluorobenzamide;
2-chloro-N-(3,4-dihydro-2H-benzo[b][1,4]oxazin-7-yl)-4-(3,3-dimethylbut-1-ynyl)benzamide;
2-chloro-4-(cyclopropylethynyl)-N-(3,4-dihydro-2H-benzo[b][1,4]oxazin-7-yl)benzamide;
(E)-2-chloro-N-(3,4-dihydro-2H-benzo[b][1,4]oxazin-7-yl)-4-(3,3,3-trifluoroprop-1-enyl)benzamide;
4-(cyclopropylethynyl)-N-(2,3-dihydrobenzo[b][1,4]dioxin-6-yl)-2-methylbenzamide;
4-(cyclopropylethynyl)-N-(3,4-dihydro-2H-benzo[b][1,4]oxazin-7-yl)-2-methylbenzamide;
N-(2,3-dihydrobenzo[b][1,4]dioxin-6-yl)-4-(3,3-dimethylbut-1-ynyl)-2-fluorobenzamide;
4-(cyclopentylethynyl)-N-(2,3-dihydrobenzo[b][1,4]dioxin-6-yl)-2-(methylsulfonyl)benzamide;
(E)-2-fluoro-N-(isoquinolin-5-yl)-4-(3,3,3-trifluoroprop-1-enyl)benzamide;
2-chloro-4-(3,3-dimethylbut-1-ynyl)-N-(isoquinolin-5-yl)benzamide;
4-(3,3-dimethylbut-1-ynyl)-2-methyl-N-(2-methylbenzo[d]thiazol-5-yl)benzamide;
4-(3,3-dimethylbut-1-ynyl)-N-(isoquinolin-5-yl)-2-methylbenzamide;
2-chloro-4-(3,3-dimethylbut-1-ynyl)-N-(quinolin-3-yl)benzamide;
2-chloro-4-(3,3-dimethylbut-1-ynyl)-N-(2-methylbenzo[d]thiazol-5-yl)benzamide;
4-(3,3-dimethylbut-1-ynyl)-2,6-difluoro-N-(isoquinolin-5-yl)benzamide;
4-(3,3-dimethylbut-1-ynyl)-2-fluoro-N-(2-methylbenzo[d]thiazol-5-yl)benzamide;
4-(3,3-dimethylbut-1-ynyl)-2-fluoro-N-(quinolin-3-yl)benzamide;
4-(3,3-dimethylbut-1-ynyl)-2-fluoro-N-(isoquinolin-5-yl)benzamide;
4-(3,3-dimethylbut-1-ynyl)-2-fluoro-N-(isoquinolin-5-yl)-3-methoxybenzamide;
(E)-2-fluoro-N-(2-methylbenzo[d]thiazol-5-yl)-4-(3,3,3-trifluoroprop-1-enyl)benzamide;
(E)-2-fluoro-N-(quinolin-3-yl)-4-(3,3,3-trifluoroprop-1-enyl)benzamide;
4-(3,3-dimethylbut-1-ynyl)-2,6-difluoro-N-(quinolin-3-yl)benzamide;
4-(3,3-dimethylbut-1-ynyl)-2,6-difluoro-N-(2-methylbenzo[d]thiazol-5-yl)benzamide;
4-(3,3-dimethylbut-1-ynyl)-2-methyl-N-(quinolin-3-yl)benzamide;
(R)-4-(3,3-dimethylbut-1-ynyl)-N-(3-(hydroxymethyl)-3,4-dihydro-2H-benzo[b][1,4]oxazin-7-yl)-2-methylbenzamide;
(E)-N-(2-(hydroxymethyl)-2,3-dihydrobenzo[b][1,4]dioxin-6-yl)-2-methyl-4-(3,3,3-trifluoroprop-1-enyl)benzamide;
4-(3,3-dimethylbut-1-ynyl)-N-(2-(hydroxymethyl)-2,3-dihydrobenzo[b][1,4]dioxin-6-yl)-2-methylbenzamide;
(E)-2-methyl-N-(quinolin-3-yl)-4-(3,3,3-trifluoroprop-1-enyl)benzamide;
(E)-2-methyl-N-(2-methylbenzo[d]thiazol-5-yl)-4-(3,3,3-trifluoroprop-1-enyl)benzamide;
(E)-N-(3-hydroxy-2,3,4,5-tetrahydrobenzo[b][1,4]oxazepin-8-yl)-2-methyl-4-(3,3,3-trifluoroprop-1-enyl)benzamide;
(E)-N-(2,3-dihydrobenzo[b][1,4]dioxin-6-yl)-2-methyl-4-(3,3,3-trifluoroprop-1-enyl)benzamide;
2-chloro-4-(3,3-dimethylbut-1-ynyl)-5-fluoro-N-(2-(hydroxymethyl)-2,3-dihydrobenzo[b][1,4]dioxin-6-yl)benzamide;
4-(3,3-dimethylbut-1-ynyl)-N-(1H-indol-7-yl)-2-methylbenzamide;
(S,E)-2-fluoro-N-(3-(hydroxymethyl)-3,4-dihydro-2H-benzo[b][1,4]oxazin-7-yl)-4-(3,3,3-trifluoroprop-1-enyl)benzamide;
(S)-4-(3,3-dimethylbut-1-ynyl)-N-(3-(hydroxymethyl)-3,4-dihydro-2H-benzo[b][1,4]oxazin-7-yl)-2-methylbenzamide;
(S,E)-N-(3-(hydroxymethyl)-3,4-dihydro-2H-benzo[b][1,4]oxazin-7-yl)-2-methyl-4-(3,3,3-trifluoroprop-1-enyl)benzamide;
(E)-2-methyl-N-(3-methylisoquinolin-5-yl)-4-(3,3,3-trifluoroprop-1-enyl)benzamide;
4-(3,3-dimethylbut-1-ynyl)-N-(2-(hydroxymethyl)benzo[d]thiazol-5-yl)-2-methylbenzamide;
N-(benzo[d]thiazol-5-yl)-4-(3,3-dimethylbut-1-ynyl)-2-methylbenzamide;
N-(1-chloroisoquinolin-5-yl)-4-(3,3-dimethylbut-1-ynyl)-2-methylbenzamide;
(E)-N-(1-chloroisoquinolin-5-yl)-2-methyl-4-(3,3,3-trifluoroprop-1-enyl)benzamide;
(E)-4-(3,3-dimethylbut-1-enyl)-2-methyl-N-(quinolin-3-yl)benzamide;
4-(3,3-dimethylbut-1-ynyl)-2-methyl-N-(1H-pyrrolo[2,3-b]pyridin-5-yl)benzamide;
4-(3,3-dimethylbut-1-ynyl)-N-(1,3-dioxoisoindolin-5-yl)-2-methylbenzamide;
N-(1H-benzo[d]imidazol-5-yl)-4-(3,3-dimethylbut-1-ynyl)-2-methylbenzamide;
4-(3,3-dimethylbut-1-ynyl)-2-methyl-N-(2-methyl-1H-benzo[d]imidazol-5-yl)benzamide;
4-(3,3-dimethylbut-1-ynyl)-N-(7-(hydroxymethyl)quinolin-3-yl)-2-methylbenzamide;
4-(3,3-dimethylbut-1-ynyl)-2-methyl-N-(2-oxo-2,3-dihydrobenzo[d]oxazol-6-yl)benzamide;
4-(3,3-dimethylbut-1-ynyl)-N-(2-(hydroxymethyl)-3H-imidazo[4,5-b]pyridin-6-yl)-2-methylbenzamide;
(E)-2-methyl-N-(quinolin-8-yl)-4-(3,3,3-trifluoroprop-1-enyl)benzamide;
(E)-2-methyl-N-(quinolin-6-yl)-4-(3,3,3-trifluoroprop-1-enyl)benzamide;
(E)-2-methyl-N-(quinolin-5-yl)-4-(3,3,3-trifluoroprop-1-enyl)benzamide;
(E)-2-methyl-N-(quinolin-7-yl)-4-(3,3,3-trifluoroprop-1-enyl)benzamide;
(E)-N-(2-(hydroxymethyl)benzo[d]thiazol-5-yl)-2-methyl-4-(3,3,3-trifluoroprop-1-enyl)benzamide;
(E)-N-(3-(hydroxymethyl)quinolin-7-yl)-2-methyl-4-(3,3,3-trifluoroprop-1-enyl)benzamide;
(E)-2-methyl-N-(5,6,7,8-tetrahydro-1,6-naphthyridin-3-yl)-4-(3,3,3-trifluoroprop-1-enyl)benzamide;
(E)-2-methyl-N-(1H-pyrrolo[2,3-b]pyridin-5-yl)-4-(3,3,3-trifluoroprop-1-enyl)benzamide;
(E)-N-(3-(hydroxymethyl)-1H-indazol-6-yl)-2-methyl-4-(3,3,3-trifluoroprop-1-enyl)benzamide;
(E)-N-(1H-indol-6-yl)-2-methyl-4-(3,3,3-trifluoroprop-1-enyl)benzamide;
(E)-N-(3-(hydroxymethyl)-3,4-dihydro-2H-pyrido[3,2-b][1,4]oxazin-7-yl)-2-methyl-4-(3,3,3-trifluoroprop-1-enyl)benzamide;
(E)-N-(3-(hydroxymethyl)-1H-indazol-5-yl)-2-methyl-4-(3,3,3-trifluoroprop-1-enyl)benzamide;
(E)-N-(2-(hydroxymethyl)-1H-indol-5-yl)-2-methyl-4-(3,3,3-trifluoroprop-1-enyl)benzamide;
(E)-N-(2,3-dihydro-1H-inden-4-yl)-2-methyl-4-(3,3,3-trifluoroprop-1-enyl)benzamide;
(E)-2-methyl-N-(5,6,7,8-tetrahydronaphthylen-1-yl)-4-(3,3,3-trifluoroprop-1-enyl)benzamide; and
(E)-2-methyl-N-(2-methylquinolin-6-yl)-4-(3,3,3-trifluoroprop-1-enyl)benzamide;
or a pharmaceutically acceptable salt, a solvate, a stereoisomer, an isotopic variant, or a tautomer thereof.

106. A compound selected from the group consisting of: 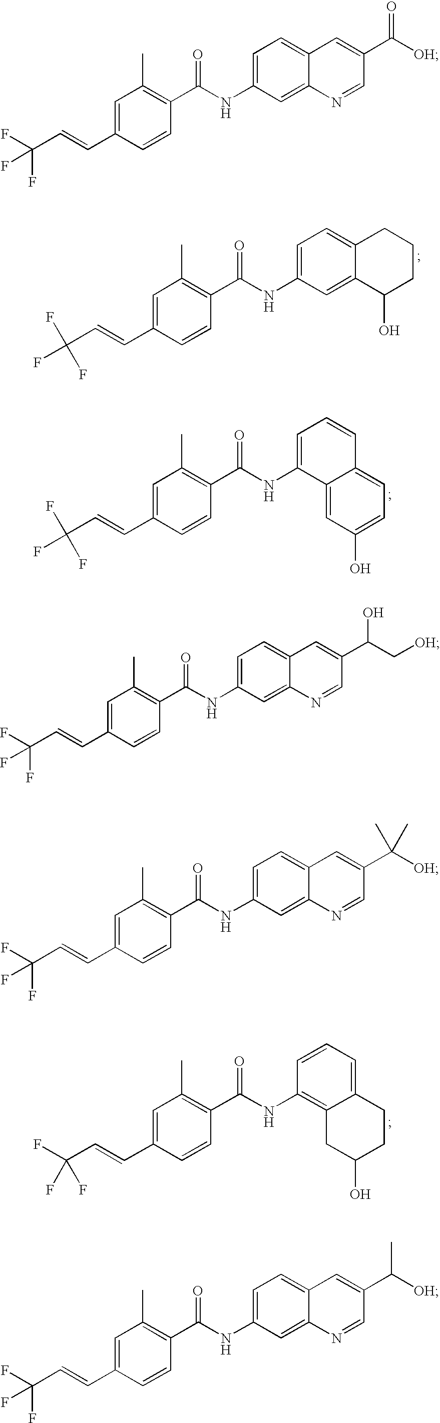 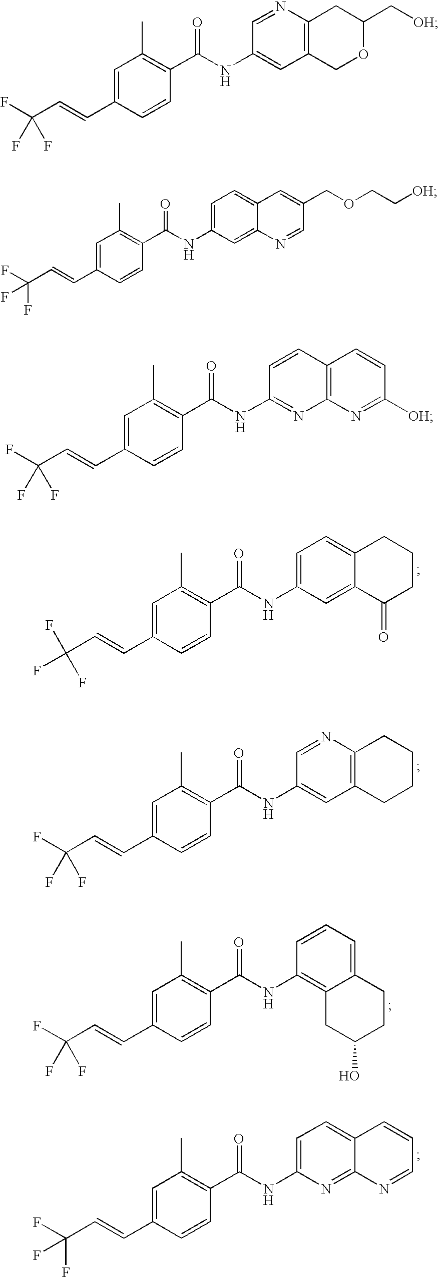 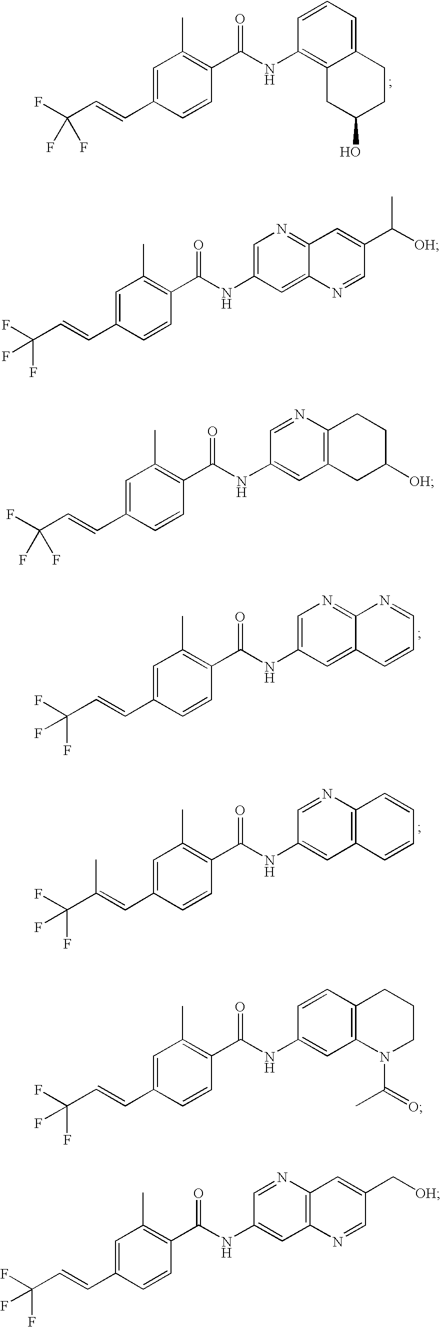 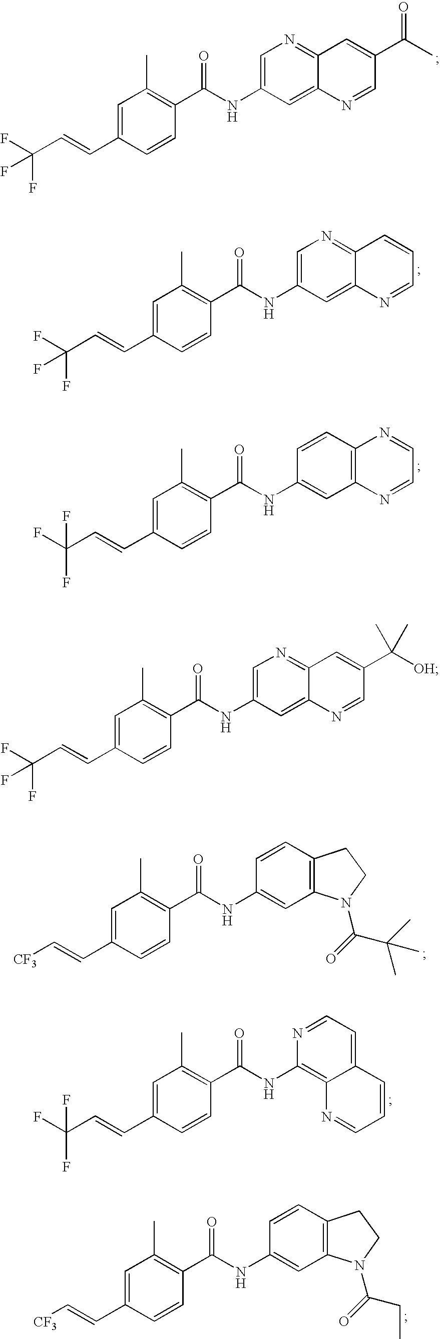 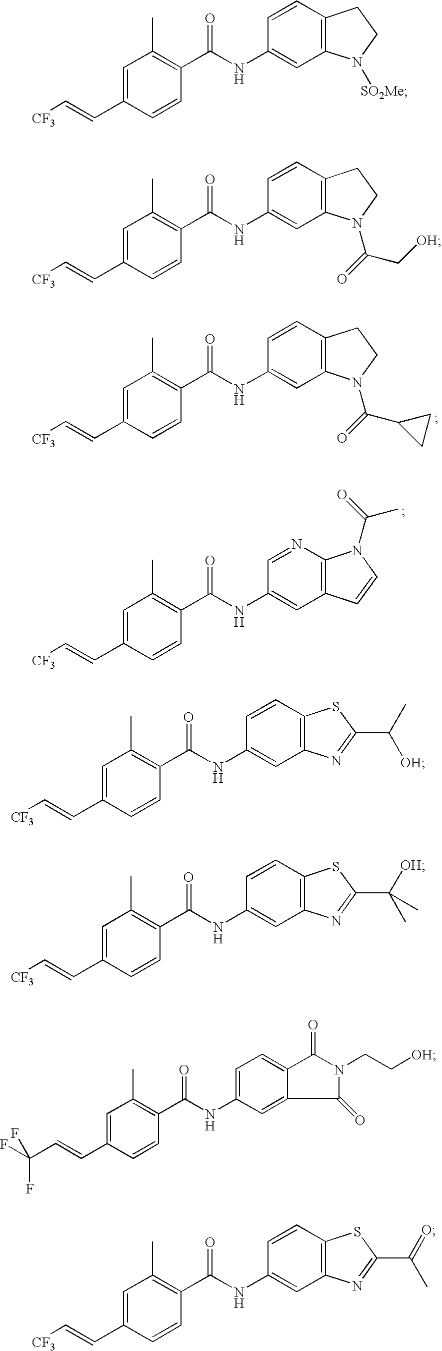 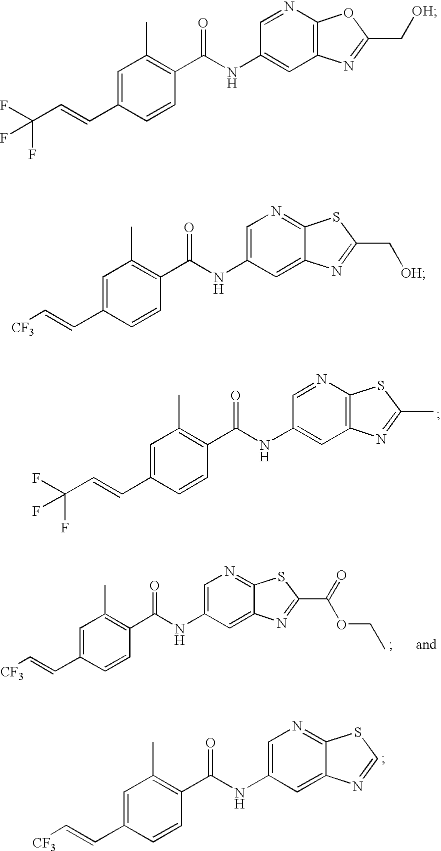
or a pharmaceutically acceptable salt, a solvate, a stereoisomer, an isotopic variant, or a tautomer thereof.

107. A compound selected from the group consisting of:
(E)-7-(2-methyl-4-(3,3,3-trifluoroprop-1-enyl)benzamido)quinoline-3-carboxylic acid;
(E)-N-(7-hydroxynaphthalen-1-yl)-2-methyl-4-(3,3,3-trifluoroprop-1-enyl)benzamide;
(E)-N-(3-(2-hydroxypropan-2-yl)quinolin-7-yl)-2-methyl-4-(3,3,3-trifluoroprop-1-enyl)benzamide;
(E)-N-(3-(1-hydroxyethyl)quinolin-7-yl)-2-methyl-4-(3,3,3-trifluoroprop-1-enyl)benzamide;
(E)-N-(3-((2-hydroxyethoxy)methyl)quinolin-7-yl)-2-methyl-4-(3,3,3-trifluoroprop-1-enyl)benzamide;
(E)-2-methyl-N-(8-oxo-5,6,7,8-tetrahydronaphthylen-2-yl)-4-(3,3,3-trifluoroprop-1-enyl)benzamide;
(E)-N-(8-hydroxy-5,6,7,8-tetrahydronaphthylen-2-yl)-2-methyl-4-(3,3,3-trifluoroprop-1-enyl)benzamide;
(E)-N-(3-(1,2-dihydroxyethyl)quinolin-7-yl)-2-methyl-4-(3,3,3-trifluoroprop-1-enyl)benzamide;
(E)-N-(7-hydroxy-5,6,7,8-tetrahydronaphthylen-1-yl)-2-methyl-4-(3,3,3-trifluoroprop-1-enyl)benzamide;
(E)-N-(7-(hydroxymethyl)-7,8-dihydro-5H-pyrano[4,3-b]pyridin-3-yl)-2-methyl-4-(3,3,3-trifluoroprop-1-enyl)benzamide;
(E)-N-(7-hydroxy-1,8-naphthyridin-2-yl)-2-methyl-4-(3,3,3-trifluoroprop-1-enyl)benzamide;
(E)-2-methyl-N-(5,6,7,8-tetrahydroquinolin-3-yl)-4-(3,3,3-trifluoroprop-1-enyl)benzamide;
(S,E)-N-(7-hydroxy-5,6,7,8-tetrahydronaphthylen-1-yl)-2-methyl-4-(3,3,3-trifluoroprop-1-enyl)benzamide;
(R,E)-N-(7-hydroxy-5,6,7,8-tetrahydronaphthylen-1-yl)-2-methyl-4-(3,3,3-trifluoroprop-1-enyl)benzamide;
(E)-N-(6-hydroxy-5,6,7,8-tetrahydroquinolin-3-yl)-2-methyl-4-(3,3,3-trifluoroprop-1-enyl)benzamide;
(E)-2-methyl-N-(quinolin-3-yl)-4-(3,3,3-trifluoro-2-methylprop-1-enyl)benzamide;
(E)-N-(7-(hydroxymethyl)-1,5-naphthyridin-3-yl)-2-methyl-4-(3,3,3-trifluoroprop-1-enyl)benzamide;
(E)-2-methyl-N-(1,5-naphthyridin-3-yl)-4-(3,3,3-trifluoroprop-1-enyl)benzamide;
(E)-2-methyl-N-(1,8-naphthyridin-2-yl)-4-(3,3,3-trifluoroprop-1-enyl)benzamide;
(E)-N-(7-(1-hydroxyethyl)-1,5-naphthyridin-3-yl)-2-methyl-4-(3,3,3-trifluoroprop-1-enyl)benzamide;
(E)-2-methyl-N-(1,8-naphthyridin-3-yl)-4-(3,3,3-trifluoroprop-1-enyl)benzamide;
(E)-N-(1-acetyl-1,2,3,4-tetrahydroquinolin-7-yl)-2-methyl-4-(3,3,3-trifluoroprop-1-enyl)benzamide;
(E)-N-(7-acetyl-1,5-naphthyridin-3-yl)-2-methyl-4-(3,3,3-trifluoroprop-1-enyl)benzamide;
(E)-2-methyl-N-(quinoxalin-6-yl)-4-(3,3,3-trifluoroprop-1-enyl)benzamide;
(E)-N-(7-(2-hydroxypropan-2-yl)-1,5-naphthyridin-3-yl)-2-methyl-4-(3,3,3-trifluoroprop-1-enyl)benzamide;
(E)-2-methyl-N-(1,7-naphthyridin-8-yl)-4-(3,3,3-trifluoroprop-1-enyl)benzamide;
(E)-2-methyl-N-(1-(methylsulfonyl)indolin-6-yl)-4-(3,3,3-trifluoroprop-1-enyl)benzamide;
(E)-N-(1-(cyclopropanecarbonyl)indolin-6-yl)-2-methyl-4-(3,3,3-trifluoroprop-1-enyl)benzamide;
(E)-N-(2-(1-hydroxyethyl)benzo[d]thiazol-5-yl)-2-methyl-4-(3,3,3-trifluoroprop-1-enyl)benzamide;
(E)-N-(2-(2-hydroxyethyl)-1,3-dioxoisoindolin-5-yl)-2-methyl-4-(3,3,3-trifluoroprop-1-enyl)benzamide;
(E)-2-methyl-N-(1-pivaloylindolin-6-yl)-4-(3,3,3-trifluoroprop-1-enyl)benzamide;
(E)-2-methyl-N-(1-propionylindolin-6-yl)-4-(3,3,3-trifluoroprop-1-enyl)benzamide;
(E)-N-(1-(2-hydroxyacetyl)indolin-6-yl)-2-methyl-4-(3,3,3-trifluoroprop-1-enyl)benzamide;
(E)-N-(1-acetyl-1H-pyrrolo[2,3-b]pyridin-5-yl)-2-methyl-4-(3,3,3-trifluoroprop-1-enyl)benzamide;
(E)-N-(2-(2-hydroxypropan-2-yl)benzo[d]thiazol-5-yl)-2-methyl-4-(3,3,3-trifluoroprop-1-enyl)benzamide;
(E)-N-(2-acetylbenzo[d]thiazol-5-yl)-2-methyl-4-(3,3,3-trifluoroprop-1-enyl)benzamide;
(E)-N-(2-(hydroxymethyl)oxazolo[5,4-b]pyridin-6-yl)-2-methyl-4-(3,3,3-trifluoroprop-1-enyl)benzamide;
(E)-2-methyl-N-(2-methylthiazolo[5,4-b]pyridin-6-yl)-4-(3,3,3-trifluoroprop-1-enyl)benzamide;
(E)-N-(2-(hydroxymethyl)thiazolo[5,4-b]pyridin-6-yl)-2-methyl-4-(3,3,3-trifluoroprop-1-enyl)benzamide;
(E)-ethyl 6-(2-methyl-4-(3,3,3-trifluoroprop-1-enyl)benzamido)thiazolo[5,4-b]pyridine-2-carboxylate; and
(E)-2-methyl-N-(thiazolo[5,4-b]pyridin-6-yl)-4-(3,3,3-trifluoroprop-1-enyl)benzamide,
or a pharmaceutically acceptable salt, a solvate, a stereoisomer, an isotopic variant, or a tautomer thereof.

108. A pharmaceutical composition comprising a pharmaceutically acceptable carrier, excipient or diluent and a pharmaceutically effective amount of a compound of any one of claims 85, 87, 88 and 104-107, or a pharmaceutically acceptable salt, a solvate, a stereoisomer, an isotopic variant, or a tautomer thereof.

109. The pharmaceutical composition of claim 108 wherein the carrier is a parenteral carrier, an oral carrier or a topical carrier.

110. A method for treating a disease or condition which comprises administering to a patient in need of such treatment a therapeutically effective amount of a compound of any one of claims 85, 87, 88 and 104-107, wherein the disease or condition is selected from the group consisting of acute cerebral ischemia, pain, post herpetic neuralgia, neuralgia, nerve injury, burn, headache, migraine, carpal tunnel syndrome, fibromyalgia, neuritis, sciatica, pelvic hypersensitivity, incontinence, micturition disorder, renal colic, cystitis, rheumatoid arthritis, osteoarthritis, multiple sclerosis, pulmonary disease, gastroesophageal reflux disease (GERD), dysphagia, ulcer, irritable bowel syndrome (IBS), inflammatory bowel disease (IBD), colitis, Crohn's disease, ischemia, cerebrovascular ischemia, emesis and obesity.

111. The method of claim 110, wherein the disease or condition is a pain selected from the group consisting of chronic pain, acute pain, nociceptive pain, neuropathic pain, inflammatory pain, rheumatoid arthritic pain, osteoarthritic pain, back pain, visceral pain, dental pain, pelvic pain, menstrual pain and post stroke pain.

112. The method of claim 110, wherein the disease or condition is a pulmonary disease selected from the group consisting of asthma, cough, chronic obstructive pulmonary disease (COPD) and broncho constriction.

113. The method of claim 110 wherein the compound is administered in combination with another pharmacologically active agent.
